(19) United States
(12) Patent Application Publication
(10) Pub. No.: US 2026/0049342 A1
(43) Pub. Date: Feb. 19, 2026

(54) METHODS AND COMPOSITIONS RELATING TO THE SYNTHESIS OF THE QS-7 MOLECULE

(71) Applicant: PLANT BIOSCIENCE LIMITED, Norwich, Norfolk (GB)

(72) Inventors: Anne OSBOURN, Norwich Norfolk (GB); James REED, Norwich Norfolk (GB); Anastasia ORME, Norwich Norfolk (GB); Laetitia MARTIN, Norwich Norfolk (GB)

(21) Appl. No.: 18/874,879

(22) PCT Filed: Dec. 23, 2022
(86) PCT No.: PCT/GB2022/053383

§ 371 Date: Dec. 13, 2024

(30) Foreign Application Priority Data
Jun. 29, 2022 (GB) 2209588.9

(51) Int. Cl. C12P 19/56; A61K 39/00; A61K 39/39; C12N 9/10; C12N 15/81; C12N 15/82; C12P 33/00

(52) U.S. Cl. CPC C12P 19/56; A61K 39/39; C12N 9/1051; C12N 15/81; C12N 15/82; C12P 33/00; A61K 2039/55572; A61K 2039/55577

(57) ABSTRACT

The present invention relates to a biosynthetic route to precursors of the QS-7 molecule, as well as routes to make the QS-7 molecule, enzymes involved, the products produced and uses of the product.

### METHODS AND COMPOSITIONS

The present invention relates to a biosynthetic route to precursors of the QS-7 molecule, as well as routes to make the QS-7 molecule, enzymes involved, the products produced and uses of the product.

### BACKGROUND

QS-7 is a natural saponin extract from the bark of the Chilean ‘soapbark’ tree, Quillaja saponaria. The QS-7 extract was originally identified as a purified fraction of a crude bark extract of Quillaja Saponaria Molina obtained by RP-HPLC purification (peak 7) (Kensil et al. 1991). The QS-7 molecule incorporates a central triterpene core backbone (quillaic acid), to which a branched trisaccharide is attached at the triterpene C-3 oxygen functionality, and a sugar chain is linked to the triterpene C-28 carboxylate group. QS-7 and QS-21 differ in the structure of the sugar chain at the C-28 position (see FIG. 1). The QS-21 structure displays a linear tetrasaccharide consisting of fucose, rhamnose, xylose and xylose (or apiose) as the terminal sugar. The QS-7 structure includes an identical linear tetrasaccharide, wherein the terminal sugar is apiose, and on which 2 additional sugars are incorporated (resulting in a branched hexasaccharide): (i) a rhamnose residue is incorporated at the C-3 position of the fucose residue of the linear tetrasaccharide and (ii) a glucose residue is incorporated at the C-3 position of the rhamnose residue of the linear tetrasaccharide. An additional difference between the two is that, instead of incorporating an acyl chain on the fucose residue (QS-21), QS-7 incorporates an acetyl moiety at the C-4 position of this sugar residue (see FIG. 1).

Saponins from Q. saponaria, including QS-7, have been known for many years to have potent immunostimulatory properties, capable of enhancing antibody production and specific T-cell responses. QS-7 shows similar potency to QS-21 and has reduced toxicity (Kensil et al. 1991). These properties have resulted in the development of Quillaja saponin-based adjuvants for vaccines. Of particular note, QS-7 is present in Novavax's ‘Matrix-M’ (as part of the saponin fraction named ‘Fraction A’-see e.g. WO 2017/161151), utilized in the NVX-CoV2373 COVID-19 vaccine.

The present invention describes methods to synthesise precursors of the QS-7 molecule, the QS-7 molecule per se as well as variants thereof, other than by purification from the native Q. saponaria plant. The present invention also describes the resulting products, which are useful as an adjuvant in vaccine formulations. The present invention also relates to enzymes involved in the methods, vectors, host cells and biological systems to produce the products.

### BRIEF DESCRIPTION OF THE INVENTION

The present invention relates to the formation of the branched acetylated hexasaccharide of the QS-7 molecule. In particular, it relates to the addition of (i) a glucose (G) residue at the C-3 position of the rhamnose residue of the linear tetrasaccharide sugar chain at the C-28 position of QA, (ii) a rhamnose (R) residue at the C-3 position of the D-fucose (F) of the linear tetrasaccharide sugar chain at the C-28 position of QA and (iii) an acetyl (Ac) moiety at the C-4 position of the D-fucose (F) of the linear tetrasaccharide sugar chain at the C-28 position of QA (see FIG. 1). The resulting QA derivatives are collectively referred to as QA-Tri(X/R)-F*-GR-Ac.

For simplicity, the linear tetrasaccharide sugar chain at the C-28 position of QA and its precursors (i.e. the sugar chain at the C-28 position with only two or three sugars) will be referred to as F* throughout the rest of the specification. Accordingly, in the sense of the present invention, “F*” is to be understood as FR, FRX, FRXA and/or FRXX (for further simplicity, FRXA and FRXX may also be designated as FRX(X/A)) (see the Abbreviation list herein).

The invention includes the biosynthetic preparation of QA-Tri(X/R)-F*-GR-Ac as well as precursors thereof. The invention also relates to the uses of QA-Tri(X/R)-F*-GR-Ac, such as the QS-7 molecule (QA-TriX-FRXA-GR-Ac) and precursors and variants thereof, e.g. as adjuvants.

### QA Synthesis

QA derives from the simple triterpene β-amyrin, which is synthesised through cyclisation of the universal linear precursor 2,3-oxidosqualene (OS) by an oxidosqualene cyclase (OSC). This biosynthesis is known in the art, such as in WO2019/122259, the content of which is incorporated by reference. This β-amyrin scaffold is further oxidised with a carboxylic acid, alcohol and aldehyde at the C-28, C-16a and C-23 positions, respectively, by a series of three cytochrome P450 monooxygenases, forming quillaic acid (QA). The OSC and C-28, C16a and C-23 oxidases are referred to herein as QsbAS (β-amyrin synthase), QsCYP716-C-28, QsCYP716-C-16a and QsCYP714-C-23 oxidases, respectively. A biosynthetic pathway for this is given in FIG. 2.

### C-3 Branched Trisaccharide Synthesis

The C-3 branched trisaccharide chain is initiated with a D-glucopyranuronic acid (D-GlcpA) residue attached with a β-linkage at the C-3 position of the QA backbone. The D-GlcpA residue has two sugars linked to it: a D-galactopyranose (D-Galp) residue attached with a β-1,2-linkage and either a D-xylopyranose (D-Xylp) moiety or an L-rhamnopyranose (L-Rhap) residue attached with a β-1,3-linkage or an α-1,3-linkage, respectively. A schematic for the glycosylation of QA to 3-O-{α-L-rhamnopyranosyl-(1->3)-[β-D-galactopyranosyl-(1->2)]-β-D-glucopyranosiduronic acid}-quillaic acid (QA-TriR) or 3-O-{β-D-xylopyranosyl-(1->3)-[β-D-galactopyranosyl-(1->2)]-β-D-glucopyranosiduronic acid}-quillaic acid (QA-TriX) is shown in FIG. 3.

Seven enzymes have been identified that have activity relevant to the production of the QA 3-O trisaccharide (QA-TriX or QA-TriR), such as in WO2020/260475, the content of which is incorporated by reference. These include two functionally-redundant glucuronosyltransferases, CSL1 and CsIG2, that can add the initial β-D-glucopyranuronic acid moiety at the C-3 position of quillaic acid; a galactosyltransferase, Qs-3-O-GalT, that adds the β-D-galactopyranose residue to the C-2 position of the β-D-glucopyranuronic acid; a xylosyltransferase, Qs_0283870, that adds the β-D-xylopyranose residue at the C-3 position of the β-D-glucopyranuronic acid; two rhamnosyltransferases, DN20529_c0_g2_i8 and Qs_0283850, that add an α-L-rhamnopyranose residue at the C-3 position of the β-D-glucopyranuronic acid; and a bifunctional enzyme, Qs-3-O-RhaT/XyIT that can add either a β-D-xylopyranose residue or a α-L-rhamnopyranose residue to the C-3 position of the β-D-glucopyranuronic acid (see FIG. 3).

For simplicity, throughout the application, a QA derivative including the branched trisaccharide at position C-3 may be designated as “QA-TriX”, “QA-TriR” or “QA-Tri(X/R)” (see the Abbreviation list herein).

### C-28 Linear Tetrasaccharide Synthesis

F* is initiated by attaching a D-fucose residue with a β-linkage at the C-28 position of the QA backbone. This step is followed by attaching an L-rhamnose residue with an α-linkage to the C-2 position of the fucose residue, then attaching a D-xylose residue with a β-linkage to the C-4 position of the rhamnose residue. Finally, a D-xylose residue or a D-apiose residue is attached with a β-linkage to the C-3 position of the xylose residue.

Ten enzymes have been identified that have activity relevant to the production of F*, such as reported in PCT/EP2021/087323. These include Qs-28-O-FucT (SEQ ID NO 2), which transfers a D-fucose residue with a β-linkage to the C-28 position of the QA backbone; Qs-28-O-RhaT (SEQ ID NO 4) which transfers an L-rhamnose residue to a D-fucose moiety; Qs-28-O-XyIT3 (SEQ ID NO 6) which transfers a D-xylose moiety to a L-rhamnose residue; Qs-28-O-XyIT4 (SEQ ID NO 8) which attaches a β-D-xylose residue to a β-D-xylose residue; Qs-28-O-ApiT4 (SEQ ID NO 10) which attaches a β-D-apiose residue to a β-D-xylose residue (FIG. 4). An oxidoreductase enzyme QsFucSyn (SEQ ID No. 12), and QsFucSyn-Like enzymes, such as QsFSL-1 (SEQ ID No. 48), QsFSL-2 (SEQ ID No 50) or SoFSL-1 (SEQ ID No 52) which may increase the production of UDP-D-fucose and/or reduce the 4-keto group of 4-keto-6-deoxy-glucose after it has been added to the QA backbone have also been identified that have activity relevant to the production of F*. A UDP-apiose/UDP-xylose synthase enzyme QsAXS1 (SEQ ID NO 14) which enhances the activity of an apiosyltransferase by increasing the availability of the UDP-α-D-apiose has also been identified previously.

### C-28 Acetylated Branched Hexasaccharide Synthesis

The present invention describes, for the first time, the biosynthetic route for the addition of a glucose residue at the C-3 position of the rhamnose residue of F*, a rhamnose residue at the C-3 position of the D-fucose of F* and an acetyl moiety at the C-4 position of the D-fucose residue of F*, to form the QS-7 molecule and precursors and variants thereof. As a result, the QS-7 molecule comprises a branched hexasaccharide chain at the C-28 position, with an acetyl moiety at the C-4 position of the D-fucose residue of F* (see FIG. 1).

Accordingly, the present invention provides methods for making QS-7, and precursors and variants thereof. Also provided are enzymes used in the methods, polynucleotides encoding the enzymes, vectors comprising the polynucleotides, host cells transformed with the vectors and uses of the QS-7 molecule, precursors and variants thereof, as an adjuvant.

### DESCRIPTION OF THE FIGURES

FIG. 1 shows the structure of QS-7 and QS-21. Both share a backbone formed from the triterpene quillaic acid (QA). The C-3 position of QA features a branched trisaccharide consisting of β-D-glucopyranuronic acid (D-GlcpA), β-D-galactopyranose (D-Galp) and a β-D-xylopyranose (D-xylp). The C-28 position features a linear sugar chain consisting of 3-D-fucopyranose (D-fucp), α-L-rhamnopyranose, 3-D-xylopyranose and a terminal β-D-apiofuranose (D-apif) (for QS-21 and QS-7) or β-D-xylopyranose (for QS-21). In QS-7, a glucose residue is incorporated at the C-3 position of the rhamnose residue of the linear sugar chain at the C-28 position, and a rhamnose residue is incorporated at the C-3 position of the D-fucose of the linear sugar chain at the C-28 position. The D-fucose also features an acetyl moiety at the C-4 position.

FIG. 2 shows the production of quillaic acid (QA) from 2,3-oxidosqualene via β-amyrin. The pathway from β-amyrin requires oxidation at three (C-28, C-23 and C-16a) positions. These oxidation steps are shown in a linear fashion for simplicity; however, they could occur in any order.

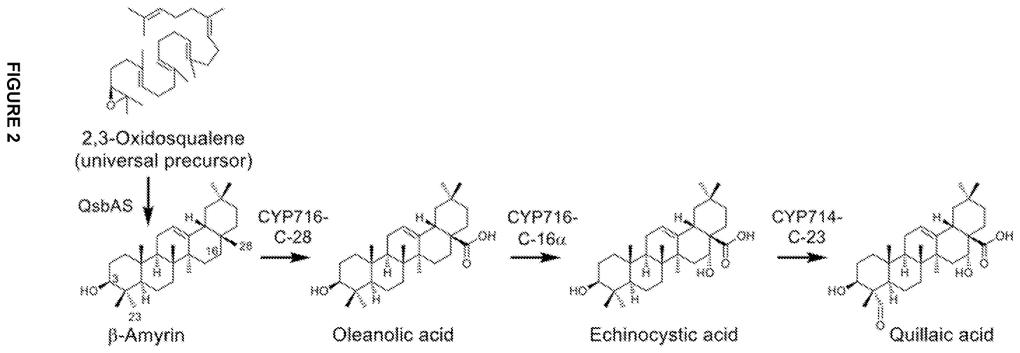

FIG. 3 shows the production of QA-TriR or QA-Trix from quillaic acid (QA). A β-D-glucopyranuronic acid (β-D-GlcpA) is added, by either of the glucuronosyltransferases QsCLS1 or QsCsIG2, to the C-3 position of QA to form QA-Mono. The galactosyltransferase Qs-3-O-GalT adds a β-D-galactopyranose (β-D-Galp) to the C-2 position of the glucopyranuronic acid to form QA-Di. An α-L-rhamnopyranose (α-L-Rhap) can be attached to the C-3 position of the glucopyranuronic acid by the single-function rhamnosyltransferases, DN20529_c0_g2_i8 or Qs_0283850, or by the dual-function Qs-3-O-RhaT/XyIT, to form QA-TriR. Alternatively, a 3-D-xylopyranose (β-D-Xylp) can be attached to the C-3 position of the glucopyranuronic acid to form QA-TriX, either by the single-function xylosyltransferase Qs_0283870 or by the dual-function Qs-3-O-RhaT/XyIT.

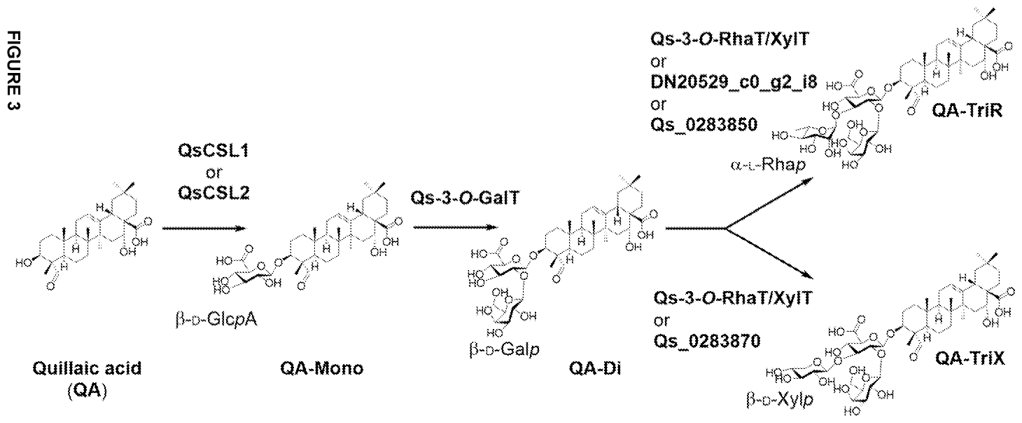

FIG. 4 shows the production of the QA-Tri(X/R)-FRX(X/A) from QA-Tri(X/R). The chain is initiated with a β-D-fucopyranose (β-D-Fucp) attached to the C-28 of QA via an ester linkage, followed by the attachment of an α-1,2-L-rhamnopyranose (α-L-Rhap) and the attachment of a β-1,4-D-xylopyranose (β-D-Xylp). The terminal sugar of the chain can be either β-1,3-D-xylopyranose (β-D-Xylp) or 3-1,3-D-apiofuranose (β-D-Apif). For simplicity, the resulting QA derivative may be designated as QA-Tri(X/R)-FRX(X/A).

FIG. 5 shows the production of QA-TriX-FRXA-G in Nicotiana benthamiana. The gene set for production of the QA-TriX-FRXA product (tHMGR/QsbAS/CYP716-C-28/CYP716-C-16a/CYP714-C23/Csl1/C3-GalT/C3-XyIT/QsFucSyn/C-28-FucT/C28-RhaT/C28-XylT3/C28-ApiT4) was transiently expressed in N. benthamiana along with UGT-BI. LC-MS analysis of leaf extracts revealed the presence of a product with a mass consistent with the addition of a hexose residue, anticipated to be glucose. The new product was designated as QA-TriX-FRXA glucoside (QA-TriX-FRXA-G).

FIG. 6 shows the production of QA-TriX-FRXA-Ac in N. benthamiana. The gene set for production of the QA-TriX-FRXA product (tHMGR/QsbAS/CYP716-C-28/CYP716-C-16a/CYP714-C23/Csl1/C3-GalT/C3-XyIT/QsFucSyn/C-28-FucT/C28-RhaT/C28-XyIT3/C28-ApiT4) was transiently expressed in N. benthamiana along with ACT-19′. LC-MS analysis of leaf extracts revealed the presence of a product with a mass consistent with the addition of an acetyl group. The new product was designated as QA-TriX-FRXA acetyl (QA-TriX-FRXA-Ac).

FIG. 7 shows the production of QA-TriX-FRXA-R-Ac in N. benthamiana. The gene set for production of the QA-TriX-FRXA-Ac product (tHMGR/QsbAS/CYP716-C-28/CYP716-C-16a/CYP714-C23/Csl1/C3-GalT/C3-XyIT/QsFucSyn/C-28-FucT/C28-RhaT/C28-XyIT3/C28-ApiT4/ACT-19′) was transiently expressed in N. benthamiana along with UGT-0023500. LC-MS analysis of leaf extracts revealed the presence of a product with a mass consistent with the addition of a deoxyhexose, anticipated to be rhamnose. The new product was designated as QA-TriX-FRXA-Ac rhamnoside (QA-TriX-FRXA-R-Ac).

FIG. 8 shows the production of QS-7 (QA-TriX-FRXA-GR-Ac) in N. benthamiana. The gene set for production of the QA-TriX-FRXA product (tHMGR/QsbAS/CYP716-C-28/CYP716-C-16a/CYP714-C23/Cs/1/C3-GalT/C3-XyIT/C-28-FucT/C28-RhaT/C28-XylT3/C28-ApiT4) were transiently expressed in N. benthamiana along with the GlcT, RhaT and AcetylT genes needed to convert this precursor to QS-7. LC-MS analysis of leaf extracts expressing all QS-7 genes revealed the presence of a product with the same retention time and mass as an authentic QS-7 standard. This peak was absent in the control samples where one of the GlcT, RhaT or AcetylT was absent.

FIG. 9 shows ¹H, ¹³C NMR spectral data for quillaic acid (QA) triterpene core of semi-purified QS-7 (QA-TriX-FRXA-GR-Ac) in MeOH-d₄ (600, 150 MHZ).

FIG. 10 shows ¹H, ¹³C NMR spectral data for C₃, C₂₈ oligosaccharides of semi-purified QS-7 (QA-TriX-FRXA-GR-Ac) in MeOH-d₄ (600, 150 MHZ).

### DETAILED DESCRIPTION OF THE INVENTION

A first aspect of the invention is a method of making QA-Tri(X/R)-F*-GR-Ac, wherein the acetyl (Ac) moiety is attached to the C-4 position of the D-fucose of F*, the rhamnose (R) residue is attached to the C-3 position of the D-fucose of F* and the glucose (G) residue is attached to the C-3 position of the rhamnose residue of F*. The method comprises combining QA-Tri(X/R)-F* with
i. the enzyme quillaic acid 28-O-fucoside [1,2]-rhamnoside [1,3] glucosyltransferase (QS-7-GlcT) having the amino acid sequence of SEQ ID NO 56, or an enzyme having an amino acid sequence with at least 70% sequence identity to SEQ ID NO 56;
ii. one or more enzymes selected from the enzyme quillaic acid 28-O-fucoside [1,4] acetyltransferase (QS-7-AcetylT) having the amino acid sequence of SEQ ID NO 60 or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme (3S,5S,6S)-3,5-dihydroxy-6-methyloctanoyl-CoA transferase 9 (DMOT9) having the amino acid sequence of SEQ ID NO 64 or an enzyme having an amino acid sequence with at least 25% sequence identity to SEQ ID NO 64, and
iii. the enzyme quillaic acid 28-O-fucoside [1,3] rhamnosyltransferase (QS-7-RhaT) having the amino acid sequence of SEQ ID NO 58, or an enzyme having an amino acid sequence with at least 70% sequence identity to SEQ ID NO 58,
   to form QA-Tri(X/R)-F*-GR-Ac. In this aspect of the invention, QA is quillaic acid; Tri(X/R) is a branched trisaccharide at position C-3 of the QA backbone which terminates in either a xylose residue (X) or a rhamnose residue (R);
   F* is a disaccharide of a β-D-fucose residue (F) and R, also referred to as FR; a trisaccharide of F, R and X, also referred to as FRX; a tetrasaccharide of F, R, X and X, also referred to as FRXX or a tetrasaccharide of F, R, X and a β-D-apiose residue (A), also referred to as FRXX;
   G is a glucose residue; and
   Ac is an acetyl moiety.

A second aspect of the invention is a method of making a biosynthetic QA-Tri(X/R)-F*-GR-Ac in a host. The method comprises the steps of:
a) expressing genes required for the biosynthesis of QA-TriR-F* and/or QA-TriX-F*, and
b) introducing a polynucleotide encoding:
   i. the enzyme QS-7-GlcT having the amino acid sequence of SEQ ID NO 56, or an enzyme having an amino acid sequence with at least 70% sequence identity to SEQ ID NO 56;
   ii. one or more enzymes selected from the enzyme QS-7-AcetylT having the amino acid sequence of SEQ ID NO 60, or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, and
   iii. the enzyme QS-7-RhaT having the amino acid sequence of SEQ ID NO 58, or an enzyme having an amino acid sequence with at least 70% sequence identity to SEQ ID NO 58,
into the host. In this aspect of the invention, QA is quillaic acid; Tri(X/R) is a branched trisaccharide at position C-3 of the QA backbone which terminates in either a xylose residue (X) or a rhamnose residue (R);
   F* is a disaccharide of a β-D-fucose residue (F) and R, also referred to as FR; a trisaccharide of F, R and X, also referred to as FRX; a tetrasaccharide of F, R, X and X, also referred to as FRXX; or a tetrasaccharide of F, R, X and a β-D-apiose residue (A), also referred to as FRXA;
   G is a glucose residue; and
   Ac is an acetyl moiety.

A third aspect of the invention is a method of making a biosynthetic QA-TriX-F*-GR-Ac in a host. The method comprises the steps of:
a) expressing genes required for the biosynthesis of QA-TriX-F*, and
b) introducing a polynucleotide encoding:
   i. the enzyme QS-7-GlcT having the amino acid sequence of SEQ ID NO 56, or an enzyme having an amino acid sequence with at least 70% sequence identity to SEQ ID NO 56;
   ii. one or more enzymes selected from the enzyme QS-7-AcetylT having the amino acid sequence of SEQ ID NO 60 or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, and
   iii. the enzyme QS-7-RhaT having the amino acid sequence of SEQ ID NO 58, or an enzyme having an amino acid sequence with at least 70% sequence identity to SEQ ID NO 58,
      into the host. In this aspect of this invention, QA-TriX is 3-O-{β-D-xylopyranosyl-(1->3)-[β-D-galactopyranosyl-(1->2)]-β-D-glucopyranosiduronic acid}-quillaic acid;
      F* is a disaccharide of a β-D-fucose residue (F) and a rhamnose residue (R), also referred to as FR; a trisaccharide of F, R and a xylose residue (X), also referred to as FRX; a tetrasaccharide of F, R, X and X, also referred to as FRXX or a tetrasaccharide of F, R, X and a β-D-apiose residue (A), also referred to as FRXA;
      G is a glucose residue; and
      Ac is an acetyl moiety.

In the third aspect of the invention, F* may be FRXA. Accordingly, the invention includes a method of making a biosynthetic QA-TriX-FRXA-GR-Ac in a host. The method comprises the steps of:
a) expressing genes required for the biosynthesis of QA-TriX-FRXA, and
b) introducing a polynucleotide encoding:
   i. the enzyme QS-7-GlcT having the amino acid sequence of SEQ ID NO 56, or an enzyme having an amino acid sequence with at least 70% sequence identity to SEQ ID NO 56;
   ii. one or more enzymes selected from the enzyme QS-7-AcetylT having the amino acid sequence of SEQ ID NO 60 or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, and
   iii. the enzyme QS-7-RhaT having the amino acid sequence of SEQ ID NO 58, or an enzyme having an amino acid sequence with at least 70% sequence identity to SEQ ID NO 58,
      into the host.

A fourth aspect of the invention is a method of making a biosynthetic QA-TriR-F*-GR-Ac in a host. The method comprises the steps of:
a) expressing genes required for the biosynthesis of QA-TriR-F*, and
b) introducing a polynucleotide encoding:
   i. the enzyme QS-7-GlcT having the amino acid sequence of SEQ ID NO 56, or an enzyme having an amino acid sequence with at least 70% sequence identity to SEQ ID NO 56;
   ii. one or more enzymes selected from the enzyme QS-7-AcetylT having the amino acid sequence of SEQ ID NO 60, or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, and
   iii. the enzyme QS-7-RhaT having the amino acid sequence of SEQ ID NO 58, or an enzyme having an amino acid sequence with at least 70% sequence identity to SEQ ID NO 58,
      into the host. In this aspect of the invention, QA-TriR is 3-O-{α-L-rhamnopyranosyl-(1->3)-[3-D-galactopyranosyl-(1->2)]-β-D-glucopyranosiduronic acid}-quillaic acid;
      F* is a disaccharide of a β-D-fucose residue (F) and a rhamnose residue (R), also referred to as FR; a trisaccharide of F, R and a xylose residue (X), also referred to as FRX; a tetrasaccharide of F, R, X and X, also referred to as FRXX; a tetrasaccharide of F, R, X and a β-D-apiose residue (A), also referred to as FRXA;
      G is a glucose residue; and
      Ac is an acetyl moiety.

In the first aspect of the invention steps (i), (ii) and (iii) may occur in that order. QA-Tri(X/R)-F* is first combined with the enzyme QS-7-GlcT having the amino acid sequence of SEQ ID NO 56, or an enzyme having an amino acid sequence with at least 70% sequence identity to SEQ ID NO 56, to form QA-Tri(X/R)-F*-G. In particular, F* may be FRX. F* may also be FRX(X/A). Then QA-Tri(X/R)-F*-G is combined with one or more enzymes selected from the enzyme QS-7-AcetylT having the amino acid sequence of SEQ ID NO 60 or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, to form QA-Tri(X/R)-F*-G-Ac. In particular, F* may be FRX. F* may also be FRX(X/A). Finally, QA-Tri(X/R)-F*-G-Ac is combined with the enzyme QS-7-RhaT having the amino acid sequence of SEQ ID NO 58, or an enzyme having an amino acid sequence with at least 70% sequence identity to SEQ ID NO 58, to form QA-Tri(X/R)-F*-GR-Ac. In particular, F* may be FRX. F* may also be FRX(X/A). In steps (i), (ii) and (iii), F* may be FRX.

The steps of this aspect of the invention may also occur in the order (ii), (iii) then (i). QA-Tri(X/R)-F* is first combined with one or more enzymes selected from the enzyme QS-7-AcetylT having the amino acid sequence of SEQ ID NO 60 or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, to form QA-Tri(X/R)-F*-Ac. In particular, F* may be FR. F* may also be FRX. F* may also be FRX(X/A). Then QA-Tri(X/R)-F*-Ac is combined with the enzyme QS-7-RhaT having the amino acid sequence of SEQ ID NO 58, or an enzyme having an amino acid sequence with at least 70% sequence identity to SEQ ID NO 58, to form QA-Tri(X/R)-F*-R-Ac. In particular, F* may be FR. F* may also be FRX. F* may also be FRX(X/A). Finally, QA-Tri(X/R)-F*-R-Ac is combined with the enzyme QS-7-GlcT having the amino acid sequence of SEQ ID NO 56, or an enzyme having an amino acid sequence with at least 70% sequence identity to SEQ ID NO 56, to form QA-Tri(X/R)-F*-GR-Ac. F* may be FRX. F* may also be FRX(X/A). In steps (i) and (ii) F* may be FR and in step (iii), F* may be FRX.

The steps of this aspect of the invention may also occur in the order (ii), (i) then (iii). QA-Tri(X/R)-F* is first combined with one or more enzymes selected from the enzyme QS-7-AcetylT having the amino acid sequence of SEQ ID NO 60 or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, to form QA-Tri(X/R)-F*-Ac. F* may be FR. F* may also be FRX. F* may also be FRX(X/A). Then QA-Tri(X/R)-F*-Ac is combined with the enzyme QS-7-GlcT having the amino acid sequence of SEQ ID NO 56, or an enzyme having an amino acid sequence with at least 70% sequence identity to SEQ ID NO 56, to form QA-Tri(X/R)-F*-G-Ac. F* may be FRX. F* may also be FRX(X/A). Finally, QA-Tri(X/R)-F*-G-Ac is combined with the enzyme QS-7-RhaT having the amino acid sequence of SEQ ID NO 58, or an enzyme having an amino acid sequence with at least 70% sequence identity to SEQ ID NO 58, to form QA-Tri(X/R)-F*-GR-Ac. F* may be FRX. F* may also be FRX(X/A). In step (i) F* may be FR and in steps (ii) and (iii), F* may be FRX.

In the first aspect of the invention Tri(X/R) may be TriX and F* may be FRXA. Accordingly, the invention includes a method of making QA-TriX-FRXA-GR-Ac, wherein the acetyl (Ac) moiety is attached to the C-4 position of the D-fucose of FRXA, the rhamnose (R) residue is attached to the C-3 position of the D-fucose of FRXA and the glucose (G) residue is attached to the C-3 position of the rhamnose residue of FRXA, wherein the method comprises combining QA-TriX-FRXA with
i. the enzyme QS-7-GlcT having the amino acid sequence of SEQ ID NO 56, or an enzyme having an amino acid sequence with at least 70% sequence identity to SEQ ID NO 56;
ii. one or more enzymes selected from the enzyme QS-7-AcetylT having the amino acid sequence of SEQ ID NO 60 or an enzyme having an amino acid sequence with at least 70% sequence identity to SEQ ID NO 60; the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64; and
iii. the enzyme QS-7-RhaT having the amino acid sequence of SEQ ID NO 58, or an enzyme having an amino acid sequence with at least 70% sequence identity to SEQ ID NO 58,
to form QA-TriX-FRXA-GR-Ac.

The steps of the first aspect of the invention when Tri(X/R) is TriX and F* is FRXA may occur in the order (i), (ii) then (iii). The steps may also occur in the order (ii), (iii) then (i). The steps may also occur in the order (ii), (i) then (iii).

As defined herein, F* may be FR, FRX, FRXA and/or FRXX. The sugars of the F* chain are added at the C-28 position of QA-Tri(X/R). When F* is a mixture comprising FRXX and FRXA, the ratio of FRXX to FRXA may vary. The ratio of FRXX to FRXA within the mixture may vary in percentage. Suitably, the mixture comprises from 10 to 90% of FRXX, such as 10%, 20%, 30%, 40%, 50%, 60%, 70%, 80%, or 90% and from 90 to 10% of FRXA, such as 90%, 80%, 70%, 60%, 50%, 40%, 30%, 20%, or 10%. Preferably, the mixture comprises 60% of FRXX and 40% of FRXA, or 50% of each.

In embodiments where an acetyl (Ac) moiety is attached to the C-4 position of the D-fucose of F*, F* is FR, FRX, FRXA and/or FRXX. In particular F* may be FRXA. Similarly, in embodiments where QA-Tri(X/R)-F* is combined with one or more enzymes selected from the enzyme QS-7-AcetylT having the amino acid sequence of SEQ ID NO 60 or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, to form QA-Tri(X/R)-F*-Ac, F* is FR, FRX, FRXA and/or FRXX. In particular F* may be FRXA. In embodiments where QA-Tri(X/R)-F*-G is combined with one or more enzymes selected from the enzyme QS-7-AcetylT having the amino acid sequence of SEQ ID NO 60 or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, to form QA-Tri(X/R)-F*-G-Ac, F* is FRX, FRXA and/or FRXX, in particular F* may be FRXA

In embodiments where a rhamnose (R) residue is attached to the C-3 position of the D-fucose of F*, F* is FR, FRX, FRXA and/or FRXX and the acetyl moiety must be attached to the C-4 position of the D-fucose of F*. In embodiments where QA-Tri(X/R)-F*-Ac is combined with the enzyme QS-7-RhaT having the amino acid sequence of SEQ ID NO 58, or an enzyme having an amino acid sequence with at least 70% sequence identity to SEQ ID NO 58, to form QA-Tri(X/R)-F*-R-Ac, F* is FR, FRX, FRXA and/or FRXX. In particular F* may be FRXA. In embodiments where QA-Tri(X/R)-F*-G-Ac is combined with the enzyme QS-7-RhaT having the amino acid sequence of SEQ ID NO 58, or an enzyme having an amino acid sequence with at least 70% sequence identity to SEQ ID NO 58, to form QA-Tri(X/R)-F*-GR-Ac, F* is FRX, FRXA and/or FRXX. In particular F* may be FRXA.

In embodiments where a glucose (G) residue is attached to the C-3 position of the rhamnose moiety of F*, F* is FRX, FRXA and/or FRXX. In particular F* may be FRXA. In embodiments where QA-Tri(X/R)-F* is combined with the enzyme QS-7-GlcT having the amino acid sequence of SEQ ID NO 56, or an enzyme having an amino acid sequence with at least 70% sequence identity to SEQ ID NO 56, to form QA-Tri(X/R)-F*-G, F* is FRX, FRXA and/or FRXX. In particular F* may be FRXA. When QA-Tri(X/R)-F*-Ac is combined with the enzyme QS-7-GlcT having the amino acid sequence of SEQ ID NO 56, or an enzyme having an amino acid sequence with at least 70% sequence identity to SEQ ID NO 56, to form QA-Tri(X/R)-F*-G-Ac, F* is FRX, FRXA and/or FRXX. In particular F* may be FRXA. In embodiments where QA-Tri(X/R)-F*-R-Ac is combined with the enzyme QS-7-GlcT having the amino acid sequence of SEQ ID NO 56, or an enzyme having an amino acid sequence with at least 70% sequence identity to SEQ ID NO 56, to form QA-Tri(X/R)-F*-GR-Ac, F* is FRX, FRXA and/or FRXX. In particular F* may be FRXA.

As defined herein, the QA-Tri(X/R)-F* derivative may be QA-Tri(X/R)-FR, QA-Tri(X/R)-FRX, QA-Tri(X/R)-FRXX, QA-Tri(X/R)-FRXA, QA-TriR-FR, QA-TriR-FRX, QA-TriR-FRXX, QA-TriR-FRXA, QA-TriX-FR, QA-TriX-FRX, QA-TriX-FRXX or QA-TriX-FRXA.

When QA-Tri(X/R) is a mixture comprising QA-TriX and QA-TriR, the ratio of QA-TriX to QA-TriR may vary. The ratio of QA-Trix to QA-TriR within the mixture may vary in percentage. Suitably, the mixture comprises from 10 to 90% of QA-TriX, such as 10%, 20%, 30%, 40%, 50%, 60%, 70%, 80%, or 90% and from 90 to 10% of QA-TriR, such as 90%, 80%, 70%, 60%, 50%, 40%, 30%, 20%, or 10%.

### C-28 Acetylated Branched Hexasaccharide Synthesis

The QS-7 molecule incorporates a glucose residue at the C-3 position of the rhamnose residue of F*, a rhamnose residue at the C-3 position of the D-fucose of F* and an acetyl moiety at the C-4 position of the D-fucose of F*. The inventors identified enzymes which allowed the glucose residue, the rhamnose residue and the acetyl moiety to be added to the core molecule in the required positions, in vitro and in vivo. By “core molecule”, it is meant one or more of the following QA derivatives: QA-TriX-FR, QA-TriX-FRX, QA-TriX-FRXX, QA-TriX-FRXA, QA-TriR-FR, QA-TriR-FRX, QA-TriR-FRXA, QA-TriR-FRXX, QA-Tri(X/R)-FR, QA-Tri(X/R)-FRX, QA-Tri(X/R)-FRXA, QA-Tri(X/R)-FRXX.

In the methods of the invention the steps of adding the glucose and rhamnose residues and the acetyl moiety can be performed in a specific order or in any order or simultaneously.

In the methods of the invention the transfer of an acyl moiety to the C-4 position of the D-fucose of F* (e.g. the transfer of an acyl moiety to QA-Tri(X/R)-F* to form QA-Tri(X/R)-F*-Ac) may be carried out by the enzyme QS-7-AcetylT (SEQ ID NO 60), or an enzyme having at least 70% sequence identity to the sequence for QS-7-AcetylT, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64. These enzymes are capable of transferring an acyl unit to the C-4 position of the D-fucose of the F* chain. The function of the enzyme can be determined for example as described in Example 2.

The function of QS-7-AcetylT, SOAP10 or DMOT9 may be determined by expressing in a heterologous host such as N. benthamiana or yeast the enzymes necessary to generate QA-Tri(X/R)-F* and the QS-7-AcetylT, SOAP10 or DMOT9 candidate. The presence of the expected product may be assessed by LC-MS analysis, eventually complemented by NMR analysis. Alternatively, in vitro testing may be preferred in which QA-Tri(X/R)-F* is either purified from a plant extract or generated in vitro in an assay containing quillaic acid and the glycosyl transferases necessary to generate QA-Tri(X/R)-F*, or β-amyrin and the enzymes necessary to produce QA-Tri(X/R)-F*. The activity of the candidate QS-7-AcetylT, SOAP10 or DMOT9 is then tested in vitro on the QA-Tri(X/R)-F* substrate and the product formation is determined by LC-MS analysis.

Throughout this description when referring to an enzyme (SEQ ID NO), this is referring to an enzyme according to that SEQ ID NO. For example, “QS-7-AcetylT (SEQ ID NO 60)” means the enzyme QS-7-AcetylT according to SEQ ID NO 60.

Enzymes for use in the present invention may include one or more conservative amino acid substitutions, such that the resulting enzyme has a similar amino acid sequence and/or retains the same function. The skilled person is aware that various amino acids have similar biochemical properties and thus are “conservative”. One or more such amino acids of a protein (e.g. enzyme), polypeptide or peptide can often be substituted by one or more other such amino acids without eliminating a desired activity of that protein, polypeptide or peptide.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). It should be appreciated that amino acid substitutions within the scope of the present invention can be made using naturally occurring or non-naturally occurring amino acids. For example, the methyl group on an alanine may be replaced with an ethyl group, and/or minor changes may be made to the peptide backbone. Whether or not natural or synthetic amino acids are used, it is preferred that only L-amino acids are present. Substitutions of this nature are often referred to as “conservative” amino acid substitutions.

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exists a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score.

The percentage of identity of two amino acid sequences or of two polynucleotide sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percentage of identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to nucleic acid molecules. BLAST protein searches can be performed with the XBLAST program, score=50, wordlength=3 to obtain amino acid sequences homologous to protein molecules for use in the invention. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

Mutations, including conservation substitutions, insertions and deletions, may be introduced into the sequences using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning-A Laboratory Manual (3^{rd} Ed.) CSHL Press. Further information on ligation independent cloning (LIC) procedures can be found in Rashtchian, (1995) Curr Opin Biotechnol 6(1): 30-6.

In the methods of the invention, the transfer of an acyl moiety to the C-4 position of the D-fucose of F* may be carried out by the enzyme QS-7-AcetylT (SEQ ID NO 60), or an enzyme having at least 70% sequence identity to the sequence for QS-7-AcetylT (SEQ ID No 60). The amino acid sequence of the QS-7-AcetylT enzyme may have at least 70%, 75%, 80%, 85%, 90%, 95%, 96%, 97%, 98% or 99% identity to SEQ ID NO 56. Accordingly, in some embodiments, the QS-7-AcetylT has at least 70%, 75%, 80%, 85%, 90%, 95%, 96%, 97%, 98% or 99% identity to SEQ ID NO 60, suitably at least 90%, more suitably at least 95%. In respect of the enzymes defined here in terms of sequence identity, they typically retain the function of transferring an acyl unit to the C-4 position of the D-fucose of F*.

The transfer of an acyl moiety to the C-4 position of the D-fucose of F* may also be carried out by the enzyme SOAP10 (SEQ ID NO 62), or an enzyme having at least 70% sequence identity to the sequence for SOAP10 (SEQ ID NO 62). The amino acid sequence of the SOAP10 enzyme may have at least 70%, 75%, 80%, 85%, 90%, 95%, 96%, 97%, 98% or 99% identity to SEQ ID NO 62. Accordingly, in some embodiments, the SOAP10 has at least 70%, 75%, 80%, 85%, 90%, 95%, 96%, 97%, 98% or 99% identity to SEQ ID NO 62, suitably at least 90%, more suitably at least 95%. In respect of the enzymes defined here in terms of sequence identity, they typically retain the function of transferring an acyl unit to the C-4 position of the D-fucose F*.

The transfer of an acyl moiety to the C-4 position of the D-fucose of F* may also be carried out by the enzyme DMOT9 (SEQ ID NO 64), or an enzyme having at least 25% sequence identity to the sequence for DMOT9 (SEQ ID NO 64). The amino acid sequence of the DMOT9 enzyme may have at least 25%, 30%, 35%, 40%, 45%, 50%, 55%, 60%, 65%, 70%, 75%, 80%, 85%, 90%, 95%, 96%, 97%, 98% or 99% identity to SEQ ID NO 64. Accordingly, in some embodiments, the DMOT9 enzyme has at least 25%, 30%, 35%, 40%, 45%, 50%, 55%, 60%, 65%, 70%, 75%, 80%, 85%, 90%, 95%, 96%, 97%, 98% or 99% identity to SEQ ID NO 64, suitably at least 90%, more suitably at least 95%. In respect of the enzymes defined here in terms of sequence identity, they typically retain the function of transferring an acyl unit to the C-4 position of the D-fucose of F*.

The percentage sequence identities discussed in this application are the percentage sequence identities across the full length of the sequences identified by the SEQ. ID NOs. This may include shortened sequences which have the same sequence identity measured across the length of the shortened sequence. The shortened sequences may have the same homology of the percentage sequence identity of the SEQ ID NO regardless of the length of the shortened sequence. The shortened sequence may be at least half the length of the full-length sequence, preferably at least three quarters of the length of the full sequence.

In the methods of the invention the transfer of a rhamnose residue to the C-3 position of the D-fucose of F* may be carried out by the enzyme QS-7-RhaT (SEQ ID NO 58), or an enzyme having at least 70% sequence identity to the sequence for QS-7-RhaT. The enzyme is capable of transferring a rhamnose moiety to the C-3 position of the D-fucose of the F* chain. The function of the enzyme can be determined for example as described in Example 3.

The function of QS-7-RhaT may be determined by expressing in a heterologous host such as N. benthamiana or yeast the enzymes necessary to generate QA-Tri(X/R)-F*-Ac and the QS-7-RhaT candidate. The presence of the expected product may be assessed by LC-MS analysis, eventually complemented by NMR analysis. Alternatively, in vitro testing may be preferred in which QA-Tri(X/R)-F* is either purified from a plant extract or generated in vitro in an assay containing quillaic acid and the glycosyl transferases necessary to generate QA-Tri(X/R)-F*, or β-amyrin and the enzymes necessary to produce QA-Tri(X/R)-F*. The activity of the candidate QS-7-RhaT is then tested in vitro on the QA-Tri(X/R)-F* substrate and the product formation is determined by LC-MS analysis.

In the methods of the invention, the transfer of a rhamnose residue to the C-3 position of the D-fucose of F* may be carried out by the enzyme QS-7-RhaT (SEQ ID NO 58), or an enzyme having at least 70% sequence identity to the sequence for QS-7-RhaT (SEQ ID No 58). The amino acid sequence of the QS-7-RhaT enzyme may have at least 70%, 75%, 80%, 85%, 90%, 95%, 96%, 97%, 98% or 99% identity to SEQ ID NO 58. Accordingly, in some embodiments, the QS-7-RhaT has at least 70%, 75%, 80%, 85%, 90%, 95%, 96%, 97%, 98% or 99% identity to SEQ ID NO 58, suitably at least 90%, more suitably at least 95%. In respect of the enzymes defined here in terms of sequence identity, they typically retain the function of transferring a rhamnose moiety to the C-3 position of the D-fucose of F*.

In the methods of the invention the transfer of a glucose residue to a molecule comprising QA-Tri(X/R)-F*-R-Ac to form QA-Tri(X/R)-F*-GR-Ac is carried out by the enzyme QS-7-GlcT (SEQ ID NO 56), or an enzyme having at least 70% sequence identity to SEQ ID NO 56. The enzymes are capable of transferring a glucose residue to the C-3 position of the rhamnose residue of F*. The function of the enzyme can be determined for example as described in Example 1.

The function of QS-7-GlcT may be determined by expressing in a heterologous host such as N. benthamiana or yeast the enzymes necessary to generate QA-Tri(X/R)-F*-R-Ac and the QS-7-GlcT candidate. The presence of the expected product may be assessed by LC-MS analysis, eventually complemented by NMR analysis. Alternatively, in vitro testing may be preferred in which QA-Tri(X/R)-F*-R-Ac is either purified from a plant extract or generated in vitro in an assay containing quillaic acid and the glycosyl transferases necessary to generate QA-Tri(X/R)-F*-R-Ac, or β-amyrin and the enzymes necessary to produce QA-Tri(X/R)-F*-R-Ac. The activity of the candidate QS-7-GlcT is then tested in vitro on the QA-Tri(X/R)-F*-R-Ac substrate and the product formation is determined by LC-MS analysis.

In the methods of the invention, the transfer of a glucose residue to a molecule comprising QA-Tri(X/R)-F*-R-Ac to form QA-Tri(X/R)-F*-GR-Ac may be carried out by the enzyme QS-7-GlcT (SEQ ID NO 56), or an enzyme having at least 70% sequence identity to the sequence for QS-7-GlcT (SEQ ID No 56). The amino acid sequence of the QS-7-GlcT enzyme may have at least 70%, 75%, 80%, 85%, 90%, 95%, 96%, 97%, 98% or 99% identity to SEQ ID NO 56. Accordingly, in some embodiments, the QS-7-GlcT has at least 70%, 75%, 80%, 85%, 90%, 95%, 96%, 97%, 98% or 99% identity to SEQ ID NO 56, suitably at least 90%, more suitably at least 95%. In respect of the enzymes defined here in terms of sequence identity, they typically retain the function of transferring a glucose residue to a molecule comprising QA-Tri(X/R)-F*-R-Ac to form QA-Tri(X/R)-F*-GR-Ac.

The percentage sequence identity of the sequences to QS-7-RhaT, QS-7-GlcT, QS-7-AcetylT, DMOT9 and SOAP10 may all be the same or different.

As mentioned above, the methods of the invention comprise adding an acyl moiety, glucose moiety and a rhamnose moiety to QA-Tri(X/R)-F*. QA-Tri(X/R)-F* is described above. An additional feature of the methods of the invention is the steps for making the QA backbone, the branched trisaccharide at the C-3 position of the molecule comprising a QA backbone (QA-Tri(X/R)) and the linear sugar chain at the C-28 position (F*) of the molecule comprising a QA backbone (QA-Tri(X/R)-F*).

### QA Synthesis

One step of the method of forming the QA backbone of a molecule comprising QA-Tri(X/R)-F* is the cyclisation of 2,3-oxidosqualene to form a molecule comprising triterpene β-amyrin. This step is carried out by an oxidosqualene cyclase. In particular the oxidosqualene cyclase may be an enzyme according to QsbAS (SEQ ID NO 18) or a sequence with at least 50% sequence identity to SEQ ID NO 18. The oxidosqualene cyclase may be encoded by the polynucleotide sequence of SEQ ID NO 17.

This step encompasses oxidosqualene cyclase enzymes having at least 50% sequence identity to the sequence for QsbAS (SEQ ID NO 18). The amino acid sequence of the QsbAS enzyme may have at least 50%, 55%, 65%, 70%, 75%, 80%, 85%, 90%, 95%, 96%, 97%, 98% or 99% identity to SEQ ID NO 18. Accordingly, in some embodiments, the QsbAS has at least 50%, 55%, 65%, 70%, 75%, 80%, 85%, 90%, 95%, 96%, 97%, 98% or 99% identity to SEQ ID NO 18, suitably at least 90%, more suitably at least 95%. In respect of the enzymes defined here in terms of sequence identity, they typically retain the function of the cyclisation of 2,3-oxidosqualene to form a molecule comprising triterpene β-amyrin.

The molecule comprising the β-amyrin scaffold is further oxidised to a carboxylic acid, alcohol and aldehyde at the C-28, C-16a and C-23 positions, respectively. Another step of this feature of the invention is the oxidation of the molecule comprising the β-amyrin scaffold to form a carboxylic acid at the C-28 position. This step is carried out by a cytochrome P450 monooxygenase. The cytochrome P450 monooxygenase is a C-28 oxidase QsCYP716-C-28. For example, the C-28 oxidase QsCYP716-C-28 may be according to SEQ ID NO 20 or a sequence with at least 50% sequence identity to SEQ ID NO 20. QsCYP716-C-28 may be encoded by the polynucleotide sequence of SEQ ID NO 19 or a sequence with at least 50% sequence identity to SEQ ID NO 19.

This step encompasses cytochrome P450 monooxygenases having at least 50% sequence identity to the sequence for QsCYP716-C-28 (SEQ ID NO 20). The amino acid sequence of the QsCYP716-C-28 enzyme may have at least 50%, 55%, 65%, 70%, 75%, 80%, 85%, 90%, 95%, 96%, 97%, 98% or 99% identity to SEQ ID NO 20. Accordingly, in some embodiments, the QsCYP716-C-28 has at least 50%, 55%, 65%, 70%, 75%, 80%, 85%, 90%, 95%, 96%, 97%, 98% or 99% identity to SEQ ID NO 20, suitably at least 90%, more suitably at least 95%. In respect of the enzymes defined here in terms of sequence identity, they typically retain the function of oxidising a molecule comprising the β-amyrin scaffold to form a carboxylic acid at the C-28 position.

Another step of this feature of the invention is the oxidation of the molecule comprising the β-amyrin scaffold to form an alcohol at the C-16 position. This step is performed by a cytochrome P450 monooxygenase. The cytochrome P450 monooxygenase is a C-16a oxidase QsCYP716-C-16a. For example, the C-16a oxidase QsCYP716-C-16a may be according to SEQ ID NO 22 or a sequence with at least 50% sequence identity to SEQ ID NO 22. QsCYP716-C-16a may be encoded by the polynucleotide sequence of SEQ ID NO 21 or a sequence with at least 50% sequence identity to SEQ ID NO 21.

This step encompasses cytochrome P450 monooxygenases having at least 50% sequence identity to the sequence for QsCYP716-C-16a (SEQ ID NO 22). The amino acid sequence of the QsCYP716-C-16a enzyme may have at least 50%, 55%, 65%, 70%, 75%, 80%, 85%, 90%, 95%, 96%, 97%, 98% or 99% identity to SEQ ID NO 22. Accordingly, in some embodiments, the QsCYP716-C-16a has at least 50%, 55%, 65%, 70%, 75%, 80%, 85%, 90%, 95%, 96%, 97%, 98% or 99% identity to SEQ ID NO 22, suitably at least 90%, more suitably at least 95%. In respect of the enzymes defined here in terms of sequence identity, they typically retain the function of oxidising a molecule comprising the β-amyrin scaffold to form an alcohol at the C-16 position.

A further step of this feature of the invention is the oxidation of the molecule comprising the β-amyrin scaffold to form an aldehyde at the C-23 position. This step is performed by a cytochrome P450 monooxygenase. The cytochrome P450 monooxygenase is a C-23 oxidase QsCYP714-C-23. For example, the C-23 oxidase QsCYP714-C-23 may be according to SEQ ID NO 24 or a sequence with at least 50% sequence identity to SEQ ID NO 24. QsCYP714-C-23 may be encoded by the polynucleotide sequence of SEQ ID NO 23 or a sequence with at least 50% sequence identity to SEQ ID NO 23.

This step encompasses cytochrome P450 monooxygenases having at least 50% sequence identity to the sequence for QsCYP714-C-23 (SEQ ID NO 24). The amino acid sequence of the QsCYP714-C-23 enzyme may have at least 50%, 55%, 65%, 70%, 75%, 80%, 85%, 90%, 95%, 96%, 97%, 98% or 99% identity to SEQ ID NO 24. Accordingly, in some embodiments, the QsCYP714-C-23 has at least 50%, 55%, 65%, 70%, 75%, 80%, 85%, 90%, 95%, 96%, 97%, 98% or 99% identity to SEQ ID NO 24, suitably at least 90%, more suitably at least 95%. In respect of the enzymes defined here in terms of sequence identity, they typically retain the function of oxidising a molecule comprising the β-amyrin scaffold to form an aldehyde at the C-23 position.

These steps form the QA backbone.

This feature of the invention relates to a method of making a molecule comprising the QA backbone involving a number of steps. The steps can be performed in a specific order or in any order or simultaneously. Preferably, this molecule is formed by the production of the β-amyrin scaffold followed by the sequential oxidation at the C-28, C-16a and C-23 positions respectively. The steps of this feature of these aspects of the invention are described for the preferable situation mentioned above. However, the steps may occur in any order.

The sugar units forming the C-3 branched trisaccharide and F* are then added. Preferably the molecule comprising the QA backbone is made, then the steps for adding the C-3 chain are carried out, followed by the steps for adding F*. However, these steps can be performed in a specific order or in any order or simultaneously.

### C-3 Branched Trisaccharide Synthesis

The steps of the formation of Tri(X/R) of a molecule comprising QA-Tri(X/R)-F* are described for the situation when the branched trisaccharide at the C-3 position of the molecule comprising the QA backbone is initiated by attaching a 3-D-glucopyranuronic acid moiety to a molecule comprising QA to form a molecule comprising QA-Mono. However, the steps may occur in any order.

The first step of forming the C-3 chain is attaching a β-D-glucopyranuronic acid moiety to a molecule comprising QA to form a molecule comprising QA-Mono. The step may be carried out by an enzyme QsCSL1 according to SEQ ID NO 26 or an enzyme QsCsIG2 according to SEQ ID NO 28, or a sequence with at least 70% sequence identity to SEQ ID NO 26 or 28. QsCSL1 may be encoded by the polynucleotide sequence of SEQ ID NO 25 or a sequence with at least 70% sequence identity to SEQ ID NO 25. QsCsIG2 may be encoded by the polynucleotide sequence of SEQ ID NO 27 or a sequence with at least 70% sequence identity to SEQ ID NO 27.

This step encompasses enzymes having at least 70% sequence identity to the sequences for QsCSL1 and QsCsIG2 (SEQ ID NO 26 or 28 respectively). The amino acid sequence of the QsCSL1 enzyme may have at least 70%, 75%, 80%, 85%, 90%, 95%, 96%, 97%, 98% or 99% identity to SEQ ID NO 26. The amino acid sequence of the QsCsIG2 enzyme may have at least 70%, 75%, 80%, 85%, 90%, 95%, 96%, 97%, 98% or 99% identity to SEQ ID NO 28. Accordingly, in some embodiments, the QsCSL1 and/or QsCsIG2 has at least 70%, 75%, 80%, 85%, 90%, 95%, 96%, 97%, 98% or 99% identity to SEQ ID NO 26 or 28, suitably at least 90%, more suitably at least 95%. In respect of the enzymes defined here in terms of sequence identity, they typically retain the function of attaching a β-D-glucopyranuronic acid moiety to a molecule comprising QA to form a molecule comprising QA-Mono.

Another step of the method of forming the C-3 chain is attaching a D-galactopyranose moiety to a β-D-glucopyranuronic acid moiety on a molecule comprising QA-Mono to form a molecule comprising QA-Di. The step may be carried out by an enzyme Qs-3-O-GalT according to SEQ ID NO 30 or a sequence with at least 70% sequence identity to SEQ ID NO 30. Qs-3-O-GalT may be encoded by the polynucleotide sequence of SEQ ID NO 29 or a sequence with at least 70% sequence identity to SEQ ID NO 29.

This step encompasses enzymes having at least 70% sequence identity to the sequence for Qs-3-O-GalT (SEQ ID NO 30). The amino acid sequence of the Qs-3-O-GalT enzyme may have at least 70%, 75%, 80%, 85%, 90%, 95%, 96%, 97%, 98% or 99% identity to SEQ ID NO 30. Accordingly, in some embodiments, the Qs-3-O-GalT has at least 70%, 75%, 80%, 85%, 90%, 95%, 96%, 97%, 98% or 99% identity to SEQ ID NO 30, suitably at least 90%, more suitably at least 95%. In respect of the enzymes defined here in terms of sequence identity, they typically retain the function of attaching a D-galactopyranose moiety to a 3-D-glucopyranuronic acid moiety on a molecule comprising QA-Mono to form a molecule comprising QA-Di.

A further step of the method of forming the C-3 chain is attaching a L-rhamnopyranose moiety to a β-D-glucopyranuronic acid moiety on a molecule comprising QA-Di, to form a molecule comprising QA-TriR. The step may be carried out by an enzyme DN20529_c0_g2_i8 according to SEQ ID NO 36, Qs_0283850 according to SEQ ID NO 34, or an enzyme Qs-3-O-RhaT/XyIT according to SEQ ID NO 32, or a sequence with at least 70% sequence identity to SEQ ID NO 36, 34 or 32. DN20529_c0_g2_i8 may be encoded by the polynucleotide sequence of SEQ ID NO 35 or a sequence with at least 70% sequence identity to SEQ ID NO 35. Qs_0283850 may be encoded by the polynucleotide sequence of SEQ ID NO 33 or a sequence with at least 70% sequence identity to SEQ ID NO 33. Qs-3-O-RhaT/XyIT may be encoded by the polynucleotide sequence of SEQ ID NO 31 or a sequence with at least 70% sequence identity to SEQ ID NO 31.

This step encompasses enzymes having at least 70% sequence identity to the sequences for DN20529_c0_g2_i8, Qs_0283850, or Qs-3-O-RhaT/XyIT (SEQ ID NO 36, 34 or 32 respectively). The amino acid sequence of the DN20529_c0_g2_i8 enzyme may have at least 70%, 75%, 80%, 85%, 90%, 95%, 96%, 97%, 98% or 99% identity to SEQ ID NO 36. The amino acid sequence of the Qs_0283850 enzyme may have at least 70%, 75%, 80%, 85%, 90%, 95%, 96%, 97%, 98% or 99% identity to SEQ ID NO 34. The amino acid sequence of the Qs-3-O-RhaT/XyIT enzyme may have at least 70%, 75%, 80%, 85%, 90%, 95%, 96%, 97%, 98% or 99% identity to SEQ ID NO 32. Accordingly, in some embodiments, the DN20529_c0_g2_i8, Qs_0283850, and/or Qs-3-O-RhaT/XyIT has at least 70%, 75%, 80%, 85%, 90%, 95%, 96%, 97%, 98% or 99% identity to SEQ ID NO 36, 34 or 32, suitably at least 90%, more suitably at least 95%. In respect of the enzymes defined here in terms of sequence identity, they typically retain the function of attaching a L-rhamnopyranose moiety to a β-D-glucopyranuronic acid moiety on a molecule comprising QA-Di, to form a molecule comprising QA-TriR.

Yet a further step of the method of forming the C-3 chain is attaching a β-D-xylopyranose moiety to a β-D-glucopyranuronic acid moiety on a molecule comprising QA-Di, to form a molecule comprising QA-Trix. This step may be carried out by an enzyme Qs_0283870 according to SEQ ID NO 38, or an enzyme Qs-3-O-RhaT/XyIT according to SEQ ID NO 32, or a sequence with at least 70% sequence identity to SEQ ID NO 38 or 32. Qs_0283870 may be encoded by the polynucleotide sequence of SEQ ID NO 37 or a sequence with at least 70% sequence identity to SEQ ID NO 37. Qs-3-O-RhaT/XyIT may be encoded by the polynucleotide sequence of SEQ ID NO 31 or a sequence with at least 70% sequence identity to SEQ ID NO 31.

This step encompasses enzymes having at least 70% sequence identity to the sequences for Qs_0283870 or Qs-3-O-RhaT/XyIT (SEQ ID NO 38 or 32 respectively). The amino acid sequence of the Qs_0283870 enzyme may have at least 70%, 75%, 80%, 85%, 90%, 95%, 96%, 97%, 98% or 99% identity to SEQ ID NO 38. The amino acid sequence of the Qs-3-O-RhaT/XyIT enzyme may have at least 70%, 75%, 80%, 85%, 90%, 95%, 96%, 97%, 98% or 99% identity to SEQ ID NO 32. Accordingly, in some embodiments, the Qs_0283870 and/or Qs-3-O-RhaT/XyIT has at least 70%, 75%, 80%, 85%, 90%, 95%, 96%, 97%, 98% or 99% identity to SEQ ID NO 38 or 32, suitably at least 90%, more suitably at least 95%. In respect of the enzymes defined here in terms of sequence identity, they typically retain the function of attaching a β-D-xylopyranose moiety to a β-D-glucopyranuronic acid moiety on a molecule comprising QA-Di, to form a molecule comprising QA-TriX.

These steps form the C-3 chain on the QA backbone.

### C-28 Linear Tetrasaccharide Synthesis

The steps of the formation of F* of a molecule comprising QA-Tri(X/R)-F* are described for the situation when F* of the molecule comprising the QA backbone is initiated by attaching UDP-α-D-fucose moiety to a molecule comprising QA-Tri(X/R) to form a molecule comprising QA-Tri(X/R)-F. However, the steps may occur in any order. For example, F* may be produced and then attached to the QA-Tri(X/R) backbone.

The first step of forming F* may be attaching a UDP-α-D-fucose moiety to the C-28 position of a molecule comprising QA-Tri(R/X), to form a molecule comprising QA-Tri(R/X)-F. This step may be carried out by an enzyme Qs-28-O-FucT according to SEQ ID NO 2 or a sequence with at least 70% sequence identity to SEQ ID NO 2. Qs-28-O-FucT may be encoded by the polynucleotide sequence of SEQ ID NO 1 or a sequence with at least 70% sequence identity to SEQ ID NO 1. The first step of forming F* may also be attaching UDP-4-keto, 6-deoxy-D-glucose to a molecule comprising QA-Tri(R/X), to form a molecule comprising QA-Tri(R/X)-F. This step may be carried out by the enzymes Qs-28-O-FucT according to SEQ ID NO 2 or a sequence with at least 70% sequence identity to SEQ ID NO 2 and QsFucSyn according to SEQ ID NO 12 or a sequence with at least 45% sequence identity to SEQ ID NO 12. Qs-28-O-FucT may be encoded by the polynucleotide sequence of SEQ ID NO 1 or a sequence with at least 70% sequence identity to SEQ ID NO 1. QsFucSyn may be encoded by the polynucleotide sequence of SEQ ID NO 11 or a sequence with at least 45% sequence identity to SEQ ID NO 11.

This step encompasses enzymes having at least 70% sequence identity to the sequence for Qs-28-O-FucT (SEQ ID NO 2). The amino acid sequence of the Qs-28-O-FucT enzyme may have at least 70%, 75%, 80%, 85%, 90%, 95%, 96%, 97%, 98% or 99% identity to SEQ ID NO 2. Accordingly, in some embodiments, the Qs-28-O-FucT has at least 70%, 75%, 80%, 85%, 90%, 95%, 96%, 97%, 98% or 99% identity to SEQ ID NO 2, suitably at least 90%, more suitably at least 95%. In respect of the enzymes defined here in terms of sequence identity, they typically retain the function of attaching a UDP-α-D-fucose moiety to the C-28 position of a molecule comprising QA-Tri(R/X), to form a molecule comprising QA-Tri(R/X)-F; or attaching UDP-4-keto, 6-deoxy-D-glucose to a molecule comprising QA-Tri(R/X), to form a molecule comprising QA-Tri(R/X)-F.

This step also encompasses enzymes having at least 45% sequence identity to the sequence for QsFucSyn (SEQ ID NO 12). The amino acid sequence of the QsFucSyn enzyme may have at least 45%, 50%, 55%, 60%, 65%, 70%, 75%, 80%, 85%, 90%, 95%, 96%, 97%, 98% or 99% identity to SEQ ID NO 12. Accordingly, in some embodiments, the QsFucSyn has at least 45%, 50%, 55%, 60%, 65%, 70%, 75%, 80%, 85%, 90%, 95%, 96%, 97%, 98% or 99% identity to SEQ ID NO 12, suitably at least 90%, more suitably at least 95%. In respect of the enzymes defined here in terms of sequence identity, they typically retain the function of attaching a UDP-α-D-fucose moiety to the C-28 position of a molecule comprising QA-Tri(R/X), to form a molecule comprising QA-Tri(R/X)-F; or attaching UDP-4-keto, 6-deoxy-D-glucose to a molecule comprising QA-Tri(R/X), to form a molecule comprising QA-Tri(R/X)-F.

Another step of forming the F* is attaching a UDP-β-L-rhamnose moiety to a UDP-α-D-fucose moiety on a molecule comprising QA-Tri(R/X)-F, to form a molecule comprising QA-Tri(R/X)-FR. This step may be carried out by an enzyme Qs-28-O-RhaT according to SEQ ID NO 4 or a sequence with at least 70% sequence identity to SEQ ID NO 4. Qs-28-O-RhaT may be encoded by the polynucleotide sequence of SEQ ID NO 3 or a sequence with at least 70% sequence identity to SEQ ID NO 3.

This step also encompasses enzymes having at least 70% sequence identity to the sequence for Qs-28-O-RhaT (SEQ ID NO 4). The amino acid sequence of the Qs-28-O-RhaT enzyme may have at least 70%, 75%, 80%, 85%, 90%, 95%, 96%, 97%, 98% or 99% identity to SEQ ID NO 4. Accordingly, in some embodiments, the Qs-28-O-RhaT has at least 70%, 75%, 80%, 85%, 90%, 95%, 96%, 97%, 98% or 99% identity to SEQ ID NO 4, suitably at least 90%, more suitably at least 95%. In respect of the enzymes defined here in terms of sequence identity, they typically retain the function of attaching a UDP-β-L-rhamnose moiety to a UDP-α-D-fucose moiety on a molecule comprising QA-Tri(R/X)-F, to form a molecule comprising QA-Tri(R/X)-FR.

A further step for forming F* is attaching a UDP-α-D-xylose moiety to a UDP-β-L-rhamnose moiety on a molecule comprising QA-Tri(R/X)-FR, to form a molecule comprising QA-Tri(R/X)-FRX. This step may be carried out by an enzyme Qs-28-O-XyIT3 according to SEQ ID NO 6 or a sequence with at least 70% sequence identity to SEQ ID NO 6. Qs-28-O-XyIT3 may be encoded by the polynucleotide sequence of SEQ ID NO 5 or a sequence with at least 70% sequence identity to SEQ ID NO 5.

This step also encompasses enzymes having at least 70% sequence identity to the sequence for Qs-28-O-XyIT3 (SEQ ID NO 6). The amino acid sequence of the Qs-28-O-XyIT3 enzyme may have at least 70%, 75%, 80%, 85%, 90%, 95%, 96%, 97%, 98% or 99% identity to SEQ ID NO 6. Accordingly, in some embodiments, the Qs-28-O-XyIT3 has at least 70%, 75%, 80%, 85%, 90%, 95%, 96%, 97%, 98% or 99% identity to SEQ ID NO 6, suitably at least 90%, more suitably at least 95%. In respect of the enzymes defined here in terms of sequence identity, they typically retain the function of attaching a UDP-α-D-xylose moiety to a UDP-β-L-rhamnose moiety on a molecule comprising QA-Tri(R/X)-FR, to form a molecule comprising QA-Tri(R/X)-FRX.

An optional step for forming F* may be attaching a UDP-α-D-xylose moiety to a UDP-α-D-xylose moiety on a molecule comprising QA-Tri(R/X)-FRX to form a molecule comprising QA-Tri(R/X)-FRXX. This step may be carried out by an enzyme Qs-28-O-XyIT4 according to SEQ ID NO 8 or a sequence with at least 70% sequence identity to SEQ ID NO 8. Qs-28-O-XyIT4 may be encoded by the polynucleotide sequence of SEQ ID NO 7 or a sequence with at least 70% sequence identity to SEQ ID NO 7.

This optional step also encompasses enzymes having at least 70% sequence identity to the sequence for Qs-28-O-XyIT4 (SEQ ID NO 8). The amino acid sequence of the Qs-28-O-XyIT4 enzyme may have at least 70%, 75%, 80%, 85%, 90%, 95%, 96%, 97%, 98% or 99% identity to SEQ ID NO 8. Accordingly, in some embodiments, the Qs-28-O-XyIT4 has at least 70%, 75%, 80%, 85%, 90%, 95%, 96%, 97%, 98% or 99% identity to SEQ ID NO 8, suitably at least 90%, more suitably at least 95%. In respect of the enzymes defined here in terms of sequence identity, they typically retain the function of attaching a UDP-α-D-xylose moiety to a UDP-α-D-xylose moiety on a molecule comprising QA-Tri(R/X)-FRX to form a molecule comprising QA-Tri(R/X)-FRXX.

Another optional step for forming the F* may be attaching a UDP-α-D-apiose moiety to a UDP-α-D-xylose moiety on a molecule comprising QA-Tri(R/X)-FRX to form a molecule comprising QA-Tri(R/X)-FRXA. This step may be carried out by an enzyme Qs-28-O-ApiT4 according to SEQ ID NO 10 or a sequence with at least 70% sequence identity to SEQ ID NO 10. Qs-28-O-ApiT4 may be encoded by the polynucleotide sequence of SEQ ID NO 9 or a sequence with at least 70% sequence identity to SEQ ID NO 9.

This step also encompasses enzymes having at least 70% sequence identity to the sequence for Qs-28-O-ApiT4 (SEQ ID NO 10). The amino acid sequence of the Qs-28-O-ApiT4 enzyme may have at least 70%, 75%, 80%, 85%, 90%, 95%, 96%, 97%, 98% or 99% identity to SEQ ID NO 10. Accordingly, in some embodiments, the Qs-28-O-ApiT4 has at least 70%, 75%, 80%, 85%, 90%, 95%, 96%, 97%, 98% or 99% identity to SEQ ID NO 10, suitably at least 90%, more suitably at least 95%. In respect of the enzymes defined here in terms of sequence identity, they typically retain the function of attaching a UDP-α-D-apiose moiety to a UDP-α-D-xylose moiety on a molecule comprising QA-Tri(R/X)-FRX to form a molecule comprising QA-Tri(R/X)-FRXA.

These steps form the F* on the QA backbone.

The method of the second aspect of the invention is carried out in a biological system or host. The polynucleotides encoding for one or more of the above enzymes are introduced and expressed in the biological system. In most cases, the biological system will not naturally express any of the enzymes of the second aspect of the invention and thus the biological system will be engineered to express all the enzymes.

The biological system may be a plant or a microorganism. When the biological system is a plant, the plant may be row crops for example sunflower, potato, canola, dry bean, field pea, flax, safflower, buckwheat, cotton, maize, soybeans and sugar beets. The plant may also be corn, wheat, oilseed rape and rice. Preferably the plant may be Nicotiana benthamiana.

In certain embodiments of the methods of the second aspect of the invention, the biological system is not Quillaja saponaria.

When the biological system is a microorganism, the microorganism may be bacteria or yeast.

Yeast (Saccharomyces cerevisiae) is a heterologous host used for the production of high value small molecules, including terpenes. Like plants, yeast endogenously produces the triterpenoid precursor 2,3-oxidosqualene, and so is a promising host for industrial-scale production of triterpenoids. It is also a highly effective host for the functional expression of plant CYPs at endoplasmic reticulum membranes. There is minimal modification of triterpenoid scaffolds by endogenous yeast enzymes, facilitating product purification. Yeast can be a production host producing triterpenes with diverse glycoside conjugates comprising multiple types of sugars in linear and branched configuration. Glycosylation reactions in yeast are restricted by the limited palette of endogenous sugar donors. By expressing genes from higher plants, however, the nucleotide sugar metabolism of yeast can be expanded beyond UDP-glucose and UDP-galactose, to include UDP-rhamnose, -glucuronic acid, -xylose, -arabinose and others.

The method of the first aspect of the invention may be performed in vitro. By “in vitro”, it is meant in the sense of the present invention to have appropriate QA-Tri(X/R)-F* derivatives enzymatically treated with appropriate enzymes of the invention. QA-Tri(X/R)-F* derivatives may be either biosynthetically produced or chemically synthesized. Enzymes may be either cloned or purified from their native environment. It is within the skilled person's ambit to determine the optimal conditions (e.g. duration, temperature, buffer etc). of the enzymatic treatment.

The identity of the QA derivative can be confirmed, for example, by elucidating its structure by NMR as described in Materials and Methods.

In the second, third and fourth aspects of the invention, amino acid sequence SEQ ID NO 60 is encoded by polynucleotide sequence SEQ ID NO 59; amino acid sequence SEQ ID NO 58 is encoded by polynucleotide sequence SEQ ID NO 57; and amino acid sequence SEQ ID NO 56 is encoded by polynucleotide sequence SEQ ID NO 55.

The methods of the second, third and fourth aspects of the invention include transforming the host with polynucleotides by introducing the polynucleotides required for the biosynthesis of a molecule comprising QA-Tri(X/R)-F*-GR-Ac, into the host cells via a vector. Recombination may occur between the vector and the host cell genome to introduce the polynucleotides into the host cell genome.

A fifth aspect of the invention is a glucosyltransferase enzyme according to SEQ ID NO 56 (QS-7-GlcT) or an enzyme having a sequence with at least 70% sequence identity to SEQ ID NO 56. The enzyme is capable of transferring a glucose residue to the C-3 position of the rhamnose residue of the F* of a QS-7 precursor. This enzyme is as described in relation to the methods of the first to fourth aspects of the invention and has the same properties and function as described in relation to the method of the first to fourth aspects of the invention.

The glucosyltransferase enzyme may be encoded by a polynucleotide of SEQ ID NO 55 or a polynucleotide molecule which also encodes for the amino acid according to the fifth aspect of the invention. The QS-7-GlcT enzyme may, for example, be encoded by the polynucleotide sequence according to SEQ ID NO 55 or by a sequence which, by virtue of the degenerative code, also encodes an enzyme according to the fifth aspect of the invention.

The fifth aspect of the invention encompasses glucosyltransferase enzymes having at least 70% sequence identity to the sequence for QS-7-GlcT (SEQ ID NO 56). The amino acid sequence of the QS-7-GlcT enzyme may have at least 70%, 75%, 80%, 85%, 90%, 95%, 96%, 97%, 98% or 99% identity to SEQ ID NO 56. Accordingly, in some embodiments, the QS-7-GlcT has at least 70%, 75%, 80%, 85%, 90%, 95%, 96%, 97%, 98% or 99% identity to SEQ ID NO 56, suitably at least 90%, more suitably at least 95%. In respect of the enzymes defined here in terms of sequence identity, they typically retain the function of transferring a glucose moiety to a molecule comprising QA-Tri(X/R)-F* to form QA-Tri(X/R)-F*G.

A sixth aspect of the invention is a rhamnosyltransferase enzyme according to SEQ ID NO 58 (QS-7-RhaT) or an enzyme having a sequence with at least 70% sequence identity to SEQ ID NO 58. The enzyme is capable of transferring a rhamnose moiety to the C-3 position of the D-fucose of F* of a QS-7 precursor. This enzyme is as described in the methods of the first to fourth aspects of the invention and has the same properties and function as described in relation to the methods of the first to fourth aspects of the invention.

The rhamnosyltransferase enzyme may be encoded by a polynucleotide of SEQ ID NO 57 or a polynucleotide molecule which also encodes for the amino acid according to the sixth aspect of the invention. The QS-7-RhaT enzyme may, for example, be encoded by the polynucleotide sequence according to SEQ ID NO 57 or by a sequence which, by virtue of the degenerative code, also encodes an enzyme according to the sixth aspect of the invention.

The sixth aspect of the invention encompasses rhamnosyltransferase enzymes having at least 70% sequence identity to the sequence for QS-7-RhaT (SEQ ID NO 58). The amino acid sequence of the QS-7-RhaT enzyme may have at least 70%, 75%, 80%, 85%, 90%, 95%, 96%, 97%, 98% or 99% identity to SEQ ID NO 58. Accordingly, in some embodiments, the QS-7-RhaT has at least 70%, 75%, 80%, 85%, 90%, 95%, 96%, 97%, 98% or 99% identity to SEQ ID NO 58, suitably at least 90%, more suitably at least 95%. In respect of the enzymes defined here in terms of sequence identity, they typically retain the function of transferring a rhamnose moiety to the C-3 position of the D-fucose of the F* of a QS-7 precursor.

A seventh aspect of the invention is an acetyltransferase enzyme according to SEQ ID NO 60 (QS-7-AcetylT) or an enzyme having a sequence with at least 70% sequence identity to SEQ ID NO 60. The enzyme is capable of transferring an acyl unit to the C-4 position of the D-fucose of the F* of a QS-7 precursor. This enzyme is as described in the methods of the first to fourth aspects of the invention and has the same properties and function as described in relation to the methods of the first to fourth aspects of the invention.

The acetyltransferase enzyme may be encoded by a polynucleotide of SEQ ID NO 59 or a polynucleotide molecule which also encodes for the amino acid according to the seventh aspect of the invention. The QS-7-AcetylT enzyme may, for example, be encoded by the polynucleotide sequence according to SEQ ID NO 59 or by a sequence which, by virtue of the degenerative code, also encodes an enzyme according to the seventh aspect of the invention.

The seventh aspect of the invention encompasses acetyltransferase enzymes having at least 70% sequence identity to the sequence for QS-7-AcetylT (SEQ ID NO 60). The amino acid sequence of the QS-7-AcetylT enzyme may have at least 70%, 75%, 80%, 85%, 90%, 95%, 96%, 97%, 98% or 99% identity to SEQ ID NO 60. Accordingly, in some embodiments, the QS-7-AcetylT has at least 70%, 75%, 80%, 85%, 90%, 95%, 96%, 97%, 98% or 99% identity to SEQ ID NO 60, suitably at least 90%, more suitably at least 95%. In respect of the enzymes defined here in terms of sequence identity, they typically retain the function of transferring an acyl unit to the C-4 position of the D-fucose of F* of a QS-7 precursor.

Any sequence identity percentage of the fifth, sixth and seventh aspects of the invention can be combined with any other sequence identity percentage of the fifth, sixth and seventh aspects of the invention.

An eighth aspect of the invention is a polynucleotide which encodes one or more of the enzymes of the fifth to seventh aspects of the invention.

A ninth aspect of the present invention is a vector comprising one or more of the polynucleotides according to the eighth aspect of the invention.

The vector may comprise, one, two or three of the polynucleotides encoding the enzymes of the fifth to seventh aspects of the invention. Preferably, the vector will comprise three of the polynucleotides encoding the enzymes of the fifth to seventh aspects of the invention or a number of vectors which, together, comprise the three polynucleotides.

A tenth aspect of the present invention is a host cell comprising one or more of the polynucleotides according to the eighth aspect of the invention.

The host cell may be a plant cell or microbial cell. When the host cell is a microbial cell it is preferably a yeast cell. When the host cell is a plant cell, the plant is preferably Nicotiana benthamiana.

An additional feature of the tenth aspect of the invention is the method of introducing the polynucleotides of the eighth aspect of the invention, into the host cell. The polynucleotides may be introduced into the host cells via a vector. Recombination may occur between the vector and host cell genome to introduce the polynucleotides into the host cell genome. Alternatively, the polynucleotides may be introduced into the host cells by co-infiltration with a plurality of recombinant vectors. The recombinant vectors may be Agrobacterium tumefaciens stains, discussed below.

An eleventh aspect of the invention is a host cell transformed with the vector according to the ninth aspect of the invention.

A twelfth aspect of the invention is a biological system of a plant or a microorganism comprising host cells as set out according to the tenth and eleventh aspects of the invention. The biological system may be a plant or a microorganism. When the biological system is a plant, it may be Nicotiana benthamiana or any of the plants described above. The method of producing the plant comprises the steps of introducing the polynucleotides of the invention into the host plant cell and regenerating a plant from the transformed host plant cell. When the biological system is a microorganism, it may be yeast.

The invention also includes the method of making each enzyme and each polynucleotide of the above aspects of the invention, as well as a method of making a vector comprising one or more of the polynucleotides of the invention, as well as the host cells of the tenth and eleventh aspects of the invention and a method of making the biological system of the twelfth aspect of the invention. These methods use techniques and products well known in the art, such as in WO2019/122259 and WO2020/260475, and are described in more detail as follows:

The polynucleotides of the invention can be included in a vector, in particular an expression vector. The vector may be any plasmid, cosmid, phage or Agrobacterium vector in double or single stranded linear or circular form which can transform a prokaryotic or eukaryotic host either by integration into the cellular genome or other. The vector may be an expression vector, including an inducible promoter, operably linked to the polynucleotide sequence. Typically, the vector may include, between the inducible promoter and the polynucleotide sequence, an enhancer sequence. The vector may also include a terminator sequences and optionally a 3′ UTR located upstream of said terminator sequence. The vector may include one or more polynucleotides encoding enzymes of the fifth to seventh aspects of the invention, preferably all sequences needed to produce one version of the molecule as set out according to the first and second aspects of the invention. The vector may be a plant vector or a microbial vector.

The polynucleotide in the vector may be under the control of, and operably linked to, an appropriate promoter or other regulatory elements for transcription in a host cell. The host cell may be a yeast cell, bacterial cell or plant cell. The vector may be a bi-functional expression vector which functions in multiple hosts. In the case of genomic DNA, this may contain its own promoter or other regulatory elements. The advantage of using a native promoter is that this may avoid pleiotropic responses. In the case of cDNA this may be under the control of an appropriate promoter or other regulatory elements for expression in the host cell

Preferred vectors for use in plants comprise border sequences which permit the transfer and integration of the expression vector into the plant genome. The vector may be a plant binary vector.

The vector may be transfected into a host cell in any biological system. The host may be a microbe, such as E. coli, or yeast. The vector may be part of an Agrobacterium tumefaciens strain and used to infect a biological plant host system. The Agrobacterium tumefaciens may each contain one of the required polynucleotides encoding for the invention and can be combined to co-infect a host cell, such that the host cell contains all the necessary polynucleotides to encode for the enzymes of the fifth to seventh aspects of the invention.

The present invention also includes the steps of culturing the host or growing the host for the production, harvest and isolation of the desired QA-Tri(X/R)-F*-GR-Ac derivative.

An additional feature of the first to fourth aspects of the invention is the step of isolating the QA-Tri(X/R)-F*-GR-Ac derivative.

The thirteenth aspect of the invention is QA-Tri(X/R)-F*-GR-Ac derivatives obtainable by the methods of the invention, in particular the methods of the first to fourth aspects of the invention. A QA-Tri(X/R)-F*-GR-Ac derivative obtainable by the methods of the invention may be isolated from the biological system. The isolated QA-Tri(X/R)-F*-GR-Ac derivative is QA-TriR-FRXGR-Ac, QA-TriR-FRXX-GR-Ac, QA-TriR-FRXA-GR-Ac, QA-TriX-FRXGR-Ac, QA-TriX-FRXX-GR-Ac, QA-TriX-FRXA-GR-Ac, QA-Tri(X/R)-FRXGR-Ac, QA-Tri(X/R)-FRXX-GR-Ac and/or QA-Tri(X/R)-FRXA-GR-Ac or mixtures thereof. The QA-Tri(X/R)-F*-GR-Ac derivative of this aspect of the invention may be obtained by the methods of the invention. The QA-Tri(X/R)-F*-GR-Ac derivative may preferably be QA-TriX-FRXA-GR-Ac.

A further aspect of the invention is a method of making a QA-Tri(X/R)-F*-GR-Ac derivative comprising the method steps of the invention, including the step of isolating the QA derivative.

The fourteenth aspect of the invention is the use of the QA-Tri(X/R)-F*-GR-Ac derivative, in particular QA-TriX-FRXA-GR-Ac as an adjuvant to be included in a vaccine composition, once isolated from the biological system. The adjuvant may be a liposomal formulation or immune stimulating complex (ISCOM) formulation.

An additional feature of the fourteenth aspect of the invention is that the adjuvant further comprises a TLR4 agonist. The TLR4 agonist may be 3D-MPL. QA-Tri(X/R)-F*-GR-Ac derivatives of the present invention may be combined with further immuno-stimulants, such as a TLR4 agonist, in particular lipopolysaccharide TLR4 agonists, such as lipid A derivatives, especially a monophosphoryl lipid A, e.g. 3-de-O-acylated monophosphoryl lipid A (3D-MPL). 3D-MPL is sold under the name ‘MPL’ by GlaxoSmithKline Biologicals N.A. See, for example, U.S. Pat. Nos. 4,436,727; 4,877,611; 4,866,034 and 4,912,094. 3D-MPL can be produced according to the methods described in GB 2 220 211 A. Chemically, it is a mixture of 3-deacylated monophosphoryl lipid A with 4, 5 or 6 acylated chains.

Other TLR4 agonists which may be combined with QA derivatives of the invention include Glucopyranosyl Lipid Adjuvant (GLA) such as described in WO2008/153541 or WO2009/143457 or literature articles (Coler et al. 2011 and Arias et al. 2012).

An additional feature of the fourteenth aspect of the invention is that the QA-Tri(X/R)-F*GR-Ac derivative, such as for example QA-Tri(X/R)-FRXA-GR-Ac is combined with QS-21, whether as a fraction purified from the bark of Quillaja saponaria or biosynthetically produced.

Adjuvants of the invention may also be formulated into a suitable carrier, such as an emulsion (e.g. an oil-in-water emulsion), liposomes, or immune stimulating complexes (ISCOMs), as described below.

### Liposomes

The term liposome is well known in the art and defines a general category of vesicles which comprise one or more lipid bilayers surrounding an aqueous space. Liposomes thus consist of one or more lipid and/or phospholipid bilayers and can contain other molecules, such as proteins or carbohydrates, in their structure. Because both lipid and aqueous phases are present, liposomes can encapsulate or entrap water-soluble material, lipid-soluble material, and/or amphiphilic compounds. A method for making such liposomes is described in WO2013/041572.

Liposome size may vary from 30 nm to several um depending on the phospholipid composition and the method used for their preparation.

The liposome size will be in the range of 50 nm to 200 nm, especially 60 nm to 180 nm, such as 70-165 nm. Optimally, the liposomes should be stable and have a diameter of 100 nm to allow convenient sterilization by filtration.

Structural integrity of the liposomes may be assessed by methods such as dynamic light scattering (DLS) measuring the size (Z-average diameter, Zav) and polydispersity of the liposomes, or, by electron microscopy for analysis of the structure of the liposomes. The average particle size may be between 95 and 120 nm, and/or, the polydispersity (Pdl) index may not be more than 0.3 (such as not more than 0.2).

### ISCOMs

The term immune stimulating complex (ISCOM) is well known in the art and defines a delivery system for antigen and adjuvant together in the same particle. ISCOMs are spherical, hollow, cage-like self-assembled particles.

Saponin-based adjuvants can be formulated in ISCOMs and/or ISCOM-Matrix structures. ISCOMs may be prepared as described in EP0109942B1, WO87/02250 and EP0180546BI. A transport and/or a passenger antigen may be used, as described in WO9730728A1.

The ISCOM may be an ISCOM matrix complex which comprises at least one saponin fraction and a lipid. The lipid may be a sterol, such as cholesterol. The ISCOM matrix complex may also contain a phospholipid, for example phosphatidylcholine. The ISCOM matrix complex may also contain one or more other immunomodulatory (adjuvant-active) substances, and may be produced as described in EP0436620B1. The ISCOM matrix may be formulated as an admixture with an antigen and the association between ISCOM matrix particles and antigen is mediated by electrostatic and/or hydrophobic interactions.

The ISCOM may be an ISCOM complex which contains at least one saponin, at least one lipid, and at least one type of antigen or epitope. The ISCOM complex contains antigen associated by detergent treatment such that a portion of the antigen integrates into the particle.

In some embodiments the saponin fraction or at least one additional adjuvant is selected from a QA derivative QA-Tri(X/R)-F*-GR-Ac (e.g. QA-Tri(X/R)-FRXA-GR-Ac), or QS-21, a semipurified preparation of Quillaja saponaria, a purified preparation of Quillaja saponaria, or any purified sub-fraction.

Each ISCOM particle may contain one or at least two saponin fractions. The ISCOM particle may contain the same or different weight % of the at least two saponin fractions. For example, the particle may contain any weight % of a QA derivative QA-Tri(X/R)-F*-GR-Ac and any weight % of another saponin fraction, such as QS-21. Accordingly, each ISCOM matrix particle or each ISCOM complex particle may contain from 0.1 to 99.9 by weight, 5 to 95% by weight, 10 to 90% by weight 15 to 85% by weight, 20 to 80% by weight, 25 to 75% by weight, 30 to 70% by weight, 35 to 65% by weight, 40 to 60% by weight, 45 to 55% by weight, 40 to 60% by weight, or 50% by weight of one saponin fraction, e.g. QA derivative QA-Tri(X/R)-F*-GR-Ac and the rest up to 100% in each case of another saponin e.g. QS-21. The weight is calculated as the total weight of the saponin fractions. Examples of ISCOM matrix complex and ISCOM complex adjuvants are disclosed in U.S. Application Publication No. 2013/0129770.

The ISCOM matrix or ISCOM complex may comprise from 5-99% by weight of one fraction, e.g. QA derivative QA-Tri(X/R)-F*-GR-Ac and the rest up to 100% of weight of another fraction e.g. QS-21. The ISCOM matrix or ISCOM complex may contain the same or different weight % of the at least two saponin fractions. The weight is calculated as the total weight of the saponin fractions. The ISCOM matrix or ISCOM complex may comprise from 40% to 99% by weight of one fraction, e.g. QA derivative QA-Tri(X/R)-F*-GR-Ac and from 1% to 60% by weight of another fraction, e.g. QS-21. The ISCOM matrix or ISCOM complex may comprise from 70% to 95% by weight of one fraction e.g., QA derivative QA-Tri(X/R)-F*-GR-Ac, and from 30% to 5% by weight of another fraction, e.g., QS-21.

ISCOM matrix particles and ISCOM complex particles may each be formed using only one saponin fraction. Compositions may contain multiple particles and each particle may contain only one saponin fraction. The compositions may contain one or more different types of particles (e.g. ISCOM-matrix complexes particles, ISCOM complexes particles), wherein each individual particle contains one saponin fraction. The saponin fraction in one particle may be different from the saponin fraction in the other particles.

One type of saponin fraction or a crude saponin fraction may be integrated into one ISCOM matrix complex or particle and another type of saponin fraction, or a crude saponin fraction, may be integrated into another ISCOM matrix complex or particle. A composition or vaccine may comprise at least two types of complexes or particles each type having one type of saponins integrated into physically different particles.

In the compositions, mixtures of ISCOM matrix complex particles and/or ISCOM complex particles may be used in which two saponin fractions are separately incorporated into different ISCOM matrix complex particles and/or ISCOM complex particles.

A composition may contain ISCOM matrix or ISCOM complex particles, which each have one saponin fraction. The composition can comprise the particles in different or the same weight %. For example, a composition may contain 0.1% to 99.9% by weight, 5% to 95% by weight, 10% to 90% by weight, 15% to 85% by weight, 20% to 80% by weight, 25% to 75% by weight, 30% to 70% by weight, 35% to 65% by weight, 40% to 60% by weight, 45% to 55% by weight, 40 to 60% by weight, or 50% by weight, of an ISCOM matrix or complex containing a first saponin fraction with the remaining portion made up by an ISCOM matrix or complex containing a different saponin fraction.

The saponin fraction in a first ISCOM matrix or ISCOM complex particle may be a QA derivative QA-Tri(X/R)-F*-GR-Ac, and the saponin fraction in a second ISCOM matrix or ISCOM complex particle may be QS-21.

Preferred compositions comprise a first ISCOM matrix containing QA derivative QA-Tri(X/R)-F*-GR-Ac, and a second ISCOM matrix containing QS-21, wherein the first ISCOM matrix constitutes about 70% per weight of the total saponin adjuvant, and the second ISCOM matrix constitutes about 30% per weight of the total saponin adjuvant. Another preferred composition comprises a first ISCOM matrix containing QA derivative QA-Tri(X/R)-F*-GR-Ac, and a second ISCOM matrix containing QS-21, wherein the first ISCOM matrix constitutes about 85% per weight of the total saponin adjuvant, and the second ISCOM matrix constitutes about 15% per weight of the total saponin adjuvant. Thus, in certain compositions, the first ISCOM matrix is present in a range of about 70% to about 85%, and the second ISCOM matrix is present in a range of about 15% to about 30%, of the total weight amount of saponin adjuvant in the composition.

The saponin-based adjuvant may be a Matrix-M™ adjuvant. The Matrix-M™ adjuvant may be extracted from the Quillaja saponaria Molina tree. The adjuvant can be formulated and purified with cholesterol and phospholipid. Matrix-M™ adjuvant may consist of two populations of individually formed particles which may have complementary properties. The particles may be about 25-55 nm, about 30-50 nm, or about 35-45 nm, preferably the particle is 40 nm.

One particle of the Matrix-M™ can be QA-derivative QA-Tri(X/R)-F*-GR-Ac (particle 1), and the other particle can be QS-21 (particle 2). Matrix-M™ may include the two particles in the ratios required to maintain high-adjuvant activity with optimal safety margin. For example, Matrix-M™ comprises 85% particle 1 and 15% particle 2. Matrix-M™ comprises 92% particle 1 and 8% particle 2.

The administration dose of Matrix-M™ adjuvant can be about 1 to about 100 μg, about 5 to about 95 μg, about 10 to about 90 μg, about 15 to about 85 μg, about 20 to about 80 μg, about 25 to about 75 μg, about 30 to about 70 μg, about 35 to about 65 μg, about 40 to about 60 μg, about 45 to about 55 μg about 50 μg, or any values in between.

The Matrix-M™ adjuvant can induce high and long-lasting levels of broadly reacting antibodies supported by a balanced TH1 and TH2 type of response, including biologically active antibody isotypes such as murine IgG2a, multifunctional T cells and cytotoxic T lymphocytes. Generally, Matrix-M™ adjuvant can enhance immune response and promote rapid and profound effects on cellular drainage to local lymph nodes creating a milieu of activated cells including T cells, B cells, natural killer cells, neutrophils, monocytes, and dendritic cells. Matrix-M™ can enhance the combination of antibody and cellular immune response.

A fifteenth aspect of the invention is an adjuvant composition comprising the QA-Tri(X/R)-F*-GR-Ac derivative, or QA-TriX-FRXA-GR-Ac according to the thirteenth aspect of the invention.

### EXAMPLES

The present invention is described with reference to the following, non-limiting examples:

### Example 1-Identification of QS-7-GlcT

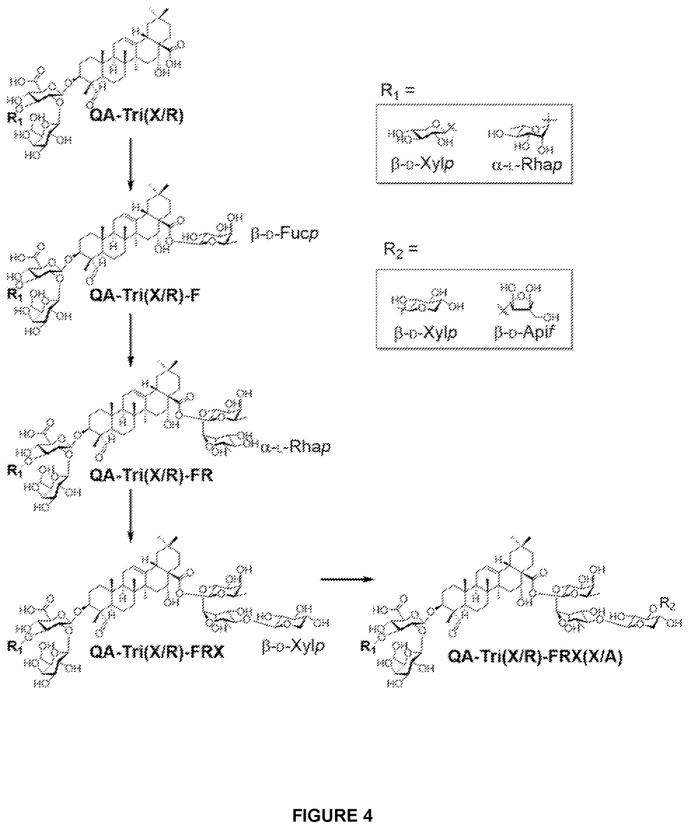

Previously, a series of genomic and transcriptomic sequence resources were generated for Q. saponaria and used to identify the genes required for production of the saponin QA-Tri(X/R)-FRX (A/X) (FIG. 4). This saponin serves as a common precursor between different immunostimulatory saponins produced by Q. saponaria including QS-21 and QS-7. Through these sequence resources, it has been established that genes required for biosynthesis of this scaffold show co-expression between different Q. saponaria tissues and are highly expressed in the leaf primordia.

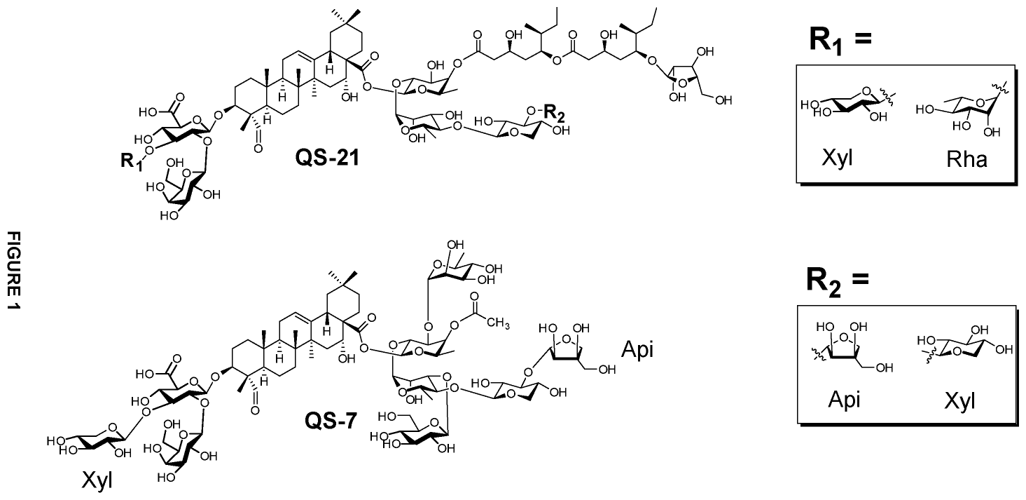

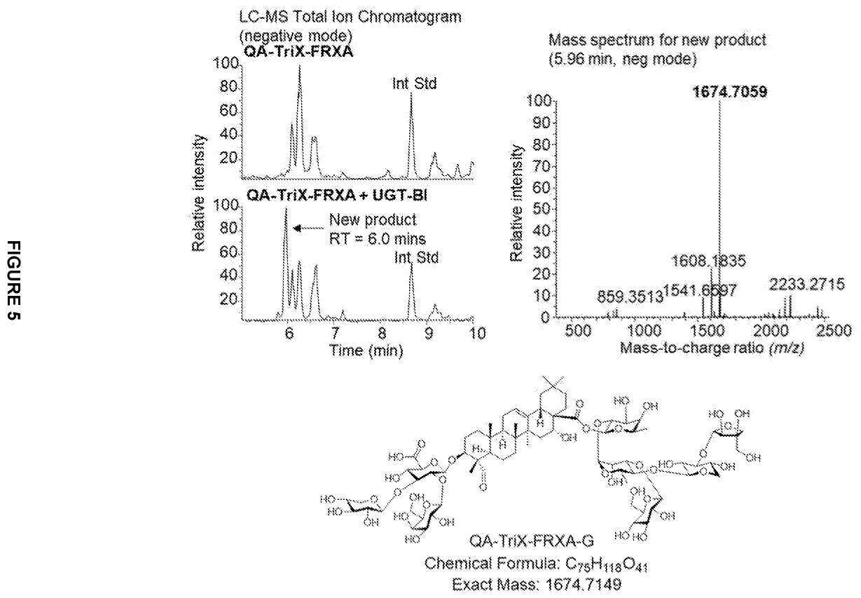

UDP-dependent glycosyltransferases (UGT) are commonly associated with glycosylation of plant natural products (Louveau & Osbourn, 2019) and several such enzymes are known to be required for the production of QA-Tri(X/R)-FRX (A/X). Using the sequence resources described above, one UGT was identified (QsUGT-BI) which showed a similar expression pattern to the previously characterised enzymes. Transient expression of QsUGT-BI with the genes from Q. saponaria required for biosynthesis of the QA-TriX-FRXA scaffold (Table 2) resulted in identification of a new product by LC-MS with a mass that suggested the addition of a hexose residue (FIG. 5). Several characterised saponins from Q. saponaria are known to feature glucose residues attached to the C-28 saccharide chain (Fleck et al., 2019), suggesting that the hexose added by QsUGT-BI was likely to be a glucose. One such saponin is QS-7, which features a D-glucose attached to the C-3 position of the rhamnose residue at C-28 (FIG. 1). The resulting product, putatively assigned as a QA-TriX-FRXA glucoside (QA-TriX-FRXA-G) was considered to be a precursor to QS-7. The putative glucosyltransferase QsUGT-BI is also referred to herein as QS-7-GlcT.

### Example 2-Identification of QS-7-AcetylT

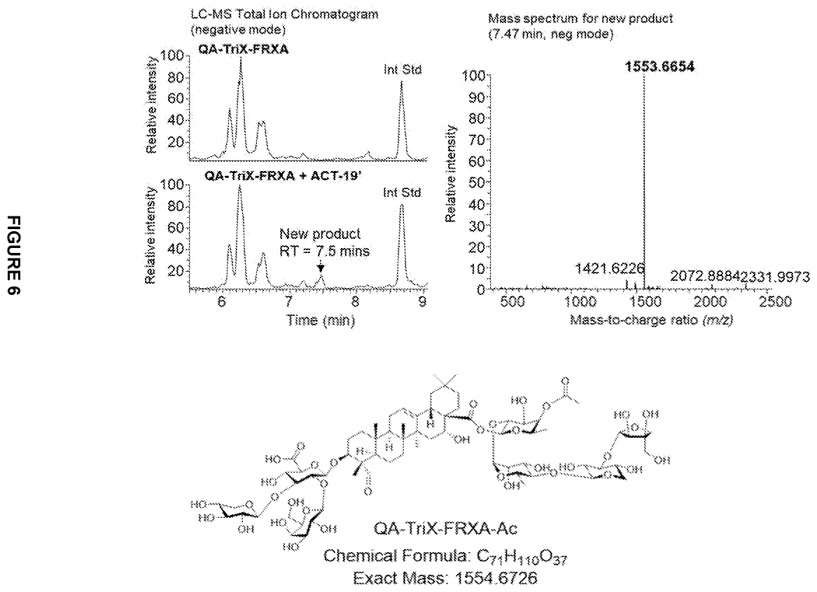

QS-7 features an acetyl group attached to the C-4 position of D-fucose (FIG. 1). BAHD acyltransferases are known to be commonly involved in acylation of various plant specialised metabolites. Consequently, a series of BAHD acyltransferases (ACTs) that showed co-expression to the known QA-TriX-FRXA-G pathway genes were cloned and tested in N. benthamiana by co-infiltrating the ACT candidates with the genes necessary to biosynthesise the QA-TriX-FRXA scaffold. Following LC-MS analysis of the leaf extracts, a new product was detected in the sample expressing the candidate “QsACT-19′”. This product was found to have the mass of QA-TriX-FRXA plus an acetyl group (QA-TriX-FRXA-Ac) indicating that the QsACT-19′ was an acetyltransferase (FIG. 6). The putative QA-TriX-FRXA-Ac product was therefore assumed to be a QS-7 precursor. The QsACT-19′ is also referred to herein as QS-7-AcetylT.

### Example 3-Identification of QS-7-RhaT

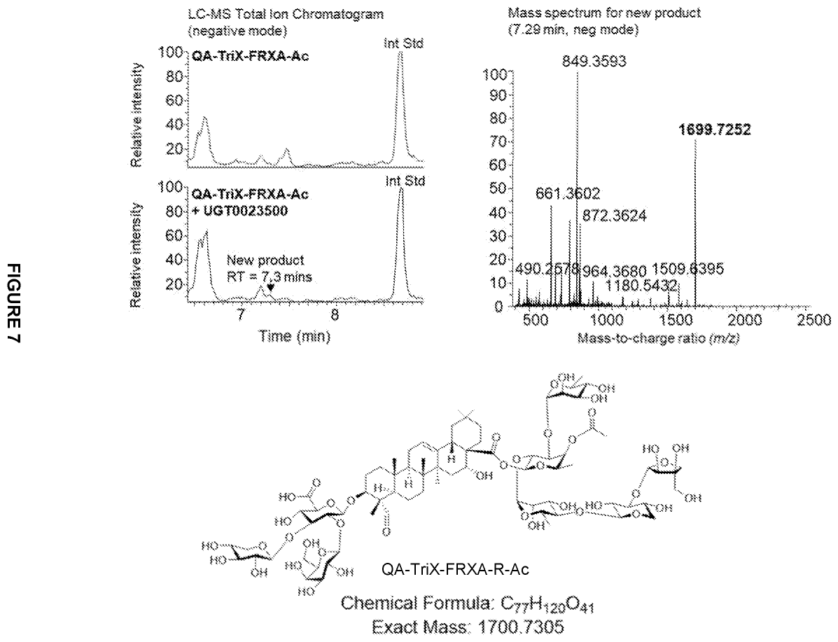

Analysis of the known saponins from Q. saponaria revealed that the presence of the additional rhamnose as found in QS-7 (attached to the C-3 position of D-fucose) appears to be dependent on the presence of the acetyl group attached to C-4 of D-fucose. The product of the QsACT-19′ enzyme as described above (QA-TriX-FRXA-Ac) featuring the acetyl group in the same position as QS-7 was used as a scaffold to screen a further pool of UGTs by transient expression. Amongst the candidates, one enzyme (QsUGT-0023500) resulted in appearance of a new peak that was consistent with addition of a deoxyhexose (such as rhamnose) to the QA-TriX-FRXA-Ac product (FIG. 7). The resulting product was putatively assigned as a QA-TriX-FRXA-Ac rhamnoside (QA-TriX-FRXA-R-Ac). QsUGT-0023500 is also referred to herein as Qs-7-RhaT.

### Example 4-Production of QS-7 (QA-TriX-FRXA-GR-Ac)

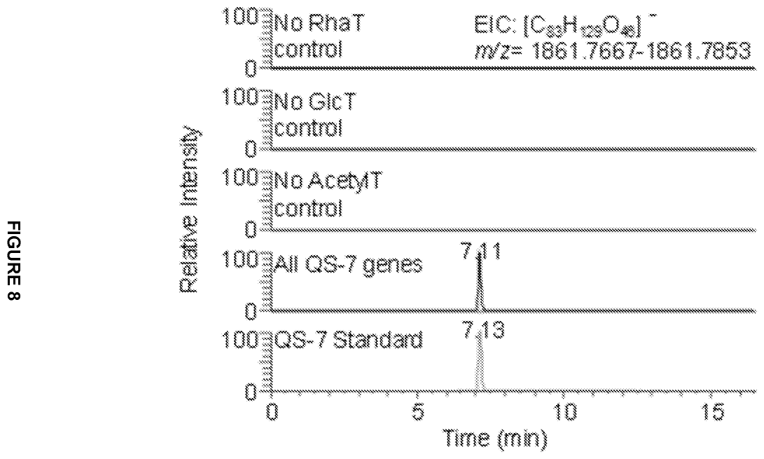

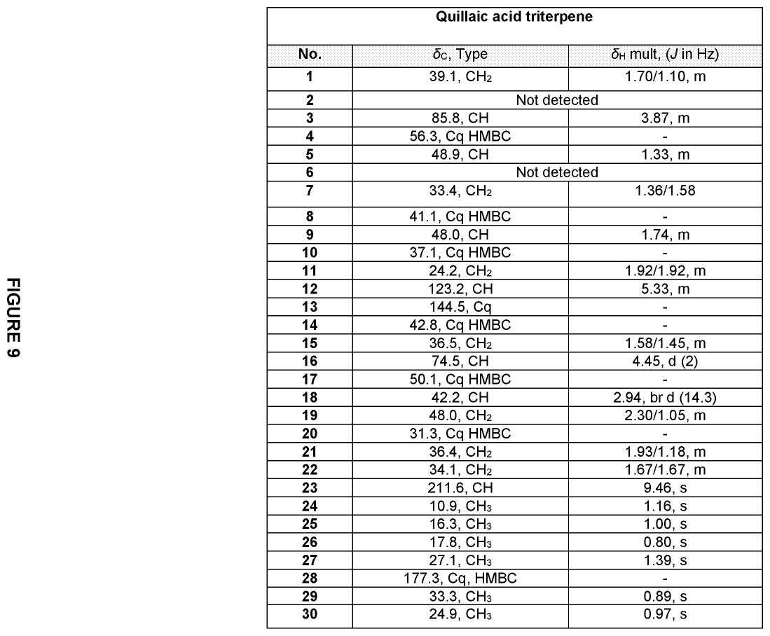

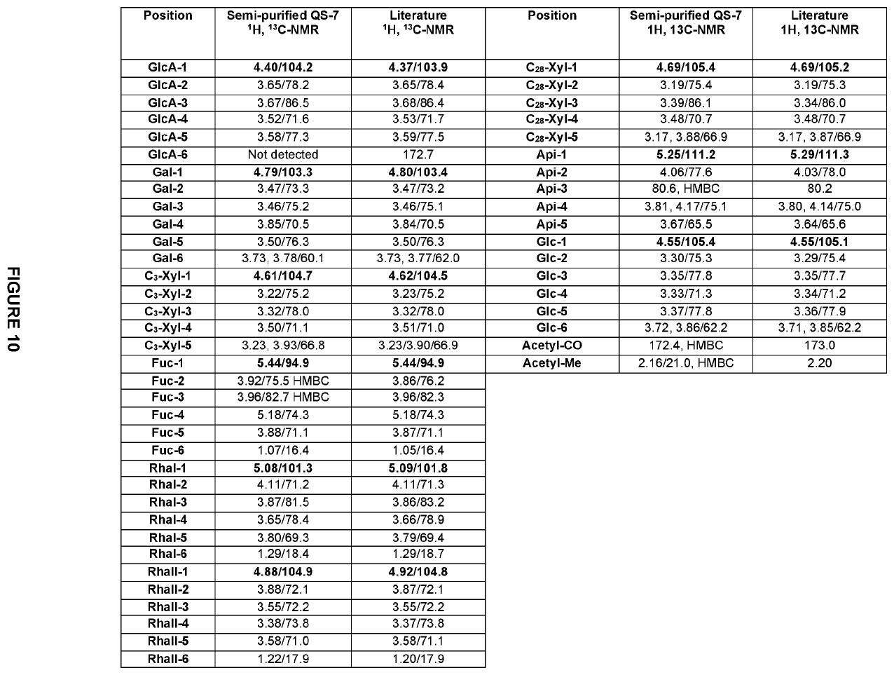

Following the identification of QsUGT-BI, QsUGT-0023500 and QsACT-19′, a further round of transient expression was performed combining the constructs necessary for production of QA-TriX-FRXA with the three newly identified genes. Subsequent LC-MS analysis of the leaf extracts revealed a peak which matched the retention time and mass spectrum of a QS-7 standard which is a fraction purified from the bark of Q. saponaria (FIG. 8) This peak was not present in any control samples in which any one of the three newly identified enzymes were absent. This appeared to confirm the relevance of these enzymes for QS-7 production. Nevertheless, to determine unequivocal QS-7 production, a large-scale infiltration was performed using a total of 410 plants. Extraction of the leaf material allowed for isolation of the new product by semi-preparative HPLC. The identity of the new product was confirmed by ¹H NMR analysis (FIGS. 9 and 10).

### Materials and Methods

### Primers and Cloning

The genes encoding the enzymes described herein (QS-7-RhaT/QsUGT-0023500, QS-7-GlcT/QsUGT-BI and QsAcetylT/QsACT-19′) were amplified by PCR from cDNA derived from leaf tissue of Q. saponaria. PCR was performed using the primers detailed in Table 1 and iProof polymerase with thermal cycling according to the manufacturer's recommendations. The resultant PCR products were purified (Qiagen PCR cleanup kit) and each cloned into the pDONR207 vector using BP clonase according to the manufacturer's instructions. The BP reaction was transformed into E. coli and the resulting transformants were cultured and the plasmids isolated by miniprep (Qiagen). The isolated plasmids were sequenced (Eurofins) to verify the presence of the correct genes. Next, each of the three genes were further subcloned into the pEAQ-HT-DEST1 expression vector using LR clonase. The resulting vectors were used to transform A. tumefaciens LBA4404 by flash freezing in liquid N2.

TABLE 1
Primers used to clone the genes described herein.
Gene specific sequences are shown in black, while
the attB sites required for Gateway® cloning are
shown in grey.

| Name | Sequence |
|---|---|
| UGT-BI_attB1F | GGGGACAAGTTTGTACAAAAAAGCAGGCTT |
| | AATGGCGGATCGAGTCATAAACAG |
| UGT-BI_attB2R | GGGGACCACTTTGTACAAGAAAGCTGGGTA |
| | TCAATTAGCTGCATTCGTGATATGC |
| UGT-0023500_attB1F | GGGGACAAGTTTGTACAAAAAAGCAGGCTT |
| | AATGACCAGTAATAATAGCCAACTCC |
| UGT-0023500_attB2R | GGGGACCACTTTGTACAAGAAAGCTGGGTA |
| | TTAATGCTTAAGAGATTTAAGACTCAACTC |
| Qs-ACT-19'_attB1F | GGGGACAAGTTTGTACAAAAAAGCAGGCTT |
| | AATGAAGATAGAAACCATTTCCACAAATTG |
| | C |
| Qs-ACT-19'_attB2R | GGGGACCACTTTGTACAAGAAAGCTGGGTA |
| | TTAAATAACAATAGGAATGGGATTAACAGT |
| | AGCAAATTG |

Agroinfiltration of N. benthamiana Leaves

Agroinfiltration was performed using a needleless syringe as previously described (Reed et al., 2017). All genes were expressed from pEAQ-HT-DEST1 binary expression vectors (Sainsbury et al., 2009) in A. tumefaciens LBA4404 as described above. In some cases multiple genes were integrated into a single Golden Gate binary vector for ease of infiltration. Cultivation of bacteria and plants is as described in (Reed et al., 2017).

Preparation of N. benthamiana Leaf Extracts for LC-MS Analysis

Leaves were harvested 5 days after agroinfiltration and lyophilised. Dried leaf material (10 mg per sample) was disrupted with tungsten beads at 1000 rpm for 1 min (Geno/Grinder 2010, Spex SamplePrep). Metabolites were extracted in 550 μL 80% methanol containing 20 μg/mL of internal standard (digitoxin (Sigma-Aldrich)) and incubated for 20 min at 18° C., with shaking at 1400 rpm (Thermomixer Comfort, Eppendorf). Each sample was defatted by partitioning twice with 400 μL hexane. The upper phase was discarded and the lower aqueous phase was dried under vacuum at 40° C. for 1 hour (EZ-2 Series Evaporator, Genevac). Dried material was resuspended in 75 μL of 100% methanol and filtered at 12, 500×g for 30 sec (0.2 μm, Spin-X, Costar). The filtrate (50 μL) was combined with 50 μL 50% methanol in glass vials and analysed as detailed below.

HPLC-CAD-MS Analysis of N. benthamiana Leaf Extracts

Sample analysis was performed using a QExactive mass spectrometer (Thermo Fisher) fitted with a 50×2.1 mm 2.6μ Kinetix XB-C18 column (Phenomenex). The solvent system consisted of water [A] and acetonitrile [B] both containing 0.1% formic acid with a flow rate of 0.6 mL/min. The program consisted of 15% [B] for the 0.75 min, followed by a gradient up to 60% [B] until 13 minutes. The percentage of [B] was increased to 100% over 0.25 min and held for 1 minute before returning to 15% over 0.25 seconds and held for 2 minutes. Samples were monitored by CAD and MS set to negative mode ranging from 400-2500 m/z.

Large Scale Vacuum Infiltration of N. benthamiana

Plants were infiltrated by vacuum as previously described (Reed et al., 2017; Stephenson et al., 2018) with A. tumefaciens LBA4404 strains carrying PEAQ-HT-DEST1 expression vectors harbouring relevant genes as detailed in Table 2.

Purification of QS-7 from Large Scale Infiltrations of N. benthamiana

A series of A. tumefaciens cultures containing the constructs relevant to QS-7 production were co-infiltrated into N. benthamiana by large scale vacuum infiltration. A total of 410 plants were agroinfiltrated and leaves were harvested after five days and lyophilised to give 104 g of dry material. The leaf material was initially defatted with hexane followed by subsequent exhaustive extraction using methanol. The methanol extracts were combined and evaporated under reduced pressure. The dried extract was dissolved in the least amount of methanol and diluted with an equivalent volume of water, before partitioning in a separatory funnel using a series of hexane, dichloromethane, ethyl acetate and n-butanol. The butanol layer was collected and dried over anhydrous NaSO₄, evaporated under reduced pressure, and re-dissolved in the least amount of methanol. Purification of QS-7 was performed using reverse phase semipreparative HPLC with a Luna C18 column 250×10 mm (particle size 5 μm) (Phenomenex). The mobile phase consisted of a weak solvent [A] (20 mM NH₄HCO₃ (pH 8.6) and strong solvent acetonitrile [B]. The separation program consisted of 25% [B] for an initial 2 mins, followed by a gradient from 25-70% over 18 minutes and held at 70% for 5 minutes. The column was equilibrated for 2 minutes at 25% [B] between runs. This afforded pure a semi-purified fraction (approx. 12 mg of pale brown amorphous material) that contained 3-5% QS-7 based on ¹H NMR analysis.

### NMR Analysis

1D and 2D NMR spectra were recorded on Bruker Avance 600 MHz spectrometer equipped with a BBFO Plus Smart probe and a triple resonance TCI cryoprobe, respectively (JIC, UK). The chemical shifts are relative to the residual signal solvent (MeOH-d4: δH 3.31; δC 49.15).

TABLE 2

| Constructs | Compound |
|---|---|
| tHMGR/QsbAS/Qs-CYP716-C8/Qs-CYP716-C- | QA-TriR-FRXA |
| 16/QsCYP714-C23/QsCSL1/QsC3-GalT/QsC3- | |
| RhaT/QsC28-FucT/Qs-C28-RhaT/QsC28- | |
| XylT3/QsC28ApiT4 | |
| tHMGR/QsbAS/Qs-CYP716-C8/Qs-CYP716-C- | QA-TriR-FRXA-G |
| 16/QsCYP714-C23/QsCSL1/QsC3-GalT/QsC3- | |
| RhaT/QsC28-FucT/Qs-C28-RhaT/QsC28- | |
| XylT3/QsC28ApiT4/QsUGT-Bl | |
| tHMGR/QsbAS/Qs-CYP716-C8/Qs-CYP716-C- | QA-TriR-FRXA-Ac |
| 16/QsCYP714-C23/QsCSL1/QsC3-GalT/QsC3- | |
| RhaT/QsC28-FucT/Qs-C28-RhaT/QsC28- | |
| XylT3/QsC28ApiT4/QsACT-19′ | |
| tHMGR/QsbAS/Qs-CYP716-C8/Qs-CYP716-C- | QA-TriR-FRXA-R-Ac |
| 16/QsCYP714-C23/QsCSL1/QsC3-GalT/QsC3- | |
| RhaT/QsC28-FucT/Qs-C28-RhaT/QsC28- | |
| XylT3/QsC28ApiT4/QsACT-19′/QsUGT- | |
| 0023500 | |
| tHMGR/QsbAS/Qs-CYP716-C8/Qs-CYP716-C- | QS-7 |
| 16/QsCYP714-C23/QsCSL1/QsC3-GalT/QsC3- | |
| RhaT/QsC28-FucT/Qs-C28-RhaT/QsC28- | |
| XylT3/QsC28ApiT4/QsUGT-BI/QsACT- | |
| 19′/QsUGT-0023500 | |

### CLAUSES

Embodiments of the invention are set out in the claims and in the clauses below.
1. A method of making a biosynthetic QA-Tri(X/R)-F*-GR-Ac in a host, which method comprises the steps of:
   a) expressing genes required for the biosynthesis of QA-TriR-F* and/or QA-TriX-F* into the host, and
   b) introducing a polynucleotide encoding:
      i. the enzyme QS-7-GlcT having the amino acid sequence of SEQ ID NO 56, or an enzyme having an amino acid sequence with at least 70% sequence identity to SEQ ID NO 56;
      ii. the enzyme QS-7-AcetylT having the amino acid sequence of SEQ ID NO 60, or an enzyme having an amino acid sequence with at least 70% sequence identity to SEQ ID NO 60, and
      iii. the enzyme QS-7-RhaT having the amino acid sequence of SEQ ID NO 58, or an enzyme having an amino acid sequence with at least 70% sequence identity to SEQ ID NO 58, into the host.
2. The method of clause 1, wherein Tri(X/R) is TriX and F* is FRXA.
3. The method of clause 1, wherein step a) comprises:
   1) expressing genes required for the biosynthesis of QA-TriR and/or QA-Trix into the host, and
   2) introducing a polynucleotide encoding:
      i. quillaic acid 28-O-fucosyltransferase (Qs-28-O-FucT, SEQ ID NO 2) or an enzyme with a sequence with at least 70% sequence identity to SEQ ID NO 2, optionally an enzyme from Q. saponaria boosting the production of fucosylated saponins (QsFucSyn, SEQ ID NO 12) or an enzyme with a sequence with at least 45% sequence identity to SEQ ID NO 12;
      ii. quillaic acid 28-O-fucoside [1,2]-rhamnosyltransferase (Qs-28-O-RhaT, SEQ ID NO 4) or an enzyme with a sequence with at least 70% sequence identity to SEQ ID NO 4;
      iii. quillaic acid 28-O-fucoside [1,2]-rhamnoside [1,4] xylosyltransferase (Qs-28-O-XyIT3, SEQ ID NO 6) or an enzyme with a sequence with at least 70% sequence identity to SEQ ID NO 6; and
      iv. optionally quillaic acid 28-O-fucoside [1,2]-rhamnoside [1,4] xyloside [1,3] xylosyltransferase (Qs-28-O-XyIT4, SEQ ID NO 8) or an enzyme with a sequence with at least 70% sequence identity to SEQ ID NO 8 and/or quillaic acid 28-O-fucoside [1,2]-rhamnoside [1,4] xyloside [1,3] apiosyltransferase (Qs-28-O-ApiT4, (SEQ ID NO 10) or an enzyme with a sequence with at least 70% sequence identity to SEQ ID NO 10 into the host.
4. The method of clause 1, wherein step a) comprises:
   1) expressing genes required for the biosynthesis of QA-TriR and/or QA-Trix into the host, and
   2) introducing a polynucleotide encoding:
      i. Qs-28-O-FucT (SEQ ID NO 2) or an enzyme with a sequence with at least 70% sequence identity optionally QsFucSyn (SEQ ID NO 12) or an enzyme with a sequence with at least 45% sequence identity;
      ii. Qs-28-O-RhaT (SEQ ID NO 4) or an enzyme with a sequence with at least 70% sequence identity;
      iii. Qs-28-O-XylT3 (SEQ ID NO 6) or an enzyme with a sequence with at least 70% sequence identity; and
      iv. optionally Qs-28-O-ApiT4 (SEQ ID NO 10) or an enzyme with a sequence with at least 70% sequence identity to SEQ ID NO 10.
into the host.
5. The method of clause 1, wherein step a) comprises:
   1) expressing genes required for the biosynthesis of QA-TriR and/or QA-Trix into the host, and
   2) introducing a polynucleotide encoding:
      i. Qs-28-O-FucT (SEQ ID NO 2) or an enzyme with a sequence with at least 70% sequence identity optionally QsFucSyn (SEQ ID NO 12) or an enzyme with a sequence with at least 45% sequence identity;
      ii. Qs-28-O-RhaT (SEQ ID NO 4) or an enzyme with a sequence with at least 70% sequence identity;
      iii. Qs-28-O-XyIT3 (SEQ ID NO 6) or an enzyme with a sequence with at least 70% sequence identity; and
      iv. optionally Qs-28-O-XyIT4 (SEQ ID NO 8) or an enzyme with a sequence with at least 70% sequence identity to SEQ ID NO 8.
into the host.
6. The method of clause 2, wherein step a) comprises:
   1) expressing genes required for the biosynthesis of QA-TriX into the host, and 2) introducing a polynucleotide encoding:
      i. Qs-28-O-FucT (SEQ ID NO 2) or an enzyme with a sequence with at least 70% sequence identity optionally QsFucSyn (SEQ ID NO 12) or an enzyme with a sequence with at least 45% sequence identity;
      ii. Qs-28-O-RhaT (SEQ ID NO 4) or an enzyme with a sequence with at least 70% sequence identity;
      iii. Qs-28-O-XyIT3 (SEQ ID NO 6) or an enzyme with a sequence with at least 70% sequence identity; and
      iv. Qs-28-O-ApiT4 (SEQ ID NO 10) or an enzyme with a sequence with at least 70% sequence identity to SEQ ID NO 10.
into the host.
7. The method of any one of clauses 3 to 5, wherein step 1) comprises introducing a polynucleotide encoding:
   i. quillaic acid 3-O-glucuronosyltransferase (QsCSL1, SEQ ID NO 26) or quillaic acid 3-O-glucuronosyltransferase (QsCsIG2, SEQ ID NO 28), or an enzyme with a sequence with at least 70% sequence identity to SEQ ID NO 26 or 28;
   ii. Q. saponaria QA-Mono β-1,2-D-galactosyltransferase (Qs-3-O-GalT, SEQ ID NO 30) or an enzyme with a sequence with at least 70% sequence identity; and
   iii. Q. saponaria QA-Di α-1,3-L-rhamnosyltransferase (DN20529_c0_g2_i8, SEQ ID NO 36), Q. saponaria QA-Di α-1,3-L-rhamnosyltransferase (Qs_0283850, SEQ ID NO 34), or Q. saponaria QA-Di dual 3-1,3-D-xylosyltransferase/α-1,3-L-rhamnosyltransferase (Qs-3-O-RhaT/XyIT, SEQ ID NO 32) or an enzyme with a sequence with at least 70% sequence identity to SEQ ID No 36, 34 or 32, and/or Q. saponaria QA-Di β-1,3-D-xylosyltransferase (Qs_0283870, SEQ ID NO 38) or Qs-3-O-RhaT/XyIT (SEQ ID NO 32) or an enzyme with a sequence with at least 70% sequence identity to SEQ ID NO 38 or 32;
into the host.
8. The method of any one of clauses 3 to 5, wherein step 1) comprises introducing a polynucleotide encoding:
   i. QsCSL1 (SEQ ID NO 26) or QsCsIG2 (SEQ ID NO 28), or an enzyme with a sequence with at least 70% sequence identity to SEQ ID NO 26 or 28;
   ii. Qs-3-O-GalT (SEQ ID NO 30) or an enzyme with a sequence with at least 70% sequence identity; and
   iii. DN20529_c0_92_i8 (SEQ ID NO 36), Qs_0283850 (SEQ ID NO 34), or Qs-3-O-RhaT/XyIT (SEQ ID NO 32) or an enzyme with a sequence with at least 70% sequence identity to SEQ ID No 36, 34 or 32 into the host.
9. The method of any one of clauses 2 to 6, wherein step 1) further comprises introducing a polynucleotide encoding:
   i. QsCSL1 (SEQ ID NO 26) or QsCsIG2 (SEQ ID NO 28), or an enzyme with a sequence with at least 70% sequence identity to SEQ ID NO 26 or 28;
   ii. Qs-3-O-GalT (SEQ ID NO 30) or an enzyme with a sequence with at least 70% sequence identity;
   iii. Qs_0283870 (SEQ ID NO 38) or Qs-3-O-RhaT/XyIT (SEQ ID NO 32) or an enzyme with a sequence with at least 70% sequence identity to SEQ ID NO 38 or 32
into the host.
10. The method of any one of clauses 2 to 9, wherein step a)-1) further comprises introducing a polynucleotide encoding:
   i. Q. saponaria β-amyrin synthase (QsbAS, SEQ ID NO 18) or an enzyme with a sequence with at least 50% sequence identity to SEQ ID NO 18;
   ii. Q. saponaria quillaic acid C-28 oxidase (QsCYP716-C-28, SEQ ID NO 20), or an enzyme with a sequence with at least 50% sequence identity to SEQ ID NO 20;
   iii. Q. saponaria quillaic acid C-16a oxidase (QsCYP716-C-16a, SEQ ID NO 22), or an enzyme with a sequence with at least 50% sequence identity to SEQ ID NO 22; and
   iv. Q. saponaria quillaic acid C-23 oxidase (QsCYP714-C-23, SEQ ID NO 24), or an enzyme with a sequence with at least 50% sequence identity to SEQ ID NO 24;
into the host.
11. The method of any one of clauses 2 to 10, wherein
   amino acid SEQ ID NO 2 is encoded by polynucleotide SEQ ID NO 1;
   amino acid SEQ ID NO 4 is encoded by polynucleotide SEQ ID NO 3;
   amino acid SEQ ID NO 6 is encoded by polynucleotide SEQ ID NO 5;
   amino acid SEQ ID NO 8 is encoded by polynucleotide SEQ ID NO 7;
   amino acid SEQ ID NO 10 is encoded by polynucleotide SEQ ID NO 9.
12. The method of any one of clauses 5 to 11, wherein:
   amino acid SEQ ID NO 26 is encoded by polynucleotide SEQ ID NO 25;
   amino acid SEQ ID NO 28 is encoded by polynucleotide SEQ ID NO 27;
   amino acid SEQ ID NO 30 is encoded by polynucleotide SEQ ID NO 29;
   amino acid SEQ ID NO 32 is encoded by polynucleotide SEQ ID NO 31;
   amino acid SEQ ID NO 34 is encoded by polynucleotide SEQ ID NO 33;
   amino acid SEQ ID NO 36 is encoded by polynucleotide SEQ ID NO 35;
   amino acid SEQ ID NO 38 is encoded by polynucleotide SEQ ID NO 37.
13. A method of making QA-Tri(X/R)-F*-GR-Ac, wherein the acetyl (Ac) moiety is attached to the C-4 position of the D-fucose of F*, the rhamnose (R) moiety is attached to the C-3 position of the D-fucose of F* and the glucose (G) moiety is attached to the C-3 position of the rhamnose moiety of F*, wherein the method comprises combining QA-Tri(X/R)-F* with
   i. the enzyme QS-7-GlcT having the amino acid sequence of SEQ ID NO 56, or an enzyme having an amino acid sequence with at least 70% sequence identity to SEQ ID NO 56;
   ii. one or more enzymes selected from the enzyme QS-7-AcetylT having the amino acid sequence of SEQ ID NO 60 or an enzyme having an amino acid sequence with at least 70% sequence identity to SEQ ID NO 60; the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64; and
   iii. the enzyme QS-7-RhaT having the amino acid sequence of SEQ ID NO 58, or an enzyme having an amino acid sequence with at least 70% sequence identity to SEQ ID NO 58,
to form QA-Tri(X/R) F*-GR-Ac.
14. The method of clause 13, wherein the method further comprises combining with:
   i. Qs-28-O-FucT (SEQ ID NO 2) or an enzyme with a sequence with at least 70% sequence identity, optionally QsFucSyn (SEQ ID NO 12) or an enzyme with a sequence with at least 45% sequence identity to SEQ ID NO 12;
   ii. Qs-28-O-RhaT (SEQ ID NO 4) or an enzyme with a sequence with at least 70% sequence identity to SEQ ID NO 4;
   iii. Qs-28-O-XylT3 (SEQ ID NO 6) or an enzyme with a sequence with at least 70% sequence identity to SEQ ID NO 6; and
   iv. optionally Qs-28-O-XyIT4 (SEQ ID NO 8) or an enzyme with a sequence with at least 70% sequence identity to SEQ ID NO 8 and/or Qs-28-O-ApiT4 (SEQ ID NO 10) or an enzyme with a sequence with at least 70% sequence identity to SEQ ID NO 10.
15. The method of clause 13, wherein the method further comprises combining with:
   i. Qs-28-O-FucT (SEQ ID NO 2) or an enzyme with a sequence with at least 70% sequence identity to SEQ ID NO 2, optionally QsFucSyn (SEQ ID NO 12) or an enzyme with a sequence with at least 45% sequence identity to SEQ ID NO 12;
   ii. Qs-28-O-RhaT (SEQ ID NO 4) or an enzyme with a sequence with at least 70% sequence identity to SEQ ID NO 4;
   iii. Qs-28-O-XyIT3 (SEQ ID NO 6) or an enzyme with a sequence with at least 70% sequence identity to SEQ ID NO 6; and
   iv. optionally Qs-28-O-ApiT4 (SEQ ID NO 10) or an enzyme with a sequence with at least 70% sequence identity to SEQ ID NO 10.
16. The method of clause 13, wherein the method further comprises combining with:
   i. Qs-28-O-FucT (SEQ ID NO 2) or an enzyme with a sequence with at least 70% sequence identity to SEQ ID NO 2, optionally QsFucSyn (SEQ ID NO 12) or an enzyme with a sequence with at least 45% sequence identity to SEQ ID NO 12;
   ii. Qs-28-O-RhaT (SEQ ID NO 4) or an enzyme with a sequence with at least 70% sequence identity to SEQ ID NO 4;
   iii. Qs-28-O-XyIT3 (SEQ ID NO 6) or an enzyme with a sequence with at least 70% sequence identity to SEQ ID NO 6; and
   iv. optionally Qs-28-O-XyIT4 (SEQ ID NO 8) or an enzyme with a sequence with at least 70% sequence identity to SEQ ID NO 8.
17. The method of clause 13, wherein Tri(X/R) is TriX and F* is FRXA.
18. The method of any clause 17, wherein the method further comprises combining with:
   i. Qs-28-O-FucT (SEQ ID NO 2) or an enzyme with a sequence with at least 70% sequence identity to SEQ ID NO 2, optionally QsFucSyn (SEQ ID NO 12) or an enzyme with a sequence with at least 45% sequence identity to SEQ ID NO 12;
   ii. Qs-28-O-RhaT (SEQ ID NO 4) or an enzyme with a sequence with at least 70% sequence identity to SEQ ID NO 4;
   iii. Qs-28-O-XyIT3 (SEQ ID NO 6) or an enzyme with a sequence with at least 70% sequence identity to SEQ ID NO 6; and
   iv. Qs-28-O-ApiT4 (SEQ ID NO 10) or an enzyme with a sequence with at least 70% sequence identity to SEQ ID NO 10.
19. The method of any one of clauses 13 to 16, wherein the method further comprises combining with:
   i. QSCSL1 (SEQ ID NO 26) or QSCSIG2 (SEQ ID NO 28), or an enzyme with a sequence with at least 70% sequence identity to SEQ ID NO 26 or 28;
   ii. Qs-3-O-GalT (SEQ ID NO 30) or an enzyme with a sequence with at least 70% sequence identity to SEQ ID NO 30; and
   iii. DN20529_c0_g2_i8 (SEQ ID NO 36), Qs_0283850 (SEQ ID NO 34), or Qs-3-O-RhaT/XyIT (SEQ ID NO 32) or an enzyme with a sequence with at least 70% sequence identity to SEQ ID No 36, 34 or 32, and/or Qs_0283870 (SEQ ID NO 38) or Qs-3-O-RhaT/XyIT (SEQ ID NO 32) or an enzyme with a sequence with at least 70% sequence identity to SEQ ID NO 38 or 32.
20. The method of any one of clauses 13 to 16, wherein the method further comprises combining with:
   i. QsCSL1 (SEQ ID NO 26) or QsCsIG2 (SEQ ID NO 28), or an enzyme with a sequence with at least 70% sequence identity to SEQ ID NO 26 or 28;
   ii. Qs-3-O-GalT (SEQ ID NO 30) or an enzyme with a sequence with at least 70% sequence identity to SEQ ID NO 30; and
   iii. DN20529_c0_92_i8 (SEQ ID NO 36), Qs_0283850 (SEQ ID NO 34), or Qs-3-O-RhaT/XyIT (SEQ ID NO 32) or an enzyme with a sequence with at least 70% sequence identity to SEQ ID No 36, 34 or 32.
21. The method of any one of clauses 13 to 18, wherein the method further comprises combining with:
   i. QsCSL1 (SEQ ID NO 26) or QsCsIG2 (SEQ ID NO 28), or an enzyme with a sequence with at least 70% sequence identity to SEQ ID NO 26 or 28;
   ii. Qs-3-O-GalT (SEQ ID NO 30) or an enzyme with a sequence with at least 70% sequence identity to SEQ ID NO 30; and/or
   iii. Qs_0283870 (SEQ ID NO 38) or Qs-3-O-RhaT/XyIT (SEQ ID NO 32) or an enzyme with a sequence with at least 70% sequence identity to SEQ ID NO 38 or 32.
22. The method of any one of clauses 13 to 21, wherein the method further comprises combining with:
   i. QsbAS (SEQ ID NO 18) an enzyme with a sequence with at least 50% sequence identity to SEQ ID NO 18;
   ii. QsCYP716-C-28 (SEQ ID NO 20) an enzyme with a sequence with at least 50% sequence identity to SEQ ID NO 20;
   iii. QsCYP716-C-16a (SEQ ID NO 22) an enzyme with a sequence with at least 50% sequence identity to SEQ ID NO 22, and iv. QsCYP714-C-23 (SEQ ID NO 24) an enzyme with a sequence with at least 50% sequence identity to SEQ ID NO 24.
23. The method of any preceding clause, wherein the method further comprises the step of isolating the QA-Tri(X/R)-F*-GR-Ac derivative.
24. The method of clause 23, wherein QA-Tri(X/R)-F*-GR-Ac is QA-TriR-FRXGR-Ac, QA-TriR-FRXX-GR-Ac, QA-TriR-FRXA-GR-Ac, QA-TriX-FRXGR-Ac, QA-TriX-FRXX-GR-Ac, QA-TriX-FRXA-GR-Ac, QA-Tri(X/R)-FRXGR-Ac, QA-Tri(X/R)-FRXX-GR-Ac and/or QA-Tri(X/R)-FRXA-GR-Ac or mixtures thereof
25. The method of clause 24, wherein QA-Tri(X/R)-F*-GR-Ac is QA-TriX-FRXA-GR-Ac.
26. The QA-Tri(X/R)-F*-GR-Ac obtainable by the method of clause 23.
27. The QA-Tri(X/R)-F*-GR-Ac of clause 26 wherein QA-Tri(X/R)-F*-GR-Ac is QA-TriR-FRXGR-Ac, QA-TriR-FRXX-GR-Ac, QA-TriR-FRXA-GR-Ac, QA-TriX-FRXGR-Ac, QA-TriX-FRXX-GR-Ac, QA-TriX-FRXA-GR-Ac, QA-Tri(X/R)-FRXGR-Ac, QA-Tri(X/R)-FRXX-GR-Ac and/or QA-Tri(X/R)-FRXA-GR-Ac, or mixtures thereof
28. The QA-Tri(X/R)-F*-GR-Ac of clause 27 wherein QA-Tri(X/R)-F*-GR-Ac is QA-TriX-FRXA-GR-Ac.

### Abbreviations

Apif—D-Apiofuranose
DMOT9—(3S,5S,6S)-3,5-dihydroxy-6-methyloctanoyl-CoA transferase 9
DN20529_c0_g2_i8—Q. saponaria QA-Di α-1,3-L-rhamnosyltransferase
FR—a disaccharide of a β-D-fucose (F) and a α-L-rhamnose (R) residue
FRX—a trisaccharide of a β-D-fucose (F), α-L-rhamnose (R) and a β-D-xylose (X) residue
FRXX—a tetrasaccharide of β-D-fucose (F), α-L-rhamnose (R), and two β-D-xylose (X, X) residues
FRXA—a tetrasaccharide of β-D-fucose (F), α-L-rhamnose (R), β-D-xylose (X) and a β-D-apiose (A) residue
FRXX/A—a tetrasaccharide which is FRXX or FRXA.
Fucp—D-Fucopyranose
FucSyn—enzyme boosting the production of fucosylated saponins
FSL—QsFucSyn-Like
Galp—D-Galactopyranose
GlcpA—D-Glucopyranuronic acid
Glcp—D-Glucopyranose
OS—2,3-oxidosqualene
OSC—oxidosqualene cyclase
QA—Quillaic acid
QA derivative
   QA-Di—3-O-{β-D-galactopyranosyl-(1->2)-β-D-glucopyranosiduronic acid}-quillaic acid
   QA-Di-F—3-O-{β-D-galactopyranosyl-(1->2)-β-D-glucopyranosiduronic acid}-28-O-{3-D-fucopyranosyl ester}-quillaic acid
   QA-Di-FR—3-O-{β-D-galactopyranosyl-(1->2)-β-D-glucopyranosiduronic acid}-28-O-{α-L-rhamnopyranosyl-(1->2)-β-D-fucopyranosyl ester}-quillaic acid
   QA-Di-FRX—3-O-{3-D-galactopyranosyl-(1->2)-β-D-glucopyranosiduronic acid}-28-O-{β-D-xylopyranosyl-(1->4)-α-L-rhamnopyranosyl-(1->2)-β-D-fucopyranosyl ester}-quillaic acid
   QA-Di-FRXA—3-O-{β-D-galactopyranosyl-(1->2)-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid
   QA-Di-FRXX—3-O-{β-D-galactopyranosyl-(1->2)-β-D-glucopyranosiduronic acid}-28-O-{β-D-xylopyranosyl-(1->3)-β-D-xylopyranosyl-(1->4)-α-L-rhamnopyranosyl-(1->2)-β-D-fucopyranosyl ester}-quillaic acid
   QA-Mono—3-O-{β-D-glucopyranosiduronic acid}-quillaic acid
   QA-Mono-F—3-O-{β-D-glucopyranosiduronic acid}-28-O-{β-D-fucopyranosyl ester}-quillaic acid
   QA-Mono-FR—3-O-{β-D-glucopyranosiduronic acid}-28-O-{α-L-rhamnopyranosyl-(1->2)-β-D-fucopyranosyl ester}-quillaic acid
   QA-Mono-FRX—3-O-{β-D-glucopyranosiduronic acid}-28-O-{3-D-xylopyranosyl-(1->4)-α-L-rhamnopyranosyl-(1->2)-β-D-fucopyranosyl ester}-quillaic acid
   QA-Mono-FRXA—3-O-{3-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid
   QA-Mono-FRXX—3-O-{β-D-glucopyranosiduronic acid}-28-O-{3-D-xylopyranosyl-(1->3)-β-D-xylopyranosyl-(1->4)-α-L-rhamnopyranosyl-(1->2)-β-D-fucopyranosyl ester}-quillaic acid
   QA-TriR—3-O-{α-L-rhamnopyranosyl-(1->3)-[3-D-galactopyranosyl-(1->2)]-β-D-glucopyranosiduronic acid}-quillaic acid
   QA-TriR-F—3-O-{α-L-rhamnopyranosyl-(1->3)-[3-D-galactopyranosyl-(1->2)]-β-D-glucopyranosiduronic acid}-28-O-{3-D-fucopyranosyl ester}-quillaic acid
   QA-TriR-FR—3-O-{α-L-rhamnopyranosyl-(1->3)-[3-D-galactopyranosyl-(1->2)]-β-D-glucopyranosiduronic acid}-28-O-{α-L-rhamnopyranosyl-(1->2)-β-D-fucopyranosyl ester}-quillaic acid
   QA-TriR-FRX—3-O-{α-L-rhamnopyranosyl-(1->3)-[β-D-galactopyranosyl-(1->2)]-β-D-glucopyranosiduronic acid}-28-O-{3-D-xylopyranosyl-(1->4)-α-L-rhamnopyranosyl-(1->2)-β-D-fucopyranosyl ester}-quillaic acid
   QA-TriR-FRXA—3-O-{α-L-rhamnopyranosyl-(1->3)-[3-D-galactopyranosyl-(1->2)]-β-D-glucopyranosiduronic acid}-28-O-{3-D-apiofuranosyl-(1->3)-β-D-xylopyranosyl-(1->4)-α-L-rhamnopyranosyl-(1->2)-β-D-fucopyranosyl ester}-quillaic acid
   QA-TriR-FRXX—3-O-{α-L-rhamnopyranosyl-(1->3)-[3-D-galactopyranosyl-(1->2)]-β-D-glucopyranosiduronic acid}-28-O-{3-D-xylopyranosyl-(1->3)-β-D-xylopyranosyl-(1->4)-α-L-rhamnopyranosyl-(1->2)-β-D-fucopyranosyl ester}-quillaic acid
   QA-TriX—3-O-{β-D-xylopyranosyl-(1->3)-[β-D-galactopyranosyl-(1->2)]-β-D-glucopyranosiduronic acid}-quillaic acid
   QA-TriX-F—3-O-{3-D-xylopyranosyl-(1->3)-[3-D-galactopyranosyl-(1->2)]-β-D-glucopyranosiduronic acid}-28-O-{β-D-fucopyranosyl ester}-quillaic acid
   QA-TriX-FR—3-O-{β-D-xylopyranosyl-(1->3)-[β-D-galactopyranosyl-(1->2)]-β-D-glucopyranosiduronic acid}-28-O-{α-L-rhamnopyranosyl-(1->2)-β-D-fucopyranosyl ester}-quillaic acid
   QA-TriX-FRX—3-O-{β-D-xylopyranosyl-(1->3)-[3-D-galactopyranosyl-(1->2)]-β-D-glucopyranosiduronic acid}-28-O-{3-D-xylopyranosyl-(1->4)-α-L-rhamnopyranosyl-(1->2)-β-D-fucopyranosyl ester}-quillaic acid
   QA-TriX-FRXA—3-O-{β-D-xylopyranosyl-(1->3)-[3-D-galactopyranosyl-(1->2)]-β-D-glucopyranosiduronic acid}-28-O-{3-D-apiofuranosyl-(1->3)-β-D-xylopyranosyl-(1->4)-α-L-rhamnopyranosyl-(1->2)-β-D-fucopyranosyl ester}-quillaic acid
   QA-TriX-FRXX—3-O-{β-D-xylopyranosyl-(1->3)-[3-D-galactopyranosyl-(1->2)]-β-D-glucopyranosiduronic acid}-28-O-{3-D-xylopyranosyl-(1->3)-β-D-xylopyranosyl-(1->4)-α-L-rhamnopyranosyl-(1->2)-β-D-fucopyranosyl ester}-quillaic acid
   QA-TriX-G—3-O-{β-D-xylopyranosyl-(1->3)-[3-D-galactopyranosyl-(1->2)]-β-D-glucopyranosiduronic acid}-28-O-{3-D-glucopyranosyl ester}-quillaic acid
   QA-TriX-GR—3-O-{β-D-xylopyranosyl-(1->3)-[3-D-galactopyranosyl-(1->2)]-β-D-glucopyranosiduronic acid}-28-O-{α-L-rhamnopyranosyl-(1->2)-β-D-glucopyranosyl ester}-quillaic acid
   QA-TriX-GRX—3-O-{β-D-xylopyranosyl-(1->3)-[3-D-galactopyranosyl-(1->2)]-β-D-glucopyranosiduronic acid}-28-O-{3-D-xylopyranosyl-(1->4)-α-L-rhamnopyranosyl-(1->2)-β-D-glucopyranosyl ester}-quillaic acid
   QA-Tri(X/R)—QA glycosylated at C-3 position with a branched trisaccharide which is either QA-Trix or QA-TriR
   QA-Tri(X/R)-F—QA glycosylated at C-28 and C-3 positions, which is either QA-TriX-F or QA-TriR-F
   QA-Tri(X/R)-FR—QA glycosylated at C-28 and C-3 positions, which is either QA-TriX-FR or QA-TriR-FR
   QA-Tri(X/R)-FRX—QA glycosylated at C-28 and C-3 positions, which is either QA-TriX-FRX or QA-TriR-FRX
   QA-Tri(X/R)-FRXA—QA glycosylated at C-28 and C-3 positions, which is either QA-TriX-FRXA or QA-TriR-FRXA
   QA-Tri(X/R)-FRXX—QA glycosylated at C-28 and C-3 positions, which is either QA-TriX-FRXX or QA-TriR-FRXX
   QA-Tri(X/R)-FRX(X/A)—QA glycosylated at C-28 and C-3 positions, which is either QA-TriX-FRXX, QA-TriX-FRXA, QA-TriR-FRXX or QA-TriR-FRXA
   QA-F—QA mono-glycosylated at the C-28 position.
   QA-FR—QA di-glycosylated at the C-28 position.
   QA-FRX—QA tri-glycosylated at the C-28 position.
   QA-FRXA—QA tetra-glycosylated at the C-28 position.
   QA-FRXX—QA tetra-glycosylated at the C-28 position.
   QA-FRX(X/A)—QA glycosylated at the C-28 position, which is either QA-FRXX or QA-FRXA.
   QA-TriX-FRXA-Ac—3-O-{β-D-xylopyranosyl-(1->3)-[β-D-galactopyranosyl-(1->2)]-β-D-glucopyranosiduronic acid}-28-O-{3-D-apiofuranosyl-(1->3)-β-D-xylopyranosyl-(1->4)-α-L-rhamnopyranosyl-(1->2)-β-D-fucopyranosyl ester}-quillaic acid acetylated at the C-4 position of the D-fucose of the C-28 chain.
   QA-TriX-FRXA-R-Ac—3-O-{β-D-xylopyranosyl-(1->3)-[β-D-galactopyranosyl-(1->2)]-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid acetylated at the C-4 position of the D-fucose of the core C-28 chain and with a rhamnose moiety attached to the C-3 position of the D-fucose of the C-28 chain.
   QA-TriX-FRXA-G—3-O-{β-D-xylopyranosyl-(1->3)-[β-D-galactopyranosyl-(1->2)]-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid glycosylated at the C-3 position of the core C-28 rhamnose moiety.
   QS-7 (or QA-TriX-FRXA-GR-Ac)—3-O-{3-D-xylopyranosyl-(1->3)-[β-D-galactopyranosyl-(1->2)]-β-D-glucopyranosiduronic acid}-28-O-{3-D-apiofuranosyl-(1->3)-β-D-xylopyranosyl-(1->4)-α-L-rhamnopyranosyl-(1->2)-β-D-fucopyranosyl ester}-quillaic acid acetylated at the C-4 position of the D-fucose of the core C-28 chain and with a rhamnose moiety attached to the C-3 position of the D-fucose of the C-28 chain and a glucose moiety attached to the C-3 position of the core C-28 rhamnose moiety.
Qs_0283850—Q. saponaria QA-Di α-1,3-L-rhamnosyltransferase
Qs_0283870—Q. saponaria QA-Di β-1,3-D-xylosyltransferase Qs-28-O-ApiT4—Quillaic acid 28-O-fucoside [1,2]-rhamnoside [1,4] xyloside [1,3] apiosyltransferase
Qs-28-O-FucT—Quillaic acid 28-O-fucosyltransferase
Qs-28-O-RhaT—Quillaic acid 28-O-fucoside [1,2]-rhamnosyltransferase
Qs-28-O-XyIT3—Quillaic acid 28-O-fucoside [1,2]-rhamnoside [1,4] xylosyltransferase
Qs-28-O-XyIT4—Quillaic acid 28-O-fucoside [1,2]-rhamnoside [1,4] xyloside [1,3] xylosyltransferase
Qs-3-O-GalT—Q. saponaria QA-Mono β-1,2-D-galactosyltransferase
Qs-3-O-RhaT—Q. saponaria QA-Di α-1,3-L-rhamnosyltransferase
Qs-3-O-RhaT/XyIT—Q. saponaria QA-Di dual β-1,3-D-xylosyltransferase/α-1,3-L-rhamnosyltransferase
Qs-3-O-XyIT—Q. saponaria QA-Di β-1,3-D-xylosyltransferase
QS-7-AcetylT—Quillaic acid 28-O-fucoside [1,4] acetyltransferase (also referred to as QsACT-19′).
QS-7-Glcτ-Quillaic acid 28-O-fucoside [1,2]-rhamnoside [1,3] glucosyltransferase (also referred to as QsUGT-BI)
QS-7-RhaT—Quillaic acid 28-O-fucoside [1,3] rhamnosyltransferase (also referred to as QsUGT-23500)
QsAXS1—UDP-D-apiose/UDP-D-xylose synthase
QsACT-19′—Quillaic acid 28-O-fucoside [1,4] acetyltransferase (also referred to as Qs-7-AcetylT)
QsbAS—Q. saponaria β-amyrin synthase
QsCSL1—Q. saponaria cellulose synthase-like enzyme (quillaic acid 3-O-glucuronosyltransferase)
QsCsIG2—Q. saponaria cellulose synthase-like enzyme (quillaic acid 3-O-glucuronosyltransferase)
QsCYP716-C-28—Q. saponaria quillaic acid C-28 oxidase
QsCYP716-C-16α—Q. saponaria quillaic acid C-16a oxidase
QsCYP714-C-23—Q. saponaria quillaic acid C-23 oxidase
QsFSL-1—Enzyme from Q. saponaria boosting the production of fucosylated saponins
QsFSL-2—Enzyme from Q. saponaria boosting the production of fucosylated saponins QsFucSyn-Enzyme from Q. saponaria boosting the production of fucosylated saponins
QsUGT-BI—, Quillaic acid 28-O-fucoside [1,2]-rhamnoside [1,3] glucosyltransferase (also referred to as QS-7-GlcT)
QsUGT-0023500—Quillaic acid 28-O-fucoside [1,3] rhamnosyltransferase (also referred to as QS-7-RhaT)
Rhap—L-Rhamnopyranose
SoFSL-1—Enzyme from S. officinalis boosting the production of fucosylated saponins
UDP-sugar—Uridine diphosphate sugar
UGT—UDP-dependent glycosyltransferases
Xylp—D-Xylopyranose

### REFERENCES

Fleck J D, Betti A H, da Silva F P, Troian E A, Olivaro C, Ferreira F, Verza S G. 2019. Saponins from Quillaja saponaria and Quillaja brasiliensis: Particular Chemical Characteristics and Biological Activities. Molecules 24 (1).
Kensil C R, Patel U, Lennick M, and Marciani D, 1991. Separation and characterization of saponins with adjuvant activity from Quillaja saponaria Molina cortex, J Immunol. 146 (2) 431-437.
Louveau T, Osbourn A. 2019. The Sweet Side of Plant-Specialized Metabolism. Cold Spring Harb Perspect Biol (In Press).
Reed J, Osbourn A. 2018. Engineering terpenoid production through transient expression in Nicotiana benthamiana. Plant Cell Reports.
Reed J, Stephenson M J, Miettinen K, Brouwer B, Leveau A, Brett P, Goss R J M, Goossens A, O'Connell M A, Osbourn A. 2017. A translational synthetic biology platform for rapid access to gram-scale quantities of novel drug-like molecules. Metab Eng 42: 185-193.
Sainsbury F, Thuenemann E C, Lomonossoff G P. 2009. PEAQ: versatile expression vectors for easy and quick transient expression of heterologous proteins in plants. Plant Biotechnol J 7 (7): 682-693.

Stephenson M J, Reed J, Brouwer B, Osbourn A. 2018. Transient Expression in Nicotiana Benthamiana Leaves for Triterpene Production at a Preparative Scale. Journal of visualized experiments: JoVE (138): 58169.

P202303GB - Annex A
A nucleic acid sequence which encodes the
enzyme according to SEQ ID NO 2.
SEQ ID NO 1
ATGGAGAATGGGAGAGTTTACAAATCCCATGTCGTGGTGCTCGCATTTCA
CGGGCAAGGCCATATTGTTCCGTTAATCCAATTATCCAGACGATTGGCCT
GGAAAGGCATCAAAATCACATTTGCTACAACACATTCTTGCACCAAGGCC
ATTCAAACAGGAAGTGATTCAATTTCACTTTTATCAATTTATGATGACAT
AACTGATGGTGGGTTTCAAGGAGAAGGAGGATTCAAGGGCTTCCTTCAGA
GATTTGAAGCCAGTACCACAAGGATCTTACACGAATTCGTCAAGAATCAT
GAAAACTCAAAGAACCCAGTAAAATGCTTAATATATGATGCTAACTTAAT
ATGGGCTCTGGAAATGGCAAAGCAATTGGGTATTGCTACTGCTGCATTTG
TGTTTCCTTCTTGGGCTGCCATTGCCACCTACTATCCCTTTTATTTAGAG
GTGTATGCGGATCAGCAGATAAAGAAGGTAGATCCTTTCACAATGCCTGA
CTTACCTCCACAACTTGGACTTCCAAATATGGCATCTCTCGGTTCAGATT
CGGGTCAACACTCCCCCATACTCAAACTCATGTTGCAACAGTTAGAAAAT
TTTGGGAAAGCTGACTGGATCCTGTCTCACGCATTTGAACAGTTTGAACA
AGAGGTACTTGACTGGATGAGAAATATCAGCCCAGTAACAACAATTGGTC
CAACTCTGCCATCTGTTTATCTTGATGGTAGGCTAAAAGATGACACAGAT
TACGGTTACAATTTGTACAAGCCAGATAGTGATACCTGCATGAAGTGGCT
AGACACTAAGGAAACTGAATCAGTGGTTTATATATCATTTGGCAGTGTTG
CAGATTTGATCCCAGAACAGATGACAGAAATAACAAACTCCCTGAAGAAA
ATGAGCAGCAACTTTCTGTGGGTGGTGAAGGAAACTGAAAAAAACAACCT
CCCTAGCAGCTTTGTTGAGGAGACAAAAGAAAAGGGATTGGTAGTGACTT
GGTGCCCCCAGTTGAAGGTGTTGTCTCATCCTGCAGTGGGTTGTTTCATT
ACACACTGTGGAACAAATTCCATATTTGAGTCAGTATGCTTTGCAGTGCC
AATGGTGGGAATGCCACAGTTTTGTGATCAAATGCCTAATGCATATTTCA
TGGAGAAGGTCTGGAAAGTAGGTGTTAGGCCAAGTTTGGATGACAATGGT
GTCGTCACTGGAGAAGAAATTGAGCGATGTATAAAAGTAGTTACCGAAGG
AGAGAGTGGGCAAGAGATTAAGAAGAAACTTGTGCAGTGGAAAGAGCTTG
CAAAAGAGGCAGTGGACGAGGGTGGAAGTTCAGATAAGCACATTGATGAA
TTCATTGCTGGAATCACAACTTGA*
A fucosyltransferase enzyme capable of
transferring β-D-fucopyranose to the C-28
position of Quallic acid (Qs-28-O-FucT).
SEQ ID NO 2
MENGRVYKSHVVVLAFHGQGHIVPLIQLSRRLAWKGIKITFATTHSCTKA
IQTGSDSISLLSIYDDITDGGFQGEGGFKGFLQRFEASTTRILHEFVKNH
ENSKNPVKCLIYDANLIWALEMAKQLGIATAAFVFPSWAAIATYYPFYLE
VYADQQIKKVDPFTMPDLPPQLGLPNMASLGSDSGQHSPILKLMLQQLEN
FGKADWILSHAFEQFEQEVLDWMRNISPVTTIGPTLPSVYLDGRLKDDTD
YGYNLYKPDSDTCMKWLDTKETESVVYISFGSVADLIPEQMTEITNSLKK
MSSNFLWVVKETEKNNLPSSFVEETKEKGLVVTWCPQLKVLSHPAVGCFI
THCGTNSIFESVCFAVPMVGMPQFCDQMPNAYFMEKVWKVGVRPSLDDNG
VVTGEEIERCIKVVTEGESGQEIKKKLVQWKELAKEAVDEGGSSDKHIDE
FIAGITT*
A nucleic acid sequence which encodes
the enzyme according to SEQ ID NO 4.
SEQ ID NO 3
ATGGCAAAAACTGATAAGCAGCTTCACATCGCCATGTTCCCATGGCTAGC
TATGGGTCATATATTCCCAAACTTTGAGCTCGCTAAGCTCTTTGCTCAAA
AGGGTCACTCAATTACTTTAATCTCCACCCCACGAAACATCAGTCGTCTC
CCTCAAATCCCTACACATTTAGAGCAATTGATTAAATTAGTCAGCTTGCC
TATATTACCCAAACACAAAGCAAATCTCCCAGAGAATGCAGAGTCCACCA
TGGACGTTACCCCCAATAAAGTCCCATACCTTAAGATGGCCTATGATGGT
CTTCAGGAGTCGTTGACTCAATTACTGAAATCTTCAGCTCCCGATTGGAT
TCTATATGACTTTGCTGCTGACTGGTTACCACCACTTGTTCACAGCCTTC
AAATCCGCTGTGTTTTCTTCGTGGTATCCCCTGCGTGGAATCTTTGCTTC
TTTGACACTCCCAAACCACAGTTGGGCAGTGCTGCTGTTTTTCGAACAAA
GCCTGAAGACTATCTTCGCCCTCCCAGTTGGGTTCCTTTCCATTCAAATA
TTGGGCTAAAGCTTCACGAGGTGAAGAAAATGTTTGAAGGGGTTTCAGAT
AAAGAAACAGGGGTCACTGTAAGTTTTAACTTCAACAAAGCAGTTTCGAG
CTGTGACTTGTTTTCTTTCCGCAGCTGCTATGAACTCGAATCAGAATGGC
TGAACCTGGTGGAGGATATTTACAAGAGGCCTGTAGTTCCAGTGGGCGTA
ATTCCACCCTCTTTTCAAGTCAGAATTGTGAATGAAGAAGACAACAAACC
AGAGTGGTTAAAGATCCAATCTTGGTTAGATAAACAAGAGCAAGGATCGG
TGGTATACATAGCATTTGGCAGTGAGCTTAAGCTGGGCCAACAAGATCTC
ACCGAATTAGCTCTTGGACTTGAGCTTTCTGGGTTGCCATTCTTTTGGGC
ACTTAGAAAGCAGCAAGACAGCTCATCAGTAGATTTACCAGATGGGTTTG
AGGACCGAGTCAGTGATCGTGGAGTTGTTTGCAGAGACTGGGTGCCCCAA
CTTAAGATCCTAGCTCACGGGTCAATTGGGGGTTATTTGACTCACTGTGG
TTCAGGTTCAGTGATAGAGGGACTTCATTTTGGGCGTGTTCTTGTTATGC
TGCCCTATTTACTAGACCAAGCATTATATGCTAGAGTATTGGAGGAGAAA
AAGCTGGGGGTTGAGATACCAAGGAACGAACAAGATGGGTCTTTTACTAG
GAGCTCAGTGGCCAAGTCTGTGAAGTTGGCCATAGTGGATGAGGGGGGAA
GTATTTACAGGGACAAAGCCAAAGAGATGGGCTTGGTATTCAGTGACAAA
GATCGTCATGAACAATACATTGAGAATTTCCTTCAACACCTTCAACACAA
AAGGGAACCTTTCCAAATTTAA
A rhamnosyltransferase enzyme, capable of
transferring α-1,2-I-rhamnopyranose to
QA-F (Qs-28-O-RhaT).
SEQ ID NO 4
MAKTDKQLHIAMFPWLAMGHIFPNFELAKLFAQKGHSITLISTPRNISRL
PQIPTHLEQLIKLVSLPILPKHKANLPENAESTMDVTPNKVPYLKMAYDG
LQESLTQLLKSSAPDWILYDFAADWLPPLVHSLQIRCVFFVVSPAWNLCF
FDTPKPQLGSAAVFRTKPEDYLRPPSWVPFHSNIGLKLHEVKKMFEGVSD
KETGVTVSFNFNKAVSSCDLFSFRSCYELESEWLNLVEDIYKRPVVPVGV
IPPSFQVRIVNEEDNKPEWLKIQSWLDKQEQGSVVYIAFGSELKLGQQDL
TELALGLELSGLPFFWALRKQQDSSSVDLPDGFEDRVSDRGVVCRDWVPQ
LKILAHGSIGGYLTHCGSGSVIEGLHFGRVLVMLPYLLDQALYARVLEEK
KLGVEIPRNEQDGSFTRSSVAKSVKLAIVDEGGSIYRDKAKEMGLVFSDK
DRHEQYIENFLQHLQHKREPFQI*
A nucleic acid sequence which encodes the
enzyme according to SEQ ID NO 6.
SEQ ID NO 5
ATGGCTGCTGCAGCTCCCAATCACAGGCTCCACATAGCATTCTTCCCATG
GTTAGCATTTGGTCACATAAACCCTTTCTTTGAGCTTGCGAAGCTCATTG
CTCAAAAGGGTCATCATATTTCTTTCATTTCCACCCCAAGAAACATCCAA
CGCCTTTCACAAGTTCCTCCACAATTAGCAGATTCCATAGATCTAGTGAG
CTTACCAGTAATCCATAATTCAAACCTCCCAGAAAACGCAGAGTCCACCA
TGGACATTCCACCTGATAAAACCCCTTACCTTGGGATGCTTCACGACAGT
CTCAAAGAACCCCTTACTCAATTCCTTCAAACTCATTCCCCTGATTGGAT
TCTGTATGACTTTTCAGCTGGTTGGCTTGCTGCCATAGTAGAAGACCTTG
GTATCTCCCACGGCTACTTTTCTATCATTCCCTGTTGGAACATAGGCTTC
AATGGACGCCAAATGAATGGTTTCCAAAAGCCAGATATTTCATTGCCCTC
CGCCGTGTCGCTTAAGAAATATGAGGTGAAGAAAATCATGGATTTGGTCA
AATCTTTTCCCAAAATTTTGGATGAGTCGGCCACTAAATCCATAGCTTCG
CATTCGACCTGTGAAGTAATTTTTATACGAAATTGCCCCGAGATTGAAGC
AGATTGGTTCGACTATACTTCGAAAATTTTCGATAAACCAGTGGTTCCGG
TGGGCGTAGTGCCCCCATCTGTGCACATAACTAACAAAGAGAAGGACGAG
CATTTCAACAAATGGTTGGAGATCAAAGAATGGTTGGATCAACAAGACAG
AGGTTCTGTAATTTATATAGCTTTTGGAACTGAATCACTGCCAAATCAGG
ATGAAATCACCATGCTTGCTCAAGGGCTTGAGCTATGTGGGCTTCCTTTC
TTTTGGGCATTAAGGAAGTCTAATGTGGCTTCTGATCAGCCAAATTCAGA
CTCAGTTGAGCTACCGGAGGGATTTGAAGAACAAACCAAAGGTCGTGGAA
TTGTGTGGACGAGTTGGGCACCTCAACAGAGAATTCTGGGTCACAATTCA
ATTGGGGGTTTTGTGACTCACTGTGGTTGGAGTTCAGTAATAGAAGGAAT
TCACTATGGACGGCCACTGATTATGTTCCCTCTAACAGTCGAACAGTCTC
TGAATGCTAGGATTTTGGGGGAGAAGAAGTTGGGTATGGAAGTACCCAGA
GAAGATGATGGGTCTTTTACAGGTGAAGTTGTGGCAGAGACATTGAAGCT
GGTATTGCTGGACCAAGATGGGAAAGTTTACAGGGACAAGGTAACAGAGA
TGAGTAAGGTATTTGGGGACAAAGACAAACATGAGAAATACATGGGTGAT
CTACTTGAATTCTTCAAAAATTACAGGTCTCTTAAGAGGAATTAG
A xylosyltransferase enzyme capable of
transferring β-1,4-D-xylopyranose to
QA-FR (Qs-28-O-XylT3)
SEQ ID NO 6
MAAAAPNHRLHIAFFPWLAFGHINPFFELAKLIAQKGHHISFISTPRNIQ
RLSQVPPQLADSIDLVSLPVIHNSNLPENAESTMDIPPDKTPYLGMLHDS
LKEPLTQFLQTHSPDWILYDFSAGWLAAIVEDLGISHGYFSIIPCWNIGF
NGRQMNGFQKPDISLPSAVSLKKYEVKKIMDLVKSFPKILDESATKSIAS
HSTCEVIFIRNCPEIEADWFDYTSKIFDKPVVPVGVVPPSVHITNKEKDE
HFNKWLEIKEWLDQQDRGSVIYIAFGTESLPNQDEITMLAQGLELCGLPF
FWALRKSNVASDQPNSDSVELPEGFEEQTKGRGIVWTSWAPQQRILGHNS
IGGFVTHCGWSSVIEGIHYGRPLIMFPLTVEQSLNARILGEKKLGMEVPR
EDDGSFTGEVVAETLKLVLLDQDGKVYRDKVTEMSKVFGDKDKHEKYMGD
LLEFFKNYRSLKRN*
A nucleic acid sequence which encodes
the enzyme according to SEQ ID NO 8.
SEQ ID NO 7
ATGGACTCCACCCACTTGCAGCCGGCCACTACTCCTCTGAAAATTCACTT
CATACCTTTCATATCTCCGGGTCACATCATCCCACTCTCTGAATTGGCTC
GTATCTTTGCTTCACGCGGTGAGCATGTGACTATCATTACCACTCCTTTC
AACGCTGACCTACTTCAGAAATCCATTGACGAAGACAGAGATTCCGGCGA
GCACATTGGCATCCACACCGTTGAATGGTCAGCCACAGAGCTAGGCCTTC
CTCATGGGATTGAAAATCTCAGCAACGTCACTGACTTGGAGACCGCCATT
AAGCTACACAGGGCCCTCATGCTAATGCAGAAACAGATGGAAGATTTCAT
GACCCGAAATCCACCCGATTGTATAATTGCCGACACGTTTTACCCGTGGG
CCTCCGAATTTGCTAATCGGATGGGTATCCCGAGACTCATTTTCTACCCG
TGGAGTACTTTCGCGCTCTGTTTGATGGAATCTATTCGATCTCCCGACTC
CCCGCACCGAAGATTGAGTTCGGATTCGGATCCATTTGTGGTTCCGGGTC
TTCCTCACCCGATCATCTTGACCCGTTCTCAGCTTCCGGAACACGATCGA
AAGGACATAGCGGACCCGGCTGCCCAACTCATGGATCAGCATAAAGAAAC
TGAGATGAAGAGCTACGGAATTATTCTCAACAATTTCGCGGAGATCGAAA
CAGAATACACAGAGCATTACAAGAAAATAACGGGTCACAAGGTTTGGCAC
ATTGGACCTGCCGCAGCAATTGTTCACCGAAATGCCAAAGAGAAGGCAGA
GAGGGTATTCAAGAGTGATGAGCATGACAATAACCTTGTCATCAATTGGC
TCAACTCGAAGGAACCAAACTCAGTTGTTTATGTTTGTTTCGGCAGCGGA
TGTCAATTCCCTGATAAACAACTCTATGAGATTGCATGCGGGTTAGAGTT
ATCTGGGCATCAATTTGTTTGGGTGGTTCGCGGAAAAGATAAACAAATCG
ATGTTAATGACGATGGGGAGAAGACATGGTTGCCTAAAGGGTTTGAGGAA
AGAATGAAAACAGAAAATAAAGGTTTGATTGTAAGGGGATGGGCCCCACA
GGTGCTGGTTTTGGATCATCCATCATTGGGATGTTTCTTGACGCATTGCG
GCTGGAATTCTACGATTGAGGGAATCACAGCAGGCGTTCCTTTGATCACG
TGGCCAGTATTCGCCGAGCAATTCTATAATGAGAAGCTAATCACGCAGGT
GCATGGGAATGGGGTGGTGGTTGGTTCAGAGGAGTGGATCATGTTGTTCA
CCGTCGCTAAAAGCTTGGTAAGTAGAGACAAAATTGAGAATGCTGTGAGG
AAGATAATGGACGGTGGTGATGAGGCTGTACAAATCAGAAGGCGGGCCCG
GGAACTTGGAGAAAAAGCTTGGAAAGCTGCTTCAACTGGGGGGTCCTCCT
ACAATAATCTAACCGCAGCAATTGAAGACCTTAAGCGGTTGAGAGAGGAC
CGTTCGAAGCTGAAAACAAAAACAATTTGA
A xylosyltransferase enzyme capable of
transferring β-1,3-D-xylopyranose to
QA-FRX (Qs-28-O-XylT4)
SEQ ID NO 8
MDSTHLQPATTPLKIHFIPFISPGHIIPLSELARIFASRGEHVTIITTPF
NADLLQKSIDEDRDSGEHIGIHTVEWSATELGLPHGIENLSNVTDLETAI
KLHRALMLMQKQMEDFMTRNPPDCIIADTFYPWASEFANRMGIPRLIFYP
WSTFALCLMESIRSPDSPHRRLSSDSDPFVVPGLPHPIILTRSQLPEHDR
KDIADPAAQLMDQHKETEMKSYGIILNNFAEIETEYTEHYKKITGHKVWH
IGPAAAIVHRNAKEKAERVFKSDEHDNNLVINWLNSKEPNSVVYVCFGSG
CQFPDKQLYEIACGLELSGHQFVWVVRGKDKQIDVNDDGEKTWLPKGFEE
RMKTENKGLIVRGWAPQVLVLDHPSLGCFLTHCGWNSTIEGITAGVPLIT
WPVFAEQFYNEKLITQVHGNGVVVGSEEWIMLFTVAKSLVSRDKIENAVR
KIMDGGDEAVQIRRRARELGEKAWKAASTGGSSYNNLTAAIEDLKRLRED
RSKLKTKTI*
A nucleic acid sequence which encodes
the enzyme according to SEQ ID NO 10.
SEQ ID NO 9
ATGGACTCCACCCACTTGCAGCCGGCCACTACTCCTCTGAAAATTCACGT
CATACCTTTCATAGCTCCGGGTCACATTATCCCACTCTCTGAATTGGCTC
GTATCTTTGCTTCACGCGGTGAGCATGTGACTATCATAACCACTCCTTTC
AACGCTGACCTACTTCAGAAATCCATTGACGAAGACAGAGATTCCGGCGA
GCACATTGGCATCCACACCGTTGAATGGTCAGCCACAGAGCTAGGCCTTC
CTCATGGGGTTGAAAATCTCAGCAACGTCACTGACTTGGAGACCGGCATT
AAGCTACACAGGGCCCTCGTGCTAATGCAGAAACAGATGGAAGATTTCAT
GACCCGAAATCCACCCGATTGTATAATTGCCGACACGTTTTACCCGTGGG
CCTCCGAATTTGCTAATCGGATGGGTATCCCGAGACTCATTTTCTTCCCG
GGGTGTACTTTCGCGCTCTGTTTGATGGAATCTATTCGATCTCCCGACTC
CCCGCACCGAAGATTGAGTTCGGATTCGGACCCATTTGTGGTTCCGGGTC
TTCCTCACCCGATCATCTTGACCCGTTCTCAACTTCCGGAACACGATCGA
GAGGACATAGCGGACCCGGCTGCCCAATTCATGGATCAGTGTAAAGAAGC
TGCGATGAAGAGCTACGGAATTATTCTCAACAATTTCGCGGAGATCGAAA
CAGAATACACAGAGCATTACAAGAAAATAACGGGTCACAAGGTTTGGCAC
ATTGGACCTGCCGCAGCAATTGTTCACCGAAATGCCAAAGAGAAGGCAGA
GGGGCTTTTCAAAAGTGACGAGCATGACAATAACCTTGTCATCAATTGGC
TCAACTCGAAGGAACCAAACTCAGTTGTTTATGTTTGTTTCGGCAGCGGA
TGTCAATTCCCTGATAAACAACTCTATGAGATTGCATGCGGGTTAGAGTT
ATCTGGGCATCAATTTATTTGGGTGGTTCGCGGCAAAGATAAACAAATCG
ATGTTAATGACGATGAGGAGAAGACATGGTTGCCTAAAGGGTTTGAGGAA
AGAATGAAAACAGAAAATAAAGGTTTGATTGTAAGGGGATGGGCCCCACA
GGTGCTGGTTTTGGATCATCCATCGTTGGGATGTTTCTTGACGCATTGCG
GCTGGAATTCTACGATTGAGGGAATCACAGCAGGTGTTCCTTTGATCACG
TGGCCAGTATACTCCGAGCAATTCTATAATGAGAAGCTAATCACGCAGGT
GCATGGTAATGGGGTGGGGGTTGGTTCAGAGGAGTGGATCATGCTGTTCA
GCGTCGCTAAAAGTTTGGTAAGTAGAGACAAAATTGAGAATGCTGTGAGG
AAGATAATGGACGGTGGTGATGAGGCTTTAGAAATCAGAAGGCGGGCCCG
GGAACTTGGAGAAAAAGCTAGGAAAGCTGCTTCAATTGGGGGGTCCTCCG
ACAATAATCTAACCGCAGCAATTGAAGACCTTAAGCGGTTGAGAGAGGAC
CGTTCGAAGCTGAAAACAAAAACAATTTGA
An apiosyltransferase enzyme capable of
transferring β-1,3-D-apiofuranose to
QA-FRX (Qs-28-O-ApiT4).
SEQ ID NO 10
MDSTHLQPATTPLKIHVIPFIAPGHIIPLSELARIFASRGEHVTIITTPF
NADLLQKSIDEDRDSGEHIGIHTVEWSATELGLPHGVENLSNVTDLETGI
KLHRALVLMQKQMEDFMTRNPPDCIIADTFYPWASEFANRMGIPRLIFFP
GCTFALCLMESIRSPDSPHRRLSSDSDPFVVPGLPHPIILTRSQLPEHDR
EDIADPAAQFMDQCKEAAMKSYGIILNNFAEIETEYTEHYKKITGHKVWH
IGPAAAIVHRNAKEKAEGLFKSDEHDNNLVINWLNSKEPNSVVYVCFGSG
CQFPDKQLYEIACGLELSGHQFIWVVRGKDKQIDVNDDEEKTWLPKGFEE
RMKTENKGLIVRGWAPQVLVLDHPSLGCFLTHCGWNSTIEGITAGVPLIT
WPVYSEQFYNEKLITQVHGNGVGVGSEEWIMLFSVAKSLVSRDKIENAVR
KIMDGGDEALEIRRRARELGEKARKAASIGGSSDNNLTAAIEDLKRLRED
RSKLKTKTI
A nucleic acid sequence which encodes
the enzyme according to SEQ ID NO 12.
SEQ ID NO 11
ATGGCAGAAGCAACGCAGAGGTATGCTGTTGTGACAGGATCTAATAAGGG
AATTGGATTTGGGATATGCAAGCAGTTGGCTTCTAAGGGAATCAAAGTAG
TGTTAACAGCTAGAGATGAGAAGAGAGGTCTTGAAGCAGTTGAGAAATTG
AAAGAAATTAGTCTGGCTGGTCATGTGGTTTTTCATCAACTCGATGTGTC
TGATCCTGCTAGTGTTACTAGCCTTGAAGATTTCATCAAAACCCAGTTTG
GGAAGCTAGATATTCTGGTAAACAATGCTGGGATAACAGGAACAACTGTA
GATGCTGATGCTTTAGCAGCTTCAGGCTTCGGTACAGGGGGTGAACGTAA
GCCTATTGATTGGAGTAAGTTAGTGATACAGACTTATGAATCAGTTGAAA
AAGCTTTCAACACAAACTATTACGGTGGCAAAAGAATGACAGAAGCACTT
ATACCCCTCCTCCAGCTATCAGACTCACCCAGGATTGTTAATGTTTCCTC
TGCTATGGGACAGTTAGAGAATATACCTAGTGGATGGGCAAAGGAAGTGC
TCACAGATGTTGATAACCTAACAGAAGGAAAATTGGATGAGGTTTCAACC
CAGTTTTTGAAAGATTTCAAAGAGGGTTCATTGGAAACCAAAGGCTGGCC
TAGTCTTATGTCTTCTTATATAGTCTCAAAAGCTGTTTTAAATGCCTACA
CAAGGATTCTTGCTAAGAAATACCCAGCTTTCTGCATCAATTGTGTAGAT
CCTGGCTATGTGAAGACAGACATAAACCATCATACTGGCCAATTAAGTGT
TGATGAAGGTGCTGAAAGTCCTGTAAGACTGGCCTTGCTGCCTAATGGTG
GTCCTTCTGGCGTGTTCTTCTCCAGGACAGAAGAAGCACCATTTTGA
An oxidoreductase enzyme capable
of enhancing the activity of a
fucosyltransferase (QsFucSyn)
SEQ ID NO 12
MAEATQRYAVVTGSNKGIGFGICKQLASKGIKVVLTARDEKRGLEAVEKL
KEISLAGHVVFHQLDVSDPASVTSLEDFIKTQFGKLDILVNNAGITGTTV
DADALAASGFGTGGERKPIDWSKLVIQTYESVEKAFNTNYYGGKRMTEAL
IPLLQLSDSPRIVNVSSAMGQLENIPSGWAKEVLTDVDNLTEGKLDEVST
QFLKDFKEGSLETKGWPSLMSSYIVSKAVLNAYTRILAKKYPAFCINCVD
PGYVKTDINHHTGQLSVDEGAESPVRLALLPNGGPSGVFFSRTEEAPF*
A nucleic acid sequence which encodes
the enzyme according to SEQ ID NO 14.
SEQ ID NO 13
ATGCCAACTTTGTACAAAAAAGCAGGCTTAATGGCGTCGGCGTCAAGGGT
AGATCTGGATGGTAATCAGATAAAGCCGATGACAATTTGCATGATCGGTG
CCGGTGGGTTCATTGGGTCCCACCTCTGCGAGAAGATTATGGCGGAGACA
CCGCACAAGGTTCTGGCATTAGATGTCTACAATGACAAGATCAAGCACTT
ACTGGAGCCGGATTCTCTTCAATGGAAAGATCGCATCCAATTCCACCGCA
TCAACATTAAGCACGATTCGAGGCTCGAAGGTCTCATCAAGATGGCAGAT
CTGACTATAAATCTGGCTGCTATTTGTACTCCCGCGGATTACAACACCCG
TCCTCTGGACACAATTTATAGCAATTTCATTGACGCTCTTCCTGTGGTAA
AGTACTGTTCGGAGAATAACAAGCGTCTCATTCATTTCTCTACGTGTGAA
GTGTATGGGAAAACGATTGGGAGCTTTCTCCCAAAAGACAGCCCTCTTCT
AAAGGATCCTGAATATTTTGTTCTTAAAGAAGATGCCTCCCCATGCATAT
TTGGTCCTATTGAAAAGCAGAGATGGTCCTACGCATGTGCAAAGCAATTG
ATTGAGAGGCTGGTTTATGCTGAGGGTGCTGAGAATGGCCTTGAGTTCAC
TATTGTGCGACCTTTCAATTGGATTGGCCCCAGAATGGATTTCATACCTG
GCATTGATGGTCCAAGTGAAGGTGTTCCACGGGTTCTGGCATGCTTTAGT
AACAATCTCCTTCGCGGTGAGCCACTCAAACTCGTTGATGGTGGCCAATC
CCAGAGAACTTTTGTTTATATCAAGGATGCAATTGAAGCTGTTTTGTTGA
TGATTGAAAACCCTGCCAGGGCTAATGGTCATATTTTTAATGTGGGTAAC
CCTCACAATGAAGTTACAGTTCGGCAACTTGCTGAAATGATGACCGAGGT
CTATTCTAAGGTAAGTGGAGAACCGTCTCTTGAGGTGCCTACCATTGATG
TAAGCTCCAAAGAATTTTATGGTGAAGGATACGATGATAGTGACAAGAGA
ATTCCTGACATGACCATAATCAACAGGCAACTTGGCTGGAACCCTAAGAC
ATCGCTCTGGGATCTTCTTGAGTCGACCCTCACCTATCAACATAGGACAT
ATGCAGAAGCTATTAAGAAATCAATTGCGAAACCAGTTGCCAGCTAG
An enzyme capable of enhancing the
activity of an apiosyltransferase
(QsAXS1).
SEQ ID NO 14
MPTLYKKAGLMASASRVDLDGNQIKPMTICMIGAGGFIGSHLCEKIMAET
PHKVLALDVYNDKIKHLLEPDSLQWKDRIQFHRINIKHDSRLEGLIKMAD
LTINLAAICTPADYNTRPLDTIYSNFIDALPVVKYCSENNKRLIHFSTCE
VYGKTIGSFLPKDSPLLKDPEYFVLKEDASPCIFGPIEKQRWSYACAKQL
IERLVYAEGAENGLEFTIVRPFNWIGPRMDFIPGIDGPSEGVPRVLACFS
NNLLRGEPLKLVDGGQSQRTFVYIKDAIEAVLLMIENPARANGHIFNVGN
PHNEVTVRQLAEMMTEVYSKVSGEPSLEVPTIDVSSKEFYGEGYDDSDKR
IPDMTIINRQLGWNPKTSLWDLLESTLTYQHRTYAEAIKKSIAKPVAS*
A nucleic acid sequence which encodes
the enzyme according to SEQ ID NO 16.
SEQ ID 15
ATGGCGCCCGAGAAAATGCCCGAGGAGGACGAGGAAATCGTCGCCGGGGT
CGTCGCAGGGAAGATCCCCTCCTACGTGCTCGAGACCAGGCTAGGCGACT
GCCGCAGGGCAGCCGGGATCCGCCGCGAGGCGCTGCGCCGGATCACCGGC
AGGGAGATCGACGGCCTTCCCCTCGACGGCTTCGACTACGACTCGATTCT
CGGACAGTGCTGCGAGATGCCCGTCGGGTACGTGCAGCTGCCGGTCGGCG
TCGCGGGGCCGCTCGTCCTCGACGGCCGCCGCATATACGTCCCGATGGCC
ACCACGGAGGGCTGCCTAATCGCCAGCACCAACCGCGGATGCAAGGCCAT
TGCCGAGTCCGGAGGCGCATCCAGCGTCGTGTACCGCGACGGGATGACCC
GCGCCCCCGTAGCCCGCTTCCCCTCCGCACGACGCGCCGCAGAGCTCAAG
GGCTTCCTGGAGAATCCGGCCAACTACGACACCCTGTCCGTGGTCTTTAA
CAGATCAAGCAGATTTGCAAGGCTGCAGGGGGTCAAGTGCGCCATGGCTG
GGAGGAACTTGTACATGAGGTTCACCTGCAGCACCGGGGATGCCATGGGG
ATGAACATGGTCTCCAAGGGCGTCCAAAATGTGCTCGACTATCTGCAGGA
GGACTTCCCTGACATGGACGTTGTCAGCATCTCAGGCAACTTTTGTTCCG
ACAAGAAATCAGCTGCTGTAAACTGGATTGAAGGCCGTGGAAAGTCCGTG
GTTTGTGAGGCAGTAATCAGAGAGGAAGTTGTCCACAAGGTTCTCAAGAC
CAACGTTCAGTCACTCGTGGAGTTGAATGTGATCAAGAACCTTGCTGGCT
CAGCAGTTGCTGGTGCTCTTGGGGGTTTCAACGCCCACGCAAGCAACATC
GTAACGGCTATCTTCATTGCCACTGGTCAGGATCCTGCACAGAATGTGGA
GAGCTCACAGTGTATCACTATGTTGGAAGCTGTAAATGATGGCAGAGACC
TTCACATCTCCGTTACAATGCCATCTATCGAGGTGGGCACAGTTGGTGGA
GGCACGCAGCTGGCCTCACAGTCGGCCTGCTTGGACCTACTGGGCGTCAA
AGGCGCCAACAGGGAATCTCCGGGGTCGAACGCTAGGCTGCTGGCCACGG
TGGTGGCTGGTGCCGTCCTAGCTGGGGAGCTGTCCCTCATCTCCGCCCAA
GCTGCCGGCCATCTGGTCCAGAGCCACATGAAATACAACAGATCCAGCAA
GGACATGTCCAAGATCGCCTGCTGA
AstHMGR (Avena strigosa truncated
HMG-COA reductase) translated
nucleotide sequence (424aa):
SEQ ID 16
MAPEKMPEEDEEIVAGVVAGKIPSYVLETRLGDCRRAAGIRREALRRITG
REIDGLPLDGFDYDSILGQCCEMPVGYVQLPVGVAGPLVLDGRRIYVPMA
TTEGCLIASTNRGCKAIAESGGASSVVYRDGMTRAPVARFPSARRAAELK
GFLENPANYDTLSVVFNRSSRFARLQGVKCAMAGRNLYMRFTCSTGDAMG
MNMVSKGVQNVLDYLQEDFPDMDVVSISGNFCSDKKSAAVNWIEGRGKSV
VCEAVIREEVVHKVLKTNVQSLVELNVIKNLAGSAVAGALGGFNAHASNI
VTAIFIATGQDPAQNVESSQCITMLEAVNDGRDLHISVTMPSIEVGTVGG
GTQLASQSACLDLLGVKGANRESPGSNARLLATVVAGAVLAGELSLISAQ
AAGHLVQSHMKYNRSSKDMSKIAC
A nucleic acid sequence which encodes
the enzyme according to SEQ ID NO 18.
SEQ ID NO 17
ATGTGGAGGCTGAAGATAGCAGAAGGTGGTTCCGATCCATATCTGTTCAG
CACAAACAACTTCGTGGGTCGCCAGACATGGGAGTTCGAACCGGAGGCCG
GCACACCTGAGGAGCGAGCAGAGGTCGAAGCTGCCCGCCAAAACTTTTAC
AACAACCGTTACCAGGTCAAGCCCTGTGACGACCTCCTTTGGAGATATCA
GTTCCTGAGAGAGAAGAATTTCAAACAAACAATACCGCCTGTCAAGGTTG
AAGATGGCCAAGAAATTACTTATGAGATGGCCACAACCTCAATGCAGAGG
GCGGCCCGTCACCTATCAGCCTTGCAGGCCAGCGATGGCCATTGGCCAGC
TCAAATTGCTGGCCCCTTGTTCTTCATGCCACCCTTGGTCTTTTGTGTGT
ACATTACTGGGCATCTTAATACAGTATTCCCATCTGAACATCGCAAAGAA
ATCCTTCGTTACATGTACTATCACCAGAACGAAGATGGTGGGTGGGGACT
GCACATAGAGGGTCACAGCACCATGTTTTGCACAGCACTCAACTACATTT
GTATGCGTATCCTTGGGGAAGGACCAGAGGGGGGTCAAGACAATGCTTGT
GCCAGAGCACGAATGTGGATTCTTGATCATGGTGGTGTAACACATATTCC
ATCTTGGGGAAAGACCTGGCTTTCGATACTTGGTCTATTTGAGTGGTCTG
GAAGCAATCCAATGCCTCCAGAGTTTTGGATCCTTCCTTCATTTCTTCCT
ATGCATCCAGCAAAAATGTGGTGCTATTGCCGGATGGTTTACATGCCCAT
GTCTTATTTATATGGGAAAAGGTTTGTTGGCCCAATCACGCCTCTCATTG
TTCAGTTAAGAGAGGAAATACACACTCAAAATTACCATGAAATCAACTGG
AAGTCAGTCCGCCATCTATGTGCAAAGGAGGATATCTACTATCCCCATCC
ACTCATCCAAGATTTGATTTGGGACAGTTTGTACATACTAACGGAGCCTC
TTCTCACTCGCTGGCCCTTGAACAAGTTGGTGCGGGAGAGGGCTCTCCAA
GTAACAATGAAGCATATCCACTATGAAGATGAAAATAGTCGATACATAAC
CATTGGATGTGTGGAAAAGGTGTTATGTATGCTTGCTTGTTGGGTTGATG
ATCCAAATGGAGATGCTTTCAAGAAGCACCTTGCTCGAGTCCCAGATTAC
GTATGGGTCTCTGAAGATGGAATTACTATGCAGAGTTTTGGTAGTCAAGA
ATGGGATGCTGGCTTTGCCGTCCAGGCTCTGCTTGCTTCTAATCTTACCG
AGGAACTTGGCCCTGCTCTTGCCAAAGGACATGACTTCATAAAGCAATCT
CAGGTTAAGGACAATCCTTCAGGTGACTTCAAAAGCATGTATCGTCACAT
TTCTAGAGGATCATGGACCTTCTCTGACCAAGATCATGGATGGCAAGTTT
CTGATTGCACTGCAGAAGGTCTGAAGTGTTGCCTGCTTTTGTCGATGTTG
CCACCAGAAATTGTTGGTGAAAAAATGGAACCACAAAGGCTATTTGATTC
TGTCAATGTGCTGCTCTCTCTACAGAGCAAAAAAGGTGGTTTAGCTGCCT
GGGAGCCAGCAGGGGCGCAAGATTGGTTGGAATTACTCAATCCCACAGAA
TTTTTTGCGGACATTGTCGTTGAGCATGAATATGTTGAATGTACTGGATC
AGCAATTCAGGCATTAGTTTTGTTCAAGAAGCTGTATCCGGGGCACAGGA
AAAAAGAGATTGACAGTTTCATTACAAATGCTGTCCGGTTCCTTGAGAAT
ACACAAACGGCAGATGGCTCTTGGTATGGAAACTGGGGAGTTTGCTTCAC
CTATGGTTGTTGGTTCGCACTGGGAGGGCTAGCAGCAGCTGGCAAGACTT
ACAACAACTGTCCTGCAATACGCAAAGCTGTTAATTTCCTACTTACAACA
CAAAGAGAAGACGGTGGTTGGGGAGAAAGCTATCTTTCAAGCCCAAAAAA
GATATATGTACCCCTGGAAGGAAGCCGATCAAATGTGGTACATACTGCAT
GGGCTATGATGGGTCTAATTCATGCTGGGCAGGCTGAAAGAGACTCAACT
CCTCTTCATCGTGCAGCAAAGTTGATCATCAATTATCAACTAGAAAATGG
CGATTGGCCGCAACAGGAAATCACTGGAGTATTCATGAAAAACTGCATGT
TACATTACCCTATGTACAGAAACATCTACCCAATGTGGGCTCTTGCAGAA
TACCGGAGGCGGGTTCCATTGCCTTAA
An enzyme involved in making β-amyrin
from 2,3-oxidosqualene (QsbAS)
SEQ ID NO 18
MWRLKIAEGGSDPYLFSTNNFVGRQTWEFEPEAGTPEERAEVEAARQNFY
NNRYQVKPCDDLLWRYQFLREKNFKQTIPPVKVEDGQEITYEMATTSMQR
AARHLSALQASDGHWPAQIAGPLFFMPPLVFCVYITGHLNTVFPSEHRKE
ILRYMYYHQNEDGGWGLHIEGHSTMFCTALNYICMRILGEGPEGGQDNAC
ARARMWILDHGGVTHIPSWGKTWLSILGLFEWSGSNPMPPEFWILPSFLP
MHPAKMWCYCRMVYMPMSYLYGKRFVGPITPLIVQLREEIHTQNYHEINW
KSVRHLCAKEDIYYPHPLIQDLIWDSLYILTEPLLTRWPLNKLVRERALQ
VTMKHIHYEDENSRYITIGCVEKVLCMLACWVDDPNGDAFKKHLARVPDY
VWVSEDGITMQSFGSQEWDAGFAVQALLASNLTEELGPALAKGHDFIKQS
QVKDNPSGDFKSMYRHISRGSWTFSDQDHGWQVSDCTAEGLKCCLLLSML
PPEIVGEKMEPQRLFDSVNVLLSLQSKKGGLAAWEPAGAQDWLELLNPTE
FFADIVVEHEYVECTGSAIQALVLFKKLYPGHRKKEIDSFITNAVRFLEN
TQTADGSWYGNWGVCFTYGCWFALGGLAAAGKTYNNCPAIRKAVNFLLTT
QREDGGWGESYLSSPKKIYVPLEGSRSNVVHTAWAMMGLIHAGQAERDST
PLHRAAKLIINYQLENGDWPQQEITGVFMKNCMLHYPMYRNIYPMWALAE
YRRRVPLP*
A nucleic acid sequence which encodes
the enzyme according to SEQ ID NO 20.
SEQ ID NO 19
ATGGAGCACTTGTATCTCTCCCTTGTGCTCCTGTTTGTTTCCTCAATCTC
CCTCTCCCTCTTCTTCCTGTTCTACAAACACAAATCTATGTTCACCGGGG
CCAACCTACCACCTGGTAAAATCGGTTACCCATTGATCGGAGAGAGCTTG
GAGTTCTTGTCCACGGGATGGAAGGGCCACCCGGAGAAATTCATCTTCGA
TCGCATGAGCAAGTACTCATCCCAAATCTTCAAGACCTCGATTTTAGGGG
AACCAACGGCGGTGTTCCCGGGAGCCGTATGCAACAAGTTCCTCTTCTCC
AACGAGAACAAGCTGGTGAATGCATGGTGGCCTGCCTCCGTGGACAAGAT
CTTTCCTTCCTCACTCCAGACATCCTCCAAAGAAGAGGCCAAGAAGATGA
GGAAGTTGCTTCCTCAGTTTCTCAAGCCCGAAGCTCTGCACCGCTACATT
GGTATTATGGATTCTATTGCCCAGAGACACTTTGCCGATAGCTGGGAAAA
CAAAAACCAAGTCATTGTCTTTCCTCTAGCAAAGAGGTATACTTTCTGGC
TGGCTTGCCGTTTGTTCATTAGCGTCGAGGATCCGACCCACGTATCCAGA
TTTGCTGACCCGTTCCAACTTTTGGCCGCCGGAATCATATCAATCCCAAT
CGACTTGCCAGGGACACCGTTCCGCAAGGCAATCAATGCGTCCCAGTTCA
TCAGGAAGGAATTGTTGGCCATCATCAGGCAGAGAAAGATCGATTTGGGT
GAAGGGAAGGCATCTCCGACGCAGGACATACTGTCTCACATGTTGCTCAC
ATGCGACGAGAACGGACAATACATGAATGAATTGGACATTGCCGACAAGA
TTCTTGGCTTGTTGGTCGGCGGACATGACACTGCCAGTGCCGCTTGCACT
TTCATTGTCAAGTTCCTCGCTGAGCTTCCCCACATTTATGAACAAGTCTA
CAAGGAGCAAATGGAGATTGCAAAATCAAAAGTGCCAGGAGAGTTGTTGA
ATTGGGAGGACATCCAAAAGATGAAATATTCGTGGAACGTAGCTTGTGAA
GTGATGAGACTTGCCCCTCCACTCCAAGGAGCTTTCAGGGAAGCCATTAC
TGACTTCGTCTTCAACGGTTTCTCCATTCCAAAAGGCTGGAAGTTGTACT
GGAGCGCAAATTCCACCCACAAAAGTCCGGATTATTTCCCTGAGCCCGAC
AAGTTCGACCCAACTAGATTCGAAGGAAATGGACCTGCGCCTTACACCTT
TGTTCCATTTGGGGGAGGACCCAGGATGTGCCCGGGCAAAGAGTATGCCC
GATTGGAAATACTTGTGTTCATGCATAACTTGGTGAAGAGGTTCAAGTGG
GAGAAATTGGTTCCTGATGAAAAGATTGTGGTTGATCCAATGCCCATTCC
AGCAAAGGGTCTTCCTGTTCGCCTTTATCCTCACAAAGCTTGA
An enzyme involved in making Oleanolic
acid from β-amyrin (QsCYP716-C-28)
SEQ ID NO 20
MEHLYLSLVLLFVSSISLSLFFLFYKHKSMFTGANLPPGKIGYPLIGESL
EFLSTGWKGHPEKFIFDRMSKYSSQIFKTSILGEPTAVFPGAVCNKFLFS
NENKLVNAWWPASVDKIFPSSLQTSSKEEAKKMRKLLPQFLKPEALHRYI
GIMDSIAQRHFADSWENKNQVIVFPLAKRYTFWLACRLFISVEDPTHVSR
FADPFQLLAAGIISIPIDLPGTPFRKAINASQFIRKELLAIIRQRKIDLG
EGKASPTQDILSHMLLTCDENGQYMNELDIADKILGLLVGGHDTASAACT
FIVKFLAELPHIYEQVYKEQMEIAKSKVPGELLNWEDIQKMKYSWNVACE
VMRLAPPLQGAFREAITDFVFNGFSIPKGWKLYWSANSTHKSPDYFPEPD
KFDPTRFEGNGPAPYTFVPFGGGPRMCPGKEYARLEILVFMHNLVKRFKW
EKLVPDEKIVVDPMPIPAKGLPVRLYPHKA*
A nucleic acid sequence which encodes
the enzyme according to SEQ ID NO 22.
SEQ ID NO 21
ATGATATATAATAATGATAGTAATGATAATGAATTAGTAATCAGCTCAGT
TCAGCAACCATCCATGGATCCTTTCTTCATTTTTGGCTTACTTCTCTTGG
CTCTCTTTCTCTCTGTTTCTTTTCTTCTCTACCTTTCCCGTAGAGCCTAT
GCTTCTCTCCCCAACCCTCCGCCGGGGAAGCTCGGCTTCCCCGTCGTCGG
CGAGAGTCTCGAATTTCTCTCCACCCGACGCAAAGGTGTTCCTGAGAAAT
TCGTCTTCGACAGAATGGCCAAATACTGTCGGGATGTCTTTAAGACATCA
ATATTGGGAGCAACCACCGCCGTCATGTGCGGCACCGCCGGTAACAAATT
CTTGTTCTCCAACGAGAAAAAACACGTCACTGGTTGGTGGCCGAAATCTG
TAGAGCTGATTTTCCCAACCTCACTTGAGAAATCATCCAACGAAGAATCC
ATCATGATGAAACAATTCCTTCCCAACTTCTTGAAACCAGAACCTTTGCA
GAAGTACATACCCGTTATGGACATAATTACCCAAAGACACTTCAATACAA
GCTGGGAAGGACGCAACGTGGTCAAAGTGTTTCCTACGGCTGCCGAATTC
ACCACGTTGCTGGCTTGTCGGGTATTCCTCAGTGTTGAGGATCCCATTGA
AGTAGCCAAGATTTCAGAGCCATTTGAAATCTTAGCTGCTGGGTTTCTTT
CAATACCCATAAATCTTCCGGGTACCAAATTAAATAAAGCGGTTAAGGCA
GCGGATCAGATTAGAGACGCAATTGTACAGATTTTGAAACGGAGAAGGGT
TGAAATTGCGGAGAATAAAGCAAATGGAATGCAAGATATAGCGTCCATGT
TGTTGACGACACCAACTAATGCTGGGTTTTATATGACCGAGGCTCACATT
TCTGAGAAAATTTTGGGTATGATTGTTGGTGGCCGTGATACTGCTAGTAC
TGTTATCACCTTCATCATCAAGTATTTGGCAGAGAATCCTGAAATTTATA
ATAAGGTCTATGAGGAGCAAATGGAAGTGGTAAAGTCAAAGAAACCAGGT
GAGTTGCTGAACTGGGAAGATGTGCAGAAAATGAAGTACTCTTGGTGCGT
AGCATGTGAAGCTATGCGACTTGCTCCTCCTGTTCAAGGTGGTTTCAAGG
TGGCCATTAATGACTTTGTGTATTCTGGGTTCAACATTCGCAAGGGTTGG
AAGTTATATTGGAGTGCCATTGCAACACACATGAATCCAGAATATTTCCC
AGAACCTGAGAAATTCAACCCCTCAAGGTTTGAAGGGAAGGGACCAGTAC
CTTACAGCTTCGTACCCTTCGGAGGCGGACCTCGGATGTGTCCCGGGAAA
GAGTATTCCCGGCTGGAAACACTTGTTTTCATGCATCATTTGGTGACGAG
GTACAATTGGGAGAAAGTGTATCCCACAGAGAAGATAACAGTGGATCCAA
TGCCATTCCCTGTCAACGGCCTCCCCATTCGCCTTATTCCTCACAAGCAC
CAATGA
An enzyme involved in making Echinocystic
acid from Oleanolic acid (QsCYP716-C-16α)
SEQ ID NO 22
MIYNNDSNDNELVISSVQQPSMDPFFIFGLLLLALFLSVSFLLYLSRRAY
ASLPNPPPGKLGFPVVGESLEFLSTRRKGVPEKFVFDRMAKYCRDVFKTS
ILGATTAVMCGTAGNKFLFSNEKKHVTGWWPKSVELIFPTSLEKSSNEES
IMMKQFLPNFLKPEPLQKYIPVMDIITQRHENTSWEGRNVVKVFPTAAEF
TTLLACRVFLSVEDPIEVAKISEPFEILAAGFLSIPINLPGTKLNKAVKA
ADQIRDAIVQILKRRRVEIAENKANGMQDIASMLLTTPTNAGFYMTEAHI
SEKILGMIVGGRDTASTVITFIIKYLAENPEIYNKVYEEQMEVVKSKKPG
ELLNWEDVQKMKYSWCVACEAMRLAPPVQGGFKVAINDFVYSGFNIRKGW
KLYWSAIATHMNPEYFPEPEKFNPSRFEGKGPVPYSFVPFGGGPRMCPGK
EYSRLETLVFMHHLVTRYNWEKVYPTEKITVDPMPFPVNGLPIRLIPHKH
Q*
A nucleic acid sequence which encodes
the enzyme according to SEQ ID NO 24.
SEQ ID NO 23
ATGTGGTTCACAGTAGGATTGGTCTTGGTTTTCGCCCTATTCATACGTCT
CTACAGCAGTCTGTGGTTGAAGCCTCGTGCAACTCGGATTAAGCTTAGCA
ATCAAGGAATTAAAGGTCCAAAACCAGCATTTCTTCTGGGTAATGTTGCA
GAGATGAGAAGATTTCAATCTAAGCTTCCAAAATCTGAACTCAAACAAGG
CCAAGTTTCTCATGATTGGGCTTCTAAATCTCTGTTTCCATTTTTCAGTC
TTTGGTCCCAGAAATACGGAAATACGTTCGTGTTCTCATTGGGGAACATA
CAGGTGCTCTATGTTTCTGATCATGAGTTGGTGAAAGAAATTAATCAGAA
TACCTCTTTAGATTTGGGCAAACCCAAGTACCTGCAGAAGGAGCGTGGCC
CTTTGCTGGGACAAGGTATTTTGACCTCCAATGGACAGCTTTGGGCGTAC
CAGAGAAAAATCATGACTCCTGAACTCTACAAGGAGAAAATCAAGGGCAT
GTGCGAGTTGATGGTGGAATCTGTAGCTTGGTTGGTTGAGGAATGGGGAA
CGAAGATCCAAGCTGAGGGTGGGGCAGCAGACATTAGAATAGACGAGGAT
CTTAGAAGCTTCTCTGGTGATGTAATTTCAAAAGCTTGTTTTGGGAGCTG
CTATGCCGGAGGGAGGGAAATCTTTCTTAGGCTCAGAGCTCTTCAACACC
AAATTGCTTCCAAAGCCTTACTCATGGGCTTCCCTGGATTAAAGTACCTG
CCCATTAAGAGCAACAGAGAGATATGGAGATTGGAGAAGGAGATCTTCCA
GCTGATTATGAAGCTGGCTGAAGATAGAAAAAAAGAACAACATGAGAGAG
ACCTATTACAGATTATAATTGAGGGAGCTAAAAGTAGTGATCTGAGTTCG
GAAGCAATGGCAAAATTCATTGTGGACAACTGCAAGAATGTCTACTTGGC
TGGCCATGAAACTACTGCAATGTCTGCTGGTTGGACTTTGCTTCTCTTGG
CTAATCATCCTGAGTGGCAAGCCCGTGTCCGTGATGAGATTTTACAAGTC
ACCGAGGGCCGCAATCCTGATTTTGACATGCTGCACAAGATGAAACTGTT
AACAATGGTAATTCAGGAGGCACTGCGACTCTACCCAACAGTCATATTCA
TGTCAAGAGAAGCATTGGAAGATATTAATGTTGGAAACATCCAAGTTCCA
AAAGGTGTTAACATATGGATACCTGTGGTAAATCTTCAAAGGGACACAAC
GGTATGGGGTGCAGACGCAAACGAGTTTAATCCTGAAAGGTTTGCCAATG
GAGTTAACAATTCATGCAAGGTTCCACAACTTTACCTACCATTTGGAGCT
GGACCTCGCATTTGTCCTGGAATTAATCTGGCCATGACTGAGATCAAGAT
ACTTCTGTGTATCCTGCTCACCAAGTTTTCGTTTTCAGTTTCACCCAACT
ATCGCCACTCACCGGTGTTTAAATTGGTGCTTGAGCCTGAAAATGGAATC
AATGTCATCATGAAGAAGCTCTAA
An enzyme involved in making Quillaic
acid from Echinocystic acid
(QsCYP714-C-23).
SEQ ID NO 24
MWFTVGLVLVFALFIRLYSSLWLKPRATRIKLSNQGIKGPKPAFLLGNVA
EMRRFQSKLPKSELKQGQVSHDWASKSLFPFFSLWSQKYGNTFVFSLGNI
QVLYVSDHELVKEINQNTSLDLGKPKYLQKERGPLLGQGILTSNGQLWAY
QRKIMTPELYKEKIKGMCELMVESVAWLVEEWGTKIQAEGGAADIRIDED
LRSFSGDVISKACFGSCYAGGREIFLRLRALQHQIASKALLMGFPGLKYL
PIKSNREIWRLEKEIFQLIMKLAEDRKKEQHERDLLQIIIEGAKSSDLSS
EAMAKFIVDNCKNVYLAGHETTAMSAGWTLLLLANHPEWQARVRDEILQV
TEGRNPDFDMLHKMKLLTMVIQEALRLYPTVIFMSREALEDINVGNIQVP
KGVNIWIPVVNLQRDTTVWGADANEFNPERFANGVNNSCKVPQLYLPFGA
GPRICPGINLAMTEIKILLCILLTKFSFSVSPNYRHSPVFKLVLEPENGI
NVIMKKL*
A nucleic acid sequence which encodes
the enzyme according to SEQ ID NO 26.
SEQ ID NO 25
ATGAAATCCCCCTCTAACCCAAATCAGAAACCCATCCTCCACACTTGTAC
AATTCAGCAGCCTCGTGCTACCCTTAACAAAATTCATAGTCTTATTCATT
TCTCAGCCATACTTGTCCTATTTTATTACCGGATAACCCGTCTATTCTTC
ACCGACGATTTCAAGGTACCCAAGTTACTATGGACTCTAATGACAATCTC
CGAGTTCATTCTTGCCTTCATTTGGGTTCTCATCCAACCTTTCCGGTGGC
GACCGGTGTCCCGTTCCGTCATACCAGAGAATATGCCGAAGGACATCAGT
TTGCCGGCGGTGGACGTGTTTGTATGCACAGCTGACCCTCAAAAAGAACC
CACAGTGGAGGTGATGAACACAATTTTATCAGCCATGGCTTTAGACTACC
CGGCGGAGAAGCTCGCCGTGTATCTTTCCGATGATGGGGGTTCTGCTGTC
ACCTTATATGCTATAAAAGAAGCTTGTTGTTTTGCTAAGATGTGGCTTCC
GTTTTGTAACAAGTATGGGATCAAATCAAGGTGTCCCGAGGCTTATTTTT
CAAAGCTTGCCGCTGACGAGTGGCTTCACCGGAGTGTGGAATTCGTGGCA
GAAGAAAAGGAGGTCAAGGCTAATTATGAAGAGTTCAAGAGAAATGTGCA
GAAATTTGGTGAGCAACAAGAAAACAGTCGTGTTGTGCATGATCGTCACC
CTCATGTTGAGATTATACACAATAATTGGAATAACGAAGACCAAGCTCAT
GAGATGCCACTCCTTGTTTATGTCTCTCGTGAAAGAAGACCATCTCACCA
TCCTCGATTCAAAGCTGGAGCTCTTAACACCCTTCTTCGAGTTTCTGGCA
TCATCAGCAACAGCCCCTACATACTGGTTCTAGACTGTGACATGTACTGC
AATGACCCAACCTCAGCTAGACAAGCAATGTGCTTCCATCTTGATCCCCA
ACTGTCTAAAAATCTTGCTTTTGTACAATTCCCTCAAATATTCTATAACG
CTAGTAAGAATGACGTCTATGATGCCCAAGTCAGGGCGGCATACCAGACA
AAGTGGCAGGGTATGGATGGACTTCAAGGACCAATTTTTTCTGGCACTGG
CTTTTACTTAAAGAGGAAGGCAATGTATGGAAACCCTGATCAAGATGATA
ATTGTCTACTCAAGCCATATAAGAAATTTGGCATGTCTGGAGAATTTGTA
GAATCACTTAAGGTCCTTAACGAACAAGATGGTACCCAGAAGAAATTATT
GGATGGATTTTTACAAGAGGCCAAACTATTGGCCTCGTGTGCCTATGAAA
CAAAGACAAGTTGGGGTAAAGAGATTGGATTCTCATATGACTGTTTAATA
GAGAGCACTTTCACTGGTTATCTTTTGCACTGCAGAGGGTGGATATCTGT
TTATCTTTATCCCAAGAGACCATGTTTTTTAGGATGCTGTCCTACTGATA
TGAAGGATGCCATGGTTCAATATACCAAGTGGATGTCTGAGCTATTTTCA
ATTGCTATCTCAAGATTCAATCCTCTGCTCTATGGGGTGGCAAGAATGTC
CATTCTTCAAAGCCTGTGTTATGGATCCTTTACACTGGCGCCTATTTTGT
CATTTCCTTTGTTCTTATATGGAACGGTTCCTCAATTATGCCTCTTGAAA
GGCATATCTTTGTTTCCAAAGGTTTCGGACCCATGGTTTGCTGTGTTTGC
AGCTATCTTTGTATCCTCCCTGTGTCAACACTGGTTCGAGGTCCTCTCTT
GTGATGGTACATTTACGACTTGGTGTAATGAACAGCGGAGTTGGCTTATA
AAGTCGGTTTCCGGTAGTTTGTTTGGAGTTGTGGGCGCAATCTTGCAGCG
GCTAGGCTTGAAGACAAAGTTTAGTTTATCAAACAAAGCCATGGACAAAG
AAAAGCTGGAGAAATATGAAAAGGGTAAATTTAATTTCCAAGGGGCTGCC
ATGTTCATGGTTCCTGTGTCTATTTTAGTCATACTGAACACATTTTGCTT
CCTCGGTGGGTTTTGGAAAGTGATCATAATGAAGAATATCCTGGACATGT
TTGGACAACTTTCTCTCTCTGCCTACGTTCTGGTTCTCAGTTGTCCAGTT
CTTGAAGGGATGTTAACTAGAATCAGCAAGAAAATGGTCTGA
An enzyme involved in making QA-mono
from Quillaic acid (QsCSL1).
SEQ ID NO 26
MKSPSNPNQKPILHTCTIQQPRATLNKIHSLIHFSAILVLFYYRITRLFF
TDDFKVPKLLWTLMTISEFILAFIWVLIQPFRWRPVSRSVIPENMPKDIS
LPAVDVFVCTADPQKEPTVEVMNTILSAMALDYPAEKLAVYLSDDGGSAV
TLYAIKEACCFAKMWLPFCNKYGIKSRCPEAYFSKLAADEWLHRSVEFVA
EEKEVKANYEEFKRNVQKFGEQQENSRVVHDRHPHVEIIHNNWNNEDQAH
EMPLLVYVSRERRPSHHPRFKAGALNTLLRVSGIISNSPYILVLDCDMYC
NDPTSARQAMCFHLDPQLSKNLAFVQFPQIFYNASKNDVYDAQVRAAYQT
KWQGMDGLQGPIFSGTGFYLKRKAMYGNPDQDDNCLLKPYKKFGMSGEFV
ESLKVLNEQDGTQKKLLDGFLQEAKLLASCAYETKTSWGKEIGFSYDCLI
ESTFTGYLLHCRGWISVYLYPKRPCFLGCCPTDMKDAMVQYTKWMSELFS
IAISRFNPLLYGVARMSILQSLCYGSFTLAPILSFPLFLYGTVPQLCLLK
GISLFPKVSDPWFAVFAAIFVSSLCQHWFEVLSCDGTFTTWCNEQRSWLI
KSVSGSLFGVVGAILQRLGLKTKFSLSNKAMDKEKLEKYEKGKFNFQGAA
MFMVPVSILVILNTFCFLGGFWKVIIMKNILDMFGQLSLSAYVLVLSCPV
LEGMLTRISKKMV*
A nucleic acid sequence which encodes
the enzyme according to SEQ ID NO 28.
SEQ ID NO 27
ATGGCGACCGTCTCCTCCCTCCACACTTGCACTGTACAGCAACCCCGTGC
AGCCATTAATCGAATTCACATTTTCTTACACTTTATTGCCATACTTTTCC
TCTTTTACTACCGGGTCACCGGTCTTTTCTATGACAATGCAGTACCCACT
TTAGCTTGGTCTCTAATGACCTTAGCTGAGTTGATTTTCGCCTTCGTTTG
GGTGCTCAGCCAAGCCTTCCGGTGGCGCCCGGTGTTGCGTTCAGTTATTC
CTGAGAGGATTCCCAAAGATGTACGATTGCCCGCGGTGGATATCTTAATT
TGTACGGCTGACCCATTAAAGGAACCGACGGTGGAGGTGATGAACACTGT
CTTGTCCGCCATGGCATTGGACTATCCTGCGGAGAATCTGGCTGTATATC
TTTCTGATGACGGGGGTTCTCCGGTCACCTTATTTGCTATGAAGCAAGTG
GGTCCGTTTGCTAAGCTGTGGCTTCCGTTTTGCAACAAGTACGGAATCAA
AACAAGGCATCCTGAGTCTTTTTTCTCGGCATTTGCGGATGACGAAAGGC
TTCACCGGAGTGATGAATTCAGGGCAGAGGAGGAGGCGATCAAGGACAAA
TATGAAGAATTTAAGAGAACTATAGAGAAATATGGTGGAGAAGGAAAAAA
TAGTCATGTTGTACAAGACCGGCCTCCTCATGTGGAGATTATACATGACA
CTAGGAAGATTAGAGAGAACAGTGAAGACCAAGCTGTGCCTCTTCTTGTC
TACGTCTCTCGTGAGAAAAGACCATCCTACAATTCTCGGTTCAAAGCAGG
AGCTCTGAACACCCTTCTTCGAGTTTCTGGGGTAATCAGCAATAGCCCAT
ATGTATTGGTGTTAGACTGTGACATGTACTGCAATGATCCAACATCAGCT
AGACAAGCAATGTGCTTCCATCTTGATCCACAAATGTCTCGCACTCTCTC
TTTTGTACAATTCCCCCAGGTTTTCTACAATGTTAGTAAAAATGATATCT
ATGATGGCCAAGCTAGGGCAGCCTTTAAGACAAAGTGGCAAGGTATGGAT
GGACTACGTGGGCCACTGCTTTCTGGTACTGGCTTTTATTTGAAGAGGAA
GTCCTTGTATGGAAGTCCAAACCAAGAAGATGATTGTTTACTTGAGCCCC
ATAAGAATTTTGGAAAGTGTGACAAGCTCATAGAATCAGTAAAGGTCATT
TATGAACGTGATGTTTCAATAAAGGCAGATTCATCAGATGCCATTTTGCA
AGATGCCAAACAATTAGCATCTTGTCCCTATGAAACAAACACAAGCTGGG
GCAAAGAGGTTGGGTTCTCGTATGACTGCTTATTAGAGAGTACATTCACA
GGTTATCTGTTGCACTGCAGAGGGTGGACATCAGTTTATCTTTATCCAAA
GAAGCCATGTTTCTTAGGGTGTACTCCAGTTGATATGAAGGAAGCCATGG
TTCAGTATACGAAGTGGATTTCTGAATTATTTTTACTTGCTATCTCAAGA
TTCAACCCTCTGACATTTGGGATATCCAGAATGTCCATTCTCCAGAGCAT
GTGTTACGGATACCTTACAATCATGCCCATTTTATCTGTTGCTATGATCT
TCTATGCCACAGTTCCTCAATTGTGCCTCTTGAGAGGCGTACCTCTGTTT
CCCAAGGTTTCAGACCCATGGTTTGCAGTGTTCCTAGCAATATTTGTGTC
CTCCCTCTGTCAGCACTTAATTGAAGTCCTCACGAGTGATGGCACGCTCA
AGACTTGGTGGAATGAACAAAGAAATTGGGTGATAAAGTCTGGTTCCGGT
AGCGTATTTGGAGCTCTGAGTGGAATATTGAAGTGGTTTGGCATGAAGAT
TAAATTTGGTTTATCAAACAAAGCCGTGGACAAAGAAAAGCTTGAGAAAT
ATGAAAAGGGTAAGTTTGATTTCCAAGGGGCTGCCATGTTTATGGTTCCC
TTAACTATATCAGTCATCTTGAACACATTATGCCTTATCGGTGGTTTATG
GAGAGTAATCACACTTAAAAACTTCGAAGAGATGTCAGGGCAGTTCATCA
TCTCCTTGTACTTTCTAGCTCTCAGCTATCCAATTCTTGAAGGGTTACTA
AGAAAAGGCAAGGGAAAGGCCTAA
An enzyme involved in making QA-mono
from Quillaic acid (QsCsIG2).
SEQ ID NO 28
MATVSSLHTCTVQQPRAAINRIHIFLHFIAILFLFYYRVTGLFYDNAVPT
LAWSLMTLAELIFAFVWVLSQAFRWRPVLRSVIPERIPKDVRLPAVDILI
CTADPLKEPTVEVMNTVLSAMALDYPAENLAVYLSDDGGSPVTLFAMKQV
GPFAKLWLPFCNKYGIKTRHPESFFSAFADDERLHRSDEFRAEEEAIKDK
YEEFKRTIEKYGGEGKNSHVVQDRPPHVEIIHDTRKIRENSEDQAVPLLV
YVSREKRPSYNSRFKAGALNTLLRVSGVISNSPYVLVLDCDMYCNDPTSA
RQAMCFHLDPQMSRTLSFVQFPQVFYNVSKNDIYDGQARAAFKTKWQGMD
GLRGPLLSGTGFYLKRKSLYGSPNQEDDCLLEPHKNFGKCDKLIESVKVI
YERDVSIKADSSDAILQDAKQLASCPYETNTSWGKEVGFSYDCLLESTFT
GYLLHCRGWTSVYLYPKKPCFLGCTPVDMKEAMVQYTKWISELFLLAISR
FNPLTFGISRMSILQSMCYGYLTIMPILSVAMIFYATVPQLCLLRGVPLF
PKVSDPWFAVFLAIFVSSLCQHLIEVLTSDGTLKTWWNEQRNWVIKSGSG
SVFGALSGILKWFGMKIKFGLSNKAVDKEKLEKYEKGKFDFQGAAMFMVP
LTISVILNTLCLIGGLWRVITLKNFEEMSGQFIISLYFLALSYPILEGLL
RKGKGKA
A nucleic acid sequence which encodes
the enzyme according to SEQ ID NO 30.
SEQ ID NO 29
ATGGTGGAGTCTCCAGCAGATCATGATGTGCTCAAAATCATTGTCCTTCC
ATGGGTAACCTCAGGTCACATGATTCCCATGGTAGATGCAGCCAGACTAT
TTGCTATGCATGGTGCAGATGTTACCATCATCACCACCCCAGCTAATGCC
CTTACATTCCAGAAATCCGTCGACCGTGATTTCAATTCCGGTCGTTTAAT
CAGAACTCACACCCTTAAATTCCCTGCAGCAGAAGTTGGTGTACCTGAAG
GAGTTGAAAACTTCAACAATACTTCCCCTGAAATGACCTCCAAAGTCTAC
CTTGGAGTCTCAATGCTCCGAGAACCAACCCAACAATTGATTGAGGATCT
GCGTCCAGATTGTCTTATCACTGATATGTTCTATCCTTGGGCTGTGGATG
TTGCTGACAAATTAGGCATTCCAAGGCTAATTTTTCAAGGTCCTGGAAGT
TTTGGTTTGTCAGCTATGCATTCTATCAAACAGTATGAGCCCTTTAAGTC
AGTAACTTCAGATACTGAGACATTCCCACTACCTGGATTGCCGCATAAGG
TAGAGATGACAAGGTTGCAGATACCAAAATGGGTTCGTGAGCCAAATGGG
TACACTCAATTGATGGGCAGGGTAAAAGATTCGGAGAGAAGAAGCTATGG
GTCATTGGTGAATAGCTTTTATGACTTCGAAGGCCCTTATGAAGAGCACT
ATAGGAAGGCAACAGGACAGAGGGTTTGGAGCATTGGACCAGTTTCAGTT
TGGGTGAACCAAGATGCTGCAGATAAGGTTGGAAGAGGACAGGATCTTGT
TGCTGAAGACCAAAACAGCTGGTTGAATTGGCTCAATTCCAAAGAGAAAA
ACTCTGTTCTGTATGTAAGTTTTGGGAGCATGGCCAAGTTCCCATCTGCT
CAGCTTCTTGAAATAGCTCATGGGCTTGAAGCTTCAGGTCATAGTTTCAT
CTGGGTTGTCAGAAAAGTTGACGGGGATGATGATGTAGACGTGTGGCTTC
CAGATTTTGAGAAGAAAATGAAAGAGAACAACAAGGGTTTCATCATAAGG
AATTGGGCACCACAATTGCTCATATTGGACCATCCAGCAATTGGAGGTTT
GCTGAATCACAGTGGATGGAATTCAGTACTGGAAGGTGCTACAGCAGGCT
TGCCAATGATCACTTGGCCTCTGTATGCCGAGCATTTTTACAATGAAAGG
TTGGTTCTAGATGTGTTGAAAATTGGAGTACCAGTTGGGGTGAAGGAGTG
GAAGAACTTGCATGAGGTGGGTGAGTTGGTGAGAAGGGATGCAATTGCCA
AGGCAATTAAATTGTTAATGGGTAGTGGAGAAGAAGCTGAGGTAATGAGG
AAAAAAGCCAAAGAGCTTGGTGTTGGAGCAAAGAAAGGTATTCAGGTTGG
AGGTTCTTCTCATACCAATTTGATAGCAGTGATTGATGAGTTAAAGTCAC
TAAAGAAATCAAGAATTCAGGGTGTCTGA
An enzyme involved in making QA-Di
from QA-Mono (Qs-3-O-GalT).
SEQ ID NO 30
MVESPADHDVLKIIVLPWVTSGHMIPMVDAARLFAMHGADVTIITTPANA
LTFQKSVDRDFNSGRLIRTHTLKFPAAEVGVPEGVENFNNTSPEMTSKVY
LGVSMLREPTQQLIEDLRPDCLITDMFYPWAVDVADKLGIPRLIFQGPGS
FGLSAMHSIKQYEPFKSVTSDTETFPLPGLPHKVEMTRLQIPKWVREPNG
YTQLMGRVKDSERRSYGSLVNSFYDFEGPYEEHYRKATGQRVWSIGPVSV
WVNQDAADKVGRGQDLVAEDQNSWLNWLNSKEKNSVLYVSFGSMAKFPSA
QLLEIAHGLEASGHSFIWVVRKVDGDDDVDVWLPDFEKKMKENNKGFIIR
NWAPQLLILDHPAIGGLLNHSGWNSVLEGATAGLPMITWPLYAEHFYNER
LVLDVLKIGVPVGVKEWKNLHEVGELVRRDAIAKAIKLLMGSGEEAEVMR
KKAKELGVGAKKGIQVGGSSHTNLIAVIDELKSLKKSRIQGV*
A nucleic acid sequence which encodes
the enzyme according to SEQ ID NO 32.
SEQ ID NO 31
ATGGTCTCCGGCGACGACGATGTTTCTCGTCGGCCACTGAAAGTTTACTT
CATTGCACACCCCTCACCTGGCCATATTGCCCCTCTGACCAAAATAGCCC
ATCTCTTCGCTGCCCTCGGTGAGCACGTGACTATTCTCACTACTCCCGCC
AATGTCCACTTCCATGAGAAATCCATCGACAAAGGAAAGGCTTCCGGCTA
TCATGTTAACATCCACACCGTTAAATTTCCTTCTAAAGAGGTCGGTCTCC
CTGACGGCATCGAAAACTTCTCTTACGCCTCCGATGTTGAAACAGCAGCT
AAAATTTGGGCTGGATTCGCCATGCTACAAACTGAAATGGAGCAATATAT
GGAGCTTAACCCACCCGATTGCATCGTTGCCGACATGTTCACCTCCTGGA
CCTCCGACTTTGCTATCAAATTGGGAATCACAAGAATCGTTTTCAACGTC
TATTGTATTTTCACACGCTGTTTGGAAGAAGCCATCCGATCACCGGACTC
GCCACACTTGAACAAAGAAATCTCTGATAATGAACCGTTTGTTATCCCGG
GTCTACCAGACCCCATAACAATTACCCGAGCTCAACTGCCCGACGGTACC
TTTTCTCCCATGAAAGAACTAGCTAGAACAGCTGAGTTGAAGAGCTTTGG
AATGGTGATCAACGGGTTTTCCGAACTCGAAACCGATTACATCGAGCATT
ACAAGAAAATCATGGGTCACAAACGGATTTGGCATGTCGGACCCCTTCAG
CTAATCCACCGTAACGATGAAGACAAAATTCAGAGGAGCCACAAGACAGC
GGTGCTGAGTGATAACGATAACGAGTTAGTGAGTTGGCTTAACTCGAAGA
AACCCGACTCAGTTATTTACATTTGCTTCGGTAGTGCAACTCGTTTCTCT
AATCACCAGCTCTATGAAATCGCCTGTGGATTAGAAGCTTCCGGGCACCC
ATTTTTGTGGGGCCTACTTTGGGTGCCAGAAGATGAAGATAACGATGACG
TGGGCAACAAATGGTTGCCAGCTTTCGAAGAAAGAATTAAAAAGGAAAAT
AAGGGAATGATTTTAAGGGGGTGGGCTCCACAGATGTTAATCTTAAACCA
CCCGGCGATCGGTGGTTTCATGACGCATTGTGGTTGGAATGCGGTGGTGG
AAGCACTTTCATTCGGTGTTCCGACTATTACGCTTCCAGTTTTCTCGGAG
CAGTTTTATACTGAGAGACTGATATCACAAGTGCTCAAGACTGGTGTGGA
GGTTGGTGCAGAGAAGTGGACCTATGCATTTGATGCGGGGAAATATCCGG
TGAGTAGGGAAAAGATAGCGACGGCGGTGAAGAAGATATTAGACGATGGA
GAAGAGGCAGAAGGAATGAGAAAGCGGGCCAGGGAGATGAAAGAAAAAGC
CCAAAAAAGTGTTGAAGAAGGTGGATCCTCTTATAATAATTTAACGGCTA
TGATTGAAGATCTTAAAGAATTTAGGGCTAACAATGGCAAGGCTGCACAA
GATCATGAATCGTGA
An enzyme involved in making QA-TriR or
QA-TriX from QA-Di (Qs-3-O-RhaT/XylT).
SEQ ID NO 32
MVSGDDDVSRRPLKVYFIAHPSPGHIAPLTKIAHLFAALGEHVTILTTPA
NVHFHEKSIDKGKASGYHVNIHTVKFPSKEVGLPDGIENFSYASDVETAA
KIWAGFAMLQTEMEQYMELNPPDCIVADMFTSWTSDFAIKLGITRIVFNV
YCIFTRCLEEAIRSPDSPHLNKEISDNEPFVIPGLPDPITITRAQLPDGT
FSPMKELARTAELKSFGMVINGFSELETDYIEHYKKIMGHKRIWHVGPLQ
LIHRNDEDKIQRSHKTAVLSDNDNELVSWLNSKKPDSVIYICFGSATRFS
NHQLYEIACGLEASGHPFLWGLLWVPEDEDNDDVGNKWLPAFEERIKKEN
KGMILRGWAPQMLILNHPAIGGFMTHCGWNAVVEALSFGVPTITLPVFSE
QFYTERLISQVLKTGVEVGAEKWTYAFDAGKYPVSREKIATAVKKILDDG
EEAEGMRKRAREMKEKAQKSVEEGGSSYNNLTAMIEDLKEFRANNGKAAQ
DHES*
A nucleic acid sequence which encodes
the enzyme according to SEQ ID NO 34.
SEQ ID NO 33
ATGGTCTCCGGCGACGACGACGTTTCTCGTCGGCCACTGAAAGTTTACTT
TATTGCACACCCCTCACCTGGCCATATTGCCCCTCTAACCAAAATAGCCC
AACTCTTTGCTGCACGTGGTGAGCACGTGACTATTCTTACTACTCCCGCC
AATGTCCACTTCCATGAGAAATCCATCGACAAAGGAAAGACTTCCGGCTA
TCATGTTAACATCCACGCCGTTAAATTTCCTTCTAAAGAGGTCGGTCTCC
CCGACGGCATCGAAAACTTCTCTCACGCCTCCGATAATGAAACAGCAGCC
AAAATTTGGGCCGGATTCTCCATGCTTCAAACTGAAATGGAGCAATATAT
GGAACAAAACCCACCCGATTGCATTGTTGCCGACATGTTCAACCGCTGGA
CTTCCGACTTCGCTATCAAATTGGGAATCCCGAGAATAGTTTTCAACGTC
TACTGTATTTTCACACGCTGTTTGGAAGAAGCAATCAGATCACCTGACTC
GCCACACTTGAAACTAAACTCCGATAATGAACAGTTTATTATTCCGGGTC
TACCCGACCCCATAACAATTACCCGAGCTCAACTGCCCGACGGTGCCTTT
TCTGTCGTCAAAGAACAAGTTAGTGAAGCTGAGTTGAAAAGCTTCGGAAT
GGTGATCAACGGGTTTTCCGAACTCGAAACCGAATACATCGAGTATTACA
AGAATATCATGGGTCGAAAACGGATTTGGCATGTCGGACCCCTTCAGCTC
ATTTACCAAAACGATGACCCCAAAGTTCAGAGGAGCCAGAAGACAGCGGT
CGTGAGTGACAACGAGTTAGTGAGTTGGCTTGACTCGAAGAAACCCGACT
CAGTGATTTACATTTCCTTCGGTAGTGCAATTCGTTTCTCTAATAAGCAG
CTCTATGAAATAGCATGTGGATTAGAAGCTTCCGGCTACCCATTTTTGTG
GGCCTTACTTTGGGTGCCAGAAGATGACGACGACGTGGGCAACAAATGGT
TGCCTGATTTCGAAGAAAGAATAAAAAGAGAAAATAAGGGAATAATTTTC
AGGGGGTGGGCCCCACAGATGTTAATCTTAAACCACCCGGCGATCGGTGG
TTTCATGACGCATTGTGGTTGGAATGCGGTGGTGGAAGCGCTTTCTTTCG
GTGTTCCGACTATTACGCTTCCGGTTTTCTCGGAGCAGTTTTATACTGAG
AGACTGATATCACAAGTGCTCAAGACTGGTGTCGAGGTCGGTGCAGAGAA
GTGGACCTATGCATTTGATGCGGGGAAATATCCGGTGAGTCGGGAAAAGA
TAGCGACGGCGGTGAAGAAGATATTAGACTGTGGAGAAGAGGCAGAAGGA
ATGAGAAAGCGGGCCAGGGAGATGAAAGAAAAAGCCCAAAAAAGTGTTGA
AGAAGGTGGGTCCTCTTATAATAATTTAACGGCTATGATTGAAGATCTTA
AAGAATTTAGGGCTAACAATGGCAAGGTTGCATGA
An enzyme involved in making QA-TriR
from QA-Di (Qs_0283850).
SEQ ID NO 34
MVSGDDDVSRRPLKVYFIAHPSPGHIAPLTKIAQLFAARGEHVTILTTPA
NVHFHEKSIDKGKTSGYHVNIHAVKFPSKEVGLPDGIENFSHASDNETAA
KIWAGFSMLQTEMEQYMEQNPPDCIVADMFNRWTSDFAIKLGIPRIVFNV
YCIFTRCLEEAIRSPDSPHLKLNSDNEQFIIPGLPDPITITRAQLPDGAF
SVVKEQVSEAELKSFGMVINGFSELETEYIEYYKNIMGRKRIWHVGPLQL
IYQNDDPKVQRSQKTAVVSDNELVSWLDSKKPDSVIYISFGSAIRFSNKQ
LYEIACGLEASGYPFLWALLWVPEDDDDVGNKWLPDFEERIKRENKGIIF
RGWAPQMLILNHPAIGGFMTHCGWNAVVEALSFGVPTITLPVFSEQFYTE
RLISQVLKTGVEVGAEKWTYAFDAGKYPVSREKIATAVKKILDCGEEAEG
MRKRAREMKEKAQKSVEEGGSSYNNLTAMIEDLKEFRANNGKVA*
A nucleic acid sequence which encodes
the enzyme according to SEQ ID NO 36.
SEQ ID NO 35
ATGGTCTCCGGCGACGATACCGTTTCACGGCCACTGATAGTTTACTTTAT
TGCACACCCCTCACCTGGCCATATTGCCCCTCTAACCAAAATAGCCCAAC
TCTTCGCTGCACGTGGTGAGCACGTCACTATTCTTACTACTCCCGCCAAT
GTCCACTTCCATGAGAAATCCATCGACAAAAGAAAGAATTCCGGCTATCA
TGTTAACATCCACACCGTTAAATTTCCTTCTAAAGAGGTCGGTCTCCCTG
ACGGCATCGAAAACTTCTCTCACGCCTCCGATAATGAAACAGCAGCCAAA
ATTTGGGCCGGATTCTCCATGCTTCAAACTGAAATGGAGCAATATATGGA
ACAAAACCCACCCGATTGCATCGTTGCCGACATGTTCAACCGCTGGACTT
CCGACTTCGCTATCAAATTGGGAATCCCGAGAATAGTTTTCAACGTCTAC
TGTATTTTCACACGCTGTTTGGAAGAAGCAATCAGATCACCTGACTCGCC
ACACTTGAAACTAAACTCCGATAATGAACAGTTTATTATTCCCGGTCTAC
CCGACCCCATAACAATTACCCGAGCTCAACTCCCCGACGGTGCCTTTTCT
GTCGTCAAAGAACAAGTTAGTGAAGCTGAGTTGAAAAGCTTCGGAATGGT
GATCAACGGGTTTTCCGAACTCGAAACTGAATACATCGAGTATTACAAGA
ATATCATGGGTCGCAAACGGATTTGGCATGTCGGACCCCTTCAGCTAATT
TACCAAAACGACGACCCCAAAGTTCAGAGGAGCCAGAAGACAGCGGTCTT
GAGTGACAACGAGTTAGTGAGTTGGCTTGACTCGAAGAAACCCGACTCAG
TGATTTACATTTCCTTCGGTAGTGCAATTCGTTTCTCTAATAAGCAGCTC
TATGAAATCGCATGTGGATTAGAAGCTTCCGGCTACCCATTTTTGTGGGC
CTTACTTTGGGTGCCAGAAGATGATGACGACGTGGGCAACAAATGGTTGC
CGGGTTTCGAAGAAAGAATAAAAAGAGAAAATAAGGGAATAATTTTCAGG
GGGTGGGCCCCACAGATGTTAATCTTAAACCACCCGGCGATCGGTGGTTT
CATGACGCATTGTGGTTGGAATGCGGTGGTGGAAGCACTTTCATTCGGTG
TTCCGACTATTACGCTTCCAGTTTTCTCGGAGCAGTTTTATACTGAGAGA
CTGATATCACAAGTGCTCAAGACTGGTGTGGAGGTTGGTGCAGAGAAGTG
GACCTATGCATTTGATGCGGGGAAATATCCGGTGAGTAGGGAAAAGATAG
CGACGGCGGTGAAGAAGATATTAGACGATGGAGAAGAGGCAGAAGGAATG
AGAAAGCGGGCCAGGGAGATGAAAGAAAAAGCCCAAAAAAGTGTTGAAGA
AGGTGGATCCTCTTATAATAATTTAACGGCTATGATTGAAGATCTTAAAG
AATTTAGGGCTAACAATGGCAAGGCTGCAATGAAATCATGA
An enzyme involved in making QA-TriR
from QA-Di (DN20529_c0_g2_i8).
SEQ ID NO 36
MVSGDDTVSRPLIVYFIAHPSPGHIAPLTKIAQLFAARGEHVTILTTPAN
VHFHEKSIDKRKNSGYHVNIHTVKFPSKEVGLPDGIENFSHASDNETAAK
IWAGFSMLQTEMEQYMEQNPPDCIVADMFNRWTSDFAIKLGIPRIVFNVY
CIFTRCLEEAIRSPDSPHLKLNSDNEQFIIPGLPDPITITRAQLPDGAFS
VVKEQVSEAELKSFGMVINGFSELETEYIEYYKNIMGRKRIWHVGPLQLI
YQNDDPKVQRSQKTAVLSDNELVSWLDSKKPDSVIYISFGSAIRFSNKQL
YEIACGLEASGYPFLWALLWVPEDDDDVGNKWLPGFEERIKRENKGIIFR
GWAPQMLILNHPAIGGFMTHCGWNAVVEALSFGVPTITLPVFSEQFYTER
LISQVLKTGVEVGAEKWTYAFDAGKYPVSREKIATAVKKILDDGEEAEGM
RKRAREMKEKAQKSVEEGGSSYNNLTAMIEDLKEFRANNGKAAMKS*
A nucleic acid sequence which encodes
the enzyme according to SEQ ID NO 38.
SEQ ID NO 37
ATGGTCTCCGGCGACGACGATGTTTCTCGTCGGCCACTGAAAGTTTACTT
CATTGCACACCCCTCACCTGGCCATATTGCCCCTCTGACCAAAATAGCCC
ATCTCTTCGCTGCCCTCGGTGAGCACGTGACTATTCTCACTACTCCCGCC
AATGTCCACTTCCATGAGAAATCCATCGACAAAGGAAAGGCTTCCGGCTA
TCATGTTAACATCCACACCGTTAAATTTCCTTCTAAAGAGGTCGGTCTCC
CTGACGGCATCGAAAACTTCTCTTACGCCTCCGATGTTGAAACAGCAGCT
AAAATTTGGGCTGGATTCGCCATGCTACAAACTGAAATGGAGCAATATAT
GGAGCTTAACCCACCCGATTGCATCGTTGCCGACATGTTCACCTCCTGGA
CCTCCGACTTTGCTATCAAATTGGGAATCACAAGAATCGTTTTCAACGTC
TATTGTATTTTCACACGCTGTTTGGAAGAAGCCATCCGATCACCGGACTC
GCCACACTTGAACAAAGAAATCTCTGATAATGAACCGTTTGTTATCCCGG
GTCTACCAGACCCCATAACAATTACCCGAGCTCAACTGCCCGACGGTACC
TTTTCTCCCATGAAAGAACTAGCTAGAACAGCTGAGTTGAAGAGCTTTGG
AATGGTGATCAACGGGTTTTCCGAACTCGAAACCGATTACATCGAGCATT
ACAAGAAAATCATGGGTCACAAACGGATTTGGCATGTCGGACCCCTTCAG
CTAATCCACCGTAACGATGAAGACAAAATTCAGAGGAGCCACAAGACAGC
GGTGCTGAGTGATAACGATAACGAGTTAGTGAGTTGGCTTAACTCGAAGA
AACCCGACTCAGTTATTTACATTTGCTTCGGTAGTGCAACTCGTTTCTCT
AATCACCAGCTCTATGAAATCGCCTGTGGATTAGAAGCTTCCGGGCACCC
ATTTTTGTGGGGCCTACTTTGGGTGCCAGAAGATGAAGATAACGATGACG
TGGGCAACAAATGGTTGCCAGCTTTCGAAGAAAGAATTAAAAAGGAAAAT
AAGGGAATGATTTTAAGGGGGTGGGCTCCACAGATGTTAATCTTGAATCA
CCCGGCGATCGGTGGTTTCATGACGCATTGTGGTTGGAATGCGGCGGTGG
AGGCGCTTTCTTCCGGTGTTCCGATTATTACATTTCCGGTTTTCTCGGAT
CAGTTTTATAATGAAAGGCTGATATCACAAGTGCATAAGTGTGGGGTGGG
GGTTGGTACGGAGGCGTGGAGCTATGCATTCGATGCCGGGAAGAATCCGG
TGGGTCGGGAAAAGATAATGACGGCGGTGAAGAAGATATTAGACGGTGGA
GAAGAGGCGGAAGGAATGAGAAAGAGGGCCCGGGAGCTGAAAGAAATAGC
TAAAAGAAGTGTGGAAGAAGGTGGGTCCTCTTATAATAATTTAACGGCTA
TGATTCAAGATCTGAAAGAATTTAGAGCTAACAATGGCAAGGCTGCACAA
GATCATGAATCGTGA
An enzyme involved in making QA-TriX
from QA-Di (Qs_0283870).
SEQ ID NO 38
MVSGDDDVSRRPLKVYFIAHPSPGHIAPLTKIAHLFAALGEHVTILTTPA
NVHFHEKSIDKGKASGYHVNIHTVKFPSKEVGLPDGIENFSYASDVETAA
KIWAGFAMLQTEMEQYMELNPPDCIVADMFTSWTSDFAIKLGITRIVFNV
YCIFTRCLEEAIRSPDSPHLNKEISDNEPFVIPGLPDPITITRAQLPDGT
FSPMKELARTAELKSFGMVINGFSELETDYIEHYKKIMGHKRIWHVGPLQ
LIHRNDEDKIQRSHKTAVLSDNDNELVSWLNSKKPDSVIYICFGSATRFS
NHQLYEIACGLEASGHPFLWGLLWVPEDEDNDDVGNKWLPAFEERIKKEN
KGMILRGWAPQMLILNHPAIGGFMTHCGWNAAVEALSSGVPIITFPVFSD
QFYNERLISQVHKCGVGVGTEAWSYAFDAGKNPVGREKIMTAVKKILDGG
EEAEGMRKRARELKEIAKRSVEEGGSSYNNLTAMIQDLKEFRANNGKAAQ
DHES*
Acanthocystis turfacea chlorella virus 1
UDP-D-glucose 4,6-dehydratase (ATCV-1)
coding sequence (1053 bp):
NB: This sequence was codon-optimised for
expression in N. benthamiana. The original
sequence can be found as Genbank ID:
NC_008724.1 (see locus tag ATCV_z554R).
SEQ ID 39
ATGAATAGTCAGGAGTATACTCCTAAATCAGTCCTTGTAACTGGTGGGGC
AGGTTTCATTGGCAGCCATGTCGTGATGAAATTGGTCCAGAGGTATCCGG
AATGCAAGGTCGTTGTTCTTGACAAAATGGATTACTGCGCTACCTTAAAT
AATCTTGCTACGGTACGAGATGCCCCCAATTTTAAATTTGTAAAAGGTGA
TATACAAAGCACTGATCTTTTAGCTCACGTGCTCAAACAGGAAAAGATAG
ATACCATTATGCACTTTGCCGCCCAGACCCATGTAGATAATAGCTTTGGC
AATAGTTTAGCATTTACGATGAACAACGTATACGGCACTCACGTCCTTCT
CGAATGTGCCCGATTGTATGGTGGTGTTCAGAGATTCATTAACGTGTCAA
CTGATGAGGTCTACGGTGAGAGTTCCTTGGGAAAGAAGGAGGGGTTGGAC
GAACACTCCTCCCTCGAACCGACAAATCCTTATGCCGCCGCAAAGGCTGG
AGCTGAAATGATGGCTAGAGCATATCATACGTCATACAAACTCCCGGTAA
TAGTCACGCGTGGCAATAATGTATATGGTCCGCACCAGTTCCCCGAAAAA
ATGATCCCCAAATTTATTCTCAGGGCAACCAGAGGCCTCGATTTGCCAAT
ACATGGTGATGGCGGCGCCCTGCGATCATACCTTTACGTCGATGATGTTG
CCGAAGCTTATATTACAATTCTTTTAAAGGGCAATGTTGGAGAGACCTAT
AACATCGGCACGCAAAAGGAGCGATCCGTTGTGGACGTGGCCCATGACAT
CTGCAAGATTTTTAACCGAGACTCAGATACAGCTATATGGCACGTTAAAG
ACCGAGCCTTCAATGATCGACGTTATTTTATTTCTGATAAAAAATTACTT
GACCTCGGCTGGCAAGAAAAAACCACCTGGGAGGACGGTCTTAAGCAAAC
TGTAGGGTGGTATTTGCAACATGCAACTAGGTCCTACTGGGATCACGGCA
ACATGGAATTAGCTTTAGACGCCCATCCGACACTTCAAGTTCCTAAATTC
TAA
Acanthocystis turfacea chlorella virus 1
UDP-D-glucose 4,6-dehydratase (ATCV-1)
translated nucleotide sequence (350 aa):
SEQ ID 40
MNSQEYTPKSVLVTGGAGFIGSHVVMKLVQRYPECKVVVLDKMDYCATLN
NLATVRDAPNFKFVKGDIQSTDLLAHVLKQEKIDTIMHFAAQTHVDNSFG
NSLAFTMNNVYGTHVLLECARLYGGVQRFINVSTDEVYGESSLGKKEGLD
EHSSLEPTNPYAAAKAGAEMMARAYHTSYKLPVIVTRGNNVYGPHQFPEK
MIPKFILRATRGLDLPIHGDGGALRSYLYVDDVAEAYITILLKGNVGETY
NIGTQKERSVVDVAHDICKIFNRDSDTAIWHVKDRAFNDRRYFISDKKLL
DLGWQEKTTWEDGLKQTVGWYLQHATRSYWDHGNMELALDAHPTLQVPK
F*
Aggregatibacter actinomycetemcomitans
NDP-4-keto-6-deoxy-glucose 4-ketoreductase
(AaFCD) coding sequence (693 bp):
NB: This sequence was codon-optimised for
expression in N. benthamiana. The original
sequence can be found as Genbank ID:
AB002668.1 (sequence 15271 . . . 15963bp).
SEQ ID 41
ATGATAATTGGCAACGGTATGCTGGCAAAAGCCTTTGAATCATTCCATAA
GCGAACTTACAATTACATAATATTTGCATCCGGAGTGAGCAACTCAAACG
AAACTTCCTTCGAGAATTTCAACAGAGAGAAGGAATTGCTTCTTGAAGTC
CTGGAGCAATATAAAGACAAAACTATCGTTTACTTTAGTTCCTGCTCCAT
ATACGATTCTAGTTTGACGAATTCTTTGTATGTCTACCACAAAATGTGTA
TGGAGAGACTGGTGCGTGAAAACTCCAAGAATTATCTCATAGCCCGTCTC
CCCCAAGTTATTGGTAAAACGTATTCACCAACCATTGTCAACTTCCTTTT
TAACAAAATCAAAAATAGGGAGTGTTTCAGCATATTCGGAAAAGCTCACC
GAAATTTTATCGACGTGGATGATGTCGTTAAGGTCACCAATTACTTATTG
AAGGAGGGTCTGTTCATTAACAGTATTGTGAACTTGGCAAGCACGCACCA
TACCTCCATGTACGAATTAATCTTATATTTGGAAAAAATAAGTAATCAAC
GTGCCTTCTATAATGTTGAGAACAAAGGGTCTAGGTACTTTATTGATGTT
TCAATACTGCAGGATGTTTATCAGAAGCTGGGGATCAAATTTGACAAAGA
TTACGTAGAAAAGGTTATCAACAAGTACTACGCTATTAAGTAA
Aggregatibacter actinomycetemcomitans
NDP-4-keto-6-deoxy-glucose 4-ketoreductase
(AaFCD) translated nucleotide sequence
(230 aa):
SEQ ID 42
MIIGNGMLAKAFESFHKRTYNYIIFASGVSNSNETSFENFNREKELLLEV
LEQYKDKTIVYFSSCSIYDSSLTNSLYVYHKMCMERLVRENSKNYLIARL
PQVIGKTYSPTIVNFLFNKIKNRECFSIFGKAHRNFIDVDDVVKVTNYLL
KEGLFINSIVNLASTHHTSMYELILYLEKISNQRAFYNVENKGSRYFIDV
SILQDVYQKLGIKFDKDYVEKVINKYYAIK*
Anoxybacillus tepidamans NDP-4-keto-
6-deoxy-glucose 4-ketoreductase (AtFCD)
coding sequence (927 bp):
NB: This sequence was codon-optimised
for expression in N. benthamiana.
The original sequence can be found as
Genbank ID: AY883421.5 (sequence
13209-14135bp).
SEQ ID 43
ATGAAGAGGATACTGATACTCGGATGCGGTTACCTGGGTTTAAATCTCGC
AAACTATTTTTGTAAAAAAAATTATGATGTCTCAGTGATAGGGAGAAAGT
CTGTCTATAGCAATTTTTTGGAAGAGGAGATAGAGTTCATAGAAGATGAT
ATCAAAAATATAAATAGTTATAAGCACATGTTTAATGAGGAGACAACCGT
CATTTACGCCATAGGAAGTATTAACGCAAATAACTATTTTATGGACCTGA
GGAATGATATAGAAAACTCATACATCCCCTTCATTAACCTCCTTAACTTT
CTTTCCGAAAAGTATATTCAAAAGTTCGTCTTTCTCTCTTCAGCCGGAAC
AGTCTATGGGAACGTGAATAAGAATTATATAAGCGAGAATGAGATTCTTA
ACCCAATTTCAATCTATGGTTTGCAGAAAGCCTTCTTTGAACAACTGATA
AGGATTAAAAACAATGAGGCTAGCCATTTCAGGTATTTGATCTTCAGAAT
ATCTAACCCCTATGGGGGAATCAACATTCCGAACAAGAATCAGGGAATTA
TTCCGACGTTAGTGTACAAAGCCGTGAACAATGAGCCTTTCGAACTTTGG
GCATCAATCAATACCATCCGTGATTATATTTACATCGATGACCTTAGCGA
ATTGATCTACAAAACAATCTATCTGGACATTTATAACGAGACCCTCAATC
TCGGGTCCGGTAAAGGAACATCAATCAAGCAACTCATTAGCCTCGTGGAG
GAGATTTTGGGAAAGAAGATCACTATTCTTGAAAAGCCCCCCATAAAGAC
TAACGTTTTGAAAAATATACTTGATATTTCTAAGCTCGTCAACACCGTAG
GCTACGAACCAAAGATCAGCATTGAAGAGGGTATTAGCCGTTACATCAAC
ACTATTTTAACGAAGAACATTTTTTAA
Anoxybacillus tepidamans NDP-4-keto-
6-deoxy-glucose 4-ketoreductase (AtFCD)
translated nucleotide sequence (308 aa):
SEQ ID 44
MKRILILGCGYLGLNLANYFCKKNYDVSVIGRKSVYSNFLEEEIEFIEDD
IKNINSYKHMFNEETTVIYAIGSINANNYFMDLRNDIENSYIPFINLLNF
LSEKYIQKFVFLSSAGTVYGNVNKNYISENEILNPISIYGLQKAFFEQLI
RIKNNEASHFRYLIFRISNPYGGINIPNKNQGIIPTLVYKAVNNEPFELW
ASINTIRDYIYIDDLSELIYKTIYLDIYNETLNLGSGKGTSIKQLISLVE
EILGKKITILEKPPIKTNVLKNILDISKLVNTVGYEPKISIEEGISRYIN
TILTKNIF*
Escherichia coli NDP-4-keto-6-deoxy-
glucose 4-ketoreductase (EcFCD)
coding sequence (951 bp):
NB: This sequence was codon-optimised
for expression in N. benthamiana. The
original sequence can be found as
Genbank ID: AY528413.1 (sequence
3156-4106bp).
SEQ ID 45
ATGGATGCTCGTAAAAATGGGGTATTAATAACCGGTGGAGCTGGGTTCAT
AGGTAAAGCCTTAATAACTGAAATGGTCGAACGTCAAATTCCCCTGGTGT
CATTTGACATCAGCGATAAGCCCGACAGTTTGCCAGAGCTTTCCGAATAT
TTCAACTGGTATAAATTCTCATACCTTGAGAGTTCACAGAGGATTAAAGA
GCTTCACGAAATAGTTTCCAGGCATAACATCAAAACGGTCATCCATTTAG
CTACAACTATGTTTCCCCACGAATCCAAAAAGAACATCGATAAGGATTGC
TTAGAAAACGTTTATGCCAACGTGTGTTTCTTTAAGAATTTATATGAAAA
CGGCTGTGAAAAAATTATCTTCGCCTCATCAGGTGGCACCGTATATGGGA
AGTCTGATACACCCTTCTCCGAAGACGATGCCCTGCTTCCCGAAATTTCC
TACGGACTGTCCAAGGTTATGACTGAAACTTATCTCCGATTCATAGCCAA
GGAATTGAATGGGAAGTCCATCTCTCTCAGAATATCTAACCCCTATGGTG
AGGGGCAAAGGATTGACGGGAAACAAGGAGTCATTCCAATTTTCCTCAAT
AAAATCAGCAACGACATCCCCATCGACATCATTGGCTCTATCGAATCAAA
GCGAGACTACATTTATATTTCAGATCTCGTACAAGCTTTCATGTGCTCTC
TGGAATATGAAGGTCACGAAGACATATTTAATATAGGTTCTGGGGAAAGC
ATAACTCTGAAGAAATTGATCGAGACGATTGAGTTCAAGCTGAACAAGAA
GGCTGTGATTGGATTTCAAGATCCGATCCACACCAATGCCAATGGTATAA
TTCTCGACATCAAACGAGCCATGGCAGAACTCGGCTGGAGGCCCACCGTG
GTCCTGGATGATGGCATCGATAAATTAATCAAGAGCATTCGATGCAAGTA
A
Echerichia coli NDP-4-keto-6-deoxy-
glucose 4-ketoreductase (EcFCD)
translated nucleotide sequence
(316 aa):
SEQ ID 46
MDARKNGVLITGGAGFIGKALITEMVERQIPLVSFDISDKPDSLPELSEY
FNWYKFSYLESSQRIKELHEIVSRHNIKTVIHLATTMFPHESKKNIDKDC
LENVYANVCFFKNLYENGCEKIIFASSGGTVYGKSDTPFSEDDALLPEIS
YGLSKVMTETYLRFIAKELNGKSISLRISNPYGEGQRIDGKQGVIPIFLN
KISNDIPIDIIGSIESKRDYIYISDLVQAFMCSLEYEGHEDIFNIGSGES
ITLKKLIETIEFKLNKKAVIGFQDPIHTNANGIILDIKRAMAELGWRPTV
VLDDGIDKLIKSIRCK*
A nucleic acid sequence which encodes
the QsFSL-1 enzyme according to SEQ
ID NO 48. QsFSL-1
SEQ ID NO 47
ATGGCAGAAGCAACAGAGAGGTATGCTGTTGTGACAGGATCTAATAAAGG
AATTGGATTTGGGATATGCAAGCAGCTGGCTTCTAAGGGGATTACAGTAG
TGCTAACAGCTAGAGATGATAAGAGAGGTCTTGAAGCAGTTGAGAAATTG
AAAGAATTTGATCTGCATGGTCATGTGCTTTTTCATCAACTTGATGTGTC
TGATACAGCTAGTGTTACTAGCCTTGCAGATTTTATCAAAACCCAGTTTG
GGAAACTAGATATCTTGGTAAACAATGCAGGTATAACTGGAACCACTGTA
GATGCTGATGCTTTAGCATCTTCAGGCTATGGTACTGGGGGTGAACGTAA
ACCTATTGATTGGAATAAAATAGTGATAGAGACTTATGAATCAGTTGAAA
AAGCTATCAACACCAACTATTATGGAGCCAAAAGAATGGCTGAAGCACTT
ATACCCCTTCTTCAAGTATCAGACTCACCAAGGATTGTTAATGCTTCCTC
TCCTATGGCAAAGCTAGAGAATATTCCAAGTGGATGGGGTAAGGAAGTGC
TAAGTGATGTTGATAGCCTAACAGAAGAGAAACTTGATGAGATGTTGACC
CAATTATTGAAAGATTTCAAAGAGGGTTCATTAGAAACCAAAGGCTGGCC
TACTCTTATGTCTTCGTATATAATCTCAAAAGCTGCTTTAAATGCCTACA
CAAGGATTCTTGCTAAGAAGTACCCATCTTTCTGCATCAATTGTGTAGAC
CCTGGTCATGTGAAGACTGACATAAATCGTCACACCGGCCACTTAAGTAT
TGATGAAGGTGCTGAAAGCCATGTGAGATTGGCCCTGCTGCCTGATGGTG
GCCCTTCTGGACATTTCTTCTCCAGGACTGAAGAGACACCATTTTGA
An oxidoreductase enzyme capable of
enhancing the activity of a
fucosyltransferase (QsFSL)
SEQ ID NO 48
MAEATERYAVVTGSNKGIGFGICKQLASKGITVVLTARDDKRGLEAVEKL
KEFDLHGHVLFHQLDVSDTASVTSLADFIKTQFGKLDILVNNAGITGTTV
DADALASSGYGTGGERKPIDWNKIVIETYESVEKAINTNYYGAKRMAEAL
IPLLQVSDSPRIVNASSPMAKLENIPSGWGKEVLSDVDSLTEEKLDEMLT
QLLKDFKEGSLETKGWPTLMSSYIISKAALNAYTRILAKKYPSFCINCVD
PGHVKTDINRHTGHLSIDEGAESHVRLALLPDGGPSGHFFSRTEETPF*
A nucleic acid sequence which encodes
the enzyme according to SEQ ID NO 50.
QsFSL-2
SEQ ID NO 49
ATGGGTTCAGATGGAAGGGATGTAGCAGAGAGGTATGCAGTGGTTACAGG
TGCAAACAAAGGCATAGGCCTAGAAACCGTGCGGCAACTAGCGTCTCACG
GCATTACAGTTGTGTTGACAGCTCGAGATGAGAAGAGAGGGACTGAAGCC
ACAAGAAAGCTCCACCAGCTGGGTTTGTCAAATTTGATTTTCCATCAGCT
GGATGTTTTAGACCCTGTTAGCATTCAGTCACTGGCCAAGTTCATCCAAG
ACAAATTTGGCAGGCTTGATATCCTGGTTAATAATGCTGGAGCATCTGGA
CTTGCAGCTGATGAGAAAGCTCTGAAGGCATTAAACATAGATAATGCAGC
TTGGCTCTCAGGCAAGGCCGCCAATTTAGTTCAAGGAATTGTCACACATA
CCTATGAGCAAGGCGAAGAATGCATAAATACAAACTATTATGGTGTCAAA
AGGGTGACGGAAGCTCTCCTACCGCTGTTACAACTTTCCCCTATAGGAGC
AAGGATAATAAATGTTTCCTCTTGCAGGGGTGAGCTAAAGAGGATTCCAA
TGAACGTAAGAAATGAACTGGGCGACATCAAAGTTCTGACTGAAGGCAGA
ATAGATGCAATTTTGATGAAATTTCTACACGATTTTAAGGATAATGCACT
TGAGTCCAACGGATGGACATTGATGGGGCCTGCTTATAGCATTTCGAAGG
CCAGTCTCAATGCCTACACTAGACTTCTTGCCAAAAAGTACCCCGAGATG
CTCATTAACTGTGTTCATCCTGGTTATGTCAACACAGATATGACTTGGCA
TAGAGGGATACTGACGGTAGAAGAGGGTGCTAAAGGCCCAGCCATGCTAG
CTCTTTTGCAAGATGGAGGACCTACAGGTTGCTATTTTGATAGTACTCAA
CAGGCAGAATTTTAA
An oxidoreductase enzyme capable of
enhancing the activity of a
fucosyltransferase (QsFSL-2)
SEQ ID NO 50
MGSDGRDVAERYAVVTGANKGIGLETVRQLASHGITVVLTARDEKRGTEA
TRKLHQLGLSNLIFHQLDVLDPVSIQSLAKFIQDKFGRLDILVNNAGASG
LAADEKALKALNIDNAAWLSGKAANLVQGIVTHTYEQGEECINTNYYGVK
RVTEALLPLLQLSPIGARIINVSSCRGELKRIPMNVRNELGDIKVLTEGR
IDAILMKFLHDFKDNALESNGWTLMGPAYSISKASLNAYTRLLAKKYPEM
LINCVHPGYVNTDMTWHRGILTVEEGAKGPAMLALLQDGGPTGCYFDSTQ
QAEF*
A nucleic acid sequence which encodes
the enzyme according to SEQ ID NO 52.
SoFSL-1
SEQ ID NO 51
ATGGCTGAAGCATCCTCATTTCTTGCACAGAAAAGGTATGCGGTCGTGAC
AGGAGCAAACAAAGGACTAGGACTAGAAATATGCGGACAGCTTGCTTCAC
AGGGGGTGACGGTACTGCTGACATCCAGAGATGAAAAACGAGGCTTAGAA
GCCATTGAGGAGCTTAAGAAATCGGGGATTAATTCGGAAAATCTTGAATA
TCATCAGCTGGATGTTACTAAGCCAGCTAGTTTCGCTTCTCTGGCCGATT
TCATCAAGGCCAAATTTGGCAAGCTTGATATCCTGGTGAACAATGCAGGG
ATCAGCGGTGTTATTGTAGATTATGCAGCTTTAATGGAAGCCATTCGCCG
TCGAGGGGCAGAGATCAATTACGATGGAGTGATGAAACAGACCTACGAGC
TAGCAGAGGAATGCTTGCAAACAAATTACTATGGTGTGAAAAGAACCATT
AATGCTCTCCTTCCGCTACTTCAGTTTTCCGATTCACCAAGGATCGTCAA
TGTTTCCTCCGATGTTGGCCTCCTTAAGAAAATACCCGGCGAGAGAATCA
GAGAAGCCTTAGGCGACGTGGAAAAACTTACGGAAGAAAGCGTGGACGGG
ATTTTAGACGAGTTTCTAAGAGATTTCAAGGAAGGCAAGATCGCAGAGAA
AGGTTGGCCTACGTTTAAGAGCGCCTATTCAATCTCAAAGGCGGCGCTCA
ATTCGTACACGAGGGTTTTAGCACGGAAATACCCGTCGATCATCATCAAC
TGTGTCTGCCCGGGTGTCGTCAAAACCGATATCAATCTTAAAATGGGCCA
CTTGACGGTTGAAGAAGGCGCGGCCAGTCCCGTGAGGTTAGCACTCATGC
CCCTTGGTTCGCCTTCCGGCCTGTTCTATACTCGAAACGAAGTAACTCCA
TTTGAATGA
An oxidoreductase enzyme capable of
enhancing the activity of a
fucosyltransferase (SoFSL-1)
SEQ ID NO 52
MAEASSFLAQKRYAVVTGANKGLGLEICGQLASQGVTVLLTSRDEKRGLE
AIEELKKSGINSENLEYHQLDVTKPASFASLADFIKAKFGKLDILVNNAG
ISGVIVDYAALMEAIRRRGAEINYDGVMKQTYELAEECLQTNYYGVKRTI
NALLPLLQFSDSPRIVNVSSDVGLLKKIPGERIREALGDVEKLTEESVDG
ILDEFLRDFKEGKIAEKGWPTFKSAYSISKAALNSYTRVLARKYPSIIIN
CVCPGVVKTDINLKMGHLTVEEGAASPVRLALMPLGSPSGLFYTRNEVTP
FE*
A nucleic acid sequence which encodes
the SpolFSL enzyme according to SEQ
ID NO 54. SpolFSL
SEQ ID NO 53
ATGGCTGAACAATCCAACTTTCTGGCTGAAAAAAGGTATGCAGTAGTGAC
AGGTGCAAACAAAGGAATAGGGCTTGAAATATGCAGACAGCTTGCTTCTC
AAGGTGTGATTGTACTTATCACTTCTAGAGATGGAAAGAAAGGATTAGAA
GCCCTTAATGATCTCATTAAATCTGGAATTAGCTCTGATAATCTTCATTA
TCATCAGCTTGATGTTACTGACCCTATGAGTATTACTGCTCTTGCTGGTT
TCATCAATTCCAAATTTGGCAAGCTTGATATTCTGGTGAACAATGCTGGG
ATAGGTGGATTTATAATTGACTACGATGCTATCAAAGCAATAGGTTTTCG
CAATATCAATTATGACGAGATGATGACACAAACATATGAGCTTGCAAAAG
AATGCTTGGAAACAAACTACTATGGAGTTAAGAGAACAACTGAAGCTTTG
CTTCCTCAGCTGGAGTTATCGGATTCACCAAGGATCATCAATGTCTCCTC
TTCTACGGGGATGTTGAAGAATATACCAAATGAGAGGATCAGAGGAGTCT
TGGGTGATGCAGAGAATCTTACAGAAGAAAAAGTTGAAGCGATTTTGAAT
GAGTTACTGACAGATTTCAAAGATGGTTCATTCAAAGAGAAAGAATGGCC
TTCTAGAATGGCAGCTTATACACTGTCAAAGGCGGCTTTGAATGCATATG
CAAGAATATTGGCTAAGAAATACCCGTCAATTATCATCAGTTGTGTTTGT
CCTGGTGTTACTAAGACAGATATGAACGGAAACTTGGGACAATTAACAGT
TGAAGAAGGGGCCGCAAGTCCGGTGAGAGTAGCATTGATGCCTCATGGTT
CACCTTCCGGTCTTTTCTATGCAAGAAGCGAAGTTTCTTCATATGAATAA
An oxidoreductase enzyme capable of
enhancing the activity of a
fucosyltransferase (SpolFSL)
SEQ ID NO 54
MAEQSNFLAEKRYAVVTGANKGIGLEICRQLASQGVIVLITSRDGKKGLE
ALNDLIKSGISSDNLHYHQLDVTDPMSITALAGFINSKFGKLDILVNNAG
IGGFIIDYDAIKAIGFRNINYDEMMTQTYELAKECLETNYYGVKRTTEAL
LPQLELSDSPRIINVSSSTGMLKNIPNERIRGVLGDAENLTEEKVEAILN
ELLTDFKDGSFKEKEWPSRMAAYTLSKAALNAYARILAKKYPSIIISCVC
PGVTKTDMNGNLGQLTVEEGAASPVRVALMPHGSPSGLFYARSEVSSYE*
A nucleic acid sequence which encodes
the enzyme according to SEQ ID NO 56.
SEQ ID NO 55
ATGGCGGATCGAGTCATAAACAGCTACAAAAAGCTTCACGTAGTGCTGTT
TCCATGGCTGGCCTTTGGTCACATGATACCATTTTTAGAGCTTGCAAAGC
TGATAGCCCAAAAAGGTCACAAAATTTCCTATATCTCAACACCCAGAAAC
ATCCGACGCCTACCTAAAATTCCATCCCATTTATCCAACAACCTTAATTT
CATAGAATTCCCACTGCCTCATATACCCAATCTTCATGAAAATGTGGAAT
CAACAAACGACGTCACACATGACAATCCTATTGCCTATTATTTACTTATT
AAAGCCCTTGAGGGTTTGCAACAACCTATCACCACCTTCCTTGAAACCTC
AGATCCAGATTGGATAATTCATGACGTTTTTCCTCAATGGATAACCGCAA
CAGCCAGTAGGCTCCGCATTTCACATGCTTTTTACACAACTTCATCTGCT
TTGAGAACGGCTTCCAATTATTCTCCGTTAATGTCCTCTGAATTACCACA
GGATATTGCTACCGATTTCACTACTAAATCCACAAATCTTCTTGCTGCCA
AGGTTTTGGTTATCCGTAGCTGTCTAGAGCTTGAACCCAAGGAATTTGAA
CAGTGCAAAAATCTATGCGTGTCCAAAACGGTAATTCCATTGGGCGTTGT
CCCACCGTCAATTCAGGTGAACGATAACATTTCTAATATTAATGATGACG
ACAACGACTGGGTTAAGATTGTTGAGTGGTTAAACCAAGGGAAAGAGAAA
GGTTCTGTCATTTATGTAGCACTTGGTTCTGAGGTTTCACTGAGTGAACA
AGATTTGAAGGAGTTTGCACTTGGTTTGGAACTATCAGGGTTGTCATTCT
TTTGGGTGTTCAGGAATACTGGATCGTATGGGTTACCGGCTGGGTTTGAA
GACCGAGTTAAGGGTCGGGGAATTGTCTGGACTAGCTGGGCTCCGCAGGT
GAGTATATTAGGACACGAGTCAATTGGTGGATTCTTGAGTCATGGGGGTT
GGAGTTCTGTAATAGAAAGTCTAAGTTTTGGGATTCCACTTGTTGTTTTT
CCATTTGGAGCTGATCAAGGGATTAATGCAAAGCAATTGGAGGGTAAAAA
TGCAGGGGTGGAAATACCCAGATCAGAAGGTACTGGGTCATTTACAAGGA
AGTCTGTGGCTGATTTGTTGAGGCTGGTGGTGGTGGAGGAGGAGGGAAAG
GTTTACAGGGATGGTGCCAAGGAATTGAGGAAACTATTTGGGGACAAGGA
TTTGAACCACAAATACATTGACAACTTTGTTAAGTACATGGAAGAACATA
TCACGAATGCAGCTAATTGA*
A glucosyltransferase enzyme capable
of transferring a glucose residue to
the C-3 position of the C-28 rhamnose
residue of a QA-Tri(X/A)-F* derivative
(Qs-7-GlcT)
SEQ ID NO 56
MADRVINSYKKLHVVLFPWLAFGHMIPFLELAKLIAQKGHKISYISTPRN
IRRLPKIPSHLSNNLNFIEFPLPHIPNLHENVESTNDVTHDNPIAYYLLI
KALEGLQQPITTFLETSDPDWIIHDVFPQWITATASRLRISHAFYTTSSA
LRTASNYSPLMSSELPQDIATDFTTKSTNLLAAKVLVIRSCLELEPKEFE
QCKNLCVSKTVIPLGVVPPSIQVNDNISNINDDDNDWVKIVEWLNQGKEK
GSVIYVALGSEVSLSEQDLKEFALGLELSGLSFFWVFRNTGSYGLPAGFE
DRVKGRGIVWTSWAPQVSILGHESIGGFLSHGGWSSVIESLSFGIPLVVF
PFGADQGINAKQLEGKNAGVEIPRSEGTGSFTRKSVADLLRLVVVEEEGK
VYRDGAKELRKLFGDKDLNHKYIDNFVKYMEEHITNAAN
A nucleic acid sequence which encodes
the enzyme according to SEQ ID NO 58.
SEQ ID NO 57
ATGACCAGTAATAATAGCCAACTCCACATCTTCTTCCTTCCTGCACTGTC
TCCTGGCCACATGATACCTGTTATTGAAATGGCCAAACTAGTTGCTTCCC
GAGGTGTCATGGCAACTATAGTCACCACTGCTCACAATCTTGCTTTTGTT
TCCAGAACCATATCTACCTACAGTACTAAAATAAAAATCGTAACCATCAA
ATTCCCTTATGCAGAGGTTGGCTTACCTGAAGGATGTGAAATCATTGACT
CAGATACTCCCCCAGATATACTATTTCGTGTCATCAAAGCCCTGAGATTG
CTACAAGAACCAATGGAGCAACTATTGTCTTCTTATCAACCAGACTGCCT
TGTAGCTGATGCATTCTTCAGTTGGGCAACAAATTCTGCTGCTAAATTTA
ACATTCCAAGGCTTGTGTTCCATGTGGCATGTCTATTCTCTCTGTGTGCA
TCTCATTCTATTGAATTATATGAGCCTCAGAAGAAGGTGTCTTCTGATTC
AGAGACTTTTATTATTCCTAGTCTTCCTGGTGAAATAAAATTGACAAAGA
TGCAGCTACCTGCTGATTTACCTAAAACTGGTGTGGAGGCTGAGTACATC
AACAAAATGGTGAAAGCTGTCCATGAATCAGTGGAGAACAGCTATGGTTT
TATAATTAACAGCTTCTATGAGCTTGAGAAGGATTATGTAGATTATTATA
GAAACGTTATTGGAAGGAAAGCGTGGCATATTGGCCCACTGTCTCTATGC
CATGCGGACAATATTGAAGAAAAATCACAGCGAGGAAAAGAAAGCTCCAT
TGCTGAGAATGAATGCTTGAAGTGGCTTGACTCGAGGAAGCCAGATTCGG
TTGTTTATGTTGGTTTTGGAAGTCTGGTAAATTTCAGTGATTCCCAGCTG
ATGGAGATAGCATTGGGTCTTGAGGCTTCTGAGAAACAATTTATTTGGGT
TGTCAAGAAAAGCAAAAGAAATGAACAAGAAAAAGAAGAATGGCTACCTG
AAGGGTTTGAGAAAAGAACGGTAGGTAAGGGACTGATTATAAGAGGTTGG
GCACCCCAATTGTTAATTATGGATCATGAAGCTGTTGGAGGGTTTGTGAC
TCATTGTGGATGGAACTCAACCTTGGAAGGTGTGTGTGGTGGGGTAGTTA
TGGCCACTTGGCCAGTATCTTATGAGCAAATTTATACTGAAAAGCTGGTG
ACTGATGTTCTAAAAATTGGGGTTTCTGTAGGCGCTCAAACATGTGATGG
AATTGTTGGAGGTATTATTAAAAGTGAAGCAATAGAGAAGGCAGTGAATA
GAATAATGGAGGGGATTGAAGCAGAGGAGATGAGAAGCAGAGCAAAGGCC
TTTGCAAAAAAGGCAAGGCAGTCTGTTAAAGAGGGAGGATCCTCTTACTC
AGATTTGAACTCTTTAATTGAGGAGTTGAGTCTTAAATCTCTTAAGCATT
AA
A rhamnosyltransferase enzyme capable
of transfer a rhamnose residue to the
C-3 position of the D-fucose of the
C-28 chain of a QA-Tri(X/A)-F*
derivative (Qs-7-RhT)
SEQ ID NO 58
MTSNNSQLHIFFLPALSPGHMIPVIEMAKLVASRGVMATIVTTAHNLAFV
SRTISTYSTKIKIVTIKFPYAEVGLPEGCEIIDSDTPPDILFRVIKALRL
LQEPMEQLLSSYQPDCLVADAFFSWATNSAAKFNIPRLVFHVACLFSLCA
SHSIELYEPQKKVSSDSETFIIPSLPGEIKLTKMQLPADLPKTGVEAEYI
NKMVKAVHESVENSYGFIINSFYELEKDYVDYYRNVIGRKAWHIGPLSLC
HADNIEEKSQRGKESSIAENECLKWLDSRKPDSVVYVGFGSLVNFSDSQL
MEIALGLEASEKQFIWVVKKSKRNEQEKEEWLPEGFEKRTVGKGLIIRGW
APQLLIMDHEAVGGFVTHCGWNSTLEGVCGGVVMATWPVSYEQIYTEKLV
TDVLKIGVSVGAQTCDGIVGGIIKSEAIEKAVNRIMEGIEAEEMRSRAKA
FAKKARQSVKEGGSSYSDLNSLIEELSLKSLKH
A nucleic acid sequence which encodes
the enzyme according to SEQ ID NO 60.
SEQ ID NO 59
ATGAAGATAGAAACCATTTCCACAAATTGCATCAAACCCTCCAAGCCCAC
TCCTTCCCATCTCAGAAATATTAAGCTCTCTGATCAACACCAACATTCCC
CAGATGTCCATTCCAACTTCACCTTCTTCTACCAGTCTAATCAAATAGAT
GATGCTGTTGTTACTGTTCCCAGTGCTGCTATTGATGTTGCTCCTGCTAC
TGATGCCGCTATTGATGTTGCTGCTGCTACTGATACTGCTATTGATGGTG
CTGCTACAAATTTCTCAGTTCAATCCAAAATTCTTCATAATTGCTTGGCA
ACAACACTCACAAGCTTCTACCCTATCGCGGGTCGGTTTCAAAATGGCGA
CACTATCATTTGCAACGATGAAGGTGCCTTCTTTATCGAAGCAAAAACTG
ACATAAACATGTCCAATTTCCTTGGGCACCCAGACTTGCTTACAGTTATA
CGTGAACACTTAGTACCCGATGCTACTAACCCGGATTATAATGGTTCTAT
CCTTCTTCTCAAGTTTACATTGTTTGGCTGTGGCAGTACTGCCATAACCA
TCTCAATGTCGCACAAGATAGCCGATTTAGTTACCTTTATAACACTCCTT
AATTGCTGGACAGCTTTAGCTCGTGCTGGTGGTGGTGGTGGTATAGGTGG
TGGTTCTGATGGTTTTATTCCACCTGATTTGAATTTCCTTGGGCAGATTG
TCCCGGATTCGGATCCCTCACCAAAATCAGCAACTCCTGAATTTTTTCGA
AACAAGAAGTTTGTTACGAAAAGATTTGTTTTCAGTGCATCCAAGATCAA
AGAAGTTAAGGACAAAGTTATGAAGGAGATTAGGAAACAAGAGGATGATA
TTTTTCCTTCTCGTGTGGATGTGGTACTTGCATTAATTTGGAGAAGTACA
TTGTCAAGTTTGTCTGGATCATCTGGTAAGTTTAAACCGGCAATATTCAT
GCAAGCTGCAAATCTCAGAACGCGTACTGATCCACCATTACCGGAAACTT
CTATCGGGAACTTGGTGATACTATTTCCTTTGGTGGTTGAGAAAGAGACT
GATATAGAATTACATGAATTGGTTAACAAGTTGTTAGATGCCAAAGCATG
GGTTAACAAATTAAAGAAGAAATTCCAAGGTTATGATGGTGGTAATGATC
CTCTACAAGTTGTGGAAGCAATAAGATGTGAGGCTTTGAAAGAAATGGGT
AATGTTTGGAAGAAATCCAAGGATTTTTCGATGTATATTTCATCGAGTTT
TTGTAATTTTCTGATGAATGAGGTTGATTTTGGGTGGGGAAAGCCAGTTT
GGGTAACCAACACACCTAGAACAATCATGGCTAATACCATATATTTGTTG
GATACTAAAGAGGTGGGCGGAGTTGATGCTTTGGTACAATTTGAAGAGGA
AGAAATCACCAAATTGGAACTTAATCAAGAGCTACTCCAATTTGCTACTG
TTAATCCCATTCCTATTGTTATTTAA*
An acetyltransferase enzyme capable
of transferring an acetyl to the
C-4 position of the D-Fucose of the
C-28 chain of a QA-Tri(X/A)-F*
derivative (Qs-7-AcetylT)
SEQ ID NO 60
MKIETISTNCIKPSKPTPSHLRNIKLSDQHQHSPDVHSNFTFFYQSNQID
DAVVTVPSAAIDVAPATDAAIDVAAATDTAIDGAATNFSVQSKILHNCLA
TTLTSFYPIAGRFQNGDTIICNDEGAFFIEAKTDINMSNFLGHPDLLTVI
REHLVPDATNPDYNGSILLLKFTLFGCGSTAITISMSHKIADLVTFITLL
NCWTALARAGGGGGIGGGSDGFIPPDLNFLGQIVPDSDPSPKSATPEFFR
NKKFVTKRFVFSASKIKEVKDKVMKEIRKQEDDIFPSRVDVVLALIWRST
LSSLSGSSGKFKPAIFMQAANLRTRTDPPLPETSIGNLVILFPLVVEKET
DIELHELVNKLLDAKAWVNKLKKKFQGYDGGNDPLQVVEAIRCEALKEMG
NVWKKSKDFSMYISSSFCNFLMNEVDFGWGKPVWVTNTPRTIMANTIYLL
DTKEVGGVDALVQFEEEEITKLELNQELLQFATVNPIPIVI
A nucleic acid sequence which encodes
the enzyme according to SEQ ID NO 62
SEQ ID NO 61
ATGGGGGAAGTCAACCATGAAGAAGTAGAAATTGAAATAATATCAATAGA
AACCATAAAACCATCATCACTACTTCCACCAAAAACTCCTCCAAAAACCA
TCACACTTTCTCACCTCGATCAAGCTGCCCCTTTGTACTACTATCCTTTA
CTTTTATACTACACTAACACTACTACTACTACCCCAACATCACAAATTCG
AGTTGACATAACAAGTACCCTAAAAACTTCACTTAGCAAAACACTTGACA
AATTCCACCCTATTGCAGGTCGATGTGTGGACGACTCTACAATTTGTTGC
AACCACCAAGGAATACCATTCATTGAAACCAAAGTTGACTCCAATATCTT
GGATGTCATGAACTCGCCTGAGAAAATGAAGTTGCTTATCAAGTTTCTCC
CTCATGCAGAGTTTCAAGATGTGACTCGACCAGTCTCGGATTTAAACCAT
TTGGCGTTTCAAGTCAATGTTTTCCGGTGTGGTGGGGTGATCATTGGCTC
CTATGTGCTCCACAAGCTCCTTGATGGAATCTCTCTTGGAACTTTCTTTA
AAAATTGGTCAACCATTGCTAATGATGAGCGAGTTAAGGACGACGACCTA
GTACAACCTGACTTTGAAGCCACTATTAAGGCGTTCCCTCCGCGTACAGC
AACTCCAATGCTTCCTCGTAATCAACAACTTCCAAAGGCGGCTGAAAAAC
CAAATAATAATCCAGTCAAAGTTCTTGTGACAAAGAGCTTCGTATTTGAC
ATTGTTTCTTTAAAGAAGATGATGTTCATGGCTAAGAGTGAATTGGTTCC
TAAACCCACCAAATTTGAGACCGTGACAGGGTTTATTTGGGAACAAACCT
TATCAACATTGCGTAATTCTGGAGTTGAAGTTGAACATACATCGCTTATA
ATACCTGTAAACATCCGCCCAAGGATGAGTCCGCCACTCCCAAGAGGATC
CATGGGTAACTTGCTCAAGAATGCAAAGGCACAGGCCAACACCAGCAGCA
GCAATGGGCTTCAAGACCTTGTTAAaGAAATCCATTCATCTTTGTCTCAA
ACAACCCAGAAAATTAATACTCCTCCTCCTCCTCCTCCTCCTCCTACTAC
TACTGCTACAACAATCCATTCATCTTTGTCTCAAACAACCCAGAAAATTA
ATACTCCTCCTCCTACTACTACAACAATCCATTCATCTTTGTCTCAAACA
ACCCAGAAAATTAATACTACTACTACTACTACAGCAGAGGTTATTTTGAC
TAAACGGAAAGTTGACAATCCAGTTACACAGAATCGAGAAGGAAACTACC
TCTTCACCAGTTGGTGCAAGATTGGGTTGGATGAGGCTGACTTCGGGTTC
GGAAAGCCCGTTTGGGTAATTCCCAACGATGGGAGACCCCCTAAGGTCAG
GAATATGATTTTCCTTACTGATTATAGGCATCCCGAAACAGGCGTTGAAG
GAATTGCAGCATGGATTACGTTGGAAGAGAAACAAATGCAATGTTTAAAG
TCAAACCCAGAATTCCTTGCTTTTGCTACTCCTAATTAG
An acetyltransferase enzyme capable
of transferring an acetyl to the C-4
position of the D-Fucose residue on
the QA-Tri(X/A)-F* scaffold (SOAP10)
SEQ ID NO 62
MGEVNHEEVEIEIISIETIKPSSLLPPKTPPKTITLSHLDQAAPLYYYPL
LLYYTNTTTTTPTSQIRVDITSTLKTSLSKTLDKFHPIAGRCVDDSTICC
NHQGIPFIETKVDSNILDVMNSPEKMKLLIKFLPHAEFQDVTRPVSDLNH
LAFQVNVFRCGGVIIGSYVLHKLLDGISLGTFFKNWSTIANDERVKDDDL
VQPDFEATIKAFPPRTATPMLPRNQQLPKAAEKPNNNPVKVLVTKSFVFD
IVSLKKMMFMAKSELVPKPTKFETVTGFIWEQTLSTLRNSGVEVEHTSLI
IPVNIRPRMSPPLPRGSMGNLLKNAKAQANTSSSNGLQDLVKEIHSSLSQ
TTQKINTPPPPPPPPTTTATTIHSSLSQTTQKINTPPPTTTTIHSSLSQT
TQKINTTTTTTAEVILTKRKVDNPVTQNREGNYLFTSWCKIGLDEADFGF
GKPVWVIPNDGRPPKVRNMIFLTDYRHPETGVEGIAAWITLEEKQMQCLK
SNPEFLAFATPN
A nucleic acid sequence which encodes
the enzyme according to SEQ ID NO 64
SEQ ID NO 63
ATGATGGAGGTACATACCACATCGGAAAATTGCATTAAGCCCTCACAACC
CACTCCTTCCCACCTTCAAAATTTGAAACTCTCTAATCATCATAGCCAAG
CACCCGATATCCGTACCAACCTCACCTTCTTCTTCTCCTCTAATTTTAAC
AATCCAGTCCAGCCTGGTGACCATGACGCTACTACCAATTTTACACTCCA
ATCCAAACTTGTTCAGAATTCATTGGCTACAACTCTCACAATTCTCTACC
CTTTTGCTGGTCGATTCCGAAACGACGACACCATCATTTGCAAAGACGAT
GGCGCCTTCTTTATTGAAGCAAAAACCGACACCAAACTTTCTGACTTTCT
TGCCCAGCCGGACTTGCCTCTAGCTATAATGGACAAATTAGTCCCCGTAG
CTACCGACGCCAAGTATAATGGTTCTCTGCTAATCCTGAAATTTACTTTG
TTTGGCTGCGGTGGCTCAGCCGTAACCATCTCAATAACTCACAAGATTTC
CGATCTAGCAACCATTTTGACACTGCTTAATTGCTGGACTGCTTTATCTC
GTGGTGGTGATGGTGGTGGTTCCAGTCCGTTTATTCAGCCTGATTTGAAT
TTCATTGGCCGCCCTGTTCCAAGTACGAGTGAGGTTCCTCCTCCATCTTC
TGGAAAGAATTTTATTCCTCCAAATAGCAAGTACGTTACCAAAAGGTTCA
TATTCAGTGCAGCAAAGATTAAAGAACTGAAAGCCAGAGTCATCAACAAG
ATTCGTAAAGAAGAAGACAATGTTTTCCCTTCCCGTGTGGATGTAGTGCT
GGCACTCATTTGGAAATGCGCTTTGGCTTCTGTTAATTCTGGTTCCAGAT
CTGGAAATGCACAAACATTTAGGCCGTCGGTAATGATGCAAGCCGTGAAC
CTCAGAAACCGTACAGATCCACCATTACCGGAATCTTCGATTGGGAACTT
GGCAATACTATTACCGGTGTGGGTGGAGAAAGAGGAAGACACGGAATTAC
ATGAACTTGTTAGCAGATTGTTAACAGTGAAAGTGCGTGCTAACAGATTG
AAGAAGAAATACCAAGGTTATGAAGATCCAGAGCAAGTTATTATTTCAAT
GGAATCTGATTCAGTGAAGGAAATAATAGAGGTTCGGAAGAAATTGAAGG
ATTTCAGTACTTATGTTGCGGCGAGTGTGGTGAATGCTCCATTGTATGAC
GTTGATTTTGGGTGGGGAAAACCAGCATGGGTGACCAGCACGCCCAACAC
AGTAATGGCGAATTCTATATATTTGTTGGATACCAAAGACGCCGGTGGGA
TTGAAGTTTTGATGAATATGTTGAAAGAGGAAGACATGATTGTCTTTGAA
AGCAATCAGGAGTTGCTTCAGTCTGCCATGGTTAATCCCACTATCATTTG
A
An acetyltransferase enzyme capable of
transferring an acetyl to the C-4
position of the D-Fucose residue on the
QA-Tri(X/A)-F* scaffold (DMOT9)
SEQ ID NO 64
MMEVHTTSENCIKPSQPTPSHLQNLKLSNHHSQAPDIRTNLTFFFSSNFN
NPVQPGDHDATTNFTLQSKLVQNSLATTLTILYPFAGRFRNDDTIICKDD
GAFFIEAKTDTKLSDFLAQPDLPLAIMDKLVPVATDAKYNGSLLILKFTL
FGCGGSAVTISITHKISDLATILTLLNCWTALSRGGDGGGSSPFIQPDLN
FIGRPVPSTSEVPPPSSGKNFIPPNSKYVTKRFIFSAAKIKELKARVINK
IRKEEDNVFPSRVDVVLALIWKCALASVNSGSRSGNAQTFRPSVMMQAVN
LRNRTDPPLPESSIGNLAILLPVWVEKEEDTELHELVSRLLTVKVRANRL
KKKYQGYEDPEQVIISMESDSVKEIIEVRKKLKDFSTYVAASVVNAPLYD
VDFGWGKPAWVTSTPNTVMANSIYLLDTKDAGGIEVLMNMLKEEDMIVFE
SNQELLQSAMVNPTII

1. A method of making QA-Tri(X/R)-F*-GR-Ac, wherein the acetyl (Ac) group is attached to the C-4 position of the D-fucose of F*, the rhamnose (R) residue is attached to the C-3 position of the D-fucose of F* and the glucose (G) residue is attached to the C-3 position of the rhamnose residue of F*, wherein the method comprises combining QA-Tri(X/R)-F* with
i. the enzyme quillaic acid 28-O-fucoside [1,2]-rhamnoside [1,3] glucosyltransferase (QS-7-GlcT) having the amino acid sequence of SEQ ID NO 56, or an enzyme having an amino acid sequence with at least 70% sequence identity to SEQ ID NO 56;
ii. one or more enzymes selected from the enzyme quillaic acid 28-O-fucoside [1,4] acetyltransferase (QS-7-AcetylT) having the amino acid sequence of SEQ ID NO 60 or an enzyme having an amino acid sequence with at least 70% sequence identity to SEQ ID NO 60; the enzyme SOAP10 having the amino acid sequence of SEQ ID NO 62 or an enzyme having an amino acid sequence with at least 70% sequence identity to SEQ ID NO 62, or the enzyme (3S,5S,6S)-3,5-dihydroxy-6-methyloctanoyl-CoA transferase 9 (DMOT9) having the amino acid sequence of SEQ ID NO 64 or an enzyme having an amino acid sequence with at least 25% sequence identity to SEQ ID NO 64; and
iii. the enzyme quillaic acid 28-O-fucoside [1,3] rhamnosyltransferase (QS-7-RhaT) having the amino acid sequence of SEQ ID NO 58, or an enzyme having an amino acid sequence with at least 70% sequence identity to SEQ ID NO 58,
to form QA-Tri(X/R) F*-GR-Ac.

2. The method of claim 1, wherein
a) QA-Tri(X/R)-F* is first combined with the enzyme QS-7-GlcT having the amino acid sequence of SEQ ID NO 56, or an enzyme having an amino acid sequence with at least 70% sequence identity to SEQ ID NO 56, to form QA-Tri(X/R)-F*-G; then
b) QA-Tri(X/R)-F*-G is combined with one or more of the enzymes selected from the enzyme QS-7-AcetylT having the amino acid sequence of SEQ ID NO 60 or an enzyme having an amino acid sequence with at least 70% sequence identity to SEQ ID NO 60; the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, to form QA-Tri(X/R)-F*-G-Ac; then
c) QA-Tri(X/R)-F*-G-Ac is combined with the enzyme QS-7-RhaT having the amino acid sequence of SEQ ID NO 58, or an enzyme having an amino acid sequence with at least 70% sequence identity to SEQ ID NO 58, to form QA-Tri(X/R)-F*-GR-Ac.

3. The method of claim 2, wherein in steps a), b) and c) F* is FRX.

4. The method of claim 1, wherein
a) QA-Tri(X/R)-F* is first combined with one or more enzymes selected from the enzyme QS-7-AcetylT having the amino acid sequence of SEQ ID NO 60 or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, to form QA-Tri(X/R)-F*-Ac; then
b) QA-Tri(X/R)-F*-Ac is combined with the enzyme QS-7-RhaT having the amino acid sequence of SEQ ID NO 58, or an enzyme having an amino acid sequence with at least 70% sequence identity to SEQ ID NO 58, to form QA-Tri(X/R)-F*-R-Ac; then
c) QA-Tri(X/R)-F*-R-Ac is combined with the enzyme QS-7-GlcT having the amino acid sequence of SEQ ID NO 56, or an enzyme having an amino acid sequence with at least 70% sequence identity to SEQ ID NO 56, to form QA-Tri(X/R)-F*-GR-Ac

5. The method of claim 4, wherein in steps a) and b) F* is FR and in step c) F* is FRX.

6. The method of claim 1, wherein
i. QA-Tri(X/R)-F* is first combined with one or more enzymes selected from the enzyme QS-7-AcetylT having the amino acid sequence of SEQ ID NO 60 or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, to form QA-Tri(X/R)-F*-Ac; then
ii. QA-Tri(X/R)-F*-Ac is combined with the enzyme QS-7-GlcT having the amino acid sequence of SEQ ID NO 56, or an enzyme having an amino acid sequence with at least 70% sequence identity to SEQ ID NO 56, to form QA-Tri(X/R)-F*-G-Ac, then
iii. QA-Tri(X/R)-F*-G-Ac is combined with the enzyme QS-7-RhaT having the amino acid sequence of SEQ ID NO 58, or an enzyme having an amino acid sequence with at least 70% sequence identity to SEQ ID NO 58, to form QA-Tri(X/R)-F*-GR-Ac.

7. The method of claim 6, wherein in step a) F* is FR and in steps b) and c) F* is FRX.

8. The method of any one of claim 1, 2, 4 or 6, wherein Tri(X/R) is TriX and F* is FRXA.

9. A method of making a biosynthetic QA-Tri(X/R)-F*-GR-Ac in a host, which method comprises the steps of:
a) expressing genes required for the biosynthesis of QA-TriR-F* and/or QA-TriX-F*, and
b) introducing a polynucleotide encoding:
i. the enzyme QS-7-GlcT having the amino acid sequence of SEQ ID NO 56, or an enzyme having an amino acid sequence with at least 70% sequence identity to SEQ ID NO 56;
ii. one or more enzymes selected from the enzyme QS-7-AcetylT having the amino acid sequence of SEQ ID NO 60, or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, and
iii. the enzyme QS-7-RhaT having the amino acid sequence of SEQ ID NO 58, or an enzyme having an amino acid sequence with at least 70% sequence identity to SEQ ID NO 58,
into the host.

10. A method of making a biosynthetic QA-TriX-F*-GR-Ac in a host, which method comprises the steps of:
a) expressing genes required for the biosynthesis of QA-TriX-F*, and
b) introducing a polynucleotide encoding:
i. the enzyme QS-7-GlcT having the amino acid sequence of SEQ ID NO 56, or an enzyme having an amino acid sequence with at least 70% sequence identity to SEQ ID NO 56;
ii. one or more enzymes selected from the enzyme QS-7-AcetylT having the amino acid sequence of SEQ ID NO 60 or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, and
iii. the enzyme QS-7-RhaT having the amino acid sequence of SEQ ID NO 58, or an enzyme having an amino acid sequence with at least 70% sequence identity to SEQ ID NO 58,
into the host.

11. The method of claim 10, wherein F* is FRXA.

12. A method of making a biosynthetic QA-TriR-F*-GR-Ac in a host, which method comprises the steps of:
a) expressing genes required for the biosynthesis of QA-TriR-F*, and
b) introducing a polynucleotide encoding:
i. the enzyme QS-7-GlcT having the amino acid sequence of SEQ ID NO 56, or an enzyme having an amino acid sequence with at least 70% sequence identity to SEQ ID NO 56;
ii. one or more enzymes selected from the enzyme QS-7-AcetylT having the amino acid sequence of SEQ ID NO 60, or an enzyme having an amino acid sequence with at least 70% sequence identity to SEQ ID NO 60, the enzyme SOAP10 having the amino acid sequence of SEQ ID NO 62 or an enzyme having an amino acid sequence with at least 70% sequence identity to SEQ ID NO 62, or the enzyme DMOT9 having the amino acid sequence of SEQ ID NO 64 or an enzyme having an amino acid sequence with at least 25% sequence identity to SEQ ID NO 64, and
iii. the enzyme QS-7-RhaT having the amino acid sequence of SEQ ID NO 58, or an enzyme having an amino acid sequence with at least 70% sequence identity to SEQ ID NO 58,
into the host.

13. The method of any one of claims 9 to 12, wherein
amino acid sequence SEQ ID NO 60 is encoded by polynucleotide sequence SEQ ID NO 59;
amino acid sequence SEQ ID NO 58 is encoded by polynucleotide sequence SEQ ID NO 57;
amino acid sequence SEQ ID NO 56 is encoded by polynucleotide sequence SEQ ID NO 55;
amino acid sequence SEQ ID NO 62 is encoded by polynucleotide sequence SEQ ID NO 61 and amino acid sequence SEQ ID NO 64 is encoded by polynucleotide sequence SEQ ID NO 63.

14. A glucosyltransferase enzyme having the amino acid sequence of SEQ ID NO 56 (QS-7-GlcT), or an enzyme having an amino acid sequence with at least 70% sequence identity to SEQ ID NO 56.

15. A rhamnosyltransferase enzyme having the amino acid sequence of SEQ ID NO 58 (QS-7-RhaT), or an enzyme having an amino acid sequence with at least 70% sequence identity to SEQ ID NO 58.

16. An acetyltransferase enzyme having the amino acid sequence of SEQ ID NO 60 (QS-7-AcetylT), or an enzyme having an amino acid sequence with at least 70% sequence identity to SEQ ID NO 60.

17. A polynucleotide which encodes any of the enzymes as claimed in any one of claims 14 to 16.

18. A vector comprising the polynucleotide according to claim 17

19. A host cell comprising the polynucleotide according to claim 17.

20. A host cell transformed with the vector according to claim 18.

21. A host cell according to claim 19 or claim 20, wherein the host cell is a plant cell or a microbial cell.

22. A biological system of a plant or a microorganism comprising host cells according to any one of claims 19 to 21.

23. A biological system according to claim 22, wherein the biological system is yeast or Nicotiana benthamiana.

24. A method according to any one of claims 1 to 13, wherein the method further includes the step of isolating the QA-Tri(X/R)-F*-GR-Ac derivative.

25. The QA-Tri(X/R)-F*-GR-Ac derivative obtainable by the method of claim 24.

26. The derivative of claim 25, wherein the derivative is 3-O-{β-D-xylopyranosyl-(1->3)-[β-D-galactopyranosyl-(1->2)]-β-D-glucopyranosiduronic acid}-28-O-{β-D-apiofuranosyl-(1->3)-β-D-xylopyranosyl-(1->4)-α-L-rhamnopyranosyl-(1->2)-β-D-fucopyranosyl ester}-quillaic acid acetylated at the C-4 position of the D-fucose of the core C-28 chain and with a rhamnose moiety attached to the C-3 position of the D-fucose of the C-28 chain and a glucose moiety attached to the C-3 position of the core C-28 rhamnose moiety (QA-TriX-FRXA-GR-Ac).

27. The use of the QA-Tri(X/R)-F*-GR-Ac derivative according to claim 25 or claim 26, as an adjuvant.

28. The use according to claim 27, wherein the adjuvant is a liposomal or immune stimulating complex (ISCOM) formulation.

29. The use according to claim 28, wherein the ISCOM formulation comprises a first ISCOM matrix containing the QA-Tri(X/R)-F*-GR-Ac derivative according to claim 25 or claim 24, and a second ISCOM matrix containing QS-21.

30. The use according to any one of claims 27 to 29, wherein the adjuvant further comprises a TLR4 agonist.

31. The use according to claim 30, wherein the TLR4 agonist is 3D-MPL.

32. An adjuvant composition comprising the QA-Tri(X/R)-F*-GR-Ac derivative according to claim 25 or claim 26.


SEQUENCE LISTING (70 sequences)

SEQ ID NO: 1 - DNA, 1374 bp, Quillaja saponaria
  source 1..1374: mol_type = other DNA
atggagaatgggagagtttacaaatcccatgtcgtggtgctcgcatttcacgggcaaggccatattgttccgttaatccaattatccagacgattggcctggaaaggcatcaaaatcacatttgctacaacacattcttgcaccaaggccattcaaacaggaagtgattcaatttcacttttatcaatttatgatgacataactgatggtgggtttcaaggagaaggaggattcaagggcttccttcagagatttgaagccagtaccacaaggatcttacacgaattcgtcaagaatcatgaaaactcaaagaacccagtaaaatgcttaatatatgatgctaacttaatatgggctctggaaatggcaaagcaattgggtattgctactgctgcatttgtgtttccttcttgggctgccattgccacctactatcccttttatttagaggtgtatgcggatcagcagataaagaaggtagatcctttcacaatgcctgacttacctccacaacttggacttccaaatatggcatctctcggttcagattcgggtcaacactcccccatactcaaactcatgttgcaacagttagaaaattttgggaaagctgactggatcctgtctcacgcatttgaacagtttgaacaagaggtacttgactggatgagaaatatcagcccagtaacaacaattggtccaactctgccatctgtttatcttgatggtaggctaaaagatgacacagattacggttacaatttgtacaagccagatagtgatacctgcatgaagtggctagacactaaggaaactgaatcagtggtttatatatcatttggcagtgttgcagatttgatcccagaacagatgacagaaataacaaactccctgaagaaaatgagcagcaactttctgtgggtggtgaaggaaactgaaaaaaacaacctccctagcagctttgttgaggagacaaaagaaaagggattggtagtgacttggtgcccccagttgaaggtgttgtctcatcctgcagtgggttgtttcattacacactgtggaacaaattccatatttgagtcagtatgctttgcagtgccaatggtgggaatgccacagttttgtgatcaaatgcctaatgcatatttcatggagaaggtctggaaagtaggtgttaggccaagtttggatgacaatggtgtcgtcactggagaagaaattgagcgatgtataaaagtagttaccgaaggagagagtgggcaagagattaagaagaaacttgtgcagtggaaagagcttgcaaaagaggcagtggacgagggtggaagttcagataagcacattgatgaattcattgctggaatcacaacttga

SEQ ID NO: 2 - protein, 457 aa, Quillaja saponaria
MENGRVYKSHVVVLAFHGQGHIVPLIQLSRRLAWKGIKITFATTHSCTKAIQTGSDSISLLSIYDDITDGGFQGEGGFKGFLQRFEASTTRILHEFVKNHENSKNPVKCLIYDANLIWALEMAKQLGIATAAFVFPSWAAIATYYPFYLEVYADQQIKKVDPFTMPDLPPQLGLPNMASLGSDSGQHSPILKLMLQQLENFGKADWILSHAFEQFEQEVLDWMRNISPVTTIGPTLPSVYLDGRLKDDTDYGYNLYKPDSDTCMKWLDTKETESVVYISFGSVADLIPEQMTEITNSLKKMSSNFLWVVKETEKNNLPSSFVEETKEKGLVVTWCPQLKVLSHPAVGCFITHCGTNSIFESVCFAVPMVGMPQFCDQMPNAYFMEKVWKVGVRPSLDDNGVVTGEEIERCIKVVTEGESGQEIKKKLVQWKELAKEAVDEGGSSDKHIDEFIAGITT

SEQ ID NO: 3 - DNA, 1422 bp, Quillaja saponaria
  source 1..1422: mol_type = other DNA
atggcaaaaactgataagcagcttcacatcgccatgttcccatggctagctatgggtcatatattcccaaactttgagctcgctaagctctttgctcaaaagggtcactcaattactttaatctccaccccacgaaacatcagtcgtctccctcaaatccctacacatttagagcaattgattaaattagtcagcttgcctatattacccaaacacaaagcaaatctcccagagaatgcagagtccaccatggacgttacccccaataaagtcccataccttaagatggcctatgatggtcttcaggagtcgttgactcaattactgaaatcttcagctcccgattggattctatatgactttgctgctgactggttaccaccacttgttcacagccttcaaatccgctgtgttttcttcgtggtatcccctgcgtggaatctttgcttctttgacactcccaaaccacagttgggcagtgctgctgtttttcgaacaaagcctgaagactatcttcgccctcccagttgggttcctttccattcaaatattgggctaaagcttcacgaggtgaagaaaatgtttgaaggggtttcagataaagaaacaggggtcactgtaagttttaacttcaacaaagcagtttcgagctgtgacttgttttctttccgcagctgctatgaactcgaatcagaatggctgaacctggtggaggatatttacaagaggcctgtagttccagtgggcgtaattccaccctcttttcaagtcagaattgtgaatgaagaagacaacaaaccagagtggttaaagatccaatcttggttagataaacaagagcaaggatcggtggtatacatagcatttggcagtgagcttaagctgggccaacaagatctcaccgaattagctcttggacttgagctttctgggttgccattcttttgggcacttagaaagcagcaagacagctcatcagtagatttaccagatgggtttgaggaccgagtcagtgatcgtggagttgtttgcagagactgggtgccccaacttaagatcctagctcacgggtcaattgggggttatttgactcactgtggttcaggttcagtgatagagggacttcattttgggcgtgttcttgttatgctgccctatttactagaccaagcattatatgctagagtattggaggagaaaaagctgggggttgagataccaaggaacgaacaagatgggtcttttactaggagctcagtggccaagtctgtgaagttggccatagtggatgaggggggaagtatttacagggacaaagccaaagagatgggcttggtattcagtgacaaagatcgtcatgaacaatacattgagaatttccttcaacaccttcaacacaaaagggaacctttccaaatttaa

SEQ ID NO: 4 - protein, 473 aa, Quillaja saponaria
MAKTDKQLHIAMFPWLAMGHIFPNFELAKLFAQKGHSITLISTPRNISRLPQIPTHLEQLIKLVSLPILPKHKANLPENAESTMDVTPNKVPYLKMAYDGLQESLTQLLKSSAPDWILYDFAADWLPPLVHSLQIRCVFFVVSPAWNLCFFDTPKPQLGSAAVFRTKPEDYLRPPSWVPFHSNIGLKLHEVKKMFEGVSDKETGVTVSFNFNKAVSSCDLFSFRSCYELESEWLNLVEDIYKRPVVPVGVIPPSFQVRIVNEEDNKPEWLKIQSWLDKQEQGSVVYIAFGSELKLGQQDLTELALGLELSGLPFFWALRKQQDSSSVDLPDGFEDRVSDRGVVCRDWVPQLKILAHGSIGGYLTHCGSGSVIEGLHFGRVLVMLPYLLDQALYARVLEEKKLGVEIPRNEQDGSFTRSSVAKSVKLAIVDEGGSIYRDKAKEMGLVFSDKDRHEQYIENFLQHLQHKREPFQI

SEQ ID NO: 5 - DNA, 1395 bp, Quillaja saponaria
  source 1..1395: mol_type = other DNA
atggctgctgcagctcccaatcacaggctccacatagcattcttcccatggttagcatttggtcacataaaccctttctttgagcttgcgaagctcattgctcaaaagggtcatcatatttctttcatttccaccccaagaaacatccaacgcctttcacaagttcctccacaattagcagattccatagatctagtgagcttaccagtaatccataattcaaacctcccagaaaacgcagagtccaccatggacattccacctgataaaaccccttaccttgggatgcttcacgacagtctcaaagaaccccttactcaattccttcaaactcattcccctgattggattctgtatgacttttcagctggttggcttgctgccatagtagaagaccttggtatctcccacggctacttttctatcattccctgttggaacataggcttcaatggacgccaaatgaatggtttccaaaagccagatatttcattgccctccgccgtgtcgcttaagaaatatgaggtgaagaaaatcatggatttggtcaaatcttttcccaaaattttggatgagtcggccactaaatccatagcttcgcattcgacctgtgaagtaatttttatacgaaattgccccgagattgaagcagattggttcgactatacttcgaaaattttcgataaaccagtggttccggtgggcgtagtgcccccatctgtgcacataactaacaaagagaaggacgagcatttcaacaaatggttggagatcaaagaatggttggatcaacaagacagaggttctgtaatttatatagcttttggaactgaatcactgccaaatcaggatgaaatcaccatgcttgctcaagggcttgagctatgtgggcttcctttcttttgggcattaaggaagtctaatgtggcttctgatcagccaaattcagactcagttgagctaccggagggatttgaagaacaaaccaaaggtcgtggaattgtgtggacgagttgggcacctcaacagagaattctgggtcacaattcaattgggggttttgtgactcactgtggttggagttcagtaatagaaggaattcactatggacggccactgattatgttccctctaacagtcgaacagtctctgaatgctaggattttgggggagaagaagttgggtatggaagtacccagagaagatgatgggtcttttacaggtgaagttgtggcagagacattgaagctggtattgctggaccaagatgggaaagtttacagggacaaggtaacagagatgagtaaggtatttggggacaaagacaaacatgagaaatacatgggtgatctacttgaattcttcaaaaattacaggtctcttaagaggaattag

SEQ ID NO: 6 - protein, 464 aa, Quillaja saponaria
MAAAAPNHRLHIAFFPWLAFGHINPFFELAKLIAQKGHHISFISTPRNIQRLSQVPPQLADSIDLVSLPVIHNSNLPENAESTMDIPPDKTPYLGMLHDSLKEPLTQFLQTHSPDWILYDFSAGWLAAIVEDLGISHGYFSIIPCWNIGFNGRQMNGFQKPDISLPSAVSLKKYEVKKIMDLVKSFPKILDESATKSIASHSTCEVIFIRNCPEIEADWFDYTSKIFDKPVVPVGVVPPSVHITNKEKDEHFNKWLEIKEWLDQQDRGSVIYIAFGTESLPNQDEITMLAQGLELCGLPFFWALRKSNVASDQPNSDSVELPEGFEEQTKGRGIVWTSWAPQQRILGHNSIGGFVTHCGWSSVIEGIHYGRPLIMFPLTVEQSLNARILGEKKLGMEVPREDDGSFTGEVVAETLKLVLLDQDGKVYRDKVTEMSKVFGDKDKHEKYMGDLLEFFKNYRSLKRN

SEQ ID NO: 7 - DNA, 1530 bp, Quillaja saponaria
  source 1..1530: mol_type = other DNA
atggactccacccacttgcagccggccactactcctctgaaaattcacttcatacctttcatatctccgggtcacatcatcccactctctgaattggctcgtatctttgcttcacgcggtgagcatgtgactatcattaccactcctttcaacgctgacctacttcagaaatccattgacgaagacagagattccggcgagcacattggcatccacaccgttgaatggtcagccacagagctaggccttcctcatgggattgaaaatctcagcaacgtcactgacttggagaccgccattaagctacacagggccctcatgctaatgcagaaacagatggaagatttcatgacccgaaatccacccgattgtataattgccgacacgttttacccgtgggcctccgaatttgctaatcggatgggtatcccgagactcattttctacccgtggagtactttcgcgctctgtttgatggaatctattcgatctcccgactccccgcaccgaagattgagttcggattcggatccatttgtggttccgggtcttcctcacccgatcatcttgacccgttctcagcttccggaacacgatcgaaaggacatagcggacccggctgcccaactcatggatcagcataaagaaactgagatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagagagggtattcaagagtgatgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttgtttgggtggttcgcggaaaagataaacaaatcgatgttaatgacgatggggagaagacatggttgcctaaagggtttgaggaaagaatgaaaacagaaaataaaggtttgattgtaaggggatgggccccacaggtgctggttttggatcatccatcattgggatgtttcttgacgcattgcggctggaattctacgattgagggaatcacagcaggcgttcctttgatcacgtggccagtattcgccgagcaattctataatgagaagctaatcacgcaggtgcatgggaatggggtggtggttggttcagaggagtggatcatgttgttcaccgtcgctaaaagcttggtaagtagagacaaaattgagaatgctgtgaggaagataatggacggtggtgatgaggctgtacaaatcagaaggcgggcccgggaacttggagaaaaagcttggaaagctgcttcaactggggggtcctcctacaataatctaaccgcagcaattgaagaccttaagcggttgagagaggaccgttcgaagctgaaaacaaaaacaatttga

SEQ ID NO: 8 - protein, 509 aa, Quillaja saponaria
MDSTHLQPATTPLKIHFIPFISPGHIIPLSELARIFASRGEHVTIITTPFNADLLQKSIDEDRDSGEHIGIHTVEWSATELGLPHGIENLSNVTDLETAIKLHRALMLMQKQMEDFMTRNPPDCIIADTFYPWASEFANRMGIPRLIFYPWSTFALCLMESIRSPDSPHRRLSSDSDPFVVPGLPHPIILTRSQLPEHDRKDIADPAAQLMDQHKETEMKSYGIILNNFAEIETEYTEHYKKITGHKVWHIGPAAAIVHRNAKEKAERVFKSDEHDNNLVINWLNSKEPNSVVYVCFGSGCQFPDKQLYEIACGLELSGHQFVWVVRGKDKQIDVNDDGEKTWLPKGFEERMKTENKGLIVRGWAPQVLVLDHPSLGCFLTHCGWNSTIEGITAGVPLITWPVFAEQFYNEKLITQVHGNGVVVGSEEWIMLFTVAKSLVSRDKIENAVRKIMDGGDEAVQIRRRARELGEKAWKAASTGGSSYNNLTAAIEDLKRLREDRSKLKTKTI

SEQ ID NO: 9 - DNA, 1530 bp, Quillaja saponaria
  source 1..1530: mol_type = other DNA
atggactccacccacttgcagccggccactactcctctgaaaattcacgtcatacctttcatagctccgggtcacattatcccactctctgaattggctcgtatctttgcttcacgcggtgagcatgtgactatcataaccactcctttcaacgctgacctacttcagaaatccattgacgaagacagagattccggcgagcacattggcatccacaccgttgaatggtcagccacagagctaggccttcctcatggggttgaaaatctcagcaacgtcactgacttggagaccggcattaagctacacagggccctcgtgctaatgcagaaacagatggaagatttcatgacccgaaatccacccgattgtataattgccgacacgttttacccgtgggcctccgaatttgctaatcggatgggtatcccgagactcattttcttcccggggtgtactttcgcgctctgtttgatggaatctattcgatctcccgactccccgcaccgaagattgagttcggattcggacccatttgtggttccgggtcttcctcacccgatcatcttgacccgttctcaacttccggaacacgatcgagaggacatagcggacccggctgcccaattcatggatcagtgtaaagaagctgcgatgaagagctacggaattattctcaacaatttcgcggagatcgaaacagaatacacagagcattacaagaaaataacgggtcacaaggtttggcacattggacctgccgcagcaattgttcaccgaaatgccaaagagaaggcagaggggcttttcaaaagtgacgagcatgacaataaccttgtcatcaattggctcaactcgaaggaaccaaactcagttgtttatgtttgtttcggcagcggatgtcaattccctgataaacaactctatgagattgcatgcgggttagagttatctgggcatcaatttatttgggtggttcgcggcaaagataaacaaatcgatgttaatgacgatgaggagaagacatggttgcctaaagggtttgaggaaagaatgaaaacagaaaataaaggtttgattgtaaggggatgggccccacaggtgctggttttggatcatccatcgttgggatgtttcttgacgcattgcggctggaattctacgattgagggaatcacagcaggtgttcctttgatcacgtggccagtatactccgagcaattctataatgagaagctaatcacgcaggtgcatggtaatggggtgggggttggttcagaggagtggatcatgctgttcagcgtcgctaaaagtttggtaagtagagacaaaattgagaatgctgtgaggaagataatggacggtggtgatgaggctttagaaatcagaaggcgggcccgggaacttggagaaaaagctaggaaagctgcttcaattggggggtcctccgacaataatctaaccgcagcaattgaagaccttaagcggttgagagaggaccgttcgaagctgaaaacaaaaacaatttga

SEQ ID NO: 10 - protein, 509 aa, Quillaja saponaria
MDSTHLQPATTPLKIHVIPFIAPGHIIPLSELARIFASRGEHVTIITTPFNADLLQKSIDEDRDSGEHIGIHTVEWSATELGLPHGVENLSNVTDLETGIKLHRALVLMQKQMEDFMTRNPPDCIIADTFYPWASEFANRMGIPRLIFFPGCTFALCLMESIRSPDSPHRRLSSDSDPFVVPGLPHPIILTRSQLPEHDREDIADPAAQFMDQCKEAAMKSYGIILNNFAEIETEYTEHYKKITGHKVWHIGPAAAIVHRNAKEKAEGLFKSDEHDNNLVINWLNSKEPNSVVYVCFGSGCQFPDKQLYEIACGLELSGHQFIWVVRGKDKQIDVNDDEEKTWLPKGFEERMKTENKGLIVRGWAPQVLVLDHPSLGCFLTHCGWNSTIEGITAGVPLITWPVYSEQFYNEKLITQVHGNGVGVGSEEWIMLFSVAKSLVSRDKIENAVRKIMDGGDEALEIRRRARELGEKARKAASIGGSSDNNLTAAIEDLKRLREDRSKLKTKTI

SEQ ID NO: 11 - DNA, 897 bp, Quillaja saponaria
  source 1..897: mol_type = other DNA
atggcagaagcaacgcagaggtatgctgttgtgacaggatctaataagggaattggatttgggatatgcaagcagttggcttctaagggaatcaaagtagtgttaacagctagagatgagaagagaggtcttgaagcagttgagaaattgaaagaaattagtctggctggtcatgtggtttttcatcaactcgatgtgtctgatcctgctagtgttactagccttgaagatttcatcaaaacccagtttgggaagctagatattctggtaaacaatgctgggataacaggaacaactgtagatgctgatgctttagcagcttcaggcttcggtacagggggtgaacgtaagcctattgattggagtaagttagtgatacagacttatgaatcagttgaaaaagctttcaacacaaactattacggtggcaaaagaatgacagaagcacttatacccctcctccagctatcagactcacccaggattgttaatgtttcctctgctatgggacagttagagaatatacctagtggatgggcaaaggaagtgctcacagatgttgataacctaacagaaggaaaattggatgaggtttcaacccagtttttgaaagatttcaaagagggttcattggaaaccaaaggctggcctagtcttatgtcttcttatatagtctcaaaagctgttttaaatgcctacacaaggattcttgctaagaaatacccagctttctgcatcaattgtgtagatcctggctatgtgaagacagacataaaccatcatactggccaattaagtgttgatgaaggtgctgaaagtcctgtaagactggccttgctgcctaatggtggtccttctggcgtgttcttctccaggacagaagaagcaccattttga

SEQ ID NO: 12 - protein, 298 aa, Quillaja saponaria
MAEATQRYAVVTGSNKGIGFGICKQLASKGIKVVLTARDEKRGLEAVEKLKEISLAGHVVFHQLDVSDPASVTSLEDFIKTQFGKLDILVNNAGITGTTVDADALAASGFGTGGERKPIDWSKLVIQTYESVEKAFNTNYYGGKRMTEALIPLLQLSDSPRIVNVSSAMGQLENIPSGWAKEVLTDVDNLTEGKLDEVSTQFLKDFKEGSLETKGWPSLMSSYIVSKAVLNAYTRILAKKYPAFCINCVDPGYVKTDINHHTGQLSVDEGAESPVRLALLPNGGPSGVFFSRTEEAPF

SEQ ID NO: 13 - DNA, 1197 bp, Quillaja saponaria
  source 1..1197: mol_type = other DNA
atgccaactttgtacaaaaaagcaggcttaatggcgtcggcgtcaagggtagatctggatggtaatcagataaagccgatgacaatttgcatgatcggtgccggtgggttcattgggtcccacctctgcgagaagattatggcggagacaccgcacaaggttctggcattagatgtctacaatgacaagatcaagcacttactggagccggattctcttcaatggaaagatcgcatccaattccaccgcatcaacattaagcacgattcgaggctcgaaggtctcatcaagatggcagatctgactataaatctggctgctatttgtactcccgcggattacaacacccgtcctctggacacaatttatagcaatttcattgacgctcttcctgtggtaaagtactgttcggagaataacaagcgtctcattcatttctctacgtgtgaagtgtatgggaaaacgattgggagctttctcccaaaagacagccctcttctaaaggatcctgaatattttgttcttaaagaagatgcctccccatgcatatttggtcctattgaaaagcagagatggtcctacgcatgtgcaaagcaattgattgagaggctggtttatgctgagggtgctgagaatggccttgagttcactattgtgcgacctttcaattggattggccccagaatggatttcatacctggcattgatggtccaagtgaaggtgttccacgggttctggcatgctttagtaacaatctccttcgcggtgagccactcaaactcgttgatggtggccaatcccagagaacttttgtttatatcaaggatgcaattgaagctgttttgttgatgattgaaaaccctgccagggctaatggtcatatttttaatgtgggtaaccctcacaatgaagttacagttcggcaacttgctgaaatgatgaccgaggtctattctaaggtaagtggagaaccgtctcttgaggtgcctaccattgatgtaagctccaaagaattttatggtgaaggatacgatgatagtgacaagagaattcctgacatgaccataatcaacaggcaacttggctggaaccctaagacatcgctctgggatcttcttgagtcgaccctcacctatcaacataggacatatgcagaagctattaagaaatcaattgcgaaaccagttgccagctag

SEQ ID NO: 14 - protein, 398 aa, Quillaja saponaria
MPTLYKKAGLMASASRVDLDGNQIKPMTICMIGAGGFIGSHLCEKIMAETPHKVLALDVYNDKIKHLLEPDSLQWKDRIQFHRINIKHDSRLEGLIKMADLTINLAAICTPADYNTRPLDTIYSNFIDALPVVKYCSENNKRLIHFSTCEVYGKTIGSFLPKDSPLLKDPEYFVLKEDASPCIFGPIEKQRWSYACAKQLIERLVYAEGAENGLEFTIVRPFNWIGPRMDFIPGIDGPSEGVPRVLACFSNNLLRGEPLKLVDGGQSQRTFVYIKDAIEAVLLMIENPARANGHIFNVGNPHNEVTVRQLAEMMTEVYSKVSGEPSLEVPTIDVSSKEFYGEGYDDSDKRIPDMTIINRQLGWNPKTSLWDLLESTLTYQHRTYAEAIKKSIAKPVAS

SEQ ID NO: 15 - DNA, 1275 bp, synthetic construct
  misc_feature 1..1275: note = A nucleic acid sequence which encodes the truncated enzyme according to SEQ ID NO 16.
  source 1..1275: mol_type = other DNA
atggcgcccgagaaaatgcccgaggaggacgaggaaatcgtcgccggggtcgtcgcagggaagatcccctcctacgtgctcgagaccaggctaggcgactgccgcagggcagccgggatccgccgcgaggcgctgcgccggatcaccggcagggagatcgacggccttcccctcgacggcttcgactacgactcgattctcggacagtgctgcgagatgcccgtcgggtacgtgcagctgccggtcggcgtcgcggggccgctcgtcctcgacggccgccgcatatacgtcccgatggccaccacggagggctgcctaatcgccagcaccaaccgcggatgcaaggccattgccgagtccggaggcgcatccagcgtcgtgtaccgcgacgggatgacccgcgcccccgtagcccgcttcccctccgcacgacgcgccgcagagctcaagggcttcctggagaatccggccaactacgacaccctgtccgtggtctttaacagatcaagcagatttgcaaggctgcagggggtcaagtgcgccatggctgggaggaacttgtacatgaggttcacctgcagcaccggggatgccatggggatgaacatggtctccaagggcgtccaaaatgtgctcgactatctgcaggaggacttccctgacatggacgttgtcagcatctcaggcaacttttgttccgacaagaaatcagctgctgtaaactggattgaaggccgtggaaagtccgtggtttgtgaggcagtaatcagagaggaagttgtccacaaggttctcaagaccaacgttcagtcactcgtggagttgaatgtgatcaagaaccttgctggctcagcagttgctggtgctcttgggggtttcaacgcccacgcaagcaacatcgtaacggctatcttcattgccactggtcaggatcctgcacagaatgtggagagctcacagtgtatcactatgttggaagctgtaaatgatggcagagaccttcacatctccgttacaatgccatctatcgaggtgggcacagttggtggaggcacgcagctggcctcacagtcggcctgcttggacctactgggcgtcaaaggcgccaacagggaatctccggggtcgaacgctaggctgctggccacggtggtggctggtgccgtcctagctggggagctgtccctcatctccgcccaagctgccggccatctggtccagagccacatgaaatacaacagatccagcaaggacatgtccaagatcgcctgctga

SEQ ID NO: 16 - protein, 424 aa, synthetic construct
  REGION 1..424: note = AstHMGR (Avena strigosa truncated HMG-CoA reductase) translated nucleotide sequence
MAPEKMPEEDEEIVAGVVAGKIPSYVLETRLGDCRRAAGIRREALRRITGREIDGLPLDGFDYDSILGQCCEMPVGYVQLPVGVAGPLVLDGRRIYVPMATTEGCLIASTNRGCKAIAESGGASSVVYRDGMTRAPVARFPSARRAAELKGFLENPANYDTLSVVFNRSSRFARLQGVKCAMAGRNLYMRFTCSTGDAMGMNMVSKGVQNVLDYLQEDFPDMDVVSISGNFCSDKKSAAVNWIEGRGKSVVCEAVIREEVVHKVLKTNVQSLVELNVIKNLAGSAVAGALGGFNAHASNIVTAIFIATGQDPAQNVESSQCITMLEAVNDGRDLHISVTMPSIEVGTVGGGTQLASQSACLDLLGVKGANRESPGSNARLLATVVAGAVLAGELSLISAQAAGHLVQSHMKYNRSSKDMSKIAC

SEQ ID NO: 17 - DNA, 2277 bp, Quillaja saponaria
  source 1..2277: mol_type = other DNA
atgtggaggctgaagatagcagaaggtggttccgatccatatctgttcagcacaaacaacttcgtgggtcgccagacatgggagttcgaaccggaggccggcacacctgaggagcgagcagaggtcgaagctgcccgccaaaacttttacaacaaccgttaccaggtcaagccctgtgacgacctcctttggagatatcagttcctgagagagaagaatttcaaacaaacaataccgcctgtcaaggttgaagatggccaagaaattacttatgagatggccacaacctcaatgcagagggcggcccgtcacctatcagccttgcaggccagcgatggccattggccagctcaaattgctggccccttgttcttcatgccacccttggtcttttgtgtgtacattactgggcatcttaatacagtattcccatctgaacatcgcaaagaaatccttcgttacatgtactatcaccagaacgaagatggtgggtggggactgcacatagagggtcacagcaccatgttttgcacagcactcaactacatttgtatgcgtatccttggggaaggaccagaggggggtcaagacaatgcttgtgccagagcacgaatgtggattcttgatcatggtggtgtaacacatattccatcttggggaaagacctggctttcgatacttggtctatttgagtggtctggaagcaatccaatgcctccagagttttggatccttccttcatttcttcctatgcatccagcaaaaatgtggtgctattgccggatggtttacatgcccatgtcttatttatatgggaaaaggtttgttggcccaatcacgcctctcattgttcagttaagagaggaaatacacactcaaaattaccatgaaatcaactggaagtcagtccgccatctatgtgcaaaggaggatatctactatccccatccactcatccaagatttgatttgggacagtttgtacatactaacggagcctcttctcactcgctggcccttgaacaagttggtgcgggagagggctctccaagtaacaatgaagcatatccactatgaagatgaaaatagtcgatacataaccattggatgtgtggaaaaggtgttatgtatgcttgcttgttgggttgatgatccaaatggagatgctttcaagaagcaccttgctcgagtcccagattacgtatgggtctctgaagatggaattactatgcagagttttggtagtcaagaatgggatgctggctttgccgtccaggctctgcttgcttctaatcttaccgaggaacttggccctgctcttgccaaaggacatgacttcataaagcaatctcaggttaaggacaatccttcaggtgacttcaaaagcatgtatcgtcacatttctagaggatcatggaccttctctgaccaagatcatggatggcaagtttctgattgcactgcagaaggtctgaagtgttgcctgcttttgtcgatgttgccaccagaaattgttggtgaaaaaatggaaccacaaaggctatttgattctgtcaatgtgctgctctctctacagagcaaaaaaggtggtttagctgcctgggagccagcaggggcgcaagattggttggaattactcaatcccacagaattttttgcggacattgtcgttgagcatgaatatgttgaatgtactggatcagcaattcaggcattagttttgttcaagaagctgtatccggggcacaggaaaaaagagattgacagtttcattacaaatgctgtccggttccttgagaatacacaaacggcagatggctcttggtatggaaactggggagtttgcttcacctatggttgttggttcgcactgggagggctagcagcagctggcaagacttacaacaactgtcctgcaatacgcaaagctgttaatttcctacttacaacacaaagagaagacggtggttggggagaaagctatctttcaagcccaaaaaagatatatgtacccctggaaggaagccgatcaaatgtggtacatactgcatgggctatgatgggtctaattcatgctgggcaggctgaaagagactcaactcctcttcatcgtgcagcaaagttgatcatcaattatcaactagaaaatggcgattggccgcaacaggaaatcactggagtattcatgaaaaactgcatgttacattaccctatgtacagaaacatctacccaatgtgggctcttgcagaataccggaggcgggttccattgccttaa

SEQ ID NO: 18 - protein, 758 aa, Quillaja saponaria
MWRLKIAEGGSDPYLFSTNNFVGRQTWEFEPEAGTPEERAEVEAARQNFYNNRYQVKPCDDLLWRYQFLREKNFKQTIPPVKVEDGQEITYEMATTSMQRAARHLSALQASDGHWPAQIAGPLFFMPPLVFCVYITGHLNTVFPSEHRKEILRYMYYHQNEDGGWGLHIEGHSTMFCTALNYICMRILGEGPEGGQDNACARARMWILDHGGVTHIPSWGKTWLSILGLFEWSGSNPMPPEFWILPSFLPMHPAKMWCYCRMVYMPMSYLYGKRFVGPITPLIVQLREEIHTQNYHEINWKSVRHLCAKEDIYYPHPLIQDLIWDSLYILTEPLLTRWPLNKLVRERALQVTMKHIHYEDENSRYITIGCVEKVLCMLACWVDDPNGDAFKKHLARVPDYVWVSEDGITMQSFGSQEWDAGFAVQALLASNLTEELGPALAKGHDFIKQSQVKDNPSGDFKSMYRHISRGSWTFSDQDHGWQVSDCTAEGLKCCLLLSMLPPEIVGEKMEPQRLFDSVNVLLSLQSKKGGLAAWEPAGAQDWLELLNPTEFFADIVVEHEYVECTGSAIQALVLFKKLYPGHRKKEIDSFITNAVRFLENTQTADGSWYGNWGVCFTYGCWFALGGLAAAGKTYNNCPAIRKAVNFLLTTQREDGGWGESYLSSPKKIYVPLEGSRSNVVHTAWAMMGLIHAGQAERDSTPLHRAAKLIINYQLENGDWPQQEITGVFMKNCMLHYPMYRNIYPMWALAEYRRRVPLP

SEQ ID NO: 19 - DNA, 1443 bp, Quillaja saponaria
  source 1..1443: mol_type = other DNA
atggagcacttgtatctctcccttgtgctcctgtttgtttcctcaatctccctctccctcttcttcctgttctacaaacacaaatctatgttcaccggggccaacctaccacctggtaaaatcggttacccattgatcggagagagcttggagttcttgtccacgggatggaagggccacccggagaaattcatcttcgatcgcatgagcaagtactcatcccaaatcttcaagacctcgattttaggggaaccaacggcggtgttcccgggagccgtatgcaacaagttcctcttctccaacgagaacaagctggtgaatgcatggtggcctgcctccgtggacaagatctttccttcctcactccagacatcctccaaagaagaggccaagaagatgaggaagttgcttcctcagtttctcaagcccgaagctctgcaccgctacattggtattatggattctattgcccagagacactttgccgatagctgggaaaacaaaaaccaagtcattgtctttcctctagcaaagaggtatactttctggctggcttgccgtttgttcattagcgtcgaggatccgacccacgtatccagatttgctgacccgttccaacttttggccgccggaatcatatcaatcccaatcgacttgccagggacaccgttccgcaaggcaatcaatgcgtcccagttcatcaggaaggaattgttggccatcatcaggcagagaaagatcgatttgggtgaagggaaggcatctccgacgcaggacatactgtctcacatgttgctcacatgcgacgagaacggacaatacatgaatgaattggacattgccgacaagattcttggcttgttggtcggcggacatgacactgccagtgccgcttgcactttcattgtcaagttcctcgctgagcttccccacatttatgaacaagtctacaaggagcaaatggagattgcaaaatcaaaagtgccaggagagttgttgaattgggaggacatccaaaagatgaaatattcgtggaacgtagcttgtgaagtgatgagacttgcccctccactccaaggagctttcagggaagccattactgacttcgtcttcaacggtttctccattccaaaaggctggaagttgtactggagcgcaaattccacccacaaaagtccggattatttccctgagcccgacaagttcgacccaactagattcgaaggaaatggacctgcgccttacacctttgttccatttgggggaggacccaggatgtgcccgggcaaagagtatgcccgattggaaatacttgtgttcatgcataacttggtgaagaggttcaagtgggagaaattggttcctgatgaaaagattgtggttgatccaatgcccattccagcaaagggtcttcctgttcgcctttatcctcacaaagcttga

SEQ ID NO: 20 - protein, 480 aa, Quillaja saponaria
MEHLYLSLVLLFVSSISLSLFFLFYKHKSMFTGANLPPGKIGYPLIGESLEFLSTGWKGHPEKFIFDRMSKYSSQIFKTSILGEPTAVFPGAVCNKFLFSNENKLVNAWWPASVDKIFPSSLQTSSKEEAKKMRKLLPQFLKPEALHRYIGIMDSIAQRHFADSWENKNQVIVFPLAKRYTFWLACRLFISVEDPTHVSRFADPFQLLAAGIISIPIDLPGTPFRKAINASQFIRKELLAIIRQRKIDLGEGKASPTQDILSHMLLTCDENGQYMNELDIADKILGLLVGGHDTASAACTFIVKFLAELPHIYEQVYKEQMEIAKSKVPGELLNWEDIQKMKYSWNVACEVMRLAPPLQGAFREAITDFVFNGFSIPKGWKLYWSANSTHKSPDYFPEPDKFDPTRFEGNGPAPYTFVPFGGGPRMCPGKEYARLEILVFMHNLVKRFKWEKLVPDEKIVVDPMPIPAKGLPVRLYPHKA

SEQ ID NO: 21 - DNA, 1506 bp, Quillaja saponaria
  source 1..1506: mol_type = other DNA
atgatatataataatgatagtaatgataatgaattagtaatcagctcagttcagcaaccatccatggatcctttcttcatttttggcttacttctcttggctctctttctctctgtttcttttcttctctacctttcccgtagagcctatgcttctctccccaaccctccgccggggaagctcggcttccccgtcgtcggcgagagtctcgaatttctctccacccgacgcaaaggtgttcctgagaaattcgtcttcgacagaatggccaaatactgtcgggatgtctttaagacatcaatattgggagcaaccaccgccgtcatgtgcggcaccgccggtaacaaattcttgttctccaacgagaaaaaacacgtcactggttggtggccgaaatctgtagagctgattttcccaacctcacttgagaaatcatccaacgaagaatccatcatgatgaaacaattccttcccaacttcttgaaaccagaacctttgcagaagtacatacccgttatggacataattacccaaagacacttcaatacaagctgggaaggacgcaacgtggtcaaagtgtttcctacggctgccgaattcaccacgttgctggcttgtcgggtattcctcagtgttgaggatcccattgaagtagccaagatttcagagccatttgaaatcttagctgctgggtttctttcaatacccataaatcttccgggtaccaaattaaataaagcggttaaggcagcggatcagattagagacgcaattgtacagattttgaaacggagaagggttgaaattgcggagaataaagcaaatggaatgcaagatatagcgtccatgttgttgacgacaccaactaatgctgggttttatatgaccgaggctcacatttctgagaaaattttgggtatgattgttggtggccgtgatactgctagtactgttatcaccttcatcatcaagtatttggcagagaatcctgaaatttataataaggtctatgaggagcaaatggaagtggtaaagtcaaagaaaccaggtgagttgctgaactgggaagatgtgcagaaaatgaagtactcttggtgcgtagcatgtgaagctatgcgacttgctcctcctgttcaaggtggtttcaaggtggccattaatgactttgtgtattctgggttcaacattcgcaagggttggaagttatattggagtgccattgcaacacacatgaatccagaatatttcccagaacctgagaaattcaacccctcaaggtttgaagggaagggaccagtaccttacagcttcgtacccttcggaggcggacctcggatgtgtcccgggaaagagtattcccggctggaaacacttgttttcatgcatcatttggtgacgaggtacaattgggagaaagtgtatcccacagagaagataacagtggatccaatgccattccctgtcaacggcctccccattcgccttattcctcacaagcaccaatga

SEQ ID NO: 22 - protein, 501 aa, Quillaja saponaria
MIYNNDSNDNELVISSVQQPSMDPFFIFGLLLLALFLSVSFLLYLSRRAYASLPNPPPGKLGFPVVGESLEFLSTRRKGVPEKFVFDRMAKYCRDVFKTSILGATTAVMCGTAGNKFLFSNEKKHVTGWWPKSVELIFPTSLEKSSNEESIMMKQFLPNFLKPEPLQKYIPVMDIITQRHFNTSWEGRNVVKVFPTAAEFTTLLACRVFLSVEDPIEVAKISEPFEILAAGFLSIPINLPGTKLNKAVKAADQIRDAIVQILKRRRVEIAENKANGMQDIASMLLTTPTNAGFYMTEAHISEKILGMIVGGRDTASTVITFIIKYLAENPEIYNKVYEEQMEVVKSKKPGELLNWEDVQKMKYSWCVACEAMRLAPPVQGGFKVAINDFVYSGFNIRKGWKLYWSAIATHMNPEYFPEPEKFNPSRFEGKGPVPYSFVPFGGGPRMCPGKEYSRLETLVFMHHLVTRYNWEKVYPTEKITVDPMPFPVNGLPIRLIPHKHQ

SEQ ID NO: 23 - DNA, 1524 bp, Quillaja saponaria
  source 1..1524: mol_type = other DNA
atgtggttcacagtaggattggtcttggttttcgccctattcatacgtctctacagcagtctgtggttgaagcctcgtgcaactcggattaagcttagcaatcaaggaattaaaggtccaaaaccagcatttcttctgggtaatgttgcagagatgagaagatttcaatctaagcttccaaaatctgaactcaaacaaggccaagtttctcatgattgggcttctaaatctctgtttccatttttcagtctttggtcccagaaatacggaaatacgttcgtgttctcattggggaacatacaggtgctctatgtttctgatcatgagttggtgaaagaaattaatcagaatacctctttagatttgggcaaacccaagtacctgcagaaggagcgtggccctttgctgggacaaggtattttgacctccaatggacagctttgggcgtaccagagaaaaatcatgactcctgaactctacaaggagaaaatcaagggcatgtgcgagttgatggtggaatctgtagcttggttggttgaggaatggggaacgaagatccaagctgagggtggggcagcagacattagaatagacgaggatcttagaagcttctctggtgatgtaatttcaaaagcttgttttgggagctgctatgccggagggagggaaatctttcttaggctcagagctcttcaacaccaaattgcttccaaagccttactcatgggcttccctggattaaagtacctgcccattaagagcaacagagagatatggagattggagaaggagatcttccagctgattatgaagctggctgaagatagaaaaaaagaacaacatgagagagacctattacagattataattgagggagctaaaagtagtgatctgagttcggaagcaatggcaaaattcattgtggacaactgcaagaatgtctacttggctggccatgaaactactgcaatgtctgctggttggactttgcttctcttggctaatcatcctgagtggcaagcccgtgtccgtgatgagattttacaagtcaccgagggccgcaatcctgattttgacatgctgcacaagatgaaactgttaacaatggtaattcaggaggcactgcgactctacccaacagtcatattcatgtcaagagaagcattggaagatattaatgttggaaacatccaagttccaaaaggtgttaacatatggatacctgtggtaaatcttcaaagggacacaacggtatggggtgcagacgcaaacgagtttaatcctgaaaggtttgccaatggagttaacaattcatgcaaggttccacaactttacctaccatttggagctggacctcgcatttgtcctggaattaatctggccatgactgagatcaagatacttctgtgtatcctgctcaccaagttttcgttttcagtttcacccaactatcgccactcaccggtgtttaaattggtgcttgagcctgaaaatggaatcaatgtcatcatgaagaagctctaa

SEQ ID NO: 24 - protein, 507 aa, Quillaja saponaria
MWFTVGLVLVFALFIRLYSSLWLKPRATRIKLSNQGIKGPKPAFLLGNVAEMRRFQSKLPKSELKQGQVSHDWASKSLFPFFSLWSQKYGNTFVFSLGNIQVLYVSDHELVKEINQNTSLDLGKPKYLQKERGPLLGQGILTSNGQLWAYQRKIMTPELYKEKIKGMCELMVESVAWLVEEWGTKIQAEGGAADIRIDEDLRSFSGDVISKACFGSCYAGGREIFLRLRALQHQIASKALLMGFPGLKYLPIKSNREIWRLEKEIFQLIMKLAEDRKKEQHERDLLQIIIEGAKSSDLSSEAMAKFIVDNCKNVYLAGHETTAMSAGWTLLLLANHPEWQARVRDEILQVTEGRNPDFDMLHKMKLLTMVIQEALRLYPTVIFMSREALEDINVGNIQVPKGVNIWIPVVNLQRDTTVWGADANEFNPERFANGVNNSCKVPQLYLPFGAGPRICPGINLAMTEIKILLCILLTKFSFSVSPNYRHSPVFKLVLEPENGINVIMKKL

SEQ ID NO: 25 - DNA, 2142 bp, Quillaja saponaria
  source 1..2142: mol_type = other DNA
atgaaatccccctctaacccaaatcagaaacccatcctccacacttgtacaattcagcagcctcgtgctacccttaacaaaattcatagtcttattcatttctcagccatacttgtcctattttattaccggataacccgtctattcttcaccgacgatttcaaggtacccaagttactatggactctaatgacaatctccgagttcattcttgccttcatttgggttctcatccaacctttccggtggcgaccggtgtcccgttccgtcataccagagaatatgccgaaggacatcagtttgccggcggtggacgtgtttgtatgcacagctgaccctcaaaaagaacccacagtggaggtgatgaacacaattttatcagccatggctttagactacccggcggagaagctcgccgtgtatctttccgatgatgggggttctgctgtcaccttatatgctataaaagaagcttgttgttttgctaagatgtggcttccgttttgtaacaagtatgggatcaaatcaaggtgtcccgaggcttatttttcaaagcttgccgctgacgagtggcttcaccggagtgtggaattcgtggcagaagaaaaggaggtcaaggctaattatgaagagttcaagagaaatgtgcagaaatttggtgagcaacaagaaaacagtcgtgttgtgcatgatcgtcaccctcatgttgagattatacacaataattggaataacgaagaccaagctcatgagatgccactccttgtttatgtctctcgtgaaagaagaccatctcaccatcctcgattcaaagctggagctcttaacacccttcttcgagtttctggcatcatcagcaacagcccctacatactggttctagactgtgacatgtactgcaatgacccaacctcagctagacaagcaatgtgcttccatcttgatccccaactgtctaaaaatcttgcttttgtacaattccctcaaatattctataacgctagtaagaatgacgtctatgatgcccaagtcagggcggcataccagacaaagtggcagggtatggatggacttcaaggaccaattttttctggcactggcttttacttaaagaggaaggcaatgtatggaaaccctgatcaagatgataattgtctactcaagccatataagaaatttggcatgtctggagaatttgtagaatcacttaaggtccttaacgaacaagatggtacccagaagaaattattggatggatttttacaagaggccaaactattggcctcgtgtgcctatgaaacaaagacaagttggggtaaagagattggattctcatatgactgtttaatagagagcactttcactggttatcttttgcactgcagagggtggatatctgtttatctttatcccaagagaccatgttttttaggatgctgtcctactgatatgaaggatgccatggttcaatataccaagtggatgtctgagctattttcaattgctatctcaagattcaatcctctgctctatggggtggcaagaatgtccattcttcaaagcctgtgttatggatcctttacactggcgcctattttgtcatttcctttgttcttatatggaacggttcctcaattatgcctcttgaaaggcatatctttgtttccaaaggtttcggacccatggtttgctgtgtttgcagctatctttgtatcctccctgtgtcaacactggttcgaggtcctctcttgtgatggtacatttacgacttggtgtaatgaacagcggagttggcttataaagtcggtttccggtagtttgtttggagttgtgggcgcaatcttgcagcggctaggcttgaagacaaagtttagtttatcaaacaaagccatggacaaagaaaagctggagaaatatgaaaagggtaaatttaatttccaaggggctgccatgttcatggttcctgtgtctattttagtcatactgaacacattttgcttcctcggtgggttttggaaagtgatcataatgaagaatatcctggacatgtttggacaactttctctctctgcctacgttctggttctcagttgtccagttcttgaagggatgttaactagaatcagcaagaaaatggtctga

SEQ ID NO: 26 - protein, 713 aa, Quillaja saponaria
MKSPSNPNQKPILHTCTIQQPRATLNKIHSLIHFSAILVLFYYRITRLFFTDDFKVPKLLWTLMTISEFILAFIWVLIQPFRWRPVSRSVIPENMPKDISLPAVDVFVCTADPQKEPTVEVMNTILSAMALDYPAEKLAVYLSDDGGSAVTLYAIKEACCFAKMWLPFCNKYGIKSRCPEAYFSKLAADEWLHRSVEFVAEEKEVKANYEEFKRNVQKFGEQQENSRVVHDRHPHVEIIHNNWNNEDQAHEMPLLVYVSRERRPSHHPRFKAGALNTLLRVSGIISNSPYILVLDCDMYCNDPTSARQAMCFHLDPQLSKNLAFVQFPQIFYNASKNDVYDAQVRAAYQTKWQGMDGLQGPIFSGTGFYLKRKAMYGNPDQDDNCLLKPYKKFGMSGEFVESLKVLNEQDGTQKKLLDGFLQEAKLLASCAYETKTSWGKEIGFSYDCLIESTFTGYLLHCRGWISVYLYPKRPCFLGCCPTDMKDAMVQYTKWMSELFSIAISRFNPLLYGVARMSILQSLCYGSFTLAPILSFPLFLYGTVPQLCLLKGISLFPKVSDPWFAVFAAIFVSSLCQHWFEVLSCDGTFTTWCNEQRSWLIKSVSGSLFGVVGAILQRLGLKTKFSLSNKAMDKEKLEKYEKGKFNFQGAAMFMVPVSILVILNTFCFLGGFWKVIIMKNILDMFGQLSLSAYVLVLSCPVLEGMLTRISKKMV

SEQ ID NO: 27 - DNA, 2124 bp, Quillaja saponaria
  source 1..2124: mol_type = other DNA
atggcgaccgtctcctccctccacacttgcactgtacagcaaccccgtgcagccattaatcgaattcacattttcttacactttattgccatacttttcctcttttactaccgggtcaccggtcttttctatgacaatgcagtacccactttagcttggtctctaatgaccttagctgagttgattttcgccttcgtttgggtgctcagccaagccttccggtggcgcccggtgttgcgttcagttattcctgagaggattcccaaagatgtacgattgcccgcggtggatatcttaatttgtacggctgacccattaaaggaaccgacggtggaggtgatgaacactgtcttgtccgccatggcattggactatcctgcggagaatctggctgtatatctttctgatgacgggggttctccggtcaccttatttgctatgaagcaagtgggtccgtttgctaagctgtggcttccgttttgcaacaagtacggaatcaaaacaaggcatcctgagtcttttttctcggcatttgcggatgacgaaaggcttcaccggagtgatgaattcagggcagaggaggaggcgatcaaggacaaatatgaagaatttaagagaactatagagaaatatggtggagaaggaaaaaatagtcatgttgtacaagaccggcctcctcatgtggagattatacatgacactaggaagattagagagaacagtgaagaccaagctgtgcctcttcttgtctacgtctctcgtgagaaaagaccatcctacaattctcggttcaaagcaggagctctgaacacccttcttcgagtttctggggtaatcagcaatagcccatatgtattggtgttagactgtgacatgtactgcaatgatccaacatcagctagacaagcaatgtgcttccatcttgatccacaaatgtctcgcactctctcttttgtacaattcccccaggttttctacaatgttagtaaaaatgatatctatgatggccaagctagggcagcctttaagacaaagtggcaaggtatggatggactacgtgggccactgctttctggtactggcttttatttgaagaggaagtccttgtatggaagtccaaaccaagaagatgattgtttacttgagccccataagaattttggaaagtgtgacaagctcatagaatcagtaaaggtcatttatgaacgtgatgtttcaataaaggcagattcatcagatgccattttgcaagatgccaaacaattagcatcttgtccctatgaaacaaacacaagctggggcaaagaggttgggttctcgtatgactgcttattagagagtacattcacaggttatctgttgcactgcagagggtggacatcagtttatctttatccaaagaagccatgtttcttagggtgtactccagttgatatgaaggaagccatggttcagtatacgaagtggatttctgaattatttttacttgctatctcaagattcaaccctctgacatttgggatatccagaatgtccattctccagagcatgtgttacggataccttacaatcatgcccattttatctgttgctatgatcttctatgccacagttcctcaattgtgcctcttgagaggcgtacctctgtttcccaaggtttcagacccatggtttgcagtgttcctagcaatatttgtgtcctccctctgtcagcacttaattgaagtcctcacgagtgatggcacgctcaagacttggtggaatgaacaaagaaattgggtgataaagtctggttccggtagcgtatttggagctctgagtggaatattgaagtggtttggcatgaagattaaatttggtttatcaaacaaagccgtggacaaagaaaagcttgagaaatatgaaaagggtaagtttgatttccaaggggctgccatgtttatggttcccttaactatatcagtcatcttgaacacattatgccttatcggtggtttatggagagtaatcacacttaaaaacttcgaagagatgtcagggcagttcatcatctccttgtactttctagctctcagctatccaattcttgaagggttactaagaaaaggcaagggaaaggcctaa

SEQ ID NO: 28 - protein, 707 aa, Quillaja saponaria
MATVSSLHTCTVQQPRAAINRIHIFLHFIAILFLFYYRVTGLFYDNAVPTLAWSLMTLAELIFAFVWVLSQAFRWRPVLRSVIPERIPKDVRLPAVDILICTADPLKEPTVEVMNTVLSAMALDYPAENLAVYLSDDGGSPVTLFAMKQVGPFAKLWLPFCNKYGIKTRHPESFFSAFADDERLHRSDEFRAEEEAIKDKYEEFKRTIEKYGGEGKNSHVVQDRPPHVEIIHDTRKIRENSEDQAVPLLVYVSREKRPSYNSRFKAGALNTLLRVSGVISNSPYVLVLDCDMYCNDPTSARQAMCFHLDPQMSRTLSFVQFPQVFYNVSKNDIYDGQARAAFKTKWQGMDGLRGPLLSGTGFYLKRKSLYGSPNQEDDCLLEPHKNFGKCDKLIESVKVIYERDVSIKADSSDAILQDAKQLASCPYETNTSWGKEVGFSYDCLLESTFTGYLLHCRGWTSVYLYPKKPCFLGCTPVDMKEAMVQYTKWISELFLLAISRFNPLTFGISRMSILQSMCYGYLTIMPILSVAMIFYATVPQLCLLRGVPLFPKVSDPWFAVFLAIFVSSLCQHLIEVLTSDGTLKTWWNEQRNWVIKSGSGSVFGALSGILKWFGMKIKFGLSNKAVDKEKLEKYEKGKFDFQGAAMFMVPLTISVILNTLCLIGGLWRVITLKNFEEMSGQFIISLYFLALSYPILEGLLRKGKGKA

SEQ ID NO: 29 - DNA, 1479 bp, Quillaja saponaria
  source 1..1479: mol_type = other DNA
atggtggagtctccagcagatcatgatgtgctcaaaatcattgtccttccatgggtaacctcaggtcacatgattcccatggtagatgcagccagactatttgctatgcatggtgcagatgttaccatcatcaccaccccagctaatgcccttacattccagaaatccgtcgaccgtgatttcaattccggtcgtttaatcagaactcacacccttaaattccctgcagcagaagttggtgtacctgaaggagttgaaaacttcaacaatacttcccctgaaatgacctccaaagtctaccttggagtctcaatgctccgagaaccaacccaacaattgattgaggatctgcgtccagattgtcttatcactgatatgttctatccttgggctgtggatgttgctgacaaattaggcattccaaggctaatttttcaaggtcctggaagttttggtttgtcagctatgcattctatcaaacagtatgagccctttaagtcagtaacttcagatactgagacattcccactacctggattgccgcataaggtagagatgacaaggttgcagataccaaaatgggttcgtgagccaaatgggtacactcaattgatgggcagggtaaaagattcggagagaagaagctatgggtcattggtgaatagcttttatgacttcgaaggcccttatgaagagcactataggaaggcaacaggacagagggtttggagcattggaccagtttcagtttgggtgaaccaagatgctgcagataaggttggaagaggacaggatcttgttgctgaagaccaaaacagctggttgaattggctcaattccaaagagaaaaactctgttctgtatgtaagttttgggagcatggccaagttcccatctgctcagcttcttgaaatagctcatgggcttgaagcttcaggtcatagtttcatctgggttgtcagaaaagttgacggggatgatgatgtagacgtgtggcttccagattttgagaagaaaatgaaagagaacaacaagggtttcatcataaggaattgggcaccacaattgctcatattggaccatccagcaattggaggtttgctgaatcacagtggatggaattcagtactggaaggtgctacagcaggcttgccaatgatcacttggcctctgtatgccgagcatttttacaatgaaaggttggttctagatgtgttgaaaattggagtaccagttggggtgaaggagtggaagaacttgcatgaggtgggtgagttggtgagaagggatgcaattgccaaggcaattaaattgttaatgggtagtggagaagaagctgaggtaatgaggaaaaaagccaaagagcttggtgttggagcaaagaaaggtattcaggttggaggttcttctcataccaatttgatagcagtgattgatgagttaaagtcactaaagaaatcaagaattcagggtgtctga

SEQ ID NO: 30 - protein, 492 aa, Quillaja saponaria
MVESPADHDVLKIIVLPWVTSGHMIPMVDAARLFAMHGADVTIITTPANALTFQKSVDRDFNSGRLIRTHTLKFPAAEVGVPEGVENFNNTSPEMTSKVYLGVSMLREPTQQLIEDLRPDCLITDMFYPWAVDVADKLGIPRLIFQGPGSFGLSAMHSIKQYEPFKSVTSDTETFPLPGLPHKVEMTRLQIPKWVREPNGYTQLMGRVKDSERRSYGSLVNSFYDFEGPYEEHYRKATGQRVWSIGPVSVWVNQDAADKVGRGQDLVAEDQNSWLNWLNSKEKNSVLYVSFGSMAKFPSAQLLEIAHGLEASGHSFIWVVRKVDGDDDVDVWLPDFEKKMKENNKGFIIRNWAPQLLILDHPAIGGLLNHSGWNSVLEGATAGLPMITWPLYAEHFYNERLVLDVLKIGVPVGVKEWKNLHEVGELVRRDAIAKAIKLLMGSGEEAEVMRKKAKELGVGAKKGIQVGGSSHTNLIAVIDELKSLKKSRIQGV

SEQ ID NO: 31 - DNA, 1515 bp, Quillaja saponaria
  source 1..1515: mol_type = other DNA
atggtctccggcgacgacgatgtttctcgtcggccactgaaagtttacttcattgcacacccctcacctggccatattgcccctctgaccaaaatagcccatctcttcgctgccctcggtgagcacgtgactattctcactactcccgccaatgtccacttccatgagaaatccatcgacaaaggaaaggcttccggctatcatgttaacatccacaccgttaaatttccttctaaagaggtcggtctccctgacggcatcgaaaacttctcttacgcctccgatgttgaaacagcagctaaaatttgggctggattcgccatgctacaaactgaaatggagcaatatatggagcttaacccacccgattgcatcgttgccgacatgttcacctcctggacctccgactttgctatcaaattgggaatcacaagaatcgttttcaacgtctattgtattttcacacgctgtttggaagaagccatccgatcaccggactcgccacacttgaacaaagaaatctctgataatgaaccgtttgttatcccgggtctaccagaccccataacaattacccgagctcaactgcccgacggtaccttttctcccatgaaagaactagctagaacagctgagttgaagagctttggaatggtgatcaacgggttttccgaactcgaaaccgattacatcgagcattacaagaaaatcatgggtcacaaacggatttggcatgtcggaccccttcagctaatccaccgtaacgatgaagacaaaattcagaggagccacaagacagcggtgctgagtgataacgataacgagttagtgagttggcttaactcgaagaaacccgactcagttatttacatttgcttcggtagtgcaactcgtttctctaatcaccagctctatgaaatcgcctgtggattagaagcttccgggcacccatttttgtggggcctactttgggtgccagaagatgaagataacgatgacgtgggcaacaaatggttgccagctttcgaagaaagaattaaaaaggaaaataagggaatgattttaagggggtgggctccacagatgttaatcttaaaccacccggcgatcggtggtttcatgacgcattgtggttggaatgcggtggtggaagcactttcattcggtgttccgactattacgcttccagttttctcggagcagttttatactgagagactgatatcacaagtgctcaagactggtgtggaggttggtgcagagaagtggacctatgcatttgatgcggggaaatatccggtgagtagggaaaagatagcgacggcggtgaagaagatattagacgatggagaagaggcagaaggaatgagaaagcgggccagggagatgaaagaaaaagcccaaaaaagtgttgaagaaggtggatcctcttataataatttaacggctatgattgaagatcttaaagaatttagggctaacaatggcaaggctgcacaagatcatgaatcgtga

SEQ ID NO: 32 - protein, 504 aa, Quillaja saponaria
MVSGDDDVSRRPLKVYFIAHPSPGHIAPLTKIAHLFAALGEHVTILTTPANVHFHEKSIDKGKASGYHVNIHTVKFPSKEVGLPDGIENFSYASDVETAAKIWAGFAMLQTEMEQYMELNPPDCIVADMFTSWTSDFAIKLGITRIVFNVYCIFTRCLEEAIRSPDSPHLNKEISDNEPFVIPGLPDPITITRAQLPDGTFSPMKELARTAELKSFGMVINGFSELETDYIEHYKKIMGHKRIWHVGPLQLIHRNDEDKIQRSHKTAVLSDNDNELVSWLNSKKPDSVIYICFGSATRFSNHQLYEIACGLEASGHPFLWGLLWVPEDEDNDDVGNKWLPAFEERIKKENKGMILRGWAPQMLILNHPAIGGFMTHCGWNAVVEALSFGVPTITLPVFSEQFYTERLISQVLKTGVEVGAEKWTYAFDAGKYPVSREKIATAVKKILDDGEEAEGMRKRAREMKEKAQKSVEEGGSSYNNLTAMIEDLKEFRANNGKAAQDHES

SEQ ID NO: 33 - DNA, 1485 bp, Quillaja saponaria
  source 1..1485: mol_type = other DNA
atggtctccggcgacgacgacgtttctcgtcggccactgaaagtttactttattgcacacccctcacctggccatattgcccctctaaccaaaatagcccaactctttgctgcacgtggtgagcacgtgactattcttactactcccgccaatgtccacttccatgagaaatccatcgacaaaggaaagacttccggctatcatgttaacatccacgccgttaaatttccttctaaagaggtcggtctccccgacggcatcgaaaacttctctcacgcctccgataatgaaacagcagccaaaatttgggccggattctccatgcttcaaactgaaatggagcaatatatggaacaaaacccacccgattgcattgttgccgacatgttcaaccgctggacttccgacttcgctatcaaattgggaatcccgagaatagttttcaacgtctactgtattttcacacgctgtttggaagaagcaatcagatcacctgactcgccacacttgaaactaaactccgataatgaacagtttattattccgggtctacccgaccccataacaattacccgagctcaactgcccgacggtgccttttctgtcgtcaaagaacaagttagtgaagctgagttgaaaagcttcggaatggtgatcaacgggttttccgaactcgaaaccgaatacatcgagtattacaagaatatcatgggtcgaaaacggatttggcatgtcggaccccttcagctcatttaccaaaacgatgaccccaaagttcagaggagccagaagacagcggtcgtgagtgacaacgagttagtgagttggcttgactcgaagaaacccgactcagtgatttacatttccttcggtagtgcaattcgtttctctaataagcagctctatgaaatagcatgtggattagaagcttccggctacccatttttgtgggccttactttgggtgccagaagatgacgacgacgtgggcaacaaatggttgcctgatttcgaagaaagaataaaaagagaaaataagggaataattttcagggggtgggccccacagatgttaatcttaaaccacccggcgatcggtggtttcatgacgcattgtggttggaatgcggtggtggaagcgctttctttcggtgttccgactattacgcttccggttttctcggagcagttttatactgagagactgatatcacaagtgctcaagactggtgtcgaggtcggtgcagagaagtggacctatgcatttgatgcggggaaatatccggtgagtcgggaaaagatagcgacggcggtgaagaagatattagactgtggagaagaggcagaaggaatgagaaagcgggccagggagatgaaagaaaaagcccaaaaaagtgttgaagaaggtgggtcctcttataataatttaacggctatgattgaagatcttaaagaatttagggctaacaatggcaaggttgcatga

SEQ ID NO: 34 - protein, 494 aa, Quillaja saponaria
MVSGDDDVSRRPLKVYFIAHPSPGHIAPLTKIAQLFAARGEHVTILTTPANVHFHEKSIDKGKTSGYHVNIHAVKFPSKEVGLPDGIENFSHASDNETAAKIWAGFSMLQTEMEQYMEQNPPDCIVADMFNRWTSDFAIKLGIPRIVFNVYCIFTRCLEEAIRSPDSPHLKLNSDNEQFIIPGLPDPITITRAQLPDGAFSVVKEQVSEAELKSFGMVINGFSELETEYIEYYKNIMGRKRIWHVGPLQLIYQNDDPKVQRSQKTAVVSDNELVSWLDSKKPDSVIYISFGSAIRFSNKQLYEIACGLEASGYPFLWALLWVPEDDDDVGNKWLPDFEERIKRENKGIIFRGWAPQMLILNHPAIGGFMTHCGWNAVVEALSFGVPTITLPVFSEQFYTERLISQVLKTGVEVGAEKWTYAFDAGKYPVSREKIATAVKKILDCGEEAEGMRKRAREMKEKAQKSVEEGGSSYNNLTAMIEDLKEFRANNGKVA

SEQ ID NO: 35 - DNA, 1491 bp, Quillaja saponaria
  source 1..1491: mol_type = other DNA
atggtctccggcgacgataccgtttcacggccactgatagtttactttattgcacacccctcacctggccatattgcccctctaaccaaaatagcccaactcttcgctgcacgtggtgagcacgtcactattcttactactcccgccaatgtccacttccatgagaaatccatcgacaaaagaaagaattccggctatcatgttaacatccacaccgttaaatttccttctaaagaggtcggtctccctgacggcatcgaaaacttctctcacgcctccgataatgaaacagcagccaaaatttgggccggattctccatgcttcaaactgaaatggagcaatatatggaacaaaacccacccgattgcatcgttgccgacatgttcaaccgctggacttccgacttcgctatcaaattgggaatcccgagaatagttttcaacgtctactgtattttcacacgctgtttggaagaagcaatcagatcacctgactcgccacacttgaaactaaactccgataatgaacagtttattattcccggtctacccgaccccataacaattacccgagctcaactccccgacggtgccttttctgtcgtcaaagaacaagttagtgaagctgagttgaaaagcttcggaatggtgatcaacgggttttccgaactcgaaactgaatacatcgagtattacaagaatatcatgggtcgcaaacggatttggcatgtcggaccccttcagctaatttaccaaaacgacgaccccaaagttcagaggagccagaagacagcggtcttgagtgacaacgagttagtgagttggcttgactcgaagaaacccgactcagtgatttacatttccttcggtagtgcaattcgtttctctaataagcagctctatgaaatcgcatgtggattagaagcttccggctacccatttttgtgggccttactttgggtgccagaagatgatgacgacgtgggcaacaaatggttgccgggtttcgaagaaagaataaaaagagaaaataagggaataattttcagggggtgggccccacagatgttaatcttaaaccacccggcgatcggtggtttcatgacgcattgtggttggaatgcggtggtggaagcactttcattcggtgttccgactattacgcttccagttttctcggagcagttttatactgagagactgatatcacaagtgctcaagactggtgtggaggttggtgcagagaagtggacctatgcatttgatgcggggaaatatccggtgagtagggaaaagatagcgacggcggtgaagaagatattagacgatggagaagaggcagaaggaatgagaaagcgggccagggagatgaaagaaaaagcccaaaaaagtgttgaagaaggtggatcctcttataataatttaacggctatgattgaagatcttaaagaatttagggctaacaatggcaaggctgcaatgaaatcatga

SEQ ID NO: 36 - protein, 496 aa, Quillaja saponaria
MVSGDDTVSRPLIVYFIAHPSPGHIAPLTKIAQLFAARGEHVTILTTPANVHFHEKSIDKRKNSGYHVNIHTVKFPSKEVGLPDGIENFSHASDNETAAKIWAGFSMLQTEMEQYMEQNPPDCIVADMFNRWTSDFAIKLGIPRIVFNVYCIFTRCLEEAIRSPDSPHLKLNSDNEQFIIPGLPDPITITRAQLPDGAFSVVKEQVSEAELKSFGMVINGFSELETEYIEYYKNIMGRKRIWHVGPLQLIYQNDDPKVQRSQKTAVLSDNELVSWLDSKKPDSVIYISFGSAIRFSNKQLYEIACGLEASGYPFLWALLWVPEDDDDVGNKWLPGFEERIKRENKGIIFRGWAPQMLILNHPAIGGFMTHCGWNAVVEALSFGVPTITLPVFSEQFYTERLISQVLKTGVEVGAEKWTYAFDAGKYPVSREKIATAVKKILDDGEEAEGMRKRAREMKEKAQKSVEEGGSSYNNLTAMIEDLKEFRANNGKAAMKS

SEQ ID NO: 37 - DNA, 1515 bp, Quillaja saponaria
  source 1..1515: mol_type = other DNA
atggtctccggcgacgacgatgtttctcgtcggccactgaaagtttacttcattgcacacccctcacctggccatattgcccctctgaccaaaatagcccatctcttcgctgccctcggtgagcacgtgactattctcactactcccgccaatgtccacttccatgagaaatccatcgacaaaggaaaggcttccggctatcatgttaacatccacaccgttaaatttccttctaaagaggtcggtctccctgacggcatcgaaaacttctcttacgcctccgatgttgaaacagcagctaaaatttgggctggattcgccatgctacaaactgaaatggagcaatatatggagcttaacccacccgattgcatcgttgccgacatgttcacctcctggacctccgactttgctatcaaattgggaatcacaagaatcgttttcaacgtctattgtattttcacacgctgtttggaagaagccatccgatcaccggactcgccacacttgaacaaagaaatctctgataatgaaccgtttgttatcccgggtctaccagaccccataacaattacccgagctcaactgcccgacggtaccttttctcccatgaaagaactagctagaacagctgagttgaagagctttggaatggtgatcaacgggttttccgaactcgaaaccgattacatcgagcattacaagaaaatcatgggtcacaaacggatttggcatgtcggaccccttcagctaatccaccgtaacgatgaagacaaaattcagaggagccacaagacagcggtgctgagtgataacgataacgagttagtgagttggcttaactcgaagaaacccgactcagttatttacatttgcttcggtagtgcaactcgtttctctaatcaccagctctatgaaatcgcctgtggattagaagcttccgggcacccatttttgtggggcctactttgggtgccagaagatgaagataacgatgacgtgggcaacaaatggttgccagctttcgaagaaagaattaaaaaggaaaataagggaatgattttaagggggtgggctccacagatgttaatcttgaatcacccggcgatcggtggtttcatgacgcattgtggttggaatgcggcggtggaggcgctttcttccggtgttccgattattacatttccggttttctcggatcagttttataatgaaaggctgatatcacaagtgcataagtgtggggtgggggttggtacggaggcgtggagctatgcattcgatgccgggaagaatccggtgggtcgggaaaagataatgacggcggtgaagaagatattagacggtggagaagaggcggaaggaatgagaaagagggcccgggagctgaaagaaatagctaaaagaagtgtggaagaaggtgggtcctcttataataatttaacggctatgattcaagatctgaaagaatttagagctaacaatggcaaggctgcacaagatcatgaatcgtga

SEQ ID NO: 38 - protein, 504 aa, Quillaja saponaria
MVSGDDDVSRRPLKVYFIAHPSPGHIAPLTKIAHLFAALGEHVTILTTPANVHFHEKSIDKGKASGYHVNIHTVKFPSKEVGLPDGIENFSYASDVETAAKIWAGFAMLQTEMEQYMELNPPDCIVADMFTSWTSDFAIKLGITRIVFNVYCIFTRCLEEAIRSPDSPHLNKEISDNEPFVIPGLPDPITITRAQLPDGTFSPMKELARTAELKSFGMVINGFSELETDYIEHYKKIMGHKRIWHVGPLQLIHRNDEDKIQRSHKTAVLSDNDNELVSWLNSKKPDSVIYICFGSATRFSNHQLYEIACGLEASGHPFLWGLLWVPEDEDNDDVGNKWLPAFEERIKKENKGMILRGWAPQMLILNHPAIGGFMTHCGWNAAVEALSSGVPIITFPVFSDQFYNERLISQVHKCGVGVGTEAWSYAFDAGKNPVGREKIMTAVKKILDGGEEAEGMRKRARELKEIAKRSVEEGGSSYNNLTAMIQDLKEFRANNGKAAQDHES

SEQ ID NO: 39 - DNA, 1053 bp, synthetic construct
  misc_feature 1..1053: note = Acanthocystis turfacea chlorella virus 1 UDP-D-glucose 4,6-dehydratase (ATCV-1) coding sequence. Codon-optimised for expression in N. benthamiana. The original sequence can be found as Genbank ID: NC_008724.1 (see locus tag ATCV_z554R).
  source 1..1053: mol_type = other DNA
atgaatagtcaggagtatactcctaaatcagtccttgtaactggtggggcaggtttcattggcagccatgtcgtgatgaaattggtccagaggtatccggaatgcaaggtcgttgttcttgacaaaatggattactgcgctaccttaaataatcttgctacggtacgagatgcccccaattttaaatttgtaaaaggtgatatacaaagcactgatcttttagctcacgtgctcaaacaggaaaagatagataccattatgcactttgccgcccagacccatgtagataatagctttggcaatagtttagcatttacgatgaacaacgtatacggcactcacgtccttctcgaatgtgcccgattgtatggtggtgttcagagattcattaacgtgtcaactgatgaggtctacggtgagagttccttgggaaagaaggaggggttggacgaacactcctccctcgaaccgacaaatccttatgccgccgcaaaggctggagctgaaatgatggctagagcatatcatacgtcatacaaactcccggtaatagtcacgcgtggcaataatgtatatggtccgcaccagttccccgaaaaaatgatccccaaatttattctcagggcaaccagaggcctcgatttgccaatacatggtgatggcggcgccctgcgatcatacctttacgtcgatgatgttgccgaagcttatattacaattcttttaaagggcaatgttggagagacctataacatcggcacgcaaaaggagcgatccgttgtggacgtggcccatgacatctgcaagatttttaaccgagactcagatacagctatatggcacgttaaagaccgagccttcaatgatcgacgttattttatttctgataaaaaattacttgacctcggctggcaagaaaaaaccacctgggaggacggtcttaagcaaactgtagggtggtatttgcaacatgcaactaggtcctactgggatcacggcaacatggaattagctttagacgcccatccgacacttcaagttcctaaattctaa

SEQ ID NO: 40 - protein, 350 aa, Chlorovirus acanthocystis turfacea chlorella virus 1 [sequence omitted]

SEQ ID NO: 41 - DNA, 693 bp, synthetic construct
  misc_feature 1..693: note = Aggregatibacter actinomycetemcomitans NDP-4-keto-6-deoxy-glucose 4-ketoreductase (AaFCD) coding sequence. Codon-optimised for expression in N. benthamiana. The original sequence can be found as Genbank ID: AB002668.1 (sequence 15271..15963bp).
  source 1..693: mol_type = other DNA
atgataattggcaacggtatgctggcaaaagcctttgaatcattccataagcgaacttacaattacataatatttgcatccggagtgagcaactcaaacgaaacttccttcgagaatttcaacagagagaaggaattgcttcttgaagtcctggagcaatataaagacaaaactatcgtttactttagttcctgctccatatacgattctagtttgacgaattctttgtatgtctaccacaaaatgtgtatggagagactggtgcgtgaaaactccaagaattatctcatagcccgtctcccccaagttattggtaaaacgtattcaccaaccattgtcaacttcctttttaacaaaatcaaaaatagggagtgtttcagcatattcggaaaagctcaccgaaattttatcgacgtggatgatgtcgttaaggtcaccaattacttattgaaggagggtctgttcattaacagtattgtgaacttggcaagcacgcaccatacctccatgtacgaattaatcttatatttggaaaaaataagtaatcaacgtgccttctataatgttgagaacaaagggtctaggtactttattgatgtttcaatactgcaggatgtttatcagaagctggggatcaaatttgacaaagattacgtagaaaaggttatcaacaagtactacgctattaagtaa

SEQ ID NO: 42 - protein, 230 aa, Aggregatibacter actinomycetemcomitans
MIIGNGMLAKAFESFHKRTYNYIIFASGVSNSNETSFENFNREKELLLEVLEQYKDKTIVYFSSCSIYDSSLTNSLYVYHKMCMERLVRENSKNYLIARLPQVIGKTYSPTIVNFLFNKIKNRECFSIFGKAHRNFIDVDDVVKVTNYLLKEGLFINSIVNLASTHHTSMYELILYLEKISNQRAFYNVENKGSRYFIDVSILQDVYQKLGIKFDKDYVEKVINKYYAIK

SEQ ID NO: 43 - DNA, 927 bp, synthetic construct
  misc_feature 1..927: note = Anoxybacillus tepidamans NDP-4-keto-6-deoxy-glucose 4-ketoreductase (AtFCD) coding sequence. Codon-optimised for expression in N. benthamiana. The original sequence can be found as Genbank ID: AY883421.5 (sequence 13209-14135bp).
  source 1..927: mol_type = other DNA
atgaagaggatactgatactcggatgcggttacctgggtttaaatctcgcaaactatttttgtaaaaaaaattatgatgtctcagtgatagggagaaagtctgtctatagcaattttttggaagaggagatagagttcatagaagatgatatcaaaaatataaatagttataagcacatgtttaatgaggagacaaccgtcatttacgccataggaagtattaacgcaaataactattttatggacctgaggaatgatatagaaaactcatacatccccttcattaacctccttaactttctttccgaaaagtatattcaaaagttcgtctttctctcttcagccggaacagtctatgggaacgtgaataagaattatataagcgagaatgagattcttaacccaatttcaatctatggtttgcagaaagccttctttgaacaactgataaggattaaaaacaatgaggctagccatttcaggtatttgatcttcagaatatctaacccctatgggggaatcaacattccgaacaagaatcagggaattattccgacgttagtgtacaaagccgtgaacaatgagcctttcgaactttgggcatcaatcaataccatccgtgattatatttacatcgatgaccttagcgaattgatctacaaaacaatctatctggacatttataacgagaccctcaatctcgggtccggtaaaggaacatcaatcaagcaactcattagcctcgtggaggagattttgggaaagaagatcactattcttgaaaagccccccataaagactaacgttttgaaaaatatacttgatatttctaagctcgtcaacaccgtaggctacgaaccaaagatcagcattgaagagggtattagccgttacatcaacactattttaacgaagaacattttttaa

SEQ ID NO: 44 - protein, 308 aa, Anoxybacillus tepidamans
MKRILILGCGYLGLNLANYFCKKNYDVSVIGRKSVYSNFLEEEIEFIEDDIKNINSYKHMFNEETTVIYAIGSINANNYFMDLRNDIENSYIPFINLLNFLSEKYIQKFVFLSSAGTVYGNVNKNYISENEILNPISIYGLQKAFFEQLIRIKNNEASHFRYLIFRISNPYGGINIPNKNQGIIPTLVYKAVNNEPFELWASINTIRDYIYIDDLSELIYKTIYLDIYNETLNLGSGKGTSIKQLISLVEEILGKKITILEKPPIKTNVLKNILDISKLVNTVGYEPKISIEEGISRYINTILTKNIF

SEQ ID NO: 45 - DNA, 951 bp, synthetic construct
  misc_feature 1..951: note = Escherichia coli NDP-4-keto-6-deoxy-glucose 4-ketoreductase (EcFCD) coding sequence. Codon-optimised for expression in N. benthamiana. The original sequence can be found as Genbank ID: AY528413.1 (sequence 3156-4106bp).
  source 1..951: mol_type = other DNA
atggatgctcgtaaaaatggggtattaataaccggtggagctgggttcataggtaaagccttaataactgaaatggtcgaacgtcaaattcccctggtgtcatttgacatcagcgataagcccgacagtttgccagagctttccgaatatttcaactggtataaattctcataccttgagagttcacagaggattaaagagcttcacgaaatagtttccaggcataacatcaaaacggtcatccatttagctacaactatgtttccccacgaatccaaaaagaacatcgataaggattgcttagaaaacgtttatgccaacgtgtgtttctttaagaatttatatgaaaacggctgtgaaaaaattatcttcgcctcatcaggtggcaccgtatatgggaagtctgatacacccttctccgaagacgatgccctgcttcccgaaatttcctacggactgtccaaggttatgactgaaacttatctccgattcatagccaaggaattgaatgggaagtccatctctctcagaatatctaacccctatggtgaggggcaaaggattgacgggaaacaaggagtcattccaattttcctcaataaaatcagcaacgacatccccatcgacatcattggctctatcgaatcaaagcgagactacatttatatttcagatctcgtacaagctttcatgtgctctctggaatatgaaggtcacgaagacatatttaatataggttctggggaaagcataactctgaagaaattgatcgagacgattgagttcaagctgaacaagaaggctgtgattggatttcaagatccgatccacaccaatgccaatggtataattctcgacatcaaacgagccatggcagaactcggctggaggcccaccgtggtcctggatgatggcatcgataaattaatcaagagcattcgatgcaagtaa

SEQ ID NO: 46 - protein, 316 aa, Escherichia coli
MDARKNGVLITGGAGFIGKALITEMVERQIPLVSFDISDKPDSLPELSEYFNWYKFSYLESSQRIKELHEIVSRHNIKTVIHLATTMFPHESKKNIDKDCLENVYANVCFFKNLYENGCEKIIFASSGGTVYGKSDTPFSEDDALLPEISYGLSKVMTETYLRFIAKELNGKSISLRISNPYGEGQRIDGKQGVIPIFLNKISNDIPIDIIGSIESKRDYIYISDLVQAFMCSLEYEGHEDIFNIGSGESITLKKLIETIEFKLNKKAVIGFQDPIHTNANGIILDIKRAMAELGWRPTVVLDDGIDKLIKSIRCK

SEQ ID NO: 47 - DNA, 897 bp, Quillaja saponaria
  source 1..897: mol_type = other DNA
atggcagaagcaacagagaggtatgctgttgtgacaggatctaataaaggaattggatttgggatatgcaagcagctggcttctaaggggattacagtagtgctaacagctagagatgataagagaggtcttgaagcagttgagaaattgaaagaatttgatctgcatggtcatgtgctttttcatcaacttgatgtgtctgatacagctagtgttactagccttgcagattttatcaaaacccagtttgggaaactagatatcttggtaaacaatgcaggtataactggaaccactgtagatgctgatgctttagcatcttcaggctatggtactgggggtgaacgtaaacctattgattggaataaaatagtgatagagacttatgaatcagttgaaaaagctatcaacaccaactattatggagccaaaagaatggctgaagcacttataccccttcttcaagtatcagactcaccaaggattgttaatgcttcctctcctatggcaaagctagagaatattccaagtggatggggtaaggaagtgctaagtgatgttgatagcctaacagaagagaaacttgatgagatgttgacccaattattgaaagatttcaaagagggttcattagaaaccaaaggctggcctactcttatgtcttcgtatataatctcaaaagctgctttaaatgcctacacaaggattcttgctaagaagtacccatctttctgcatcaattgtgtagaccctggtcatgtgaagactgacataaatcgtcacaccggccacttaagtattgatgaaggtgctgaaagccatgtgagattggccctgctgcctgatggtggcccttctggacatttcttctccaggactgaagagacaccattttga

SEQ ID NO: 48 - protein, 298 aa, Quillaja saponaria
MAEATERYAVVTGSNKGIGFGICKQLASKGITVVLTARDDKRGLEAVEKLKEFDLHGHVLFHQLDVSDTASVTSLADFIKTQFGKLDILVNNAGITGTTVDADALASSGYGTGGERKPIDWNKIVIETYESVEKAINTNYYGAKRMAEALIPLLQVSDSPRIVNASSPMAKLENIPSGWGKEVLSDVDSLTEEKLDEMLTQLLKDFKEGSLETKGWPTLMSSYIISKAALNAYTRILAKKYPSFCINCVDPGHVKTDINRHTGHLSIDEGAESHVRLALLPDGGPSGHFFSRTEETPF

SEQ ID NO: 49 - DNA, 915 bp, Quillaja saponaria
  source 1..915: mol_type = other DNA
atgggttcagatggaagggatgtagcagagaggtatgcagtggttacaggtgcaaacaaaggcataggcctagaaaccgtgcggcaactagcgtctcacggcattacagttgtgttgacagctcgagatgagaagagagggactgaagccacaagaaagctccaccagctgggtttgtcaaatttgattttccatcagctggatgttttagaccctgttagcattcagtcactggccaagttcatccaagacaaatttggcaggcttgatatcctggttaataatgctggagcatctggacttgcagctgatgagaaagctctgaaggcattaaacatagataatgcagcttggctctcaggcaaggccgccaatttagttcaaggaattgtcacacatacctatgagcaaggcgaagaatgcataaatacaaactattatggtgtcaaaagggtgacggaagctctcctaccgctgttacaactttcccctataggagcaaggataataaatgtttcctcttgcaggggtgagctaaagaggattccaatgaacgtaagaaatgaactgggcgacatcaaagttctgactgaaggcagaatagatgcaattttgatgaaatttctacacgattttaaggataatgcacttgagtccaacggatggacattgatggggcctgcttatagcatttcgaaggccagtctcaatgcctacactagacttcttgccaaaaagtaccccgagatgctcattaactgtgttcatcctggttatgtcaacacagatatgacttggcatagagggatactgacggtagaagagggtgctaaaggcccagccatgctagctcttttgcaagatggaggacctacaggttgctattttgatagtactcaacaggcagaattttaa

SEQ ID NO: 50 - protein, 304 aa, Quillaja saponaria
MGSDGRDVAERYAVVTGANKGIGLETVRQLASHGITVVLTARDEKRGTEATRKLHQLGLSNLIFHQLDVLDPVSIQSLAKFIQDKFGRLDILVNNAGASGLAADEKALKALNIDNAAWLSGKAANLVQGIVTHTYEQGEECINTNYYGVKRVTEALLPLLQLSPIGARIINVSSCRGELKRIPMNVRNELGDIKVLTEGRIDAILMKFLHDFKDNALESNGWTLMGPAYSISKASLNAYTRLLAKKYPEMLINCVHPGYVNTDMTWHRGILTVEEGAKGPAMLALLQDGGPTGCYFDSTQQAEF

SEQ ID NO: 51 - DNA, 909 bp, Salvia officinalis
  source 1..909: mol_type = other DNA
atggctgaagcatcctcatttcttgcacagaaaaggtatgcggtcgtgacaggagcaaacaaaggactaggactagaaatatgcggacagcttgcttcacagggggtgacggtactgctgacatccagagatgaaaaacgaggcttagaagccattgaggagcttaagaaatcggggattaattcggaaaatcttgaatatcatcagctggatgttactaagccagctagtttcgcttctctggccgatttcatcaaggccaaatttggcaagcttgatatcctggtgaacaatgcagggatcagcggtgttattgtagattatgcagctttaatggaagccattcgccgtcgaggggcagagatcaattacgatggagtgatgaaacagacctacgagctagcagaggaatgcttgcaaacaaattactatggtgtgaaaagaaccattaatgctctccttccgctacttcagttttccgattcaccaaggatcgtcaatgtttcctccgatgttggcctccttaagaaaatacccggcgagagaatcagagaagccttaggcgacgtggaaaaacttacggaagaaagcgtggacgggattttagacgagtttctaagagatttcaaggaaggcaagatcgcagagaaaggttggcctacgtttaagagcgcctattcaatctcaaaggcggcgctcaattcgtacacgagggttttagcacggaaatacccgtcgatcatcatcaactgtgtctgcccgggtgtcgtcaaaaccgatatcaatcttaaaatgggccacttgacggttgaagaaggcgcggccagtcccgtgaggttagcactcatgccccttggttcgccttccggcctgttctatactcgaaacgaagtaactccatttgaatga

SEQ ID NO: 52 - protein, 302 aa, Salvia officinalis
MAEASSFLAQKRYAVVTGANKGLGLEICGQLASQGVTVLLTSRDEKRGLEAIEELKKSGINSENLEYHQLDVTKPASFASLADFIKAKFGKLDILVNNAGISGVIVDYAALMEAIRRRGAEINYDGVMKQTYELAEECLQTNYYGVKRTINALLPLLQFSDSPRIVNVSSDVGLLKKIPGERIREALGDVEKLTEESVDGILDEFLRDFKEGKIAEKGWPTFKSAYSISKAALNSYTRVLARKYPSIIINCVCPGVVKTDINLKMGHLTVEEGAASPVRLALMPLGSPSGLFYTRNEVTPFE

SEQ ID NO: 53 - DNA, 900 bp, unidentified
  misc_feature 1..900: note = A nucleic acid sequence which encodes the SpolFSL enzyme according to SEQ ID NO 54.
  source 1..900: mol_type = other DNA
atggctgaacaatccaactttctggctgaaaaaaggtatgcagtagtgacaggtgcaaacaaaggaatagggcttgaaatatgcagacagcttgcttctcaaggtgtgattgtacttatcacttctagagatggaaagaaaggattagaagcccttaatgatctcattaaatctggaattagctctgataatcttcattatcatcagcttgatgttactgaccctatgagtattactgctcttgctggtttcatcaattccaaatttggcaagcttgatattctggtgaacaatgctgggataggtggatttataattgactacgatgctatcaaagcaataggttttcgcaatatcaattatgacgagatgatgacacaaacatatgagcttgcaaaagaatgcttggaaacaaactactatggagttaagagaacaactgaagctttgcttcctcagctggagttatcggattcaccaaggatcatcaatgtctcctcttctacggggatgttgaagaatataccaaatgagaggatcagaggagtcttgggtgatgcagagaatcttacagaagaaaaagttgaagcgattttgaatgagttactgacagatttcaaagatggttcattcaaagagaaagaatggccttctagaatggcagcttatacactgtcaaaggcggctttgaatgcatatgcaagaatattggctaagaaatacccgtcaattatcatcagttgtgtttgtcctggtgttactaagacagatatgaacggaaacttgggacaattaacagttgaagaaggggccgcaagtccggtgagagtagcattgatgcctcatggttcaccttccggtcttttctatgcaagaagcgaagtttcttcatatgaataa

SEQ ID NO: 54 - protein, 299 aa, unidentified
  REGION 1..299: note = An oxidoreductase enzyme capable of enhancing the activity of a fucosyltransferase (SpolFSL)
MAEQSNFLAEKRYAVVTGANKGIGLEICRQLASQGVIVLITSRDGKKGLEALNDLIKSGISSDNLHYHQLDVTDPMSITALAGFINSKFGKLDILVNNAGIGGFIIDYDAIKAIGFRNINYDEMMTQTYELAKECLETNYYGVKRTTEALLPQLELSDSPRIINVSSSTGMLKNIPNERIRGVLGDAENLTEEKVEAILNELLTDFKDGSFKEKEWPSRMAAYTLSKAALNAYARILAKKYPSIIISCVCPGVTKTDMNGNLGQLTVEEGAASPVRVALMPHGSPSGLFYARSEVSSYE

SEQ ID NO: 55 - DNA, 1320 bp, Quillaja saponaria
  source 1..1320: mol_type = other DNA
atggcggatcgagtcataaacagctacaaaaagcttcacgtagtgctgtttccatggctggcctttggtcacatgataccatttttagagcttgcaaagctgatagcccaaaaaggtcacaaaatttcctatatctcaacacccagaaacatccgacgcctacctaaaattccatcccatttatccaacaaccttaatttcatagaattcccactgcctcatatacccaatcttcatgaaaatgtggaatcaacaaacgacgtcacacatgacaatcctattgcctattatttacttattaaagcccttgagggtttgcaacaacctatcaccaccttccttgaaacctcagatccagattggataattcatgacgtttttcctcaatggataaccgcaacagccagtaggctccgcatttcacatgctttttacacaacttcatctgctttgagaacggcttccaattattctccgttaatgtcctctgaattaccacaggatattgctaccgatttcactactaaatccacaaatcttcttgctgccaaggttttggttatccgtagctgtctagagcttgaacccaaggaatttgaacagtgcaaaaatctatgcgtgtccaaaacggtaattccattgggcgttgtcccaccgtcaattcaggtgaacgataacatttctaatattaatgatgacgacaacgactgggttaagattgttgagtggttaaaccaagggaaagagaaaggttctgtcatttatgtagcacttggttctgaggtttcactgagtgaacaagatttgaaggagtttgcacttggtttggaactatcagggttgtcattcttttgggtgttcaggaatactggatcgtatgggttaccggctgggtttgaagaccgagttaagggtcggggaattgtctggactagctgggctccgcaggtgagtatattaggacacgagtcaattggtggattcttgagtcatgggggttggagttctgtaatagaaagtctaagttttgggattccacttgttgtttttccatttggagctgatcaagggattaatgcaaagcaattggagggtaaaaatgcaggggtggaaatacccagatcagaaggtactgggtcatttacaaggaagtctgtggctgatttgttgaggctggtggtggtggaggaggagggaaaggtttacagggatggtgccaaggaattgaggaaactatttggggacaaggatttgaaccacaaatacattgacaactttgttaagtacatggaagaacatatcacgaatgcagctaattga

SEQ ID NO: 56 - protein, 439 aa, Quillaja saponaria
MADRVINSYKKLHVVLFPWLAFGHMIPFLELAKLIAQKGHKISYISTPRNIRRLPKIPSHLSNNLNFIEFPLPHIPNLHENVESTNDVTHDNPIAYYLLIKALEGLQQPITTFLETSDPDWIIHDVFPQWITATASRLRISHAFYTTSSALRTASNYSPLMSSELPQDIATDFTTKSTNLLAAKVLVIRSCLELEPKEFEQCKNLCVSKTVIPLGVVPPSIQVNDNISNINDDDNDWVKIVEWLNQGKEKGSVIYVALGSEVSLSEQDLKEFALGLELSGLSFFWVFRNTGSYGLPAGFEDRVKGRGIVWTSWAPQVSILGHESIGGFLSHGGWSSVIESLSFGIPLVVFPFGADQGINAKQLEGKNAGVEIPRSEGTGSFTRKSVADLLRLVVVEEEGKVYRDGAKELRKLFGDKDLNHKYIDNFVKYMEEHITNAAN

SEQ ID NO: 57 - DNA, 1452 bp, Quillaja saponaria
  source 1..1452: mol_type = other DNA
atgaccagtaataatagccaactccacatcttcttccttcctgcactgtctcctggccacatgatacctgttattgaaatggccaaactagttgcttcccgaggtgtcatggcaactatagtcaccactgctcacaatcttgcttttgtttccagaaccatatctacctacagtactaaaataaaaatcgtaaccatcaaattcccttatgcagaggttggcttacctgaaggatgtgaaatcattgactcagatactcccccagatatactatttcgtgtcatcaaagccctgagattgctacaagaaccaatggagcaactattgtcttcttatcaaccagactgccttgtagctgatgcattcttcagttgggcaacaaattctgctgctaaatttaacattccaaggcttgtgttccatgtggcatgtctattctctctgtgtgcatctcattctattgaattatatgagcctcagaagaaggtgtcttctgattcagagacttttattattcctagtcttcctggtgaaataaaattgacaaagatgcagctacctgctgatttacctaaaactggtgtggaggctgagtacatcaacaaaatggtgaaagctgtccatgaatcagtggagaacagctatggttttataattaacagcttctatgagcttgagaaggattatgtagattattatagaaacgttattggaaggaaagcgtggcatattggcccactgtctctatgccatgcggacaatattgaagaaaaatcacagcgaggaaaagaaagctccattgctgagaatgaatgcttgaagtggcttgactcgaggaagccagattcggttgtttatgttggttttggaagtctggtaaatttcagtgattcccagctgatggagatagcattgggtcttgaggcttctgagaaacaatttatttgggttgtcaagaaaagcaaaagaaatgaacaagaaaaagaagaatggctacctgaagggtttgagaaaagaacggtaggtaagggactgattataagaggttgggcaccccaattgttaattatggatcatgaagctgttggagggtttgtgactcattgtggatggaactcaaccttggaaggtgtgtgtggtggggtagttatggccacttggccagtatcttatgagcaaatttatactgaaaagctggtgactgatgttctaaaaattggggtttctgtaggcgctcaaacatgtgatggaattgttggaggtattattaaaagtgaagcaatagagaaggcagtgaatagaataatggaggggattgaagcagaggagatgagaagcagagcaaaggcctttgcaaaaaaggcaaggcagtctgttaaagagggaggatcctcttactcagatttgaactctttaattgaggagttgagtcttaaatctcttaagcattaa

SEQ ID NO: 58 - protein, 483 aa, Quillaja saponaria
MTSNNSQLHIFFLPALSPGHMIPVIEMAKLVASRGVMATIVTTAHNLAFVSRTISTYSTKIKIVTIKFPYAEVGLPEGCEIIDSDTPPDILFRVIKALRLLQEPMEQLLSSYQPDCLVADAFFSWATNSAAKFNIPRLVFHVACLFSLCASHSIELYEPQKKVSSDSETFIIPSLPGEIKLTKMQLPADLPKTGVEAEYINKMVKAVHESVENSYGFIINSFYELEKDYVDYYRNVIGRKAWHIGPLSLCHADNIEEKSQRGKESSIAENECLKWLDSRKPDSVVYVGFGSLVNFSDSQLMEIALGLEASEKQFIWVVKKSKRNEQEKEEWLPEGFEKRTVGKGLIIRGWAPQLLIMDHEAVGGFVTHCGWNSTLEGVCGGVVMATWPVSYEQIYTEKLVTDVLKIGVSVGAQTCDGIVGGIIKSEAIEKAVNRIMEGIEAEEMRSRAKAFAKKARQSVKEGGSSYSDLNSLIEELSLKSLKH

SEQ ID NO: 59 - DNA, 1476 bp, Quillaja saponaria
  source 1..1476: mol_type = other DNA
atgaagatagaaaccatttccacaaattgcatcaaaccctccaagcccactccttcccatctcagaaatattaagctctctgatcaacaccaacattccccagatgtccattccaacttcaccttcttctaccagtctaatcaaatagatgatgctgttgttactgttcccagtgctgctattgatgttgctcctgctactgatgccgctattgatgttgctgctgctactgatactgctattgatggtgctgctacaaatttctcagttcaatccaaaattcttcataattgcttggcaacaacactcacaagcttctaccctatcgcgggtcggtttcaaaatggcgacactatcatttgcaacgatgaaggtgccttctttatcgaagcaaaaactgacataaacatgtccaatttccttgggcacccagacttgcttacagttatacgtgaacacttagtacccgatgctactaacccggattataatggttctatccttcttctcaagtttacattgtttggctgtggcagtactgccataaccatctcaatgtcgcacaagatagccgatttagttacctttataacactccttaattgctggacagctttagctcgtgctggtggtggtggtggtataggtggtggttctgatggttttattccacctgatttgaatttccttgggcagattgtcccggattcggatccctcaccaaaatcagcaactcctgaattttttcgaaacaagaagtttgttacgaaaagatttgttttcagtgcatccaagatcaaagaagttaaggacaaagttatgaaggagattaggaaacaagaggatgatatttttccttctcgtgtggatgtggtacttgcattaatttggagaagtacattgtcaagtttgtctggatcatctggtaagtttaaaccggcaatattcatgcaagctgcaaatctcagaacgcgtactgatccaccattaccggaaacttctatcgggaacttggtgatactatttcctttggtggttgagaaagagactgatatagaattacatgaattggttaacaagttgttagatgccaaagcatgggttaacaaattaaagaagaaattccaaggttatgatggtggtaatgatcctctacaagttgtggaagcaataagatgtgaggctttgaaagaaatgggtaatgtttggaagaaatccaaggatttttcgatgtatatttcatcgagtttttgtaattttctgatgaatgaggttgattttgggtggggaaagccagtttgggtaaccaacacacctagaacaatcatggctaataccatatatttgttggatactaaagaggtgggcggagttgatgctttggtacaatttgaagaggaagaaatcaccaaattggaacttaatcaagagctactccaatttgctactgttaatcccattcctattgttatttaa

SEQ ID NO: 60 - protein, 491 aa, Quillaja saponaria
MKIETISTNCIKPSKPTPSHLRNIKLSDQHQHSPDVHSNFTFFYQSNQIDDAVVTVPSAAIDVAPATDAAIDVAAATDTAIDGAATNFSVQSKILHNCLATTLTSFYPIAGRFQNGDTIICNDEGAFFIEAKTDINMSNFLGHPDLLTVIREHLVPDATNPDYNGSILLLKFTLFGCGSTAITISMSHKIADLVTFITLLNCWTALARAGGGGGIGGGSDGFIPPDLNFLGQIVPDSDPSPKSATPEFFRNKKFVTKRFVFSASKIKEVKDKVMKEIRKQEDDIFPSRVDVVLALIWRSTLSSLSGSSGKFKPAIFMQAANLRTRTDPPLPETSIGNLVILFPLVVEKETDIELHELVNKLLDAKAWVNKLKKKFQGYDGGNDPLQVVEAIRCEALKEMGNVWKKSKDFSMYISSSFCNFLMNEVDFGWGKPVWVTNTPRTIMANTIYLLDTKEVGGVDALVQFEEEEITKLELNQELLQFATVNPIPIVI

SEQ ID NO: 61 - DNA, 1539 bp, unidentified
  misc_feature 1..1539: note = A nucleic acid sequence which encodes the enzyme according to SEQ ID NO 62.
  source 1..1539: mol_type = other DNA
atgggggaagtcaaccatgaagaagtagaaattgaaataatatcaatagaaaccataaaaccatcatcactacttccaccaaaaactcctccaaaaaccatcacactttctcacctcgatcaagctgcccctttgtactactatcctttacttttatactacactaacactactactactaccccaacatcacaaattcgagttgacataacaagtaccctaaaaacttcacttagcaaaacacttgacaaattccaccctattgcaggtcgatgtgtggacgactctacaatttgttgcaaccaccaaggaataccattcattgaaaccaaagttgactccaatatcttggatgtcatgaactcgcctgagaaaatgaagttgcttatcaagtttctccctcatgcagagtttcaagatgtgactcgaccagtctcggatttaaaccatttggcgtttcaagtcaatgttttccggtgtggtggggtgatcattggctcctatgtgctccacaagctccttgatggaatctctcttggaactttctttaaaaattggtcaaccattgctaatgatgagcgagttaaggacgacgacctagtacaacctgactttgaagccactattaaggcgttccctccgcgtacagcaactccaatgcttcctcgtaatcaacaacttccaaaggcggctgaaaaaccaaataataatccagtcaaagttcttgtgacaaagagcttcgtatttgacattgtttctttaaagaagatgatgttcatggctaagagtgaattggttcctaaacccaccaaatttgagaccgtgacagggtttatttgggaacaaaccttatcaacattgcgtaattctggagttgaagttgaacatacatcgcttataatacctgtaaacatccgcccaaggatgagtccgccactcccaagaggatccatgggtaacttgctcaagaatgcaaaggcacaggccaacaccagcagcagcaatgggcttcaagaccttgttaaagaaatccattcatctttgtctcaaacaacccagaaaattaatactcctcctcctcctcctcctcctcctactactactgctacaacaatccattcatctttgtctcaaacaacccagaaaattaatactcctcctcctactactacaacaatccattcatctttgtctcaaacaacccagaaaattaatactactactactactacagcagaggttattttgactaaacggaaagttgacaatccagttacacagaatcgagaaggaaactacctcttcaccagttggtgcaagattgggttggatgaggctgacttcgggttcggaaagcccgtttgggtaattcccaacgatgggagaccccctaaggtcaggaatatgattttccttactgattataggcatcccgaaacaggcgttgaaggaattgcagcatggattacgttggaagagaaacaaatgcaatgtttaaagtcaaacccagaattccttgcttttgctactcctaattag

SEQ ID NO: 62 - protein, 512 aa, unidentified
  REGION 1..512: note = An acetyltransferase enzyme capable of transferring an acetyl to the C-4 position of the D-Fucose residue on the QA-Tri(X/A)-F* scaffold (SOAP10)
MGEVNHEEVEIEIISIETIKPSSLLPPKTPPKTITLSHLDQAAPLYYYPLLLYYTNTTTTTPTSQIRVDITSTLKTSLSKTLDKFHPIAGRCVDDSTICCNHQGIPFIETKVDSNILDVMNSPEKMKLLIKFLPHAEFQDVTRPVSDLNHLAFQVNVFRCGGVIIGSYVLHKLLDGISLGTFFKNWSTIANDERVKDDDLVQPDFEATIKAFPPRTATPMLPRNQQLPKAAEKPNNNPVKVLVTKSFVFDIVSLKKMMFMAKSELVPKPTKFETVTGFIWEQTLSTLRNSGVEVEHTSLIIPVNIRPRMSPPLPRGSMGNLLKNAKAQANTSSSNGLQDLVKEIHSSLSQTTQKINTPPPPPPPPTTTATTIHSSLSQTTQKINTPPPTTTTIHSSLSQTTQKINTTTTTTAEVILTKRKVDNPVTQNREGNYLFTSWCKIGLDEADFGFGKPVWVIPNDGRPPKVRNMIFLTDYRHPETGVEGIAAWITLEEKQMQCLKSNPEFLAFATPN

SEQ ID NO: 63 - DNA, 1401 bp, Quillaja Saponaria
  source 1..1401: mol_type = other DNA
atgatggaggtacataccacatcggaaaattgcattaagccctcacaacccactccttcccaccttcaaaatttgaaactctctaatcatcatagccaagcacccgatatccgtaccaacctcaccttcttcttctcctctaattttaacaatccagtccagcctggtgaccatgacgctactaccaattttacactccaatccaaacttgttcagaattcattggctacaactctcacaattctctacccttttgctggtcgattccgaaacgacgacaccatcatttgcaaagacgatggcgccttctttattgaagcaaaaaccgacaccaaactttctgactttcttgcccagccggacttgcctctagctataatggacaaattagtccccgtagctaccgacgccaagtataatggttctctgctaatcctgaaatttactttgtttggctgcggtggctcagccgtaaccatctcaataactcacaagatttccgatctagcaaccattttgacactgcttaattgctggactgctttatctcgtggtggtgatggtggtggttccagtccgtttattcagcctgatttgaatttcattggccgccctgttccaagtacgagtgaggttcctcctccatcttctggaaagaattttattcctccaaatagcaagtacgttaccaaaaggttcatattcagtgcagcaaagattaaagaactgaaagccagagtcatcaacaagattcgtaaagaagaagacaatgttttcccttcccgtgtggatgtagtgctggcactcatttggaaatgcgctttggcttctgttaattctggttccagatctggaaatgcacaaacatttaggccgtcggtaatgatgcaagccgtgaacctcagaaaccgtacagatccaccattaccggaatcttcgattgggaacttggcaatactattaccggtgtgggtggagaaagaggaagacacggaattacatgaacttgttagcagattgttaacagtgaaagtgcgtgctaacagattgaagaagaaataccaaggttatgaagatccagagcaagttattatttcaatggaatctgattcagtgaaggaaataatagaggttcggaagaaattgaaggatttcagtacttatgttgcggcgagtgtggtgaatgctccattgtatgacgttgattttgggtggggaaaaccagcatgggtgaccagcacgcccaacacagtaatggcgaattctatatatttgttggataccaaagacgccggtgggattgaagttttgatgaatatgttgaaagaggaagacatgattgtctttgaaagcaatcaggagttgcttcagtctgccatggttaatcccactatcatttga

SEQ ID NO: 64 - protein, 466 aa, Quillaja Saponaria
MMEVHTTSENCIKPSQPTPSHLQNLKLSNHHSQAPDIRTNLTFFFSSNFNNPVQPGDHDATTNFTLQSKLVQNSLATTLTILYPFAGRFRNDDTIICKDDGAFFIEAKTDTKLSDFLAQPDLPLAIMDKLVPVATDAKYNGSLLILKFTLFGCGGSAVTISITHKISDLATILTLLNCWTALSRGGDGGGSSPFIQPDLNFIGRPVPSTSEVPPPSSGKNFIPPNSKYVTKRFIFSAAKIKELKARVINKIRKEEDNVFPSRVDVVLALIWKCALASVNSGSRSGNAQTFRPSVMMQAVNLRNRTDPPLPESSIGNLAILLPVWVEKEEDTELHELVSRLLTVKVRANRLKKKYQGYEDPEQVIISMESDSVKEIIEVRKKLKDFSTYVAASVVNAPLYDVDFGWGKPAWVTSTPNTVMANSIYLLDTKDAGGIEVLMNMLKEEDMIVFESNQELLQSAMVNPTII

SEQ ID NO: 65 - DNA, 54 bp, synthetic construct
  misc_feature 1..54: note = PCR Primer - UGT-BI_attB1F
  source 1..54: mol_type = other DNA
ggggacaagtttgtacaaaaaagcaggcttaatggcggatcgagtcataaacag

SEQ ID NO: 66 - DNA, 55 bp, synthetic construct
  misc_feature 1..55: note = PCR Primer - UGT-BI_attB2R
  source 1..55: mol_type = other DNA
ggggaccactttgtacaagaaagctgggtatcaattagctgcattcgtgatatgc

SEQ ID NO: 67 - DNA, 56 bp, synthetic construct
  misc_feature 1..56: note = PCR Primer - UGT-0023500_attB1F
  source 1..56: mol_type = other DNA
ggggacaagtttgtacaaaaaagcaggcttaatgaccagtaataatagccaactcc

SEQ ID NO: 68 - DNA, 60 bp, synthetic construct
  misc_feature 1..60: note = PCR Primer - UGT-0023500_attB2R
  source 1..60: mol_type = other DNA
ggggaccactttgtacaagaaagctgggtattaatgcttaagagatttaagactcaactc

SEQ ID NO: 69 - DNA, 61 bp, synthetic construct
  misc_feature 1..61: note = PCR Primer - Qs-ACT-19'_attB1F
  source 1..61: mol_type = other DNA
ggggacaagtttgtacaaaaaagcaggcttaatgaagatagaaaccatttccacaaattgc

SEQ ID NO: 70 - DNA, 69 bp, synthetic construct
  misc_feature 1..69: note = PCR Primer - Qs-ACT-19'_attB2R
  source 1..69: mol_type = other DNA
ggggaccactttgtacaagaaagctgggtattaaataacaataggaatgggattaacagtagcaaattg